SEQUENCE LISTING (435 sequences)

SEQ ID NO: 1 - protein, 879 aa, Artificial Sequence
  Synthetic
MGRLASRPLLLALLSLALCRGRVVRVPTATLVRVVGTELVIPCNVSDYDGPSEQNFDWSFSSLGSSFVELASTWEVGFPAQLYQERLQRGEILLRRTANDAVELHIKNVQPSDQGHYKCSTPSTDATVQGNYEDTVQVKVLADSLHVGPSARPPPSLSLREGEPFELRCTAASASPLHTHLALLWEVHRGPARRSVLALTHEGRFHPGLGYEQRYHSGDVRLDTVGSDAYRLSVSRALSADQGSYRCIVSEWIAEQGNWQEIQEKAVEVATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 2-8 - skipped (7 entries)

SEQ ID NO: 9 - protein, 385 aa, Artificial Sequence
  Synthetic
MAAALFVLLGFALLGTHGASGAAGFVQAPLSQQRWVGGSVELHCEAVGSPVPEIQWWFEGQGPNDTCSQLWDGARLDRVHIHATYHQHAASTISIDTLVEEDTGTYECRASNDPDRNHLTRAPRVKWVRAQAVVLVLEPGTVFTTVEDLGSKILLTCSLNDSATEVTGHRWLKGGVVLKEDALPGQKTEFKVDSDDQWGEYSCVFLPEPMGTANIQLHGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSHLAALWPFLGIVAEVLVLVTIIFIYEKRRKPEDVLDDDDAGSAPLKSSGQHQNDKGKNVRQRNSS

SEQ ID NO: 10-13 - skipped (4 entries)

SEQ ID NO: 14 - protein, 613 aa, Artificial Sequence
  Synthetic
MGALRPTLLPPSLPLLLLLMLGMGCWAREVLVPEGPLYRVAGTAVSISCNVTGYEGPAQQNFEWFLYRPEAPDTALGIVSTKDTQFSYAVFKSRVVAGEVQVQRLQGDAVVLKIARLQAQDAGIYECHTPSTDTRYLGSYSGKVELRVLPDVLQVSAAPPGPRGRQAPTSPPRMTVHEGQELALGCLARTSTQKHTHLAVSFGRSVPEAPVGRSTLQEVVGIRSDLAVEAGAPYAERLAAGELRLGKEGTDRYRMVVGGAQAGDAGTYHCTAAEWIQDPDGSWAQIAEKRAVLAHVDVQTLSSQLAVTVGPGERRIGPGEPLELLCNVSGALPPAGRHAAYSVGWEMAPAGAPGPGRLVAQLDTEGVGSLGPGYEGRHIAMEKVASRTYRLRLEAARPGDAGTYRCLAKAYVRGSGTRLREAASARSRPLPVHVREEGVVLEAVAWLAGGTVYRGETASLLCNISVRGGPPGLRLAASWWVERPEDGELSSVPAQLVGGVGQDGVAELGVRPGGGPVSVELVGPRSHRLRLHSLGPEDEGVYHCAPSAWVQHADYSWYQAGSARSGPVTVYPYMHALDTLFVPLLVGTGVALVTGATVLGTITCCFMKRLRKR

SEQ ID NO: 15-16 - skipped (2 entries)

SEQ ID NO: 17 - protein, 7 aa, Artificial Sequence
  Synthetic
GGKLSKK

SEQ ID NO: 18-20 - skipped (3 entries)

SEQ ID NO: 21 - protein, 748 aa, Artificial Sequence
  Synthetic
MNLQPIFWIGLISSVCCVFAQTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECECQSEGIPESPKCHEGNGTFECGACRCNEGRVGRHCECSTDEVNSEDMDAYCRKENSSEICSNNGECVCGQCVCRKRDNTNEIYSGASNGQICNGRGICECGVCKCTDPKFQGQTCEMCQTCLGVCAEHKECVQCRAFNKGEKKDTCTQECSYFNITKVESRDKLPQPVQPDPVSHCKEKDVDDCWFYFTYSVNGNNEVMVHVVENPECPTGPDIIPIVAGVVAGIVLIGLALLLIWKLLMIIHDRREFAKFEKEKMNAKWDTGENPIYKSAVTTVVNPKYEGK

SEQ ID NO: 22 - protein, 1032 aa, Artificial Sequence [sequence omitted]

SEQ ID NO: 23 - protein, 630 aa, Artificial Sequence
  Synthetic
MELQPPEASIAVVSIPRQLPGSHSEAGVQGLSAGDDSELGSHCVAQTGLELLASGDPLPSASQNAEMIETGSDCVTQAGLQLLASSDPPALASKNAEVTGTMSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAA

SEQ ID NO: 24-32 - skipped (9 entries)

SEQ ID NO: 33 - protein, 192 aa, Artificial Sequence
  Synthetic
GPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEM

SEQ ID NO: 34-46 - skipped (13 entries)

SEQ ID NO: 47 - protein, 332 aa, Artificial Sequence
  Synthetic
MGAQFSKTAAKGEAAAERPGEAAVASSPSKANGQENGHVKVNGDASPAAAESGAKEELQANGSAPAADKEEPAAAGSGAASPSAAEKGEPAAAAAPEAGASPVEKEAPAEGEAAEPGSPTAAEGEAASAASSTSSPKAEDGATPSPSNETPKKKKKRFSFKKSFKLSGFSFKKNKKEAGEGGEAEAPAAEGGKDEAAGGAAAAAAEAGAASGEQAAAPGEEAAAGEEGAAGGDPQEAKPQEAAVAPEKPPASDETKAAEEPSKVEEKKAEEAGASAAACEAPSAAGPGAPPEQEAAPAEEPAAAAASSACAAPSQEAQPECSPEAPPAEAAE

SEQ ID NO: 48 - protein, 195 aa, Artificial Sequence
  Synthetic
MGSQSSKAPRGDVTAEEAAGASPAKANGQENGHVKSNGDLSPKGEGESPPVNGTDEAAGATGDAIEPAPPSQGAEAKGEVPPKETPKKKKKFSFKKPFKLSGLSFKRNRKEGGGDSSASSPTEEEQEQGEIGACSDEGTAQEGKAAATPESQEPQAKGAEASAASEEEAGPQATEPSTPSGPESGPTPASAEQNE

SEQ ID NO: 49 - protein, 227 aa, Artificial Sequence
  Synthetic
MGGKLSKKKKGYNVNDEKAKEKDKKAEGAATEEEGTPKESEPQAAAEPAEAKEGKEKPDQDAEGKAEEKEGEKDAAAAKEEAPKAEPEKTEGAAEAKAEPPKAPEQEQAAPGPAAGGEAPKAAEAAAAPAESAAPAAGEEPSKEEGEPKKTEAPAAPAAQETKSDGAPASDSKPGSSEAAPSSKETPAATEAPSSTPKAQGPAASAEEPKPVEAPAANSDQTVTVKE

SEQ ID NO: 50-204 - skipped (155 entries)

SEQ ID NO: 205 - protein, 4 aa, Artificial Sequence
  Synthetic
KKKK

SEQ ID NO: 206 - protein, 5 aa, Artificial Sequence
  Synthetic
KKKKK

SEQ ID NO: 207 - protein, 4 aa, Artificial Sequence
  Synthetic
RRRR

SEQ ID NO: 208 - protein, 5 aa, Artificial Sequence
  Synthetic
RRRRR

SEQ ID NO: 209 - protein, 4 aa, Artificial Sequence
  Synthetic
  misc_feature (1)..(4): wherein Xaa can be Lysine or Arginine
XXXX

SEQ ID NO: 210 - protein, 5 aa, Artificial Sequence
  Synthetic
  misc_feature (1)..(5): wherein Xaa can be Lysine or Arginine
XXXXX

SEQ ID NO: 211 - protein, 7 aa, Artificial Sequence
  Synthetic
GGKLSKK

SEQ ID NO: 212 - protein, 7 aa, Artificial Sequence
  Synthetic
GAKLSKK

SEQ ID NO: 213 - protein, 7 aa, Artificial Sequence
  Synthetic
GGKQSKK

SEQ ID NO: 214 - protein, 7 aa, Artificial Sequence
  Synthetic
GGKLAKK

SEQ ID NO: 215-245 - skipped (31 entries)

SEQ ID NO: 246 - protein, 14 aa, Artificial Sequence
  Synthetic
GGKLSKKKKGYNVN

SEQ ID NO: 247 - protein, 14 aa, Artificial Sequence
  Synthetic
GAKLSKKKKGYNVN

SEQ ID NO: 248 - protein, 14 aa, Artificial Sequence
  Synthetic
GGKQSKKKKGYNVN

SEQ ID NO: 249 - protein, 14 aa, Artificial Sequence
  Synthetic
GGKLAKKKKGYNVN

SEQ ID NO: 250 - protein, 14 aa, Artificial Sequence
  Synthetic
GGKLSKKKKGYSGG

SEQ ID NO: 251 - protein, 14 aa, Artificial Sequence
  Synthetic
GGKLSKKKKGSGGS

SEQ ID NO: 252 - protein, 14 aa, Artificial Sequence
  Synthetic
GGKLSKKKKSGGSG

SEQ ID NO: 253 - protein, 14 aa, Artificial Sequence
  Synthetic
GGKLSKKKSGGSGG

SEQ ID NO: 254 - protein, 14 aa, Artificial Sequence
  Synthetic
GGKLSKKSGGSGGS

SEQ ID NO: 255 - protein, 14 aa, Artificial Sequence
  Synthetic
GGKLSKSGGSGGSV

SEQ ID NO: 256 - protein, 14 aa, Artificial Sequence
  Synthetic
GAKKSKKRFSFKKS

SEQ ID NO: 257-262 - skipped (6 entries)

SEQ ID NO: 263 - protein, 653 aa, Artificial Sequence
  Synthetic
MADTLPSEFDVIVIGTGLPESIIAAACSRSGRRVLHVDSRSYYGGNWASFSFSGLLSWLKEYQENSDIVSDSPVWQDQILENEEAIALSRKDKTIQHVEVFCYASQDLHEDVEEAGALQKNHALVTSANSTEAADSAFLPTEDESLSTMSCEMLTEQTPSSDPENALEVNGAEVTGEKENHCDDKTCVPSTSAEDMSENVPIAEDTTEQPKKNRITYSQIIKEGRRFNIDLVSKLLYSRGLLIDLLIKSNVSRYAEFKNITRILAFREGRVEQVPCSRADVFNSKQLTMVEKRMLMKFLTFCMEYEKYPDEYKGYEEITFYEYLKTQKLTPNLQYIVMHSIAMTSETASSTIDGLKATKNFLHCLGRYGNTPFLFPLYGQGELPQCFCRMCAVFGGIYCLRHSVQCLVVDKESRKCKAIIDQFGQRIISEHFLVEDSYFPENMCSRVQYRQISRAVLITDRSVLKTDSDQQISILTVPAEEPGTFAVRVIELCSSTMTCMKGTYLVHLTCTSSKTAREDLESVVQKLFVPYTEMEIENEQVEKPRILWALYFNMRDSSDISRSCYNDLPSNVYVCSGPDCGLGNDNAVKQAETLFQEICPNEDFCPPPPNPEDIILDGDSLQPEASESSAIPEANSETFKESTNLGNLEESSE

SEQ ID NO: 264-381 - skipped (118 entries)

SEQ ID NO: 382 - protein, 21 aa, Artificial Sequence
  Synthetic
GNYTCEVTELTREGETIIELK

SEQ ID NO: 383-399 - skipped (17 entries)

SEQ ID NO: 400 - protein, 323 aa, Artificial Sequence
  Synthetic
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 401 - protein, 292 aa, Artificial Sequence
  Synthetic
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFV

SEQ ID NO: 402 - protein, 305 aa, Artificial Sequence
  Synthetic
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 403 - protein, 311 aa, Artificial Sequence
  Synthetic
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLN

SEQ ID NO: 404 - protein, 119 aa, Artificial Sequence
  Synthetic
HSDPARRGELSVCDSISEWVTAADKKTAVDMSGGTVTVLEKVPVSKGQLKQYFYETKCNPMGYTKEGCRGIDKRHWNSQCRTTQSYVRALTMDSKKRIGWRFIRIDTSCVCTLTIKRGR

SEQ ID NO: 405 - protein, 120 aa, Artificial Sequence
  Synthetic
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 406 - protein, 119 aa, Artificial Sequence [sequence omitted]

SEQ ID NO: 407 - protein, 130 aa, Artificial Sequence
  Synthetic
GVSETAPASRRGELAVCDAVSGWVTDRRTAVDLRGREVEVLGEVPAAGGSPLRQYFFETRCKADNAEEGGPGAGGGGCRGVDRRHWVSECKAKQSYVRALTADAQGRVGWRWIRIDTACVCTLLSRTGRA

SEQ ID NO: 408 - protein, 29 aa, Artificial Sequence
  Synthetic
YTIWMPENPRPGTPCDIFTNSRGKRASNG

SEQ ID NO: 409 - protein, 14 aa, Artificial Sequence
  Synthetic
SACQSQSQMRCGGG

SEQ ID NO: 410 - protein, 7 aa, Artificial Sequence
  Synthetic
QSQSQMR

SEQ ID NO: 411 - protein, 7 aa, Artificial Sequence
  Synthetic
ASGAQAR

SEQ ID NO: 412 - protein, 14 aa, Artificial Sequence
  Synthetic
TSTAPHLRLRLTSR

SEQ ID NO: 413 - protein, 7 aa, Artificial Sequence
  Synthetic
PPKKRKV

SEQ ID NO: 414 - protein, 6 aa, Artificial Sequence
  Synthetic
PKKRKV

SEQ ID NO: 415 - skipped

SEQ ID NO: 416 - protein, 39 aa, Artificial Sequence
  Synthetic
SSVIDALQYKLEGTTRLTRKRGLKLATALSLSNKFVEGS

SEQ ID NO: 417 - protein, 18 aa, Artificial Sequence
  Synthetic
LRKLRKRLLLRKLRKRLL

SEQ ID NO: 418-421 - skipped (4 entries)

SEQ ID NO: 422 - protein, 30 aa, Artificial Sequence
  Synthetic
YQQILTSMPSRNVIQISNDLENLRDLLHVL

SEQ ID NO: 423-427 - skipped (5 entries)

SEQ ID NO: 428 - protein, 13 aa, Artificial Sequence
  Synthetic
HLNILSTLWKYRC

SEQ ID NO: 429 - skipped

SEQ ID NO: 430 - protein, 12 aa, Artificial Sequence
  Synthetic
TGNYKALHPHNG

SEQ ID NO: 431 - skipped

SEQ ID NO: 432 - protein, 18 aa, Artificial Sequence
  Synthetic
RGGRLSYSRRRFSTSTGR

SEQ ID NO: 433-434 - skipped (2 entries)

SEQ ID NO: 435 - protein, 29 aa, Artificial Sequence
  Synthetic
GGSSGSGSGSGGGGSGGGGTGTSSSGTGT

(19) United States
(12) Patent Application Publication
(10) Pub. No.: US 2023/0270674 A1
(43) Pub. Date: Aug. 31, 2023

(54) TARGETED DELIVERY OF EXTRACELLULAR VESICLES

(71) Applicant: Codiak BioSciences, Inc., Cambridge, MA (US)

(72) Inventors: Ajay VERMA, Wayland, MA (US); Wei ZHANG, Cambridge, MA (US); Ke XU, Belmont, MA (US); Shailendra PATEL, Cambridge, MA (US)

(73) Assignee: Codiak BioSciences, Inc., Cambridge, MA (US)

(21) Appl. No.: 17/906,173

(22) PCT Filed: Mar. 15, 2021
(86) PCT No.: PCT/US2021/022435

§ 371 Date: Sep. 12, 2022

Related U.S. Application Data
(60) Provisional application No. 63/154,563, filed on Feb. 26, 2021.
(60) Provisional application No. 63/154,562, filed on Feb. 26, 2021.
(60) Provisional application No. 62/704,998, filed on Jun. 5, 2020.
(60) Provisional application No. 63/035,393, filed on Jun. 5, 2020.
(60) Provisional application No. 62/989,535, filed on Mar. 13, 2020.
(60) Provisional application No. 62/989,524, filed on Mar. 13, 2020.

(51) Int. Cl. A61K 9/127; A61K 9/00

(52) U.S. Cl. CPC A61K 9/1271; A61K 9/0019

(57) ABSTRACT

The present disclosure relates compartmental administration of modified extracellular vesicles, e.g., exosomes. In some aspects, the extracellular vesicle, e.g., exosome, comprises a biologically active molecule and a targeting moiety.

The content of the electronically submitted sequence listing in ASCII text file (Name: 4000-092PC03_SL_ST25.txt; Size: 87,828 bytes; and Date of Creation: Mar. 15, 2021), filed with the application, is incorporated herein by reference in its entirety.

### CROSS REFERENCE TO RELATED APPLICATIONS

This PCT application claims the priority benefit of U.S. Provisional Application Nos. 62/989,524 filed Mar. 13, 2020; 62/989,535 filed Mar. 13, 2020; 62/704,998 filed Jun. 5, 2020, 63/035,393 filed Jun. 5, 2020; 63/154,562 filed Feb. 26, 2021; and 63/154,563 filed Feb. 26, 2021, each of which is incorporated herein by reference in its entirety.

### FIELD OF DISCLOSURE

The present disclosure relates to targeted delivery of modified extracellular vesicles (EVs) (e.g., exosomes), and the use of such EVs to treat and/or prevent a range of medical disorders, such as diseases that affect the central nervous system.

### BACKGROUND OF DISCLOSURE

EVs are important mediators of intercellular communication. They are also important biomarkers in the diagnosis and prognosis of many diseases, such as cancer. As drug delivery vehicles, EVs offer many advantages over traditional drug delivery methods (e.g., peptide immunization, DNA vaccines) as a new treatment modality in many therapeutic areas. However, despite its advantages, many EVs have had limited clinical efficacy. For example, dendritic-cell derived exosomes (DEX) were investigated in a Phase II clinical trial as maintenance immunotherapy after first line chemotherapy in patients with inoperable non-small cell lung cancer (NSCLC). However, the trial was terminated because the primary endpoint (at least 50% of patients with progression-free survival (PFS) at 4 months after chemotherapy cessation) was not reached. Besse, B., et al., Oncoimmunology 5(4):e1071008 (2015).

Accordingly, new and more effective engineered-EVs, particularly those that can specifically target specific tissues, are necessary to better enable therapeutic use and other applications of EV-based technologies.

### SUMMARY OF DISCLOSURE

Certain aspects of the present disclosure are directed to a method of treating a disease or disorder in a subject in need thereof, comprising compartmentally administering to the subject an effective amount of a composition comprising an extracellular vesicle (EV) which comprises a biologically active molecule. In some aspects, the compartmental administration localizes the EV to a target tissue.

Certain aspects of the present disclosure are directed to a method of directing an extracellular vesicle (EV) which comprises a biologically active molecule to a target tissue in a subject in need thereof, comprising compartmentally administering an effective amount of a composition comprising the EV to the subject.

In some aspects, the compartmental administration comprises administering the composition by a route selected from intraperitoneal, inhalation, oral, intramuscular, intrathecal, intracranial, intraocular, intradermal, sub-cutaneous, and any combination thereof. In some aspects, the compartmental administration comprises administering the composition by a route selected from intra-cisterna magna and intra-cerebroventricular. In some aspects, intracranial administration comprises administering the composition intracranially into any normal or lesioned part of the brain. In some aspects, intracranial administration comprises administering the composition intracranially via the nasal cavity or via the inner ear.

In some aspects, the target tissue comprises the central nervous system (CNS). In some aspects, the EV is administered intrathecally. In some aspects, the EV is administered intra-cranially.

In some aspects, the target tissue comprises the eye. In some aspects, the EV is administered intraocularly. In some aspects, the intraocular administration is selected from the group consisting of intravitreal, intracameral, subconjunctival, subretinal, subscleral, intrachoroidal, suprachoroidal, and any combination thereof.

In some aspects, the target tissue comprises a muscle. In some aspects, the EV is administered intramuscularly. In some aspects, the EV is administered intra-articularlly. In some aspects, the EV is administerd intra-articularlly into a skeletal joint, tendon, ligament, bursa, or any combination thereof.

In some aspects, the target tissue comprises the lungs. In some aspects, the target tissue comprises the epithelial linings of the respiratoty tract. In some aspects, the EV is administered by inhalation. In some aspects, the EV is delivered by tracheal intubation.

In some aspects, the target tissue comprises a lymph node. In some aspects, the EV is administered intraperitoneally. In some aspects, the EV is administered intramusculary, subcutaneously or via other routes for specific routing to regional lymph nodes draining such tissue.

In some aspects, the target tissue comprises the colon. In some aspects, the EV is administered orally. In some aspects, the EV is administered rectally. In some aspects, the EV is administered intraurethally.

In some aspects, the compartmental administration comprises the injection of the composition. In some aspects, the compartmental administration comprises the implantation of a delivery device comprising the composition. In some aspects, the delivery device comprises an implanted pump or a sustained delivery device.

In some aspects, the EV comprises an exogenous targeting moiety that specifically binds to a marker present on a cell in the target tissue. In some aspects, the exogenous targeting moiety comprises a peptide, an antibody or an antigen-binding fragment thereof, a chemical compound, or any combination thereof.

In some aspects, the exogenous targeting moiety comprises an antibody or antigen-binding fragment thereof. In some aspects, the antibody or antigen-binding fragment thereof comprises a full-length antibody, a single domain antibody, a heavy chain only antibody (VHH), a single chain antibody, a shark heavy chain only antibody (VNAR), an scFv, a Fv, a Fab, a Fab′, a F(ab′)2, or any combination thereof. In some aspects, the antibody is a single chain antibody. In some aspects, the trophic ligand is derived from a biological toxin or venom with known cognate receptors in normal tissues and cells.

In some aspects, the exogenous targeting moiety comprises a microprotein, a designed ankyrin repeat protein (darpin), an anticalin, an adnectin, an aptamer, a peptide mimetic molecule, a natural ligand for a receptor, a camelid nanobody, or any combination thereof.

In some aspects, the exogenous targeting moiety specifically binds to a marker on a CNS cell. In some aspects, the CNS cell is a selected from a neuronal cell, a glial cell, and any combination thereof. In some aspects, the CNS cell is a selected from an oligodendrocyte, an astrocyte, an ependymal cell, a microglia, and any combination thereof. In some aspects, the CNS cell is a selected from a motor neuron, a sensory neuron, an interneuron, and any combination thereof.

In some aspects, the exogenous targeting moiety specifically binds to a marker on an eye cell. In some aspects, the eye cell is selected from a rod cell, a cone cell, a retinal ganglion cell, and any combination thereof.

In some aspects, the exogenous targeting moiety specifically binds to a marker on a muscle cell. In some aspects, the muscle cell is selected from a skeletal muscle cell, a smooth muscle cell, a cardiomyocyte, and any combination thereof.

In some aspects, the exogenous targeting moiety specifically binds to a marker on an immune cell. In some aspects, the immune cell is selected from the group consisting of a CD4 T cell, a CD8 T cell, a B cell, and any combination thereof. In some aspects, the exogenous targeting moiety binds CD3. In some aspects, the EV is injected directly into lymph nodes.

In some aspects, the exogenous targeting moiety comprises CD40L. In some aspects, the exogenous targeting moiety specifically binds to a marker on a macrophage. In some aspects, the exogenous targeting moiety increases uptake of the EV by a macrophage. In some aspects, uptake of the EV by the macrophage activates the macrophage. In some aspects, the biologically active molecule is capable of repolarizing a macrophage. In some aspects, the macrophage is repolarized from an M2 to an M1 phenotype. In some aspects, the macrophage is repolarized from an M1 to an M2 phenotype.

In some aspects, the EV comprises a surface antigen that inhibits uptake of the EV by a macrophage. In some aspects, the surface antigen is selected from CD47, CD24, a fragment thereof, and any combination thereof. In some aspects, the surface antigen is associated with the exterior surface of the EV.

In some aspects, the biologically active molecule, the exogenous targeting moiety, or both are linked to the EV by a scaffold protein. In some aspects, the scaffold protein is a Scaffold X protein. In some aspects, the scaffold protein is a Scaffold Y protein.

In some aspects, the composition comprising the EV further comprises a therapeutic molecule, an immune modulator, an adjuvant, or any combination thereof. In some aspects, the therapeutic molecule comprises an antigen. In some aspects, the adjuvant comprises a Stimulator of Interferon Genes (STING) agonist, a toll-like receptor (TLR) agonist, an inflammatory mediator, or any combination thereof. The method of claim 64 or 66, wherein the adjuvant comprises a STING agonist. In some aspects, the STING agonist comprises a cyclic dinucleotide STING agonist or a non-cyclic dinucleotide STING agonist.

In some aspects, the adjuvant is a TLR agonist. In some aspects, the TLR agonist comprises a TLR2 agonist (e.g., lipoteichoic acid, atypical LPS, MALP-2 and MALP-404, OspA, porin, LcrV, lipomannan, GPI anchor, lysophosphatidylserine, lipophosphoglycan (LPG), glycophosphatidylinositol (GPI), zymosan, hsp60, gH/gL glycoprotein, hemagglutinin), a TLR3 agonist (e.g., double-stranded RNA, e.g., poly(I:C)), a TLR4 agonist (e.g., lipopolysaccharides (LPS), lipoteichoic acid, β-defensin 2, fibronectin EDA, HMGB1, snapin, tenascin C), a TLR5 agonist (e.g., flagellin), a TLR6 agonist, a TLR⅞ agonist (e.g., single-stranded RNA, CpG-A, Poly G10, Poly G3, Resiquimod), a TLR9 agonist (e.g., unmethylated CpG DNA), or any combination thereof.

In some aspects, the therapeutic molecule, the immune modulator, the adjuvant, or any combination thereof, is associated with Scaffold X, Scaffold Y, or a combination thereof.

In some aspects, the immune modulator comprises a cytokine. In some aspects, the cytokine comprises an interferon.

In some aspects, the EV is an exosome.

The present disclosure provides a method of targeting an extracellular vesicle to central nervous system in a subject in need thereof comprising administering a composition comprising an extracellular vesicle (EV) which comprises a biologically active molecule to the subject, wherein the administration of the composition is intrathecal, intraocular, intracranial, intranasal, or perineural. Also provided is a method of treating a central nervous system disease in a subject in need thereof comprising administering a composition comprising an extracellular vesicle (EV) which comprises a biologically active molecule to the subject, wherein the administration of the composition is intrathecal, intraocular, intracranial, intranasal, intraneural (into nerves), or perineural.

In some aspects, the intrathecal administration is in the spinal canal and/or the subarachnoid space. In some aspects, the intrathecal administration is by injection. In some aspects, the intrathecal administration comprises the implantation of a delivery device comprising the composition. In some aspects, the delivery device is an intrathecal pump.

In some aspects, the intraocular administration is selected from the group consisting of intravitreal, intracameral, subconjunctival, subretinal, subscleral, intrachoroidal, and any combination thereof. In some aspects, the intraocular administration comprises the injection of the composition. In some aspects, the intraocular administration is intravitreal injection. In some aspects, the intraocular administration comprises the implantation of a delivery device comprising the composition. In some aspects, the delivery device is an intraocular delivery device. In some aspects, the intraocular delivery device is an intravitreal implant or a scleral plug. In some aspects, the delivery device is a sustained release delivery device.

In some aspects, the intracranial administration is intracisternal, subarachnoidal, intrahippocampal, intracerebroventricular, intracisternal, intraparenchymal, intraneural or a combination thereof. In some aspects, the intracranial administration is by injection. In some aspects, the intracranial administration is via a catheter or port. In some aspects, the catheter or port is implanted. In some aspects, a pump is connected to the catheter or port. In some aspects, the intraparenchymal administration is Convection-Enhanced Intraparenchymal administration.

In some aspects, the intranasal administration is by instillation or injection. In some aspects, the perineural administration is by facial intradermal injection. In some aspects, the facial intradermal injection targets the trigeminal substructures. In some aspects, the trigeminal substructures are selected from the group consisting of trigeminal perineurium, epineurium, perivascular spaces, neurons and Schwann cells, and combinations thereof. In some aspects, the extracellular vesicle, e.g., exosome, comprises a surface anchored anti-phagocytic signal. In some aspects, the anti-phagocytic signal is CD47, CD24, a fragment or variant thereof, or a combination thereof. In some aspects, the extracellular vesicle, e.g., exosome, comprises a tissue or cell-specific target ligand which increases extracellular vesicle, e.g., exosome, tropism to a specific CNS tissue or cell.

In some aspects, the extracellular vesicle, e.g., exosome, comprises a surface anchored anti-phagocytic signal (e.g., CD47, CD24, a fragment or variant thereof, or a combination thereof) and a tissue or cell-specific target ligand which increases extracellular vesicle, e.g., exosome, tropism to a specific CNS tissue or cell.

In some aspects, the cell is a glial cell. In some aspects, the glial cell is an oligodendrocyte, an astrocyte, an ependymal cell, a microglia cell, a Schwann cell, a satellite glial cell, an olfactory ensheathing cell, or a combination thereof. In some aspects, the cell is a neural stem cell. In some aspects, the cell is a neuron. In some aspects, the neuron is a motor neuron, a sensory neuron, or an interneuron.

In some aspects, wherein the tissue or cell-specific target ligand is a cell marker (e.g., a protein or receptor) present of the surface of a neuron. In some aspects, the tissue is selected from the group consisting of brain tissue, spinal cord tissue, retina, optic nerve (cranial nerve II), olfactory nerves (cranial nerve I), olfactory epithelium, meningeal tissue, or any combination thereof. In some aspects, the tissue is from a CNS area selected from the group consisting of cerebrum, cerebral cortex, basal ganglia, amygdala, hippocampus, thalamus, hypothalamus, cerebellum, brainstem, medulla, pons, midbrain, and reticular formation.

The present disclosure provides a method of targeting an extracellular vesicle (EV), e.g., exosome, to the central nervous system (CNS) in a subject in need thereof comprising administering a composition comprising an extracellular vesicle (EV), e.g., exosome, which comprises a biologically active molecule to the subject, wherein the administration of the composition is intrathecal, intraocular, intracranial, intranasal, or perineural, and wherein the extracellular vesicle (EV), e.g., exosome comprises (i) a surface anchored anti-phagocytic signal and (ii) a tissue or cell-specific target ligand which increases EV tropism to cells in the CNS. Also provided is method of treating a CNS disease or condition in a subject in need thereof comprising administering an extracellular vesicle (EV), e.g., exosome, to the CNS of the subject wherein an extracellular vesicle (EV), e.g., exosome, comprises a biologically active molecule to the subject, wherein the administration of the composition is intrathecal, intraocular, intracranial, intranasal, or perineural, and wherein the extracellular vesicle (EV), e.g., exosome comprises (i) a surface anchored anti-phagocytic signal and (ii) a tissue or cell-specific target ligand which increases EV tropism to cells in the CNS. In some aspects, the cells are Schwann cells or oligodendrocytes. In some aspects, the anti-phagocytic signal is CD47, CD24, a fragment or variant thereof, or a combination thereof. In some aspects, the anti-phagocytic signal is covalently attached to a Scaffold X moiety. In some aspects, the Scaffold X moiety is PTGFRN or a functional fragment thereof.

In some aspects, the tissue or cell-specific target ligand targets a CNS specific peripheral nerve. In some aspects, the tissue or cell-specific target ligand comprises a ligand that binds to a transferrin receptor (TfR), apolipoprotein D (ApoD), Galectin 1 (LGALS1), Myelin proteolipid protein (PLP), Glypican 1, or Syndecan 3. In some aspects, the TfR is TfR1. In some aspects, the ligand that binds to TfR1 is an antibody against TfR1 or transferrin. In some aspects, the transferrin is a serum transferrin, lacto transferrin (lactoferrin) ovotransferrin, or melanotransferrin. In some aspects, the transferrin is an asialo transferrin, a monosialo transferrin, a disialo transferrin, a trisialo transferrin, a tetrasialo transferrin, a pentasialo transferrin, an hexasialo transferrin, or a combination thereof. In some aspects, the tissue or cell-specific target ligand binds to a Schwann cell surface marker. In some aspects, the Schwann cell surface marker is selected from Myelin Basic Protein (MBP) and isoforms thereof, Myelin Protein Zero (P0), P75NTR, NCAM, PMP22, and combinations thereof. In some aspects, the tissue or cell-specific target ligand comprises an antibody or an antigen-binding portion thereof, a vNAR, an aptamer, or an agonist or antagonist of a receptor expressed on the surface of the Schwann cell. In some aspects, the tissue or cell-specific target ligand targets a sensory neuron. In some aspects, the tissue or cell-specific target ligand comprises a neurotrophin that binds to a tropomyosin receptor kinase (Trk) receptor. In some aspects, the Trk receptor is TrkA, TrB, TrkC, or a combination thereof. In some aspects, the neurotrophin is Nerve growth factor (NGF), Brain-derived neurotrophic factor (BDNF), Neurotrophin-3 (NT-3), Neurotrophin-4 (NT-4), or a combination thereof. In some aspects, the tissue or cell-specific target ligand targets a motor neuron. In some aspects, the tissue or cell-specific target ligand comprises a Rabies Virus Glycoprotein (RVG) peptide, a Targeted Axonal Import (TAxI) peptide, a P75R peptide, or a Tet-C peptide.

### BRIEF DESCRIPTION OF FIGURES

FIG. 1 illustrates strategies to engineer CNS tropism. EVs can be surface engineered to adjust pharmacokinetics and biodistribution, modifed to alter clearance (e.g., attachment of CD47 and/or CD24 “don’t eat me” signals, and/or half-life extension moieties), or modifed to alter tropism (e.g., via incorporation of immuno-affinity ligands or cognate receptor ligands).

FIG. 2 shows histological biodistribution in mouse of exosomes in neurons after intracranial or intravitreal administration. IHC was performed using anti-Exosome specific Antibodies such as 1G11; Exosomes = Red.

FIG. 3 shows that exosome tropism improves intrathecal compartment retention in a meningeal macrophage targeting example. The cryo-fluorescence-tomography map after intrathecal dosing shows that exosome fluorescence located to the CNS, and that it particularly targeted the cranial and spinal meninges.

FIG. 4 shows that exosome tropism improves intrathecal compartment retention in a meningeal macrophage targeting example. In vivo PET imaging shows that the ASOs administered without exosomes distributed to various tissues, especially kidney and liver. When ASOs were administered in exosomes neuroaxial retention improved.

FIG. 5 shows that exosome tropism improves intrathecal compartment retention in a meningeal macrophage targeting example. Immunohistochemistry with 1G11, an antibody specific for the exosomes used in the experiments, indicates that the exosomes effectively targeted meningeal macrophages and meningeal lymphatic endothelium (red fluorescence in right panels).

FIG. 6 is a schematic representation showing intrathecal exosome targeting towards CNS cells. In addition to the tropism for meningeal macrophages and meningeal lymphatic endothelium described in previous figures, exosomes can be routed to specific cells via targeting moieties, e.g., to Schwann cells, sensory neurons, or motor neurons.

FIGS. 7A-7D are schematic drawings of various CD47-Scaffold X fusion constructs. FIG. 7A shows constructs comprising the extracellular domain of wild-type CD47 (with a C15S substitution) fused to either a flag-tagged (1083 and 1084) or non-flag-tagged (1085 and 1086) full length Scaffold X (1083 and 1086) or a truncated Scaffold X (1084 and 1085). FIG. 7B shows constructs comprising the extracellular domain of Velcro-CD47 fused to either a flag-tagged (1087 and 1088) or non-flag-tagged (1089 and 1090) full length Scaffold X (1087 and 1090) or a truncated Scaffold X (1088 and 1089). FIG. 7C shows constructs wherein the first transmembrane domain of wild-type CD47 (with a C15S substitution; 1127 and 1128) or Velcro-CD47 (1129 and 1130) is replaced with a fragment of Scaffold X, comprising the transmembrane domain and the first extracellular motif of Scaffold X. FIG. 7D shows various constructs comprising a minimal “self” peptide (GNYTCEVTELTREGETIIELK; SEQ ID NO: 382) fused to either a flag-tagged (1158 and 1159) or non-flag-tagged (1160 and 1161) full length Scaffold X (1158 and 1161) or a truncated Scaffold X (1159 and 1160).

FIGS. 8A-8B are graphical representations of CD47 expression on exosomes as measured by ELISA (FIG. 8A) and by a SIRPα signaling reporter assay (FIG. 8B). FIG. 8A is a bar graph showing the concentration of CD47 molecules on the surface of exosomes expressing each of the constructs in FIGS. 7A-7D, as measured by ELISA. FIG. 8B is a bar graph showing the relative concentration CD47 molecules on the surface of exosomes as measured by the chemiluminescence generated in a SIRPα signaling reporter assay (DiscoverX).

FIGS. 9A-9C are graphical representations illustrating SIRPα binding by each of the CD47-Scaffold X constructs, expressed on exosomes, as measured by Octet assay. All steps are aligned by step baseline (sensor location).

FIGS. 10A-10D show the uptake of GFP-Scaffold-X exosomes by primary human monocyte-derived M0 macrophages. FIGS. 10A-10C are cell images showing IncuCyte real-time analysis for GFP-Scaffold-X surface expressing exosome localization to primary human monocyte-derived M0 macrophages as an overlay (FIG. 10A), a confluence mask (FIG. 10B), and a fluorescence mask (FIG. 10C). FIG. 10D is a graphical illustration of localization of GFP positive exosomes to primary human monocyte-derived M0 macrophages over time following exposure to exosomes at a dose of 4.4 X 10⁸ particles/mL exosomes (triangles), 1.33X 10⁹ particles/mL exosomes (squares), and 4 X 10⁹ particles/mL exosomes (circles).

FIGS. 10E-10I are graphical representations of localization of CD47 surface-expressing exosomes to primary human monocyte-derived M0 macrophages (FIGS. 10E-10G) or HEK cells (FIGS. 10H-10I) over time following exposure of the cells to exosomes at a concentration of 4X 10⁹ particles/mL (FIG. 10E), 1.33 X 10⁹ particles/mL (FIG. 10F), 4.4 X 10⁸ particles/mL exosomes (FIG. 10G), 5 X 10¹⁰ particles/mL FIG. 10I), and 1.67 X 10¹⁰ particles/mL (FIG. 10H).

FIGS. 11A-11B show the expression of various mouse CD47-Scaffold X fusion constructs (FIG. 11A) on the surface of modified exosomes. FIG. 11A shows constructs comprising the extracellular domain of wild-type murine CD47 (with a C15S substitution) fused to either a flag-tagged (1923 and 1925) or non-flag-tagged (1924 and 1922) full length Scaffold X (1923 and 1922) or a truncated Scaffold X (1925 and 1924). FIG. 11B is a graphical representation showing the number of murine CD47 particles localizing to the surface of exosomes.

FIG. 12 is a graphical representation illustrating binding of both human and mouse CD47 exosomes to mouse SIRPα, as measured by Octet assay.

FIGS. 13A-13B are graphical illustrations of localization of the various mouse CD47-Scaffold X fusion constructs (FIG. 13A) in mouse bone marrow-derived macrophages over time following exposure of the macrophages to exosomes at a concentration of 1.67 X 10¹⁰ particles/mL (FIGS. 13A-13B).

FIGS. 14A-14N show representative images of exosome localization in mouse bone marrow-derived macrophages exposed to 5 X 10¹⁰ particles/mL of Scaffold X modified exosomes (FIGS. 14A and 14B), native exosomes (FIGS. 14C and 14D), exosomes expressing the extracellular domain of murine CD47^{C155} fused to Scaffold X (FIGS. 14E and 17F), and exosomes expressing the extracellular domain of murine CD47^{C155} fused to a flag-tagged Scaffold X (FIGS. 14G and 14H), at 2 hours (FIGS. 14A, 14C, 14E, and 14G), 7.5 hours (FIGS. 14B, 14D, 14F, and 14H), and 19.5 hours (FIGS. 14K-14N). FIGS. 14I and 14J show localization of native exosomes to HEKsf cells.

FIG. 15A is a graphical representation illustrating internalization by primary human macrophages of exosomes expressing either PTGFRN or CD47 fused to PTGFRN, as indicated. FIG. 15B is a graphical representation illustrating the half-life of exosomes based on increasing concentrations of polyethylene glycol (PEG). Error bars indicate standard deviation (FIG. 15B).

FIGS. 16A-16D show the uptake of GFP-Scaffold-X exosomes by primary human monocyte-derived M0 macrophages. FIGS. 16A-16C are cell images showing IncuCyte real-time analysis for GFP-Scaffold-X surface expressing exosome localization to primary human monocyte-derived M0 macrophages as an overlay (FIG. 16A), a confluence mask (FIG. 16B), and a fluorescence mask (FIG. 16C). FIG. 16D is a graphical illustration of localization of GFP positive exosomes to primary human monocyte-derived M0 macrophages over time following exposure to exosomes at a dose of 4.4 X 10⁸ particles/mL exosomes (triangles), 1.33X 10⁹ particles/mL exosomes (squares), and 4 X 10⁹ particles/mL exosomes (circles). FIGS. 16E-16L are graphical representations of localization of CD47 surface-expressing exosomes to primary human monocyte-derived M0 macrophages (FIGS. 16E-16G) or HEK cells (FIGS. 16H-16I) over time following exposure of the cells to exosomes at a concentration of 4X 10⁹ particles/mL (FIG. 16E), 1.33 X 10⁹ particles/mL (FIG. 16F), 4.4 X 10⁸ particles/mL exosomes (FIG. 16G), 5 X 10¹⁰ particles/mL FIG. 16I), and 1.67 X 10¹⁰ particles/mL (FIG. 16H).

FIGS. 17A-17B show the expression of various mouse CD47-Scaffold X fusion constructs (FIG. 17A) on the surface of modified exosomes. FIG. 17A shows constructs comprising the extracellular domain of wild-type murine CD47 (with a C15S substitution) fused to either a flag-tagged (1923 and 1925) or non-flag-tagged (1924 and 1922) full length Scaffold X (1923 and 1922) or a truncated Scaffold X (1925 and 1924). FIG. 17B is a graphical representations showing the number of murine CD47 particles localizing to the surface of exosomes.

FIG. 18 is a graphical representation illustrating binding of both human and mouse CD47 exosomes to mouse SIRPα, as measured by Octet assay.

FIGS. 19A-19B are graphical illustrations of localization of the various mouse CD47-Scaffold X fusion constructs (FIG. 17A) mouse bone marrow-derived macrophages over time following exposure of the macrophages to exosomes at a concentration of 1.67 X 10¹⁰ particles/mL (FIGS. 19A-19B).

FIGS. 20A–20N show representative images of exosome localization in mouse bone marrow-derived macrophages exposed to 5 X 10¹⁰ particles/mL of Scaffold X modified exosomes (FIGS. 20A and 20B), native exosomes (FIGS. 20C and 20D), exosomes expressing the extracellular domain of murine CD47^{C155} fused to Scaffold X (FIGS. 20E and 20F), and exosomes expressing the extracellular domain of murine CD47^{C155} fused to a flag-tagged Scaffold X (FIGS. 9G and 9H), at 2 hours (FIGS. 20A, 20C, 20E, and 20G), 7.5 hours (FIGS. 20B, 20D, 20F, and 20H), and 19.5 hours (FIGS. 20K-20N). FIGS. 20I and 20J show localization of native exosomes to HEKsf cells.

FIGS. 21A-21E are images of live PET scanning of non-human primate (NHP; FIGS. 21A and 21D), rat (FIG. 21B), and mouse (FIGS. 21C and 21E) administered ⁸⁹Zr-labeled exosomes intravenously (FIGS. 21A-21C) or intraperitoneally (FIGS. 21D-21E).

FIGS. 22A-22I are immunohistochemical images of samples obtained from mice administered exosomes intravenously (FIG. 22A (liver) and 22B (spleen)), intraperitoneally (FIG. 22C; lymph node), or by compartmental administration (FIGS. 22D-22I). Compartmental administration: inhalation resulted in localization of the exosomes to the lungs (FIG. 22F); intramuscularly administration resulted in localization of the exosomes to muscle cells (FIG. 22G); oral administration resulted in localization of the exosomes to at least the colon (FIG. 22H); and intra-tumor delivery into a live tumor resulted in localization of the exosomes to tumor tissue (FIG. 22I).

FIGS. 23A-23C are images of live PET scanning of rat administered non-exosomal ¹²⁵I-labeled ASO (FIG. 23A) or ⁸⁹Zr-labeled exosomes (FIGS. 23B-23C) intrathecally. FIG. 3C shows labeling of localization to specific parts of the CNS, as indicated, and to the lymph nodes and intrathecal catheter. Only a fluorescence artifact is observed in the GI tract (FIG. 23C). Localization is further shown to the cranial meninges (FIG. 23D) and the spinal meninges (FIG. 23E) in dissected tissue samples.

FIGS. 24A-24F are fluorescent immunohistochemistry images showing DAPI (FIG. 24A), 1G11 antibody (FIG. 24B), CD206 (FIG. 24C), and LYVE1 (FIG. 24E) staining. FIG. 24D shows the overlay of IG11 antibody and CD206 staining, and FIG. 24F shows the overlay of 1G11 antibody and LYVE1 staining.

FIG. 25A is a bar graph illustrating localization (as represented by Cy5 MFI) to CD4 T cells and CD8 T cells, as indicated, of negative control Scaffold X expressing exosomes ( “1 PrX exo ”) and exosomes expressing an anti-CD3 antibody (“2 anti-CD3 exo”). FIG. 25B is a line graph illustrating localization to B cells of exosomes expressing PTGFRN fused to GFP or exosomes expressing CD40L fused to GFP. FIGS. 25C-25D are images of immunohistochemical staining of Neuro2A cells showing update by either exosomes expressing a neurotropic peptide fused to Scaffold X (PrX; FIG. 25C) or a negative control (FIG. 25D).

FIGS. 26A-26D show exoRVG uptake in neuro2A cells. The constructs tested were: RVG-PrX-mCherry-FLAG-HiBiT (construct 2021; FIG. 26A), linker-PrX-mCherry-FLAG-HiBiT (construct 2022; FIG. 26B), RVG-LAMP2B-mCherry-FLAG-HiBiT (construct 2023; FIG. 26C), and linker-LAMP2B-mCherry-FLAG-HiBiT (construct 2024; FIG. 26D). Only the constructs comprising RVG showed uptake by the neuro2A cells. 10⁵ EV particles per cell were used. EV uptake was observed at 5 hours. “RVG” is a tropism moiety of sequence YTIWMPENPRPGTPCDIFTNSRGKRASNG (SEQ ID NO: 408). “Linker” is a linker of sequence GGSSGSGSGSGGGGSGGGGTGTSSSGTGT (SEQ ID NO: 435). “FLAG” is a FLAG® epitope tag. “HiBiT” is the nano luciferase peptide described above. “mCherry” is a red fluorescent protein. “LAMP2B” and “PrX” are protein scaffolds. “ExoRVG” EV are exosomes comprising an RVG tropism moiety.

FIGS. 27A-27B show exoRVG uptake in neuro2A cells 18 hours after the cells were incubated with 5x10⁴ EV particles per neuro2A cell. Measurements were taken 18 hours after uptake. The constructs tested were exoRVG (construct 2021, see FIG. 15) (FIG. 27A) and exoLinker (construct 2020, see FIG. 26) (FIG. 27B). Uptake was only observed when constructs comprising the tropism moiety RVG were used,

FIGS. 28A-28X show exoRVG uptake in neuro2A cells 24 hours after incubation with EV comprising one of the four constructs described in FIG. 15. Samples used were negative control (no EV particles; FIGS. 28A-28D), E5 (10⁵ particles/cell; FIGS. 28E-28H), 5E4 (5x10⁴ particles/cell; FIGS. 28I-28L), E4 (10⁴ particles/cell; FIGS. 28M-28P), 5E3 (5x10³ particles/cell; FIGS. 8O-28T), and E3 (10³ particles/cell; FIGS. 28U-8X). The boxed data sets corresponds to the samples used in FIG. 27 measured at 24 hours after uptake.

FIG. 29 compares EV uptake in neuro2A cells corresponding to negative control (leftmost curve), exoLinker (construct 2020) (center curve), and exoRVG (construct 2021) (rightmost curve), meaured 24 hours after the cells were incubated with 5x10⁴ EV particles per neuro2A cell.

FIGS. 30A-30C show exoTransferrin uptake in HeLa cells. Three constructs were tested: Transferrin-PrX-mCherry-FLAG (human transferrin; construct 1597; FIG. 30A), mTransferrin-PrX-mCherry-FLAG (mouse transferrin; construct 1598; FIG. 30B); and linker-PrX-mCherry-FLAG-HiBiT (construct 2022; FIG. 30C). 5x10⁵ EV particles per HeLa cell were used. “ExoTransferrin” EV are exosomes comprising a transferrin tropism moiety. Uptake was measured 3 hours after EV particle incubation started. EV uptake was observed for both human and mouse transferrin-containing EVs.

FIGS. 31A-31C show exoTransferrin uptake in Hep3B cells. Three constructs were tested: Transferrin-PrX-mCherry-FLAG (human transferrin; construct 1597; FIG. 31A), mTransferrin-PrX-mCherry-FLAG (mouse transferrin; construct 1598; FIG. 31B); and linker-PrX-mCherry-FLAG-HiBiT (construct 2022; FIG. 31C). 5x10⁵ EV particles per Hep3B cell were used. Uptake was measured 3 hours after EV particle incubation started. EV uptake was observed for both human and mouse transferring-containing EVs.

FIGS. 32A-32C show exoTransferrin uptake in Hep3G2 cells. Three constructs were tested: Transferrin-PrX-mCherry-FLAG (human transferrin; construct 1597; FIG. 32A), mTransferrin-PrX-mCherry-FLAG (mouse transferrin; construct 1598; FIG. 32B); and linker-PrX-mCherry-FLAG-HiBiT (construct 2022; FIG. 32C). 5x10⁵ EV particles per HepG2 cell were used. Uptake was measured 3 hours after EV particle incubation started. EV uptake was observed for both human and mouse transferrin-containing EVs.

FIG. 33A shows a schematic diagram of exemplary extracellular vesicle (e.g., exosome) targeting Trks using neurotrophin-Scaffold X fusion construct. Neurotrophins bind to Trk receptors as a homo dimer and allow the EV to target a sensory neuron.

FIG. 33B shows a schematic diagram of exemplary extracellular vesicle (e.g., exosome) having (i) neuro-tropism as well as (ii) an anti-phagocytic signal, e.g., CD47 and/or CD24, on the exterior surface of the EV disclosed herein.

FIGS. 34A-34F shows shows the biodistribution of [⁸⁹Zr]DFO-PrX labelled exosomes in a cynomolgus macaque 0.5 hours (FIGS. 34A and 34D), six hours (FIGS. 34B and 34E), and 24 hours (FIGS. 34C and 34F) post-intrathecal (ITH) administration, with FIGS. 34A-34C showing the full torso and head and FIGS. 34D-34F showing cropped head images.

FIGS. 35A-35F shows the biodistribution of [⁸⁹Zr]DFO-PrX labelled exosomes in cynomolgus macaque 0.5 hours (FIGS. 35A and 35D), six hours (FIGS. 35B and 35E), and 24 hours (FIGS. 35C and 35F) post-intra-cisterna magna (ICM) administration, with FIGS. 35A-35C showing the full torso and head and FIGS. 35D-35F showing cropped head images.

FIGS. 36A-36F show sagittal slices for ITH-delivered (FIGS. 36A-36C) and ICM-delivered (FIGS. 36D-36F) [⁸⁹Zr]DFO-PrX exosomes at 0.5 hour (FIGS. 36A and 236D), 6 hours (FIGS. 36B and 36E), and 24 hours (FIGS. 36C and 36F) post administration in a cynomolgus macaque.

FIGS. 37A-37F show cropped head slices for ICM-delivered (FIGS. 37A-37C) and ITH-delivered (FIGS. 37D-37F) [⁸⁹Zr]DFO-PrX exosomes at 0.5 hour (FIGS. 37A and 37D), 6 hours (FIGS. 37B and 37E), and 24 hours (FIGS. 37C and 37F) post administration in a cynomolgus macaque.

FIGS. 38A-38B are fluorescent images of neuro2A cells cultuted in the presense of ExoLinker (negative control; FIG. 38A) or ExoTAxl (FIG. 38B).

FIGS. 39A-39I are fluorescent images of neuro2A cells cultuted in the presense of ExoTransferrin (1597; FIGS. 39A, 39D, and 39G), ExoTransferrin (1598; FIGS. 39B, 39E, and 39H), or ExoLinker (negative control; FIGS. 39C, 39F, and 39I) at 2 hours (FIGS. 39A-39C), 7 hours (FIGS. 39D-39F) and 24 hours (FIGS. 39G-39I).

FIGS. 40A-40C are fluorescent images of neuro2A cells cultuted in the presense of ExoTransferrin, showing Th expression (FIG. 40A) and overlay of TH expression and mCherry tagged EVs (FIGS. 40B-40C).

FIGS. 41A-41B are fluorescent images of human neuroblastoma cells (SH-SY-5Y) incubated with EV samples comprising a control (exoLinker; FIG. 41A) or exo-mTransferrin (FIG. 41B).

FIGS. 42A-42I are fluorescent images of primary mouse Schwann cells cells cultuted in the presense of exoTransferrin (1597; FIGS. 42A, 42D, and 42G), exo-mTransferrin (1598; FIGS. 42B, 42E, and 42H), or ExoLinker (negative control; FIGS. 42C, 42F, and 42I) at 2 hours (FIGS. 42A-42C), 5 hours (FIGS. 42D-42F) and 22 hours (FIGS. 42G-42I).

FIGS. 43A-43I are fluorescent images of primary human Schwann cells cells cultuted in the presense of exoTransferrin (1597; FIGS. 43A, 43D, and 43G), exo-mTransferrin (1598; FIGS. 43B, 43E, and 43H), or ExoLinker (negative control; FIGS. 43C, 43F, and 43I) at 2 hours (FIGS. 43A-43C), 5 hours (FIGS. 43D-43F) and 22 hours (FIGS. 43G-43I).

FIGS. 44A-44B are images of primary mouse Schwann cells (FIG. 44A) and primary human Schwann cells (FIG. 44B), which were cultured in the presence of exoTransferrin, fixed, and stained with anti-cytoskeleton-marker antbody and DAPI.

FIGS. 45A-45B are fluorescence images of SH-SY-SY cells cultuted in the presense of EVs expressing PrX-GFP (negative control; FIG. 44) or anti-TfnR(8D3)-PrX-GFP (FIG. 44B) overnight. FIG. 23C is a bar graph showing the transferrin copy number per EV particle for exoTransferrin (1597) small scale and large scale and exo-mTransferrin (1598) small scale and large scale.

### DETAILED DESCRIPTION OF DISCLOSURE

The present disclosure is directed to targeted delivery of an EV which comprises a biologically active molecule to the subject. In some aspects, the target delivery is achieved by compartmental administration of the EV. Some aspects of the present disclosure are directed to methods of treating a disease or disorder in a subject in need thereof, comprising compartmentally administering an effective amount of a composition comprising an EV which comprises a biologically active molecule to the subject. Non-limiting examples of the various aspects are shown in the present disclosure.

### I. Definitions

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein. It is further noted that the claims can be drafted to exclude any optional element. As such, this statement is intended to serve as antecedent basis for use of such exclusive terminology as “solely,” “only” and the like in connection with the recitation of claim elements, or use of a negative limitation.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

Nucleotides are referred to by their commonly accepted single-letter codes. Unless otherwise indicated, nucleotide sequences are written left to right in 5′ to 3′ orientation. Nucleotides are referred to herein by their commonly known one-letter symbols recommended by the IUPAC-IUB Biochemical Nomenclature Commission. Accordingly, A represents adenine, C represents cytosine, G represents guanine, T represents thymine, U represents uracil.

Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the term “extracellular vesicle” or “EV” refers to a cell-derived vesicle comprising a membrane that encloses an internal space. Extracellular vesicles comprise all membrane-bound vesicles (e.g., exosomes, nanovesicles) that have a smaller diameter than the cell from which they are derived. In some aspects, extracellular vesicles range in diameter from 20 nm to 1000 nm, and can comprise various macromolecular payload either within the internal space (i.e., lumen), displayed on the external surface of the extracellular vesicle, and/or spanning the membrane. In some aspects, the payload can comprise adeno-associated virus (AAV), nucleic acids (e.g., DNA or RNA, such as antisense oligonucleotides, siRNA, shRNA, or mRNA), morpholinos, proteins, carbohydrates, lipids, small molecules, vaccines, and/or combinations thereof. In some aspects, an EV comprises one or more payloads or other exogenous biologically active molecules. In some aspects, an EV comprises a targeting moiety that is exogenous to the EV (i.e., not naturally expressed in the EV) and that allows the EV to target a specific tissue or a specifc population of cells. In certain aspects, an extracellular vehicle can further comprise one or more scaffold moieties. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, prokaryotic or eukaryotic cells, and/or cultured cells. In some aspects, the extracellular vesicles are produced by cells that express one or more transgene products. The EVs disclosed herein have been modified and therefore, do not comprise naturally occurring EVs. In some aspects, a composition comprising an EV of the present disclosure comprises a population of exosomes, microvesicles, apoptotic bodies, and/or any combination hereof.

As used herein, the term “exosome” refers to an extracellular vesicle with a diameter between 20-300 nm (e.g., between 40-200 nm). Exosomes comprise a membrane that encloses an internal space (i.e., lumen), and, in some aspects, can be generated from a cell (e.g., producer cell) by direct plasma membrane budding or by fusion of the late endosome with the plasma membrane. In some aspects, an exosome comprises one or more exogenous biologically active molecules (e.g., as described herein). In some aspects, an exosome disclosed herein comprises a targeting moiety that is exogenous to the exosome (i.e., not naturally expressed in the exosome) and that allows the exosome to target a specific tissue or a specific population of cells. In certain aspects, an exosome further comprises one or more scaffold moieties. As described infra, exosomes can be derived from a producer cell, and isolated from the producer cell based on its size, density, biochemical parameters, or a combination thereof. In some aspects, exosomes of the present disclosure are produced by cells that express one or more transgene products. The exosomes of the present disclosure are modified and therefore, do not comprise naturally occurring exosomes.

In some aspects, EVs e.g., nanovesicles, of the present disclosure are engineered by covalently linking at least one biologically active molecule (e.g., a protein such as an antibody or ADC, a RNA or DNA such as an antisense oligonucleotide, a small molecule drug, a toxin, a PROTAC, an AAV, or a morpholino) to the EV e.g,. nanovesicle, via a maleimide moiety. In some aspects, the maleimide moiety is part of a bifunctional reagent.

In some aspects, the EVs, e.g., exosomes or nanovesicles, of the present disclosure can comprise various macromolecular payloads either within the internal space (i.e., lumen), displayed on the external (exterior) surface or internal (luminal) surface of the EV, and/or spanning the membrane. In some aspects, the payload can comprise, e.g., nucleic acids, proteins, carbohydrates, lipids, small molecules, and/or combinations thereof. In certain aspects, an EV, e.g, an exosome, comprises a scaffold moiety, e.g., Scaffold X. EVs can be derived from a living or dead organism, explanted tissues or organs, prokaryotic or eukaryotic cells, and/or cultured cells. In some aspects, the EVs are produced by cells that express one or more transgene products. In other aspects, the EVs of the present disclosure are without limitation nanovesicles, microsomes, microvesicles, extracellular bodies, or apoptotic bodies.

As used herein, the term “nanovesicle” refers to an extracellular vesicle with a diameter between about 20 nm and about 250 nm (e.g., between about 30 nm and about 150 nm) and is generated from a cell (e.g., producer cell) by direct or indirect manipulation such that the nanovesicle would not be produced by the cell without the manipulation. Appropriate manipulations of the cell to produce the nanovesicles include but are not limited to serial extrusion, treatment with alkaline solutions, sonication, or combinations thereof. In some aspects, production of nanovesicles can result in the destruction of the producer cell. In some aspects, population of nanovesicles described herein are substantially free of vesicles that are derived from cells by way of direct budding from the plasma membrane or fusion of the late endosome with the plasma membrane. In some aspects, a nanovesicle comprises one or more exogenous biologically active molecules (e.g., disclosed herein). In some aspects, a nanovesicle can further comprise a targeting moiety that is exogenous to the nanovesicle (i.e., not naturally expressed in the nanovesicle) and that allows the nanovesicle to target a specific tissue or a specific population of cells. In certain aspects, a nanovesicle further comprises one or more scaffold moieties. In certain aspects, a nanovesicle comprises a scaffold moiety, e.g., Scaffold X and/or Scaffold Y. Nanovesicles, once derived from a producer cell, can be isolated from the producer cell based on its size, density, biochemical parameters, or a combination thereof. As used herein, nanovesicles have been modified and therefore, do not comprise naturally occurring nanovesicles.

As used herein the term “surface-engineered EVs, e.g., exosomes” (e.g., Scaffold X-engineered EVs, e.g., exosomes) refers to an EV with the membrane or the surface modified in its composition, so that the membrane or the surface of the engineered EV, is different from either that of the EV prior to the modification or of the naturally occurring EV. The engineering can be on the surface of the EV or in the membrane of the EV so that the surface of the EV is changed. For example, the membrane is modified in its composition of a protein, a lipid, a small molecule, a carbohydrate, etc. The composition can be changed by a chemical, a physical, or a biological method or by being produced from a cell previously or concurrently modified by a chemical, a physical, or a biological method. Specifically, the composition can be changed by a genetic engineering or by being produced from a cell previously modified by genetic engineering. In some aspects, a surface-engineered EV comprises one or more exogenous biologically active molecules. In certain aspects, the exogenous biologically active molecules can comprise an exogenous protein (i.e., a protein that the EV does not naturally express) or a fragment or variant thereof that can be exposed to the surface of the EV or can be an anchoring point (attachment) for a moiety exposed on the surface of the EV, e.g., exosome. In other aspects, a surface-engineered EV comprises a higher expression (e.g., higher number) of a natural exosome protein (e.g., Scaffold X) or a fragment or variant thereof that can be exposed to the surface of the EV or can be an anchoring point (attachment) for a moiety exposed on the surface of the EV, e.g., exosome.

As used herein the term “lumen-engineered exosome” (e.g., Scaffold Y-engineered exosome) refers to an EV with the membrane or the lumen of the EV modified in its composition so that the lumen of the engineered EV is different from that of the EV prior to the modification or of the naturally occurring EV, e.g., exosome. The engineering can be directly in the lumen or in the membrane of the EV, e.g., exosome so that the lumen of the EV, e.g., exosome is changed. For example, the membrane is modified in its composition of a protein, a lipid, a small molecule, a carbohydrate, etc. so that the lumen of the EV, e.g., exosome is modified. The composition can be changed by a chemical, a physical, or a biological method or by being produced from a cell previously modified by a chemical, a physical, or a biological method. Specifically, the composition can be changed by a genetic engineering or by being produced from a cell previously modified by genetic engineering. In some aspects, a lumen-engineered exosome comprises one or more exogenous biologically active molecules. In certain aspects, the exogenous biologically active molecules can comprise an exogenous protein (i.e., a protein that the EV, e.g., exosome does not naturally express) or a fragment or variant thereof that can be exposed in the lumen of the EV, e.g., exosome or can be an anchoring point (attachment) for a moiety exposed on the inner layer of the EV, e.g., exosome. In other aspects, a lumen-engineered EV comprises a higher expression of a natural exosome protein (e.g., Scaffold X or Scaffold Y) or a fragment or variant thereof that can be exposed to the lumen of the exosome or can be an anchoring point (attachment) for a moiety exposed in the lumen of the exosome.

The term “modified,” when used in the context of EVs, e.g., exosomes described herein, refers to an alteration or engineering of an EV, e.g., exosome and/or its producer cell, such that the modified EV, e.g., exosome is different from a naturally-occurring EV, e.g., exosome. In some aspects, a modified EV, e.g., exosome described herein comprises a membrane that differs in composition of a protein, a lipid, a small molecular, a carbohydrate, etc. compared to the membrane of a naturally-occurring EV, e.g., exosome (e.g., membrane comprises higher density or number of natural exosome proteins and/or membrane comprises multiple (e.g., at least two) biologically active molecules that are not naturally found in exosomes (e.g., therapeutic molecules (e.g., antigen), targeting moiety, adjuvant, and/or immune modulator). As used herein, biologically active molecules that are not naturally found in exosomes are also described as “exogenous biologically active molecules.” In certain aspects, such modifications to the membrane changes the exterior surface of the EV, e.g., exosome (e.g., surface-engineered EVs, e.g., exosomes described herein). In certain aspects, such modifications to the membrane changes the lumen of the EV, e.g., exosome (e.g., lumen-engineered EVs, e.g., exosomes described herein).

As used herein the terms “modified protein” or “protein modification” refers to a protein having at least 15% identity to the non-mutant amino acid sequence of the protein. A modification of a protein includes a fragment or a variant of the protein. A modification of a protein can further include chemical, or physical modification to a fragment or a variant of the protein.

As used herein, the terms “modulate,” “modify,” and grammatical variants thereof, generally refer when applied to a specific concentration, level, expression, function or behavior, to the ability to alter, by increasing or decreasing, e.g., directly or indirectly promoting/stimulating/up-regulating or interfering with/inhibiting/down-regulating the specific concentration, level, expression, function or behavior, such as, e.g., to act as an antagonist or agonist. In some instances a modulator can increase and/or decrease a certain concentration, level, activity or function relative to a control, or relative to the average level of activity that would generally be expected or relative to a control level of activity.

As used herein, the terms “binding moiety,” “bio-distribution modifying agent,” and “targeting moiety” are interchangeable and refer to an agent that can modify the distribution of extracellular vesicles (e.g., exosomes, nanovesicles) in vivo or in vitro (e.g., in a mixed culture of cells of different varieties). The targeting moiety can be a biological molecule, such as a protein, a peptide, a lipid, or a synthetic molecule. For example, the targeting moiety can be an antibody (e.g., anti-CD22 nanobody), a synthetic polymer (e.g., PEG), a natural ligand (e.g., CD40L, albumin), a recombinant protein (e.g., XTEN), but not limited thereto. Without being bound to any particular theory, a targeting moiety disclosed herein can modify the distribution of an EV by binding to a marker (also referred to herein as a “target molecule”) expressed on a specific cell type (e.g., a CNS cell, an eye cell, a muscle cell, a macrophage, a cancer cell, or any cell specific to a certain tissue). In some aspects, a targeting moiety disclosed herein binds to a marker for a specific population of immune cells (e.g., CD4+ T cells and/or CD8+ T cells). In certain aspects, the marker is expressed only on CD4+ T cells and/or CD8+ T cells. In some aspects, a marker comprises a CD3 molecule. Accordingly, in certain aspects, a targeting moiety that can be used to increase the distribution of EVs to CD3-expressing immune cells (e.g., CD4+ T cells and/or CD8+ T cells) comprises an anti-CD3 antibody. In certain aspects, the targeting moiety is displayed on the surface of EVs. In some aspects, the targeting moiety can be displayed on the EV surface by being fused to a scaffold protein (e.g., Scaffold X) (e.g., as a genetically encoded fusion molecule). In other aspects, the targeting moiety can be displayed on the EV surface by chemical reaction attaching the targeting moiety to an EV surface molecule. A non-limiting example is PEGylation. In some aspects, a targeting moiety disclosed herein can be combined with a functional moiety, such as a small molecule (e.g., STING, ASO), a drug, and/or a therapeutic protein (e.g., anti-mesothelin antibody/pro-apoptotic proteins).

A “recombinant” polypeptide or protein refers to a polypeptide or protein produced via recombinant DNA technology. Recombinantly produced polypeptides and proteins expressed in engineered host cells are considered isolated for the purpose of the disclosure, as are native or recombinant polypeptides which have been separated, fractionated, or partially or substantially purified by any suitable technique. The polypeptides disclosed herein can be recombinantly produced using methods known in the art. Alternatively, the proteins and peptides disclosed herein can be chemically synthesized. In some aspects of the present disclosure, the Scaffold X and/or Scaffold Y proteins present in EVs are recombinantly produced by overexpressing the scaffold proteins in the producer cells, so that levels of scaffold proteins in the resulting EVs, e.g., exosomes are significantly increased with respect to the levels of scaffold proteins present in EVs of producer cells not overexpressing such scaffold proteins.

As used herein, the term “CD3” or “cluster of differentiation 3” refers to the protein complex associated with the T cell receptor (TCR). The CD3 molecule is made up of four distinct chains (CD3y, CD3δ, and two CD3ε chains). These chains associate with the T-cell receptor (TCR) and the ζ-chain to generate an activation signal in T lymphocytes. The TCR, ç-chain, and CD3 molecules together constitute the TCR complex. CD3 molecules are expressed on all T cells, including both CD4+ T cells and CD8+ T cells. Unless indicated otherwise, CD3, as used herein, can refer to CD3 from one or more species (e.g., humans, non-human primates, dogs, cats, guinea pigs, rabbits, rats, mice, horses, cattle, and bears).

As used herein, the term “CD40 ligand,” “CD40L,” “cluser of differentiation 40 ligand,” or “CD154” refers to a cytokine that acts as a ligand to CD40/tissue necrosis factor receptor SF5. CD40L is known to costimulate T-cell proliferation and cytokine production, including production of IL4 and IL10. CD40L is also capable of activating NF-Kappa-B, activating kinases MAPK8 and PAK2 in T-cells, inducing tyrosine phosphorylation of isoform 3 of CD28, mediating B-cell proliferation in the absence of co-stimulus, and mediating IgE production in the presence of IL4. Unless indicated otherwise, CD40L, as used herein, can refer to CD40L from one or more species (e.g., humans (UniProtKB - P29965), non-human primates, dogs, cats, guinea pigs, rabbits, rats, mice, horses, cattle, and bears).

As used herein, the term “CD47” or “Leukocyte surface antigen CD47” refers to a cell surface antigen that may inhibit uptake of a cell by a macrophage. CD47 is sometimes referred to as the “don’t eat me” antigen, as it is a marker of self that may play a role in preventing premature elimination of red blood cells. Unless indicated otherwise, CD47, as used herein, can refer to CD47 from one or more species (e.g., humans (UniProtKB - Q08722), non-human primates, dogs, cats, guinea pigs, rabbits, rats, mice, horses, cattle, and bears).

As used herein, the term “CD24” or “signal transducer CD24” refers to a cell surface antigen that plays a role in the control of autoimmunity. CD24 is believed to modulate B-cell activation responses, promote AG-dependent proliferation of B-cells, and prevent B-cell terminal differentiation into antibody-forming cells. In association with SIGLEC10, CD24 may be involved in the selective suppression of the immune response to danger-associated molecular patterns (DAMPs) such as HMGB 1, HSP70 and HSP90.Unless indicated otherwise, CD24, as used herein, can refer to CD24 from one or more species (e.g., humans (UniProtKB - P25063), non-human primates, dogs, cats, guinea pigs, rabbits, rats, mice, horses, cattle, and bears).

As used herein, the term “scaffold moiety” refers to a molecule that can be used to anchor a payload or any other exogenous biologically active molecule of interest (e.g., targeting moiety, adjuvant, and/or immune modulator) to the EV either on the luminal surface or on the exterior surface of the EV, e.g., exosome. In certain aspects, a scaffold moiety comprises a synthetic molecule. In some aspects, a scaffold moiety comprises a non-polypeptide moiety. In other aspects, a scaffold moiety comprises a lipid, carbohydrate, or protein that naturally exists in the EV, e.g., exosome. In some aspects, a scaffold moiety comprises a lipid, carbohydrate, or protein that does not naturally exist in the EV, e.g., exosome. In certain aspects, a scaffold moiety is Scaffold X. In some aspects, a scaffold moiety is Scaffold Y. In further aspects, a scaffold moiety comprises both Scaffold X and Scaffold Y. Non-limiting examples of other scaffold moieties that can be used with the present disclosure include: aminopeptidase N (CD13); Neprilysin, AKA membrane metalloendopeptidase (MME); ectonucleotide pyrophosphatase/phosphodiesterase family member 1 (ENPP1); Neuropilin-1 (NRP1); CD9, CD63, CD81, PDGFR, GPI anchor proteins, lactadherin, LAMP2, and LAMP2B. In some aspects, the scaffold protein is a fusion protein, comprising (i) a naturally occurring EV protein or a fragment thereof and (ii) a heterologous peptide (e.g., an antigen binding domain, a capsid protein, an Fc receptor, a binding partner of a chemically induced dimer, or any combination thereof).

As used herein, the term “binding partner” refers to one member of at least two elements that interact with each other to form a multimer (e.g., a dimer). In some aspects, the binding partner is a first binding partner that interacts with a second binding partner. In some aspects, the binding partner is a first binding partner that interacts with a second binding partner and/or a third binding partner. Any binding partners can be used in the compositions and methods disclosed herein. In some aspects, the binding partner can be a polypeptide, a polynucleotide, a fatty acid, a small molecule, or any combination thereof. In certain aspects, the binding partner (e.g., the first binding partner and/or the second binding partner) is selected from a first and a second binding partners of a chemically induced dimer selected from the group consisting of (i) FKBP and FKBP (FK1012); (ii) FKBP and CalcineurinA (CNA) (FK506); (iii) FKBP and CyP-Fas (FKCsA); (iv) FKBP and FRB (Rapamycin); (v) GyrB and GyrB (Coumermycin); (vi) GAI and GID1 (Gibberellin); (vii) Snap-tag and HaloTag (HaXS); (viii) eDHFR and HaloTag (TMP-HTag); and (ix) BCL-xL and Fab (AZ1) (ABT-737).

As used herein, the term “Scaffold X” refers to exosome proteins that have recently been identified on the surface of exosomes. See, e.g., U.S. Pat. No. 10,195,290, which is incorporated herein by reference in its entirety. Non-limiting examples of Scaffold X proteins include: prostaglandin F2 receptor negative regulator (“the PTGFRN protein”); basigin (“the BSG protein”); immunoglobulin superfamily member 2 (“the IGSF2 protein”); immunoglobulin superfamily member 3 (“the IGSF3 protein”); immunoglobulin superfamily member 8 (“the IGSF8 protein”); integrin beta-1 (“the ITGB1 protein); integrin alpha-4 (“the ITGA4 protein”); 4F2 cell-surface antigen heavy chain (“the SLC3A2 protein”); and a class of ATP transporter proteins (“the ATP1A1 protein,” “the ATP1A2 protein,” “the ATP1A3 protein,” “the ATP1A4 protein,” “the ATP1B3 protein,” “the ATP2B1 protein,” “the ATP2B2 protein,” “the ATP2B3 protein,” “the ATP2B protein”). In some aspects, a Scaffold X protein can be a whole protein or a fragment thereof (e.g., functional fragment, e.g., the smallest fragment that is capable of anchoring another moiety on the exterior surface or on the luminal surface of the EV, e.g., exosome). In some aspects, a Scaffold X can anchor an exogenous protein (e.g., those disclosed herein, e.g., targeting moiety, therapeutic molecule, adjuvant, and/or immune modulator) to the external surface or the luminal surface of the exosome.

As used herein, the term “Scaffold Y” refers to exosome proteins that were newly identified within the lumen of exosomes. See, e.g., International Publication No. WO/2019/099942, which is incorporated herein by reference in its entirety. Non-limiting examples of Scaffold Y proteins include: myristoylated alanine rich Protein Kinase C substrate (“the MARCKS protein”); myristoylated alanine rich Protein Kinase C substrate like 1 (“the MARCKSL1 protein”); and brain acid soluble protein 1 (“the BASP1 protein”). In some aspects, a Scaffold Y protein can be a whole protein or a fragment thereof (e.g., functional fragment, e.g., the smallest fragment that is capable of anchoring a moiety to the luminal surface of the exosome). In some aspects, a Scaffold Y can anchor an exogenous protein (e.g., those disclosed herein, e.g., targeting moiety, therapeutic molecule, adjuvant, and/or immune modulator) to the luminal surface of the EV, e.g., exosome.

As used herein, the term “fragment” of a protein (e.g., therapeutic protein, Scaffold X, or Scaffold Y) refers to an amino acid sequence of a protein that is shorter than the naturally-occurring sequence, N- and/or C-terminally deleted or any part of the protein deleted in comparison to the naturally occurring protein. As used herein, the term “functional fragment” refers to a protein fragment that retains protein function. Accordingly, in some aspects, a functional fragment of a Scaffold X protein retains the ability to anchor a moiety on the luminal surface or on the exterior surface of the EV, e.g., exosome. Similarly, in certain aspects, a functional fragment of a Scaffold Y protein retains the ability to anchor a moiety on the luminal surface of the EV, e.g., exosome. Whether a fragment is a functional fragment can be assessed by any art known methods to determine the protein content of EVs, e.g., exosomes including Western Blots, FACS analysis and fusions of the fragments with autofluorescent proteins like, e.g., GFP. In certain aspects, a functional fragment of a Scaffold X protein retains at least about 50%, at least about 60%, at least about 70%, at least about 80%, at least about 90% or at least about 100% of the ability, e.g., an ability to anchor a moiety, of the naturally occurring Scaffold X protein. In some aspects, a functional fragment of a Scaffold Y protein retains at least about 50%, at least about 60%, at least about 70%, at least about 80%, at least about 90% or at least about 100% of the ability, e.g., an ability to anchor another molecule, of the naturally occurring Scaffold Y protein.

As used herein, the term “variant” of a molecule (e.g., functional molecule, therapeutic molecule, Scaffold X and/or Scaffold Y) refers to a molecule that shares certain structural and functional identities with another molecule upon comparison by a method known in the art. For example, a variant of a protein can include a substitution, insertion, deletion, frameshift or rearrangement in another protein.

In some aspects, a variant of a Scaffold X, or a derivative thereof, comprises a variant having at least about 70% identity to the full-length, mature PTGFRN, BSG, IGSF2, IGSF3, IGSF8, ITGB1, ITGA4, SLC3A2, or ATP transporter proteins or a fragment (e.g., functional fragment) of the PTGFRN, BSG, IGSF2, IGSF3, IGSF8, ITGB1, ITGA4, SLC3A2, or ATP transporter proteins. In some aspects, variants or variants of fragments of PTGFRN share at least about 70%, at least about 80%, at least about 85%, at least about 90%, at least about 95%, at least about 96%, at least about 97%, at least about 98%, or at least about 99% sequence identity with PTGFRN according to SEQ ID NO: 1 or with a functional fragment thereof. In some aspects variants or variants of fragments of BSG share at least about 70%, at least about 80%, at least about 85%, at least about 90%, at least about 95%, at least about 96%, at least about 97%, at least about 98%, or at least about 99% sequence identity with BSG according to SEQ ID NO: 9 or with a functional fragment thereof. In some aspects variants or variants of fragments of IGSF2 share at least about 70%, at least about 80%, at least about 85%, at least about 90%, at least about 95%, at least about 96%, at least about 97%, at least about 98%, or at least about 99% sequence identity with IGSF2 according to SEQ ID NO: 34 or with a functional fragment thereof. In some aspects variants or variants of fragments of IGSF3 share at least about 70%, at least about 80%, at least about 85%, at least about 90%, at least about 95%, at least about 96%, at least about 97%, at least about 98%, or at least about 99% sequence identity with IGSF3 or with a functional fragment thereof. In some aspects variants or variants of fragments of IGSF8 share at least about 70%, at least about 80%, at least about 85%, at least about 90%, at least about 95%, at least about 96%, at least about 97%, at least about 98%, or at least about 99% sequence identity with IGSF8 according to SEQ ID NO: 14 or with a functional fragment thereof. In some aspects variants or variants of fragments of ITGB 1 share at least about 70%, at least about 80%, at least about 85%, at least about 90%, at least about 95%, at least about 96%, at least about 97%, at least about 98%, or at least about 99% sequence identity with ITGB1 according to SEQ ID NO: 21 or with a functional fragment thereof. In some aspects variants or variants of fragments of ITGA4 share at least about 70%, at least about 80%, at least about 85%, at least about 90%, at least about 95%, at least about 96%, at least about 97%, at least about 98%, or at least about 99% sequence identity with ITGA4 according to SEQ ID NO: 22 or with a functional fragment thereof. In some aspects variants or variants of fragments of SLC3A2 share at least about 70%, at least about 80%, at least about 85%, at least about 90%, at least about 95%, at least about 96%, at least about 97%, at least about 98%, or at least about 99% sequence identity with SLC3A2 according to SEQ ID NO: 23 or with a functional fragment thereof. In some aspects variants or variants of fragments of ATP1A1 share at least about 70%, at least about 80%, at least about 85%, at least about 90%, at least about 95%, at least about 96%, at least about 97%, at least about 98%, or at least about 99% sequence identity with ATP1A1 or with a functional fragment thereof. In some aspects variants or variants of fragments of ATP1A2 share at least about 70%, at least about 80%, at least about 85%, at least about 90%, at least about 95%, at least about 96%, at least about 97%, at least about 98%, or at least about 99% sequence identity with ATP1A2 or with a functional fragment thereof. In some aspects variants or variants of fragments of ATP1A3 share at least about 70%, at least about 80%, at least about 85%, at least about 90%, at least about 95%, at least about 96%, at least about 97%, at least about 98%, or at least about 99% sequence identity with ATP1A3 or with a functional fragment thereof. In some aspects variants or variants of fragments of ATP1A4 share at least about 70%, at least about 80%, at least about 85%, at least about 90%, at least about 95%, at least about 96%, at least about 97%, at least about 98%, or at least about 99% sequence identity with ATP1A4 or with a functional fragment thereof. In some aspects variants or variants of fragments of ATP1B3 share at least about 70%, at least about 80%, at least about 85%, at least about 90%, at least about 95%, at least about 96%, at least about 97%, at least about 98%, or at least about 99% sequence identity with ATP1B3 or with a functional fragment thereof. In some aspects variants or variants of fragments of ATP2B1 share at least about 70%, at least about 80%, at least about 85%, at least about 90%, at least about 95%, at least about 96%, at least about 97%, at least about 98%, or at least about 99% sequence identity with ATP2B1 or with a functional fragment thereof. In some aspects variants or variants of fragments of ATP2B2 share at least about 70%, at least about 80%, at least about 85%, at least about 90%, at least about 95%, at least about 96%, at least about 97%, at least about 98%, or at least about 99% sequence identity with ATP2B2 or with a functional fragment thereof. In some aspects variants or variants of fragments of ATP2B3 share at least about 70%, at least about 80%, at least about 85%, at least about 90%, at least about 95%, at least about 96%, at least about 97%, at least about 98%, or at least about 99% sequence identity with ATP2B3 or with a functional fragment thereof. In some aspects variants or variants of fragments of ATP2B4 share at least about 70%, at least about 80%, at least about 85%, at least about 90%, at least about 95%, at least about 96%, at least about 97%, at least about 98%, or at least about 99% sequence identity with ATP2B4 or with a functional fragment thereof. In some aspects, the variant or variant of a fragment of Scaffold X protein disclosed herein retains the ability to be specifically targeted to EVs, e.g., exosomes. In some aspects, the Scaffold X includes one or more mutations, for example, conservative amino acid substitutions.

In some aspects, a variant of a Scaffold Y, or a derivative thereof, comprises a variant having at least 70% identity to MARCKS, MARCKSL1, BASP1 or a fragment of MARCKS, MARCKSL1, or BASP1. In some aspects variants or variants of fragments of MARCKS share at least about 70%, at least about 80%, at least about 85%, at least about 90%, at least about 95%, at least about 96%, at least about 97%, at least about 98%, or at least about 99% sequence identity with MARCKS according to SEQ ID NO: 47 or with a functional fragment thereof. In some aspects variants or variants of fragments of MARCKSL1 share at least about 70%, at least about 80%, at least about 85%, at least about 90%, at least about 95%, at least about 96%, at least about 97%, at least about 98%, or at least about 99% sequence identity with MARCKSL1 according to SEQ ID NO: 48 or with a functional fragment thereof. In some aspects variants or variants of fragments of BASP1 share at least about 70%, at least about 80%, at least about 85%, at least about 90%, at least about 95%, at least about 96%, at least about 97%, at least about 98%, or at least about 99% sequence identity with BASP1 according to SEQ ID NO: 49 or with a functional fragment thereof. In some aspects, the variant or variant of a fragment of Scaffold Y protein, or a derivative thereof, retains the ability to be specifically targeted to the luminal surface of EVs, e.g., exosomes. In some aspects, the Scaffold Y includes one or more mutations, e.g., conservative amino acid substitutions.

In some aspects, the scaffold protein is a transmembrane protein. As used herein, a “transmembrane protein” refers to any protein that comprises an extracellular domain (e.g., at least one amino acid that is located external to the membrane of the EV e.g., extra-vesicular), a transmembrane domain (e.g., at least one amino acid that is located within the membrane of an EV, e.g., within the membrane of an exosome), and an intracellular domain (e.g., at least one amino acid that is located internal to the membrane of the EV, e.g., exosome). In some aspects, a scaffold protein described herein is a type I transmembrane protein, wherein the N-terminus of the transmembrane protein is located in the extracellular space, e.g., outside the membrane the encloses the EV e.g., extra-vesicular. In some aspects, a scaffold protein described herein is a type II transmembrane protein, wherein the N-terminus of the transmembrane protein is located in the intracellular space, e.g., inside the membrane, e.g., on the luminal side of the membrane, that encloses the EV e.g., intra-vesicular.

The term “spacer” as used herein refers to a bifunctional chemical moiety which is capable of covalently linking together two spaced moieties (e.g., a cleavable linker and a biologically active molecule) into a normally stable dipartate molecule.

The term “self-immolative spacer” as used herein refers to a spacer as defined below that will spontaneously separate from the second moiety (e.g., a biologically active molecule) if its bond to the first moiety (e.g., a cleavable linker) is cleaved.

The term “amino acid substitution” refers to replacing an amino acid residue present in a parent or reference sequence (e.g., a wild type sequence) with another amino acid residue. An amino acid can be substituted in a parent or reference sequence (e.g., a wild type polypeptide sequence), for example, via chemical peptide synthesis or through recombinant methods known in the art. Accordingly, a reference to a “substitution at position X” refers to the substitution of an amino acid present at position X with an alternative amino acid residue. In some aspects, substitution patterns can be described according to the schema AnY, wherein A is the single letter code corresponding to the amino acid naturally or originally present at position n, and Y is the substituting amino acid residue. In other aspects, substitution patterns can be described according to the schema An(YZ), wherein A is the single letter code corresponding to the amino acid residue substituting the amino acid naturally or originally present at position n, and Y and Z are alternative substituting amino acid residues that can replace A.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another aspect, a string of amino acids can be conservatively replaced with a structurally similar string that differs in order and/or composition of side chain family members.

As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art. It is understood that percentage of similarity is contingent on the comparison scale used, i.e., whether the amino acids are compared, e.g., according to their evolutionary proximity, charge, volume, flexibility, polarity, hydrophobicity, aromaticity, isoelectric point, antigenicity, or combinations thereof.

In some aspects, two or more sequences are said to be “completely conserved” or “identical” if they are 100% identical to one another. In some aspects, two or more sequences are said to be “highly conserved” if they are at least about 70% identical, at least about 80% identical, at least about 90% identical, or at least about 95% identical to one another. In some aspects, two or more sequences are said to be “conserved” if they are at least about 30% identical, at least about 40% identical, at least about 50% identical, at least about 60% identical, at least about 70% identical, at least about 80% identical, at least about 90% identical, or at least about 95% identical to one another. Conservation of sequence can apply to the entire length of an polynucleotide or polypeptide or can apply to a portion, region or feature thereof.

The term “percent sequence identity” or “percent identity” between two polynucleotide or polypeptide sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence.

As used herein, the term “homology” refers to the overall relatedness between polymeric molecules, e.g. between nucleic acid molecules (e.g. DNA molecules and/or RNA molecules) and/or between polypeptide molecules. Generally, the term “homology” implies an evolutionary relationship between two molecules. Thus, two molecules that are homologous will have a common evolutionary ancestor. In the context of the present disclosure, the term homology encompasses both to identity and similarity.

In some aspects, polymeric molecules are considered to be “homologous” to one another if at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% of the monomers in the molecule are identical (exactly the same monomer) or are similar (conservative substitutions). The term “homologous” necessarily refers to a comparison between at least two sequences (polynucleotide or polypeptide sequences).

In the context of the present disclosure, substitutions (even when they are referred to as amino acid substitution) are conducted at the nucleic acid level, i.e., substituting an amino acid residue with an alternative amino acid residue is conducted by substituting the codon encoding the first amino acid with a codon encoding the second amino acid.

The percentage of sequence identity is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences can be accomplished using readily available software both for online use and for download. Suitable software programs are available from various sources, and for alignment of both protein and nucleotide sequences. One suitable program to determine percent sequence identity is bl2seq, part of the BLAST suite of programs available from the U.S. government’s National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In certain aspects, the percentage identity (%ID) or of a first amino acid sequence (or nucleic acid sequence) to a second amino acid sequence (or nucleic acid sequence) is calculated as %ID = 100 x (Y/Z), where Y is the number of amino acid residues (or nucleobases) scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. Sequence alignments can be derived from multiple sequence alignments. One suitable program to generate multiple sequence alignments is ClustalW2, available from www.clustal.org. Another suitable program is MUSCLE, available from www.drive5.com/muscle/. ClustalW2 and MUSCLE are alternatively available, e.g., from the EBI.

It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

The polynucleotide variants can contain alterations in the coding regions, non-coding regions, or both. In one aspect, the polynucleotide variants contain alterations which produce silent substitutions, additions, or deletions, but do not alter the properties or activities of the encoded polypeptide. In another aspect, nucleotide variants are produced by silent substitutions due to the degeneracy of the genetic code. In other aspects, variants in which 5-10, 1-5, or 1-2 amino acids are substituted, deleted, or added in any combination. Polynucleotide variants can be produced for a variety of reasons, e.g., to optimize codon expression for a particular host (change codons in the human mRNA to others, e.g., a bacterial host such as E. coli).

Naturally occurring variants are called “allelic variants,” and refer to one of several alternate forms of a gene occupying a given locus on a chromosome of an organism (Genes II, Lewin, B., ed., John Wiley & Sons, New York (1985)). These allelic variants can vary at either the polynucleotide and/or polypeptide level and are included in the present disclosure. Alternatively, non-naturally occurring variants can be produced by mutagenesis techniques or by direct synthesis.

Using known methods of protein engineering and recombinant DNA technology, variants can be generated to improve or alter the characteristics of the polypeptides. For instance, one or more amino acids can be deleted from the N-terminus or C-terminus of the secreted protein without substantial loss of biological function. Ron et al., J. Biol. Chem. 268: 2984-2988 (1993), incorporated herein by reference in its entirety, reported variant KGF proteins having heparin binding activity even after deleting 3, 8, or 27 amino-terminal amino acid residues. Similarly, interferon gamma exhibited up to ten times higher activity after deleting 8-10 amino acid residues from the carboxy terminus of this protein. (Dobeli et al., J. Biotechnology 7:199-216 (1988), incorporated herein by reference in its entirety.)

Moreover, ample evidence demonstrates that variants often retain a biological activity similar to that of the naturally occurring protein. For example, Gayle and coworkers (J. Biol. Chem 268:22105-22111 (1993), incorporated herein by reference in its entirety) conducted extensive mutational analysis of human cytokine IL-1a. They used random mutagenesis to generate over 3,500 individual IL-1a mutants that averaged 2.5 amino acid changes per variant over the entire length of the molecule. Multiple mutations were examined at every possible amino acid position. The investigators found that “[m]ost of the molecule could be altered with little effect on either [binding or biological activity].” (See Abstract.) In fact, only 23 unique amino acid sequences, out of more than 3,500 nucleotide sequences examined, produced a protein that significantly differed in activity from wild-type.

As stated above, polypeptide variants include, e.g., modified polypeptides. In some aspects, variants or derivatives of, e.g., polypeptides, polynucleotides, lipids, glycoproteins, are the result of chemical modification and/or endogenous modification. In some aspects, variants or derivatives are the result of in vivo modification. In some aspects, variants or derivatives are the result of in vitro modification. In yet other aspects, variant or derivatives are the result of intracellular modification in producer cells. Modifications present in variants and derivatives include, e.g., acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation (Mei et al., Blood 116:270-79 (2010), which is incorporated herein by reference in its entirety), proteolytic processing, phosphorylation, prenylation, racemization, selenoylation, sulfation, transfer-RNA mediated addition of amino acids to proteins such as arginylation, and ubiquitination. In some aspects, Scaffold X and/or Scaffold Y is modified at any convenient location.

As used herein the terms “linked to,” “fused to,” “conjugated to,” and “anchored to” are used interchangeably and refer to a covalent or non-covalent bond formed between a first moiety and a second moiety, e.g., Scaffold X and a targeting moiety disclosed herein. The first moiety can be directly joined or juxtaposed to the second moiety or alternatively an intervening moiety can covalently join the first moiety to the second moiety. The term “linked” means not only a fusion of a first moiety to a second moiety at the C-terminus or the N-terminus, but also includes insertion of the whole first moiety (or the second moiety) into any two points, e.g., amino acids, in the second moiety (or the first moiety, respectively). In one aspect, the first moiety is linked to a second moiety by a peptide bond or a linker. The first moiety can be linked to a second moiety by a phosphodiester bond or a linker. The linker can be a peptide or a polypeptide (for polypeptide chains) or a nucleotide or a nucleotide chain (for nucleotide chains) or any chemical moiety (for polypeptide or polynucleotide chains or any chemical molecules). The term “linked” is also indicated by a hyphen (-). In some aspects, a Scaffold X protein on an EV can be linked or fused to a biologically active molecule via a maleimide moiety.

As used herein “anchoring” a biologically active molecule on the luminal or external surface of an EV of the present disclosure via, e.g., a scaffold protein, refers to attaching covalently or non-covalently the biologically active molecule to the portion of the scaffold molecule located on the luminal or external surface of the EV, respectively, or to an anchoring moiety (e.g., cholesterol).

The term “anchored,” as used herein, refers to an element that is associated with the membrane. In some aspects, the element that is anchored to the membrane is associated with a transmembrane protein, wherein the transmembrane protein anchors the element to the membrane. In some aspects, the element that is anchored to the membrane is associated with a scaffold protein that comprises a motif (e.g., a scaffold protein comprising GGKLSKK (SEQ ID NO: 17)) that interacts with the membrane, thereby anchoring the element to the membrane. In some aspects, the scaffold protein comprises a myristoylated amino acid residue at the N terminus of the scaffold protein, wherein the myristoylated amino acid anchors the scaffold protein to the membrane of the EV. An element can be anchored directly (e.g. a peptide bond) or by a linker to the membrane.

The term “encapsulated”, or grammatically different forms of the term (e.g., encapsulation, or encapsulating), refer s to a status or process of having a first moiety (e.g., exogenous biologically active molecule, e.g., therapeutic molecule, adjuvant, or immune modulator) inside a second moiety (e.g., an EV, e.g., exosome) without chemically or physically linking the two moieties. In some aspects, the term “encapsulated” can be used interchangeably with “in the lumen of”. Non-limiting examples of encapsulating a first moiety (e.g., exogenous biologically active molecule, e.g., therapeutic molecule, adjuvant, or immune modulator) into a second moiety (e.g., EVs, e.g., exosomes) are disclosed elsewhere herein.

As used herein, the term “extracellular” can be used interchangeably with the terms “external,” “exterior,” and “extra-vesicular,” wherein each term refers to an element that is outside the membrane that encloses the EV. As used herein, the term “intracellular” can be used interchangeably with the terms “internal,” “interior,” and “intra-vesicular,” wherein each term refers to an element that is inside the membrane that encloses the EV. The term “lumen” refers to the space inside the membrane enclosing the EV. Accordingly, an element that is inside the lumen of an EV can be referred to herein as being “located in the lumen” or “luminal.”

As used herein, the term “producer cell” refers to a cell used for generating an EV, e.g., exosome. A producer cell can be a cell cultured in vitro, or a cell in vivo. A producer cell includes, but not limited to, a cell known to be effective in generating EVs e.g., HEK293 cells, Chinese hamster ovary (CHO) cells, mesenchymal stem cells (MSCs), BJ human foreskin fibroblast cells, fHDF fibroblast cells, AGE.HN® neuronal precursor cells, CAP® amniocyte cells, adipose mesenchymal stem cells, RPTEC/TERT1 cells. In certain aspects, a producer cell is not an antigen-presenting cell. In some aspects, a producer cell is not a dendritic cell, a B cell, a mast cell, a macrophage, a neutrophil, Kupffer-Browicz cell, cell derived from any of these cells, or any combination thereof. In some aspects, the EVs, e.g., exosomes useful in the present disclosure do not carry an antigen on MHC class I or class II molecule exposed on the surface of the EV but instead can carry an antigen in the lumen of the EV, e.g., exosome or on the surface of the EV, e.g., exosome by attachment to Scaffold X and/or Scaffold Y.

As used herein, the terms “isolate,” “isolated,” and “isolating” or “purify,” “purified,” and “purifying” as well as “extracted” and “extracting” are used interchangeably and refer to the state of a preparation (e.g., a plurality of known or unknown amount and/or concentration) of desired EVs, that have undergone one or more processes of purification, e.g., a selection or an enrichment of the desired EV preparation. In some aspects, isolating or purifying as used herein is the process of removing, partially removing (e.g., a fraction) of the EVs from a sample containing producer cells. In some aspects, an isolated EV composition has no detectable undesired activity or, alternatively, the level or amount of the undesired activity is at or below an acceptable level or amount. In other aspects, an isolated EV composition has an amount and/or concentration of desired EVs at or above an acceptable amount and/or concentration. In other aspects, the isolated EV composition is enriched as compared to the starting material (e.g., producer cell preparations) from which the composition is obtained. This enrichment can be by 10%, 20%, 30%, 40%, 50%, 60%, 70%, 80%, 90%, 95%, 96%, 97%, 98%, 99%, 99.9%, 99.99%, 99.999%, 99.9999%, or greater than 99.9999% as compared to the starting material. In some aspects, isolated EV preparations are substantially free of residual biological products. In some aspects, the isolated EV preparations are 100% free, 99% free, 98% free, 97% free, 96% free, 95% free, 94% free, 93% free, 92% free, 91% free, or 90% free of any contaminating biological matter. Residual biological products can include abiotic materials (including chemicals) or unwanted nucleic acids, proteins, lipids, or metabolites. Substantially free of residual biological products can also mean that the EV composition contains no detectable producer cells and that only EVs are detectable.

As used herein, the term “immune modulator” refers to an agent that acts on a target (e.g., a target cell) that is contacted with the extracellular vesicle, and regulates the immune system. Non-limiting examples of immune modulator that can be introduced into an EV and/or a producer cell include agents such as, modulators of checkpoint inhibitors, ligands of checkpoint inhibitors, cytokines, derivatives thereof, or any combination thereof. The immune modulator can also include an agonist, an antagonist, an antibody, an antigen-binding fragment, a polynucleotide, such as siRNA, miRNA, lncRNA, mRNA, DNA, or a small molecule.

As used herein, the term “payload” refers to a biologically active molecule (e.g., a therapeutic agent) that acts on a target (e.g., a target cell) that is contacted with the EV of the present disclosure. Non-limiting examples of payload that can be included on the EV are a therapeutic molecule (e.g., antigen or immunosuppressive agent), an adjuvant, and/or an immune modulator. Payloads that can be introduced into an EV and/or a producer cell include agents such as, nucleotides (e.g., nucleotides comprising a detectable moiety or a toxin or that disrupt transcription), nucleic acids (e.g., DNA or mRNA molecules that encode a polypeptide such as an enzyme, or RNA molecules that have regulatory function such as miRNA, dsDNA, lncRNA, and siRNA,antisense oligonucleotide, a phosphorodiamidate morpholino oligomer (PMO), or a peptide-conjugated phosphorodiamidate morpholino oligomer (PPMO))), amino acids (e.g., amino acids comprising a detectable moiety or a toxin or that disrupt translation), polypeptides (e.g., enzymes), lipids, carbohydrates, and small molecules (e.g., small molecule drugs and toxins). In certain aspects, a payload comprises an exogenous biologically active molecule (e.g., those disclosed herein). In some aspects, the payload molecules are covalently linked to the EV via a maleimide moiety. In other aspects, a payload comprises an adjuvant.

As used herein, the term “biologically active molecule” refers to an agent that has activity in a biological system (e.g., a cell or a human subject), including, but not limited to a protein, polypeptide or peptide including, but not limited to, a structural protein, an enzyme, a cytokine (such as an interferon and/or an interleukin) an antibiotic, a polyclonal or monoclonal antibody, or an effective part thereof, such as an Fv fragment, which antibody or part thereof can be natural, synthetic or humanized, a peptide hormone, a receptor, a signaling molecule or other protein; a nucleic acid, as defined below, including, but not limited to, an oligonucleotide or modified oligonucleotide, an antisense oligonucleotide or modified antisense oligonucleotide, cDNA, genomic DNA, an artificial or natural chromosome (e.g. a yeast artificial chromosome) or a part thereof, RNA, including mRNA, tRNA, rRNA or a ribozyme, or a peptide nucleic acid (PNA); a virus or virus-like particles; a nucleotide or ribonucleotide or synthetic analogue thereof, which can be modified or unmodified; an amino acid or analogue thereof, which can be modified or unmodified; a non-peptide (e.g., steroid) hormone; a proteoglycan; a lipid; or a carbohydrate. In certain aspects, a biologically active molecule comprises a therapeutic molecule (e.g., an antigen), a targeting moiety (e.g., an antibody or an antigen-binding fragment thereof), an adjuvant, an immune modulator, or any combination thereof. In some aspects, the biologically active molecule comprises a macromolecule (e.g., a protein, an antibody, an enzyme, a peptide, DNA, RNA, or any combination thereof). In some aspects, the biologically active molecule comprises a small molecule (e.g., an antisense oligomer (ASO), an siRNA, STING, a pharmaceutical drug, or any combination thereof). In some aspects, the biologically active molecules are exogenous to the exosome, i.e., not naturally found in the exosome. In some aspects, the biologically active moiety is any molecule that can be attached to an EV via a maleimide moiety, wherein the molecule can have a therapeutic or prophylactic effect in a subject in need thereof, or be used for diagnostic purposes. In some aspects, the biologically active molecule is a detectable moiety, e.g., a radionuclide, a fluorescent molecule, or a contrast agent.

As used herein, “prophylactic” refers to a therapeutic or course of action used to prevent the onset of a disease or condition, or to prevent or delay a symptom associated with a disease or condition.

As used herein, a “prophylaxis” refers to a measure taken to maintain health and prevent or delay the onset of a bleeding episode, or to prevent or delay symptoms associated with a disease or condition.

As used herein, the term “therapeutic molecule” refers to any molecule that can treat and/or prevent a disease or disorder in a subject (e.g., human subject).

The term “polynucleotide” as used herein refers to polymers of nucleotides of any length, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the term “polynucleotide” includes polydeoxyribonucleotides (containing 2-deoxy-D-ribose), polyribonucleotides (containing D-ribose), including tRNA, rRNA, hRNA, siRNA and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA. In some aspects of the present disclosure, the biologically active molecule attached to the EV via a maleimide moiety is a polynucleotide, e.g., an antisense oligonucleotide. In particular aspects, the polynucleotide comprises an mRNA. In other aspect, the mRNA is a synthetic mRNA. In some aspects, the synthetic mRNA comprises at least one unnatural nucleobase. In some aspects, all nucleobases of a certain class have been replaced with unnatural nucleobases (e.g., all uridines in a polynucleotide disclosed herein can be replaced with an unnatural nucleobase, e.g., 5-methoxyuridine). In some aspects of the present disclosure, the biologically active molecule is a polynucleotide.

The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art. In some aspects of the present disclosure, the biologically active molecule attached to the EV via a maleimide moiety is a polypeptide, e.g., an antibody or a derivative thereof such as an ADC, a PROTAC, a toxin, a small molecule, a fusion protein, or an enzyme.

The term “polypeptide,” as used herein, refers to proteins, polypeptides, and peptides of any size, structure, or function. Polypeptides include gene products, naturally occurring polypeptides, synthetic polypeptides, homologs, orthologs, paralogs, fragments and other equivalents, variants, and analogs of the foregoing. A polypeptide can be a single polypeptide or can be a multi-molecular complex such as a dimer, trimer or tetramer. They can also comprise single chain or multichain polypeptides. Most commonly disulfide linkages are found in multichain polypeptides. The term polypeptide can also apply to amino acid polymers in which one or more amino acid residues are an artificial chemical analogue of a corresponding naturally occurring amino acid. In some aspects, a “peptide” can be less than or equal to 50 amino acids long, e.g., about 5, 10, 15, 20, 25, 30, 35, 40, 45, or 50 amino acids long.

In some aspects, a therapeutic molecule comprises an antigen. As used herein, the term “antigen” refers to any agent that when introduced into a subject elicits an immune response (cellular or humoral) to itself. In some aspects, an antigen is not expressed on major histocompatibility complex I and/or II molecules. In other aspects, while an antigen in the EV is not expressed as MHC class I or II complex, the EV can still contain MHC class I/II molecules on the surface of the EV, e.g., exosome. Accordingly, in certain aspects, EVs disclosed herein do not directly interact with T-cell receptors (TCRs) of T cells to induce an immune response against the antigen. Similarly, in certain aspects, EVs of the present disclosure do not transfer the antigen directly to the surface of the target cell (e.g., dendritic cell) through cross-dressing. Cross-dressing is a mechanism commonly used by EVs derived from dendritic cells (DEX) to induce T cell activation. See Pitt, J.M., et al., J Clin Invest 126(4): 1224-32 (2016). In other aspects, the EVs of the present disclosure are engulfed by antigen presenting cells and can be expressed on the surface of the antigen presenting cells as MHC class I and/or MHC class II complex.

As used herein, the term “agonist” refers to a molecule that binds to a receptor and activates the receptor to produce a biological response. Receptors can be activated by either an endogenous or an exogenous agonist. Non-limiting examples of endogenous agonist include hormones, neurotransmitters, and cyclic dinucleotides. Non-limiting examples of exogenous agonist include drugs, small molecules, and cyclic dinucleotides. The agonist can be a full, partial, or inverse agonist.

As used herein, the term “antagonist” refers to a molecule that blocks or dampens an agonist mediated response rather than provoking a biological response itself upon bind to a receptor. Many antagonists achieve their potency by competing with endogenous ligands or substrates at structurally defined binding sites on the receptors. Non-limiting examples of antagonists include alpha blockers, beta-blocker, and calcium channel blockers. The antagonist can be a competitive, non-competitive, or uncompetitive antagonist.

In some aspects, a therapeutic molecule comprises an immunosuppressive agent. As used herein, the term “immunosuppressive agent” refers to any agent (e.g., therapeutic molecule) that slows or halts an immune response in a subject. Immunosuppressive agents can be given to a subject to prevent the subject’s immune system from mounting an immune response after an organ transplant or for treating a disease that is caused by an overactive immune system. Examples of immunosuppressive agents include, but are not limited to, a calcineurin inhibitor, such as, but not limited to, cyclosporine, ISA(TX) 247, tacrolimus or calcineurin, a target of rapamycin, such as, but not limited to, sirolimus, everolimus, FK778 or TAFA-93, an interleukin-2 α-chain blocker, such as, but not limited to, basiliximab and daclizumab, an inhibitor of inosine monophosphate dehydrogenase, such as mycophenolate mofetil, an inhibitor of dihydrofolic acid reductase, such as, but not limited to, methotrexate, a corticosteroid, such as, but not limited to, prednisolone and methylprednisolone, or an immunosuppressive antimetabolite, such as, but not limited to, azathioprine. In certain aspects, an immunosuppressive agent comprises an antisense oligonucleotide. In some aspects, an EV disclosed herein (e.g., exosome) can comprise both an antigen and an immunosuppressive agent. Not to be bound by any one theory, an EV comprising both an antigen and an immunosuppressive agent can be used to induce tolerance to the antigen.

As used herein, the term “antibody” encompasses an immunoglobulin whether natural or partly or wholly synthetically produced, and fragments thereof. The term also covers any protein having a binding domain that is homologous to an immunoglobulin binding domain. “Antibody” further includes a polypeptide comprising a framework region from an immunoglobulin gene or fragments thereof that specifically binds and recognizes an antigen. Use of the term antibody is meant to include whole antibodies, polyclonal, monoclonal and recombinant antibodies, fragments thereof, and further includes single-chain antibodies, humanized antibodies, murine antibodies, chimeric, mouse-human, mouse-primate, primate-human monoclonal antibodies, anti-idiotype antibodies, antibody fragments, such as, e.g., scFv, (scFv)₂, Fab, Fab′, and F(ab′)₂, F(ab1)₂, Fv, dAb, and Fd fragments, diabodies, and antibody-related polypeptides. Antibody includes bispecific antibodies and multispecific antibodies so long as they exhibit the desired biological activity or function. In some aspects, the antibody or antigen-binding fragment thereof comprises a scFv, scFab, scFab-Fc, nanobody, or any combination thereof. In some aspects, the antibody or antigen-binding fragment thereof comprises an agonist antibody, a blocking antibody, a targeting antibody, a fragment thereof, or a combination thereof. In some aspects, the agonist antibody is a CD40L agonist. In some aspects, the blocking antibody binds a target protein selected from programmed death 1 (PD-1), programmed death ligand 1 (PD-L1), cytotoxic T-lymphocyte-associated protein 4, and any combination thereof.

Antibody includes bispecific antibodies and multispecific antibodies so long as they exhibit the desired biological activity or function. In some aspects of the present disclosure, the biologically active molecule is an antibody or a molecule comprising an antigen binding fragment thereof.

The term “immunoconjugate” as used herein refers to a compound comprising a binding molecule (e.g., an antibody) and one or more moieties, e.g., therapeutic or diagnostic moieties, chemically conjugated to the binding molecule. In general an immunoconjugate is defined by a generic formula: A-(L-M)n wherein A is a binding molecule (e.g., an antibody), L is an optional linker, and M is a heterologous moiety which can be for example a therapeutic agent, a detectable label, etc., and n is an integer. In some aspects, multiple heterologous moieties can be chemically conjugated to the different attachment points in the same binding molecule (e.g., an antibody). In other aspects, multiple heterologous moieties can be concatenated and attached to an attachment point in the binding molecule (e.g., an antibody). In some aspects, multiple heterologous moieties (being the same or different) can be conjugated to the binding molecule (e.g., an antibody).

Immunoconjugates can also be defined by the generic formula in reverse order. In some aspects, the immunoconjugate is an “antibody-Drug Conjugate” (“ADC”). In the context of the present disclosure the term “immunoconjugate” is not limited to chemically or enzymatically conjugates molecules. The term “immunoconjugate” as used in the present disclosure also includes genetic fusions. In some aspects of the present disclosure, the biologically active molecule is an immunoconjugate.

The terms “antibody-drug conjugate” and “ADC” are used interchangeably and refer to an antibody linked, e.g., covalently, to a therapeutic agent (sometimes referred to herein as agent, drug, or active pharmaceutical ingredient) or agents. In some aspects of the present disclosure, the biologically active molecule is an antibody-drug conjugate.

The terms “individual,” “subject,” “host,” and “patient,” are used interchangeably herein and refer to any mammalian subj ect for whom diagnosis, treatment, or therapy is desired, particularly humans. The compositions and methods described herein are applicable to both human therapy and veterinary applications. In some aspects, the subject is a mammal, and in other aspects the subject is a human. As used herein, a “mammalian subject” includes all mammals, including without limitation, humans, domestic animals (e.g., dogs, cats and the like), farm animals (e.g., cows, sheep, pigs, horses and the like) and laboratory animals (e.g., monkey, rats, mice, rabbits, guinea pigs and the like).

As used herein, the term “substantially free” means that the sample comprising EVs comprise less than 10% of macromolecules by mass/volume (m/v) percentage concentration. Some fractions can contain less than 0.001%, less than 0.01%, less than 0.05%, less than 0.1%, less than 0.2%, less than 0.3%, less than 0.4%, less than 0.5%, less than 0.6%, less than 0.7%, less than 0.8%, less than 0.9%, less than 1%, less than 2%, less than 3%, less than 4%, less than 5%, less than 6%, less than 7%, less than 8%, less than 9%, or less than 10% (m/v) of macromolecules.

As used herein, the term “macromolecule” means nucleic acids, contaminant proteins, lipids, carbohydrates, metabolites, or a combination thereof.

As used herein, the term “conventional EV protein” means a protein previously known to be enriched in EVs.

As used herein, the term “conventional exosome protein” means a protein previously known to be enriched in exosomes, including but is not limited to CD9, CD63, CD81, PDGFR, GPI anchor proteins, lactadherin LAMP2, and LAMP2B, a fragment thereof, or a peptide that binds thereto.

The term “derivative” as used herein refers to an EV component (e.g., a protein, such as Scaffold X and/or Scaffold Y, a lipid, or a carbohydrate) or to a biologically active molecule (e.g., a polypeptide, polynucleotide, lipid, carbohydrate, antibody or fragment thereof, PROTAC, etc.) that has been chemically modified to either introduce a reactive maleimide group or a thiol group susceptible of reaction with a maleimide group. For example, an antibody modified with a bifunctional reagent comprising (i) a group reacting, e.g., with free amino groups, and (ii) a maleimide group, could result in antibody derivative comprising a reactive maleimide group that can react with free thiol groups in a Scaffold X protein on the EV, e.g., exosome. Conversely, an Scaffold X on the EV could be modified with a bifunctional reagent comprising (i) a group reacting, e.g., with free amino groups, and (ii) a maleimide group, resulting in a Scaffold X derivative comprising a reactive maleimide group that can react with free thiol groups in a biologically active molecule, e.g., an antibody.

The terms “administration,” “administering,” and grammatical variants thereof refer to introducing a composition, such as an EV of the present disclosure, into a subject via a pharmaceutically acceptable route. Routes of administration can be intravenous, e.g., intravenous injection and intravenous infusion. Additional routes of administration include, e.g., subcutaneous, intramuscular, oral, nasal, and pulmonary administration. EVs, e.g., exosomes can be administered as part of a pharmaceutical composition comprising at least one excipient. The introduction of a composition, such as an EV of the present disclosure, into a subject is by any suitable route, including intratumorally, orally, pulmonarily, intranasally, parenterally (intravenously, intra-arterially, intramuscularly, intraperitoneally, or subcutaneously), rectally, intralymphatically, intrathecally, periocularly or topically. Administration includes self-administration and the administration by another. A suitable route of administration allows the composition or the agent to perform its intended function. For example, if a suitable route is intravenous, the composition is administered by introducing the composition or agent into a vein of the subject.

The terms “excipient” and “carrier” are used interchangeably and refer to an inert substance added to a pharmaceutical composition to further facilitate administration of a compound.

The terms “pharmaceutically-acceptable carrier,” “pharmaceutically-acceptable excipient,” and grammatical variations thereof, encompass any of the agents approved by a regulatory agency of the U.S. Federal government or listed in the U.S. Pharmacopeia for use in animals, including humans, as well as any carrier or diluent that does not cause the production of undesirable physiological effects to a degree that prohibits administration of the composition to a subject and does not abrogate the biological activity and properties of the administered compound. Included are excipients and carriers that are useful in preparing a pharmaceutical composition and are generally safe, non-toxic, and desirable.

As used herein, the term “pharmaceutical composition” refers to one or more of the compounds described herein, such as, e.g., an EV, such as exosome of the present disclosure, mixed or intermingled with, or suspended in one or more other chemical components, such as pharmaceutically-acceptable carriers and excipients. One purpose of a pharmaceutical composition is to facilitate administration of preparations of EVs to a subject.

As used herein, the “compartmental” administration refers to the localized delivery of a composition to a subject. For example, in some aspects, the compartmental administration comprises administering the compositions directly to the brain, e.g., by intracranial administration. In some aspects, the compartmental administration comprises administering the compositions directly to the spinal cord, e.g., by intrathecal administration. In some aspects, the compartmental administration comprises administering the compositions directly to the lungs, e.g., by inhalation. In some aspects, the compartmental administration comprises administering the compositions directly to gastrointestinal tract, e.g., by oral administration. In some aspects, the compartmental administration comprises administering the compositions directly to a muscle, e.g., by intramuscular administration. In some aspects, the compartmental administration comprises administering the compositions directly to an eye, e.g., by intraocular administration. In some aspects, the compartmental administration comprises administering the compositions directly to a lymph node, e.g., by intraperitoneal administration. In some aspects, the compartmental administration comprises administering the compositions directly any tissue in the body, e.g., by localized administration of the composition to the target tissue.

An “immune response,” as used herein, refers to a biological response within a vertebrate against foreign agents or abnormal, e.g., cancerous cells, which response protects the organism against these agents and diseases caused by them. An immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate’s body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell, a Th cell, a CD4+ cell, a CD8+ T cell, or a Treg cell, or activation or inhibition of any other cell of the immune system, e.g., NK cell. Accordingly an immune response can comprise a humoral immune response (e.g., mediated by B-cells), cellular immune response (e.g., mediated by T cells), or both humoral and cellular immune responses. In some aspects, an immune response is an “inhibitory” immune response. An inhibitory immune response is an immune response that blocks or diminishes the effects of a stimulus (e.g., antigen). In certain aspects, the inhibitory immune response comprises the production of inhibitory antibodies against the stimulus. In some aspects, an immune response is a “stimulatory” immune response. A stimulatory immune response is an immune response that results in the generation of effectors cells (e.g., cytotoxic T lymphocytes) that can destroy and clear a target antigen (e.g., tumor antigen or viruses).

As used herein, the term “immune cells” refers to any cells of the immune system that are involved in mediating an immune response. Non-limiting examples of immune cells include a T lymphocyte, B lymphocyte, natural killer (NK) cell, macrophage, eosinophil, mast cell, dendritic cell, neutrophil, or combination thereof. In some aspects, an immune cell expresses CD3. In certain aspects, the CD3-expressing immune cells are T cells (e.g., CD4+ T cells or CD8+ T cells). In some aspects, an immune cell that can be targeted with a targeting moiety disclosed herein (e.g., anti-CD3) comprises a naive CD4+ T cell. In some aspects, an immune cell comprises a memory CD4+ T cell. In some aspects, an immune cell comprises an effector CD4+ T cell. In some aspects, an immune cell comprises a naive CD8+ T cell. In some aspects, an immune cell comprises a memory CD8+ T cell. In some aspects, an immune cell comprises an effector CD8+ T cell.

As used herein, the term “T cell” or “T-cell” refers to a type of lymphocyte that matures in the thymus. T cells play an important role in cell-mediated immunity and are distinguished from other lymphocytes, such as B cells, by the presence of a T-cell receptor on the cell surface. T-cells include all types of immune cells expressing CD3, including T-helper cells (CD4+ cells), cytotoxic T-cells (CD8+ cells), natural killer T-cells, T-regulatory cells (Treg), and gamma-delta T cells.

“Treat,” “treatment,” or “treating,” as used herein, refers to, e.g., the reduction in severity of a disease or condition; the reduction in the duration of a disease course; the amelioration or elimination of one or more symptoms associated with a disease or condition; the provision of beneficial effects to a subject with a disease or condition, without necessarily curing the disease or condition. The term also include prophylaxis or prevention of a disease or condition or its symptoms thereof. In one aspect, the term “treating” or “treatment” means inducing an immune response in a subject against an antigen.

The terms “prevent,” “preventing,” and variants thereof as used herein, refer partially or completely delaying onset of an disease, disorder and/or condition; partially or completely delaying onset of one or more symptoms, features, or clinical manifestations of a particular disease, disorder, and/or condition; partially or completely delaying onset of one or more symptoms, features, or manifestations of a particular disease, disorder, and/or condition; partially or completely delaying progression from a particular disease, disorder and/or condition; and/or decreasing the risk of developing pathology associated with the disease, disorder, and/or condition. In some aspects, preventing an outcome is achieved through prophylactic treatment.

Unless otherwise indicated, reference to a compound that has one or more stereocenters intends each stereoisomer, and all combinations of stereoisomers, thereof.

### II. Methods of the Disclosure

### II.A. Methods of Compartmental Administration of Exosomes

Certain aspects of the present disclosure are directed to methods of directing a composition comprising an EV to a target tissue by compartmentally administering an effective amount of the composition to the subject. Some aspects of the present disclosure are directed to a method of treating a disease or disorder in a subject in need thereof, comprising compartmentally administering an effective amount of a composition comprising an EV to the subject. Compartmental administration of the composition facilitates localization of the EV to a target tissue.

Compartmental administration of the composition can be by any route known in the art effective for targeted delivery. In some aspects, the compartmental administration comprises administering the composition by a route selected from intraperitoneal, inhalation, oral, intramuscular, intrathecal, intracranial, intraocular, intradermal, sub-cutaneous, and any combination thereof. In some aspects, the compartmental administration comprises administering the composition using intra-cisterna magna administration. In some aspects, the compartmental administration comprises administering the composition using intra-cerebroventricular administration. In some aspects, intracranial administration comprises administering the composition intracranially into any normal or lesioned part of the brain. In some aspects, intracranial administration comprises administering the composition intracranially via the nasal cavity. In some aspects, intracranial administration comprises administering the composition intracranially via the the inner ear. In certain aspects, the composition is delivered intraperitoneally. In some aspects, the EV is administered intramusculary, subcutaneously or via other routes for specific routing to regional lymph nodes draining such tissue. In certain aspects, the composition is delivered by inhalation or via tracheal intubation. In certain aspects, the composition is delivered orally. In some aspects, the EV is administered rectally, or intraurethally. In certain aspects, the composition is delivered intramuscularly. In some aspects, the EV is administered intra-articularlly. In some aspects, the EV is administerd intra-articularlly into any and/or all skeletal joints, into tendons, into ligaments, or into bursas. In certain aspects, the composition is delivered intrathecally. In certain aspects, the composition is delivered intracranially. In certain aspects, the composition is delivered intraocularly. In certain aspects, the composition is delivered intradermally. In certain aspects, the composition is delivered subcutaneously.

Any tissue or population of cells can be targeted using the methods disclosed herein. In some aspects, the target tissue is selected from the central nervous system (CNS), the lungs, a muscle, an eye, the colon, a lymph node, or any combination thereof. In some aspects, the target tissue comprises the epithelial lining of the respiratoty tract. In certain aspects, the compartmental administration comprises delivery of the composition to the brain, e.g., by intracranial administration.

In certain aspects, the compartmental administration comprises delivery of the composition to the spinal chord, e.g., by intrathecal administration.

In some aspects, the EVs are administered by intrathecal administration, followed by application of a mechanical convective force to the torso. See, e.g., Verma et al., Alzheimer’s Dement. 12:e12030 (2020); which is incorporated by reference herein in its entirety). As such, certain aspects of the present disclosure are directed to methods of administering an EV, e.g., an exosome, to a subject in need thereof, comprising administering the EV to the subject by intrathecal injection, followed by applying a mechanical convective force to the torso of the subject. In some aspects, the mechanical convective force is achieved using a high frequency chest wall or lumbothoracic oscillating respiratory clearance device (e.g., a Smart Vest or Smart Wrap, ELECTROMED INC, New Prague, MN, USA). In some aspects, the mechanical convective force, e.g., the oscillating vest, facilitates spread of the intrathecally dosed EVs further down the nerve thus allowing for better EV delivery to nerves.

In some aspects, the intra- and trans-compartmental biodistribution of exosomes can be manipulated by exogenous extracorporeal forces acting upon a subject after compartmental delivery of exosomes. This includes the application of mechanical convection, for example by way of applying percussion, vibration, shaking, or massaging of a body compartment or the entire body. Following intrathecal dosing for example, the application of chest wall vibrations by several means including an oscillating mechanical jacket can spread the biodistribution of the EVs, e.g., exosomes along the neuraxis or along cranial and spinal nerves, which can be helpful in the treatment of nerve disorders by drug carrying exosomes.

In some aspects, the application of external mechanical convective forces via an oscillating jacket or other similar means can be used to remove EVs and other material from the cerebrospinal fluid of the intrathecal space and out to the peripheral circulation. This aspect can help remove endogenous toxic exosomes and other deleterious macromolecules such as beta-amyloid, tau, alpha-synuclein, TDP43, neurofilament and excessive cerebrospinal fluid from the intrathecal space to the periphery for elimination.

In some aspects, exosomes delivered via the intracebroventricular route can be made to translocate throughout the neuraxis by simultaneously incorporating a lumbar puncture and allowing for ventriculo-lumbar perfusion wherein additional fluid is infused into the ventricles after exosome dosing, while allowing the existing neuraxial column of CSF to exit is the lumbar puncture. Ventriculo-lumbar perfusion can allow ICV dosed EVs to spread along the entire neuraxis and completely cover the subarachnoid space in order to treat leptomeningeal cancer and other diseases.

In some aspects, the application of external extracorporeal focused ultrasound, thermal energy (heat) or cold may be used to manipulate the compartmental pharmacokinetics and drug release properties of exosomes engineered to be sensitive to these phenomena.

In some aspects, the intracompartmental behavior and biodistribution of exosomes engineered to contain paramagnetic material can be manipulated by the external application of magnets or a magnetic field.

In certain aspects, the compartmental administration comprises delivery of the composition to the lungs or the epithelial lining of the respiratoty tract, e.g., by inhalation. In certain aspects, the compartmental administration comprises delivery of the composition to a muscle, e.g., by intramuscular administration. In certain aspects, the compartmental administration comprises delivery of the composition to a lymph node, e.g., by intraperitoneal administration. In certain aspects, the compartmental administration comprises delivery of the composition to an eye, e.g., by intraocular administration. In some aspects, the intraocular administration is selected from intravitreal, intracameral, subconjunctival, subretinal, subscleral, intrachoroidal, and any combination thereof. In certain aspects, the compartmental administration comprises delivery of the composition to the gastrointestinal tract (e.g., the colon), e.g., by oral administration.

In some aspects, the target population of cells is selected from one or more cells of the CNS, muscles, eyes, colon, lungs, lymph nodes, or any combination thereof. In some aspects, the target tissue comprises the epithelial lining of the respiratoty tract. In certain aspects, the compartmental administration comprises targeted delivery of the composition to one or more CNS cell. In some aspects, the CNS cell is a selected from an oligodendrocyte, an astrocyte, an ependymal cell, a microglia, and any combination thereof. In some aspects, the CNS cell is selected from a motor neuron, a sensory neuron, an interneuron, and any combination thereof. In certain aspects, the compartmental administration comprises targeted delivery of the composition to one or more eye cell. In some aspects, the one or more eye cell is selected form a rod cell, a cone cell, a retinal ganglion cell, and any combination thereof. In certain aspects, the compartmental administration comprises targeted delivery of the composition to one or more muscle cell. In some aspects, the muscle cell is a selected from a skeletal muscle cell, a smooth muscle cell, a cardiomyocyte, and any combination thereof.

In some aspects, the compartmental administration of the composition comprising the EV comprises targeted delivery of the composition to a tumor. In some aspects, the administration is intratumoral. In some aspects, the composition is administered to the periphery of the tumor.

In some aspects, the compartmental administration comprises targeted injection of the composition. In some aspects, the compartmental administration is facilitated by the use of a delivery device. Any in vivo delivery device known in the art can be used in the methods disclosed herein. In some aspects, the delivery device is implanted in the subject. In some aspects, the delivery device is implanted at the target tissue in the subject. In some aspects, the delivery device is implanted adjacent to the target tissue in the subject. In some aspects, the delivery device comprises a pump. In some aspects the delivery device comprises a sustained delivery device.

In some aspects, compartmental administration of the composition comprising the EV increases the tissue specific effects of the composition relative to non-compartmental, e.g., systemic (e.g., intravenous), administration.

The present disclosure also provides methods of preventing and/or treating a disease or disorder in a subject in need thereof, comprising administering an EV disclosed herein to the subject. In some aspects, a disease or disorder that can be treated with the present methods comprises a cancer, a hemophilia, diabetes, a growth factor deficiency, an eye disease, a Pompe disease, Gaucher, a lysosomal storage disorder, mucovicidosis, cystic fibrosis, Duchenne and Becker muscular dystrophy, transthyretin amyloidosis, hemophilia A, hemophilia B, adenosine-deaminase deficiency, Leber’s congenital amaurosis, X-linked adrenoleukodystrophy, metachromatic leukodystrophy, OTC deficiency, glycogen storage disease 1A, Criggler-Najjar syndrome, primary hyperoxaluria type 1, acute intermittent porphyria, phenylketonuria, familial hypercholesterolemia, mucopolysaccharidosis type VI, α1 antitrypsin deficiency, Retts Syndrome, Dravet Syndrome, Angelman Syndrome, DM1 disease, Fragile X disease, Huntingtons Disease, Friedreichs ataxia, CMT disease (also known as Charcot-Marie-Tooth disease, hereditary motor and sensory neuropathy (HMSN), or peroneal muscular atrophy), CMT1X disease, catecholaminergic polymorphic ventricular tachycardia, spinocerebellar ataxia type 3 (SCA3) disease, limb-girdle muscular dystrophy, or a hypercholesterolemia. In some aspects, a disease or disorder that can be treated with the present methods comprises demyelinating neuropathy (e.g., CMT1A), a sensory neuropathy (e.g., Friedrich’s Ataxia and/or neuropathic pain), and/or a neurodegenerative motor neuropathy (e.g., ALS). In some aspects, the treatment is prophylactic.

In some aspects, the disease or disorder comprises a cancer. In some aspects, the cancer is advanced, locally advanced, or metastatic. In some aspects, the cancer is recurrent. In some aspects, the cancer is refractory to a prior therapy, e.g., a prior standard of care therapy.

In some aspects, the disease or disorder is associated with a clotting factor deficiency. In some aspects, the disease or disorder is a bleeding disease. In some aspects, the disease or disorder is a hemophilia. In some aspects, the disease or disorder is hemophilia A. In some aspects, the disease or disorder is hemophilia B. In some aspects, the disease or disorder is von Willebrand disease.

In some aspects, the disease or disorder comprises an acute neurological injury such as a subarachnoid hemorrhage, trauma, stroke, or any combination thereof.

In some aspects, the neurodegenerative disease is selected from Alzheimer’s disease, Parkinson’s disease, prion disease, motor neuron disease, Huntington’s disease, spinocerebellar ataxia, spinal muscular atrophy, and any combination thereof.

In certain aspects, the disease or disorder comprises a muscular dystrophy. In some aspects, the muscular dystrophy is selected from Duchenne type muscular dystrophy (DMD), myotonic muscular dystrophy, facioscapulohumeral muscular dystrophy (FSHD), congenital muscular dystrophy, limb-girdle muscular dystrophy (including, but not limited to, LGMD2B, LGMD2D, LGNMD2L, LGMD2C, LGMD2E and LGMD2A), and any combination thereof.

In some aspects, the disease or disorder is selected from AADC deficiency (CNS), ADA-SCID, Alpha-1 antitrypsin deficiency, β-thalassemia (severe sickle cell), Cancer (head and neck squamous cell), Niemman-Pick Type C Disease, Cerebral ALD, Choroideremia, Congestive heart failure, Cystic Fibrosis, Duchenne muscular dystrophy (DMD), Fabry disease, Glaucoma, Glioma (cancer), Hemophilia A, Hemophilia B, HoFH (hypercholesterolemia), Huntington’s Disease, Lipoprotein lipase deficiency, Leber hereditary optic neuropathy (LHON), Metachromatic leukodystrophy, MPS I (Hurler syndrome), MPS II (Hunter’s syndrome), MPS III (Sanfilippo Syndrome), Parkinson’s disease, Pompe Disease, Recessive Dystrophic Epidermolysis Bullosa, RPE65 deficiency (vision loss), Spinal Muscular Atrophy (SMA I), Wet AMD (retinal disease), Wiskott Aldrich syndrome (WAS), Mucopolysaccharidosis type IIIA (MPS IIIA), X-linked myotubular myopathy, X-linked retinitis pigmentosa, and any combination thereof.

In some aspects, the disease or disorder is selected from nephropathy, diabetes insipidus, diabetes type I, diabetes II, renal disease glomerulonephritis, bacterial or viral glomerulonephritides, IgA nephropathy, Henoch-Schonlein Purpura, membranoproliferative glomerulonephritis, membranous nephropathy, Sjogren’s syndrome, nephrotic syndrome minimal change disease, focal glomerulosclerosis and related disorders, acute renal failure, acute tubulointerstitial nephritis, pyelonephritis, GU tract inflammatory disease, Pre-clampsia, renal graft rejection, leprosy, reflux nephropathy, nephrolithiasis, genetic renal disease, medullary cystic, medullar sponge, polycystic kidney disease, autosomal dominant polycystic kidney disease, autosomal recessive polycystic kidney disease, tuberous sclerosis, von Hippel-Lindau disease, familial thin-glomerular basement membrane disease, collagen III glomerulopathy, fibronectin glomerulopathy, Alport’s syndrome, Fabry’s disease, Nail-Patella Syndrome, congenital urologic anomalies, monoclonal gammopathies, multiple myeloma, amyloidosis and related disorders, febrile illness, familial Mediterranean fever, HIV infection-AIDS, inflammatory disease, systemic vasculitides, polyarteritis nodosa, Wegener’s granulomatosis, polyarteritis, necrotizing and crecentic glomerulonephritis, polymyositis-dermatomyositis, pancreatitis, rheumatoid arthritis, systemic lupus erythematosus, gout, blood disorders, sickle cell disease, thrombotic thrombocytopenia purpura, Fanconi’s syndrome, transplantation, acute kidney injury, irritable bowel syndrome, hemolytic-uremic syndrome, acute corticol necrosis, renal thromboembolism, trauma and surgery, extensive injury, burns, abdominal and vascular surgery, induction of anesthesia, side effect of use of drugs or drug abuse, circulatory disease myocardial infarction, cardiac failure, peripheral vascular disease, hypertension, coronary heart disease, non-atherosclerotic cardiovascular disease, atherosclerotic cardiovascular disease, skin disease, psoriasis, systemic sclerosis, respiratory disease, COPD, obstructive sleep apnoea, hypoia at high altitude or erdocrine disease, acromegaly, diabetes mellitus, and diabetes insipidus, or any combination thereof.

In some aspects, the disease or condition comprises a cancer, e.g., a cancer selected from cancers of the lung, ovarian, cervical, endometrial, breast, brain, colon, prostate, gastrointestinal cancer, head and neck cancer, non-small cell lung cancer, cancer of the nervous system, kidney cancer, retina cancer, skin cancer, liver cancer, pancreatic cancer, genital-urinary cancer and bladder cancer, melanoma, leukemia, brain cancer (e.g., glioma, astrocytomas, ependymomas, oligodendrogliomas, and tumors with mixtures of two or more cell types, called mixed gliomas, Acoustic Neuroma (Neurilemmoma, Schwannoma. Neurinoma), Adenoma, Astracytoma, Low-Grade Astrocytoma, giant cell astrocytomas, Midand High-Grade Astrocytoma, Recurrent tumors, Brain Stem Glioma, Chordoma, Choroid Plexus Papilloma, CNS Lymphoma (Primary Malignant Lymphoma), Cysts, Dermoid cysts, Epidermoid cysts, Craniopharyngioma, Ependymoma Anaplastic ependymoma, Gangliocytoma (Ganglioneuroma), Ganglioglioma, Glioblastoma Multiforme (GBM), Malignant Astracytoma, Glioma, Hemangioblastoma, Inoperable Brain Tumors, Lymphoma, Medulloblastoma (MDL), Meningioma, Metastatic Brain Tumors, Mixed Glioma, Neurofibromatosis, Oligodendroglioma. Optic Nerve Glioma, Pineal Region Tumors, Pituitary Adenoma, PNET (Primitive Neuroectodermal Tumor), Spinal Tumors, Subependymoma, and Tuberous Sclerosis (Bourneville’s Disease), and any combination thereof.

In some aspects, the disease or disorder is associated with a growth factor deficiency. In some aspects, the growth factor is selected from the group consisting of adrenomedullin (AM), angiopoietin (Ang), autocrine motility factor, a bone morphogenetic protein (BMP) (e.g. BMP2, BMP4, BMP5, BMP7), a ciliary neurotrophic factor family member (e.g., ciliary neurotrophic factor (CNTF), leukemia inhibitory factor (LIF), interleukin-6 (IL-6)), a colony-stimulating factor (e.g., macrophage colony-stimulating factor (m-CSF), granulocyte colony-stimulating factor (G-CSF), granulocyte macrophage colony-stimulating factor (GM-CSF)), an epidermal growth factor (EGF), an ephrin (e.g., ephrin A1, ephrin A2, ephrin A3, ephrin A4, ephrin A5, ephrin B1, ephrin B2, ephrin B3), erythropoietin (EPO), a fibroblast growth factor (FGF) (e.g., FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, FGF23), foetal bovine somatotrophin (FBS), a GDNF family member (e.g., glial cell line-derived neurotrophic factor (GDNF), neurturin, persephin, artemin), growth differentiation factor-9 (GDF9), hepatocyte growth factor (HGF), hepatoma-derived growth factor (HDGF), insulin, an insulin-like growth factors (e.g., insulin-like growth factor-1 (IGF-1) or IGF-2, an interleukin (IL) (e.g., IL-1, IL-2, IL-3, IL-4, IL-5, IL-6, IL-7), keratinocyte growth factor (KGF), migration-stimulating factor (MSF), macrophage-stimulating protein (MSP or hepatocyte growth factor-like protein (HGFLP)), myostatin (GDF-8), a neuregulin (e.g., neuregulin 1 (NRG1), NRG2, NRG3, NRG4), a neurotrophin (e.g., brain-derived neurotrophic factor (BDNF), nerve growth factor (NGF), a neurotrophin-3 (NT-3), NT-4, placental growth factor (PGF), platelet-derived growth factor (PDGF), renalase (RNLS), T-cell growth factor (TCGF), thrombopoietin (TPO), a transforming growth factor (e.g., transforming growth factor alpha (TGF-α), TGF-β, tumor necrosis factor-alpha (TNF-α), and vascular endothelial growth factor (VEGF).

In some aspects, the disease or disorder is diabetes. In some aspects, the disease or disorder is an eye disease or disorder. In some aspects, the disease or disorder is Choroideremia (CHM).

In some aspects, the eye disease or disorder is selected from the group consisting of macular degeneration, cataract, diabetic retinopathy, glaucoma, amblyopia, strabismus, retinopathy, or any combination thereof. In some aspects, the eye disease or disorder is, e.g., age-related macular degeneration (AMD), choroidal neovascularization (CNV), retinal detachment, diabetic retinopathy, retinal pigment epithelium atrophy, retinal pigment epithelium hypertrophy, retinal vein occlusion (RVO) disease, infection, intraocular tumor, ocular trauma, dry eye, conjunctivitis, neovascular glaucoma, retinopathy of prematurity (ROP), choroidal retinal vein occlusion, macular edema, anterior neovascularization, corneal neovascularization, subretinal edema, cystoid macular edema, macular hole, vascular striae, pigmented retinitis, Stuttgart disease, inflammatory eye conditions, refractory eye abnormalities, keratoconus, laser induced AMD, optical neuropathy, or senile cataract.

In some aspects, the eye disease or disorder is an eye cancer. In some aspects, the eye cancer is a secondary eye cancer (e.g., due to breast cancer or lung cancer metastasis). In some aspects, the eye cancer is retinoblastoma, intraocular melanoma (e.g., uveal melanoma of the iris, choroid, or ciliary body, or conjunctival melanoma), non-Hodgkin primary intraocular lymphoma, medulloepithelioma, choroidal hemangioma, choroidal metastasis, choroidal nevus, choroidal osteoma, conjunctival Kaposi’s sarcoma, epibulbar dermoid, pingueculum, pterygium, squamous carcinoma, or intraepithelial neoplasia of the conjunctiva.

In some aspects, AMD is any stage of retinal disease, including but not limited to Category 2 (early stage), Category 3 (intermediate stage) and Category 4 (advanced stage) AMD.

In one aspect, AMD is generally categorized into two types: a dry form and a wet form. The term “dry form” refers to one type of AMD, where alteration of the retina is accompanied by the formation of a small yellow deposit (drusen) under the macula. In some aspects, dry form AMD is often accompanied by choroidal capillary atrophy, fibrosis, Bruch’s thickening, and macular atrophy due to atrophy of the retinal pigment epithelium.

The term “wet form” refers to AMD with abnormal blood vessels that develop under the retina around the macula. Abnormal blood vessels, when broken and bleeding, can damage the macula and dislodge the macula from its base. Symptoms of wet form AMD include Bruch’s membrane destruction, glass membrane, choroidal neovascularization (CNV), vascular invasion into the subretinal choroid, followed by serous or hemorrhagic circles This includes, but is not limited to, macular retinal pigment subepithelial or subepithelial vascular invasion, which causes plate-like detachment and eventually becomes a disc-like scar. According to clinical findings, the atrophic type can also change to a wet type.

In some aspects, wet AMD is also referred to as choroidal neovascularization (“CNV”). CNV (or wet form) can be further classified into “classic” CNV and “occult” CNV. Classic CNV is generally characterized by a bright, highly fluorescent, well-defined region spanning the angiographic transition phase with leakage in the middle and late phase frames. The occult CNV includes fibrovascular pigment epithelial detachment. Neovascularization resulting from CNV has a tendency to leak blood and body fluids, causing stigma and symptoms of metamorphosis. This new blood vessel is accompanied by the growth of fibrous tissue. This complex of neovascular and fibrous tissue can destroy photoreceptors. This lesion can continue to grow across the macula and cause progressive, severe and irreversible blindness. When one individual’s eye develops CNV, similar CNV lesions occur in the other eye with a probability of approximately 50% within 5 years.

In some aspects, a CNV lesion of the present disclosure comprises an occult CNV. In one aspect, the CNV lesion comprises, consists essentially of, or further consists of classic CNV. In another aspect, the CNV lesion includes both classic and occult CNV.

The term “macular edema” refers to the ocular diseases cystoid macular edema (CME) or diabetic macular edema (DME). CME is an ocular disease that affects the central retina or macula of the eye. When this condition is present, multiple cyst-like (cystoid) areas of fluid appear in the macula and cause retinal swelling or edema. CME can accompany a variety of diseases such as retinal vein occlusion, uveitis, and/or diabetes. CME commonly occurs after cataract surgery. DME occurs when blood vessels in the retina of patients with diabetes begin to leak into the macula. These leaks cause the macula to thicken and swell, progressively distorting acute vision. While the swelling may not lead to blindness, the effect can cause a severe loss in central vision.

The term “glaucoma” refers to an ocular disease in which the optic nerve is damaged in a characteristic pattern. This can permanently damage vision in the affected eye and lead to blindness if left untreated. It is normally associated with increased fluid pressure in the eye (aqueous humor). The term ocular hypertension is used for patients with consistently raised intraocular pressure (IOP) without any associated optic nerve damage. Conversely, the term normal tension or low tension glaucoma is used for those with optic nerve damage and associated visual field loss but normal or low IOP. The nerve damage involves loss of retinal ganglion cells in a characteristic pattern. There are many different subtypes of glaucoma, but they can all be considered to be a type of optic neuropathy. Raised intraocular pressure (e.g., above 21 mmHg or 2.8 kPa) is the most important and only modifiable risk factor for glaucoma. However, some can have high eye pressure for years and never develop damage, while others can develop nerve damage at a relatively low pressure. Untreated glaucoma can lead to permanent damage of the optic nerve and resultant visual field loss, which over time can progress to blindness.

The term “diabetic retinopathy” includes retinopathy (i.e., a disease of the retina) caused by complications of diabetes, which can eventually lead to blindness. Diabetic retinopathy can cause no symptoms, mild vision problems, or even blindness. Diabetic retinopathy is the result of microvascular retinal changes. Hyperglycemia-induced intramural pericyte death and thickening of the basement membrane lead to incompetence of the vascular walls. These damages change the formation of the blood-retinal barrier and also make the retinal blood vessels become more permeable.

### II.B. Methods of Delivery to the Central Nervous System

The present disclosure relates to methods to deliver extracellular vesicles (e.g., exosomes) comprising a biologically active molecule to the central nervous system (CNS).

The biologically active molecule can be covalently linked to the extracellular vesicle (e.g., to the internal and/or external side of the membrane) and/or encapsulated in the lumen of the extracellular vesicle (e.g., exosomes). The biologically active molecule can be useful, e.g., as an agent for the prophylaxis or treatment of cancer or neurological diseases. In some aspects, the administration of the extracellular vesicles (e.g., exosomes) is intrathecal. In some aspects, delivery to the CNS (e.g., intrathecally) is further improved by the attachment to the surface of the extracellular vesicle of an anti-phagocytic signal (e.g., CD47 and/or CD24), a half-life extension moiety (e.g., albumin or PEG), a targeting moiety for cell type-directed tropism (e.g., an immuno-affinity ligand targeting a certain neural cell type), or any combination thereof.

Extracellular vesicles (EVs) typically have 20 nm to 1000 nm in diameter; e.g., exosomes, which are small extracellular vesicles, have typically 100 to 200 nm in diameter. EVs are composed of a limiting lipid bilayer and a diverse set of proteins and nucleic acids (Maas, S.L.N., et al., Trends. Cell Biol. 27(3):172-188 (2017)). EVs exhibit preferential uptake in discrete cell types and tissues, and their tropism can be directed by adding proteins to their surface that interact with receptors on the surface of target cells (Alvarez-Erviti, L., et al., Nat. Biotechnol. 29(4):341-345 (2011)).

Unlike antibodies, EVs can accommodate large numbers of molecules attached to their surface, on the order of thousands to tens of thousands of molecules per EV. EV-drug conjugates thus represent a platform to deliver a high concentration of therapeutic compound to discrete cell types, while at the same time limiting overall systemic exposure to the compound, which in turn reduces off-target toxicity.

The methods disclosed herein can deliver EVs to the CNS, wherein the EVs comprise biologically active molecules which can be, e.g., small molecules such as cyclic dinucleotides, toxins such as monoethyl auristatin E (MMAE), antibodies (e.g., naked antibodies or antibody-drug conjugates), STING agonists, tolerizing agents, antisense oligonucleotides, PROTACs, morpholinos, etc.

The central nervous system (CNS) is the part of the nervous system consisting of the brain and spinal cord. The retina, optic nerve (cranial nerve II), as well as the olfactory nerves (cranial nerve I) and olfactory epithelium are considered as parts of the CNS because the synapse directly on brain tissue without intermediate ganglia. As such, the olfactory epithelium is the only central nervous tissue in direct contact with the environment, which opens up for therapeutic treatments. The CNS is contained within the dorsal body cavity, with the brain housed in the cranial cavity and the spinal cord in the spinal canal. In vertebrates, the brain is protected by the skull, while the spinal cord is protected by the vertebrae. The brain and spinal cord are both enclosed in the meninges. Within the CNS, the interneuronal space is filled with a large amount of supporting non-nervous cells called neuroglia or glia

The efficacy of EVs in the CNS can be increased by surface engineering to adjust pharmacokinetics and biodistribution. This can be accomplished, for example, by (i) increasing cell type-directed tropism, e.g., directing an EVs to the CNS, via the attachment of targeting ligands such a immunoaffinity-ligands (e.g., mABs, VNARs) and/or cognate receptor ligands (e.g., peptides or proteins), (ii) modifying clearance, e.g., by increasing the half-life of the EVs via attachment of half-life extension moieties, such as albumin or PEG, or by incorporating an anti-phagocytic signal (also called a “don’t eat me” signal) such as CD47 and/or CD24 to the surface of the EVs or (iii) any combination thereof.

Pharmacokinetics and biodistribution, and in particular tropism to the CNS and retention in the CNS can also be accomplish by selecting the appropriate administration route. Thus, the present disclosure provides methods to improve the pharmacokinetics and biodistribution of therapeutic and/or diagnostic agents carried by EVs of the present disclosure, via specific administration routes, which can optionally be combined with the surface engineering approaches disclosed above.

The present disclosure provides a method of targeting an extracellular vesicle to central nervous system in a subject in need thereof comprising administering a composition comprising an extracellular vesicle (EV) which comprises a biologically active molecule to the subject, wherein the administration of the composition is intrathecal, intraocular, intracranial, intranasal, or perineural. Also provided is a method of treating a central nervous system disease in a subject in need thereof comprising administering a composition comprising an extracellular vesicle (EV), e.g., an exosome, which comprises a biologically active molecule to the subject, wherein the administration of the composition is intrathecal, intraocular, intracranial, intranasal, or perineural.

The term “intrathecal” as used herein is a route of administration for an EV (e.g., an exosome) disclosed herein via an injection into the spinal canal, or into the subarachnoid space so that it reaches the cerebrospinal fluid (CSF). In some aspects, intrathecal administration refers to the administration within the cerebrospinal fluid at any levels of the cerebrospinal axis, including injection into the cerebral ventricles. The term “intraocular” as used herein refers to the administration of an EV (e.g., an exosome) disclosed herein within the eye. The term “intracranial” as used herein refers to the administration of an EV (e.g., an exosome) disclosed herein within the cranial cavity. The term “intranasal” as used herein refers to the administration of an EV (e.g., an exosome) disclosed herein within the nasal cavity. In some aspects, intranasal administration refers to the contacting an EV (e.g., an exosome) disclosed herein with the nasal epithelium. The term “perineural” as used herein refers to the administration of an EV (e.g., an exosome) disclosed herein surrounding a nerve or nerves.

In some aspects, the intrathecal administration is in the spinal canal and/or the subarachnoid space. In some aspects, the intrathecal administration is by injection. In some aspects, the intrathecal administration comprises the implantation of a delivery device comprising the composition. In some aspects, the delivery device is an intrathecal pump. An intrathecal pump is a medical device used to deliver (via an intrathecal catheter) a therapeutic or diagnostic agent (e.g., an EV, such as an exosome, of the present disclosure) directly into the space between the spinal cord and the protective sheath surrounding the spinal cord. An implantable intrathecal pump consist of a pump portion which stores and delivers the therapeutic or diagnostic agent (e.g., an EV, such as an exosome, of the present disclosure), and an intrathecal catheter which delivers the therapeutic or diagnostic agent (e.g., an EV, such as an exosome, of the present disclosure) from the pump to the intrathecal space in the spine. Two types of pumps are available: constant rate pumps, which deliver the therapeutic or diagnostic agent at a constant rate, and programmable pumps which delivery the therapeutic or diagnostic agent according to a rate determined by a computer program. Also, external pumps, with our without a subcutaneous port, can be used for intrathecal delivery.

In some aspects, the intraocular administration is selected from the group consisting of intravitreal, intracameral, subconjunctival, subretinal, subscleral, intrachoroidal, and any combination thereof. The term “intravitreal” as used herein refers to the administration of an EV (e.g., an exosome) disclosed herein within the vitreous body of the eye. The term “intracameral” as used herein refers to the administration of an EV (e.g., an exosome) disclosed herein within the anterior camera of the eye, i.e., within the area between the iris and the cornea.

The term “subconjunctival” as used herein refers to the administration of an EV (e.g., an exosome) disclosed herein beneath the conjunctiva of the eyeball (i.e., epibulbar administration) or underneath the conjunctiva lining of the eyelid (subpalpebral). In some aspects of the present disclosure, the term subconjunctival injection or subconjunctival administration refers to epibulbar administration. It has been determined that both anterior and vitreous levels of drugs can be established from subconjunctival injection. However, the subconjunctival route can only provide a limited capability for delivering sufficient levels of drugs when implanted devices are used. Only implants with high rates of release impart sufficient drug levels into the choroid and subretinal space.

The term “subretinal” as used herein refers to the administration of an EV (e.g., an exosome) disclosed herein beneath the retina. The term “subscleral” as used herein refers to the administration of an EV (e.g., an exosome) disclosed herein beneath the sclera of the eye, i.e., beneath the white of the eye, the opaque fibrous, protective, outer layer of the human eye containing mainly collagen and some elastic fiber. The term “intrachoroidal” as used herein refers to the administration of an EV (e.g., an exosome) disclosed herein into the choroid, i.e., within the pigmented vascular layer of the eyeball between the retina and the sclera.

In some aspects, the intraocular administration comprises the injection of the composition, comprising an EV (e.g., an exosome) disclosed herein. In a particular aspect, the intraocular administration is intravitreal injection. In some aspects, the intraocular administration comprises the implantation of a delivery device comprising the composition. In some aspects, the delivery device is an intraocular delivery device. In some aspects, the intraocular delivery device is an intravitreal implant or a scleral plug. An intravitreal implant is a drug delivery system, injected or surgically implanted in the vitreous of the eye, for sustained release of a drug to the posterior and intermediate segments of the eye. Intravitreal implants deliver a continuous concentration of drug over a prolonged period. A scleral plug is a device for vitreoretinal drug delivery that is implanted generally at the pars plana (part of the ciliary body in the uvea or vascular tunic, the middle layer of the three layers that comprise the eye; it is about 4 mm long, located near the junction of the iris and sclera, and is scalloped in appearance) and gradually releases effective doses of drugs to the interior of the eye for a prolonged period of time (e.g., days to months) general via degradation of a biopolymer containing the drug. In some aspects, the delivery device, e.g., an intravitreal implant or scleral plug, is a sustained release delivery device.

In some aspects, the intracranial administration is intracisternal, subarachnoidal, intrahippocampal, intracerebroventricular, intraparenchymal, intracerebral, intracaudal, or a combination thereof. The term “intracisternal” as used herein refers to the administration of an EV (e.g., an exosome) disclosed herein within the cisterna magna cerebellomedularis. The term “subarachnoidal” as used herein refers to the administration of an EV (e.g., an exosome) disclosed herein beneath the arachnoid. The term “intrahippocampal” as used herein refers to the administration of an EV (e.g., an exosome) disclosed herein within the hippocampus. The term “intracerebroventricular” as used herein refers to the administration of an EV (e.g., an exosome) disclosed herein within the brain ventricle. The term “intraparenchymal” as used herein refers to the administration of an EV (e.g., an exosome) disclosed herein within the brain parenchyma. In some aspects, the intraparenchymal administration is Convection-Enhanced Intraparenchymal administration. The term “intracerebral” as used herein refers to the administration of an EV (e.g., an exosome) disclosed herein within the cerebrum. The term “intracaudal” as used herein refers to the administration of an EV (e.g., an exosome) disclosed herein within the cauda equina.

In some aspects, the intracranial administration is by injection. In some aspects, the intracranial administration is via a catheter or port. In some aspects, the catheter or port is implanted. In some aspects, a pump is connected to the catheter or port.

In some aspects, the intranasal administration is by instillation or injection. If the nasally administered medication contacts the olfactory mucosa, molecule transport can occur directly across this tissue and into the cerebral spinal fluid. The olfactory mucosa is located in the upper nasal cavity, just below the cribriform plate of the skull. It contains olfactory cells which traverse the cribriform plate and extend up into the cranial cavity. When medication molecules come in contact with this specialized mucosa they are rapidly transported directly into the brain, skipping the blood-brain barrier, and achieving very rapid cerebrospinal fluid levels (often faster than if the drug is given intravenously). This concept of transfer of molecules from the nose to the brain is referred to as the nose-brain pathway. The nose-brain pathway leads to nearly immediate delivery of some nasal medications to the cerebral spinal fluid, by-passing the blood brain barrier.

In some aspects, the perineural administration is by facial intradermal injection. In some aspects, the facial intradermal injection targets the trigeminal substructures. In some aspects, the trigeminal substructures are selected from the group consisting of trigeminal perineurium, epineurium, perivascular spaces, neurons and Schwann cells, and combinations thereof.

In some aspects, the EV for delivery to the CNS comprises a surface anchored anti-phagocytic signal (also known as a “don’t eat me” signal). In some aspects, the anti-phagocytic signal is CD47, CD24, a fragment or variant thereof, or a combination thereof. CD47 (Cluster of Differentiation 47) also known as integrin associated protein (IAP) is a transmembrane protein that in humans is encoded by the CD47 gene. CD47 belongs to the immunoglobulin superfamily and partners with membrane integrins and also binds the ligands thrombospondin-1 (TSP-1) and signal-regulatory protein alpha (SIRPα). CD-47 acts as a don’t eat me signal to macrophages of the immune system. Signal transducer CD24 also known as cluster of differentiation 24 or heat stable antigen CD24 (HSA) is a protein that in humans is encoded by the CD24 gene. CD24 is a cell adhesion molecule. CD24 is a sialoglycoprotein expressed at the surface of most B lymphocytes and differentiating neuroblasts. It is also expressed on neutrophils and neutrophil precursors from the myelocyte stage onwards. The encoded protein is anchored via a glycosyl phosphatidylinositol (GPI) link to the cell surface. CD-47 also acts as a don’t eat me signal.

In some aspects, the EV for delivery to the CNS comprises (i) at least one payload to treat a disease or condition of the CNS, (ii) a targeting moiety or tropism moiety that specifically directs the EV to a specific CNS tissue or cell type, and (iii) a surface molecule (e.g., CD47, CD24, a fragment or variant thereof, or a combination thereof) that protects the EV from degradation by macrophages.

In some aspects, the EV for delivery to the CNS comprises a tissue or cell-specific target ligand which increases EV tropism to a specific CNS tissue or cell, i.e., a “tropism moiety.” In some aspects, the cell is a glial cell. In some aspects, the glial cell is an oligodendrocyte, an astrocyte, an ependymal cell, a microglia cell, a Schwann cell, a satellite glial cell, an olfactory ensheathing cell, or a combination thereof. In some aspects, the cell is a neural stem cell.

In some aspects, the cell is a neuron. In some aspects, the neuron is a motor neuron, a sensory neuron, or an interneuron. In some aspects, the tissue or cell-specific target ligand is a cell marker (e.g., a protein or receptor) present of the surface of a CNS cell, e.g., a neuron or a glial cell. In some aspects, the tissue is selected from the group consisting of brain tissue, spinal cord tissue, retina, optic nerve (cranial nerve II), olfactory nerves (cranial nerve I), olfactory epithelium, meningeal tissue, or any combination thereof. In some aspects, the tissue is from a CNS area selected from the group consisting of cerebrum, cerebral cortex, basal ganglia, amygdala, hippocampus, thalamus, hypothalamus, cerebellum, brainstem, medulla, pons, midbrain, and reticular formation. In some aspects, the tissue or cell are malignant.

The present disclosure also provides methods of treating a disease or condition is a subject in need thereof comprising administering a composition comprising EVs of the present disclosure to the subject, wherein the EVs are delivered to the CNS. The present disclosure also provides methods of preventing or ameliorating the symptoms of a disease or condition is a subject in need thereof comprising administering a composition comprising EVs of the present disclosure to the subject wherein the EVs are delivered to the CNS. Also provided are methods to diagnose a disease or condition in a subject in need thereof comprising administering a composition comprising EVs of the present disclosure to the subject wherein the EVs are delivered to the CNS.

In one aspect, the disease or disorder is a cancer, an inflammatory disease, a neurodegenerative disorder, a central nervous disease or a metabolic disease. In some aspects, a disease or disorder that can be treated with the present methods comprises a cancer, graft-versus-host disease (GvHD), autoimmune disease, infectious diseases, or fibrotic diseases. In some aspects, the treatment is prophylactic. In other aspects, the EVs for the present disclosure are used to induce an immune response. In other aspects, the EVs for the present disclosure are used to vaccinate a subject.

In some aspects, the disease or disorder is a cancer. When administered to a subject with a cancer, in certain aspects, EVs of the present disclosure can up-regulate an immune response and enhance the tumor targeting of the subject’s immune system. In some aspects, the cancer being treated is characterized by infiltration of leukocytes (T-cells, B-cells, macrophages, dendritic cells, monocytes) into the tumor microenvironment, or so-called “hot tumors” or “inflammatory tumors.” In some aspects, the cancer being treated is characterized by low levels or undetectable levels of leukocyte infiltration into the tumor microenvironment, or so-called “cold tumors” or “non-inflammatory tumors.” In some aspects, an EV is administered in an amount and for a time sufficient to convert a “cold tumor” into a “hot tumor,” i.e., said administering results in the infiltration of leukocytes (such as T-cells) into the tumor microenvironment. In certain aspects, cancer comprises a brain tumor. The term “distal tumor” or “distant tumor” refers to a tumor that has spread from the original (or primary) tumor to distant organs or distant tissues, e.g., melanoma metastasis to the brain. In some aspects, the EVs of the disclosure treats a tumor after the metastatic spread.

In some aspects, the CNS disease or condition that can be treated with an EV of the present disclosure formulated for administration to the CNS can be trauma (e.g., traumatic brain injury), infection, neurodegeneration, degeneration, tumor, autoimmune disorders, or stroke.

In some aspects, the disease or disorder is an neurodegenerative or autoimmune disease affecting the CNS, e.g., Alzheimer’s disease, Parkinson’s disease, or Huntington’s disease.

In some aspects, the disease or disorder is an infectious disease affecting the CNS. In certain aspects, the disease or disorder is an oncogenic virus. In some aspects, infectious diseases that can be treated with the present disclosure includes, but not limited to, Human Gamma herpes virus 4 (Epstein Barr virus), influenza A virus, influenza B virus, cytomegalovirus, staphylococcus aureus, mycobacterium tuberculosis, chlamydia trachomatis, HIV-1, HIV-2, corona viruses (e.g., MERS-CoV and SARS CoV), filoviruses (e.g., Marburg and Ebola), Streptococcus pyogenes, Streptococcus pneumoniae, Plasmodia species (e.g., vivax and falciparum), Chikunga virus, Human Papilloma virus (HPV), Hepatitis B, Hepatitis C, human herpes virus 8, herpes simplex virus 2 (HSV2), Klebsiella sp., Pseudomonas aeruginosa, Enterococcus sp., Proteus sp., Enterobacter sp., Actinobacter sp., coagulase-negative staphylococci (CoNS), Mycoplasma sp., or combinations thereof. In some aspects, the infection is cryptococcal meningitis, brain abscess, spinal epidural infection toxoplasmosis, malaria, primary amoebic meningoencephalitis, tuberculosis, leprosy, neurosyphilis, bacterial meningitis, Lyme disease, neuroborreliosis, viral meningitis, Easter equine encephalitis, St Louis encephalitis, West Nile encephalitis, tick-borne encephalitis, herpes simplex encephalitis, rabies, California encephalitis virus, Varicelle-zoster encephalitis, La Crosse encephalitis, Nipath virus encephalitis, measles encephalitis, poliomyelitis, Creutzfeldt-Jakob disease, or Kuru. In some aspects, the disease or condition is a post-infectious disease of the CNS, e.g., PANDAS, Sydenham’s chorea, acute disseminated encephalomyelitis, or Guillain-Barre syndrome.

In some aspects, the present disclosure provides a pharmaceutical composition comprising an EV of the present disclosure formulated for administration to the CNS according to the methods disclosed herein. The present disclosure also provides a kit comprising a pharmaceutical composition comprising an EV of the present disclosure formulated for administration to the CNS, and optionally instructions for use according to the methods disclosed herein, e.g., instructions to administer the pharmaceutical composition to treat a specific disease or disorder of the CNS.

The present disclosure provides a method of targeting an extracellular vesicle (EV), e.g., exosome, to the central nervous system (CNS) in a subject in need thereof comprising administering a composition comprising an extracellular vesicle (EV), e.g., exosome, which comprises a biologically active molecule to the subject, wherein the administration of the composition is intrathecal, intraocular, intracranial, intranasal, or perineural, and wherein the extracellular vesicle (EV), e.g., exosome comprises (i) a surface anchored anti-phagocytic signal and (ii) a tissue or cell-specific target ligand which increases EV tropism to cells in the CNS. Also provided is method of treating a CNS disease or condition in a subject in need thereof comprising administering an extracellular vesicle (EV), e.g., exosome, to the CNS of the subject wherein an extracellular vesicle (EV), e.g., exosome, comprises a biologically active molecule to the subject, wherein the administration of the composition is intrathecal, intraocular, intracranial, intranasal, or perineural, and wherein the extracellular vesicle (EV), e.g., exosome comprises (i) a surface anchored anti-phagocytic signal and (ii) a tissue or cell-specific target ligand which increases EV tropism to cells in the CNS. In some aspects, the cells are Schwann cells or oligodendrocytes. In some aspects, the anti-phagocytic signal is CD47, CD24, a fragment or variant thereof, or a combination thereof. In some aspects, the antiphagocytic signal is covalently attached to a Scaffold X moiety. In some aspects, the Scaffold X moiety is PTGFRN or a functional fragment thereof.

### III. Extracellular Vesicles, e.g., Exosomes

Certain aspects of the present disclosure are directed to compartmental administration of a composition comprising an EV, e.g., an exosome. EVs useful in the present disclosure have been engineered to comprise a biologically active molecule. In some aspects, the biologically active molecule is present in the lumen of the EV, e.g., exosome. In some aspects, the biologically active molecule is associated with the luminal surface of the EV, e.g., exosome. In some aspects, the biologically active molecule is present in the lumen of the EV but is not associated with the luminal surface of the EV, e.g., exosome. In some aspects, the biologically active moiety is associated with the exterior surface of the EV, e.g., exosome. In some aspects, the biologically active moiety is fused with a scaffold protein disclosed herein.

In some aspects, the EV is engineered to express a targeting moiety (i.e., exogenous targeting moiety). In some aspects, the targeting moiety allows the EV to target a specific tissue or a specific population of cells. In some aspects, the targeting moiety binds to a marker (e.g., any marker disclosed herein) that is expressed on a target cell, e.g., an immune cell. In further aspects, the marker is expressed only on the target cell, e.g., an immune cell. In still further aspects, the EVs of the present disclosure (e.g., exosomes) can comprise multiple (e.g., two or more) targeting moieties. In some aspects, the multiple targeting moieties bind to the same marker. In other aspects, the multiple targeting moieties bind to different markers.

In some aspects, an EV can comprise one or more additional exogenous biologically active molecules, e.g., an antigen, adjuvant, and/or immune modulator. Accordingly, in certain aspects, an EV disclosed herein (e.g., exosome) comprises (i) a targeting moiety (e.g., disclosed herein) and (ii) an antigen. In some aspects, an EV comprises (i) a targeting moiety and (ii) an adjuvant. In some aspects, an EV comprises (i) a targeting moiety and (ii) an immune modulator. In further aspects, an EV disclosed herein (e.g., exosome) comprises a (i) a targeting moiety, (ii) an antigen, (iii) an adjuvant, and (iv) an immune modulator.

As described supra, EVs described herein are extracellular vesicles with a diameter between about 20-300 nm. In certain aspects, an EV of the present disclosure has a diameter between about 20-290 nm, 20-280 nm, 20-270 nm, 20-260 nm, 20-250 nm, 20-240 nm, 20-230 nm, 20-220 nm, 20-210 nm, 20-200 nm, 20-190 nm, 20-180 nm, 20-170 nm, 20-160 nm, 20-150 nm, 20-140 nm, 20-130 nm, 20-120 nm, 20-110 nm, 20-100 nm, 20-90 nm, 20-80 nm, 20-70 nm, 20-60 nm, 20-50 nm, 20-40 nm, 20-30 nm, 30-300 nm, 30-290 nm, 30-280 nm, 30-270 nm, 30-260 nm, 30-250 nm, 30-240 nm, 30-230 nm, 30-220 nm, 30-210 nm, 30-200 nm, 30-190 nm, 30-180 nm, 30-170 nm, 30-160 nm, 30-150 nm, 30-140 nm, 30-130 nm, 30-120 nm, 30-110 nm, 30-100 nm, 30-90 nm, 30-80 nm, 30-70 nm, 30-60 nm, 30-50 nm, 30-40 nm, 40-300 nm, 40-290 nm, 40-280 nm, 40-270 nm, 40-260 nm, 40-250 nm, 40-240 nm, 40-230 nm, 40-220 nm, 40-210 nm, 40-200 nm, 40-190 nm, 40-180 nm, 40-170 nm, 40-160 nm, 40-150 nm, 40-140 nm, 40-130 nm, 40-120 nm, 40-110 nm, 40-100 nm, 40-90 nm, 40-80 nm, 40-70 nm, 40-60 nm, 40-50 nm, 50-300 nm, 50-290 nm, 50-280 nm, 50-270 nm, 50-260 nm, 50-250 nm, 50-240 nm, 50-230 nm, 50-220 nm, 50-210 nm, 50-200 nm, 50-190 nm, 50-180 nm, 50-170 nm, 50-160 nm, 50-150 nm, 50-140 nm, 50-130 nm, 50-120 nm, 50-110 nm, 50-100 nm, 50-90 nm, 50-80 nm, 50-70 nm, 50-60 nm, 60-300 nm, 60-290 nm, 60-280 nm, 60-270 nm, 60-260 nm, 60-250 nm, 60-240 nm, 60-230 nm, 60-220 nm, 60-210 nm, 60-200 nm, 60-190 nm, 60-180 nm, 60-170 nm, 60-160 nm, 60-150 nm, 60-140 nm, 60-130 nm, 60-120 nm, 60-110 nm, 60-100 nm, 60-90 nm, 60-80 nm, 60-70 nm, 70-300 nm, 70-290 nm, 70-280 nm, 70-270 nm, 70-260 nm, 70-250 nm, 70-240 nm, 70-230 nm, 70-220 nm, 70-210 nm, 70-200 nm, 70-190 nm, 70-180 nm, 70-170 nm, 70-160 nm, 70-150 nm, 70-140 nm, 70-130 nm, 70-120 nm, 70-110 nm, 70-100 nm, 70-90 nm, 70-80 nm, 80-300 nm, 80-290 nm, 80-280 nm, 80-270 nm, 80-260 nm, 80-250 nm, 80-240 nm, 80-230 nm, 80-220 nm, 80-210 nm, 80-200 nm, 80-190 nm, 80-180 nm, 80-170 nm, 80-160 nm, 80-150 nm, 80-140 nm, 80-130 nm, 80-120 nm, 80-110 nm, 80-100 nm, 80-90 nm, 90-300 nm, 90-290 nm, 90-280 nm, 90-270 nm, 90-260 nm, 90-250 nm, 90-240 nm, 90-230 nm, 90-220 nm, 90-210 nm, 90-200 nm, 90-190 nm, 90-180 nm, 90-170 nm, 90-160 nm, 90-150 nm, 90-140 nm, 90-130 nm, 90-120 nm, 90-110 nm, 90-100 nm, 100-300 nm, 110-290 nm, 120-280 nm, 130-270 nm, 140-260 nm, 150-250 nm, 160-240 nm, 170-230 nm, 180-220 nm, or 190-210 nm. The size of the EV described herein can be measured according to methods described, infra.

In some aspects, an EV of the present disclosure comprises a bi-lipid membrane (“EV membrane”), comprising an interior surface and an exterior surface. In certain aspects, the interior surface faces the inner core (i.e., lumen) of the EV, e.g., exosome. In certain aspects, the exterior surface can be in contact with the endosome, the multivesicular bodies, or the membrane/cytoplasm of a producer cell or a target cell.

In some aspects, the EV membrane comprises lipids and fatty acids. In some aspects, the EV membrane comprises phospholipids, glycolipids, fatty acids, sphingolipids, phosphoglycerides, sterols, cholesterols, and phosphatidylserines.

In some aspects, the EV membrane comprises an inner leaflet (i.e., luminal surface) and an outer leaflet (i.e., exterior surface). The composition of the inner and outer leaflet can be determined by transbilayer distribution assays known in the art, see, e.g., Kuypers et al., Biohim Biophys Acta 1985 819:170. In some aspects, the composition of the outer leaflet is between approximately 70-90% choline phospholipids, between approximately 0-15% acidic phospholipids, and between approximately 5-30% phosphatidylethanolamine. In some aspects, the composition of the inner leaflet is between approximately 15-40% choline phospholipids, between approximately 10-50% acidic phospholipids, and between approximately 30-60% phosphatidylethanolamine.

In some aspects, the EV membrane comprises one or more polysaccharide, such as glycan.

In some aspects, the EV membrane further comprises one or more scaffold moieties, which are capable of anchoring a biologically active molecule and/or a targeting moiety disclosed herein (e.g., on the luminal surface or on the exterior surface of the EV). Accordingly, in certain aspects, an EV disclosed herein (e.g., exosome), comprises a targeting moiety and a scaffold moiety, wherein the scaffold moiety anchors or links the targeting moiety to the EV (e.g., on the exterior surface of the EV). In some aspects, at least one of the additional exogenous biologically active molecules (e.g., antigen, adjuvant, or immune modulator) or any other exogenous biologically active molecules disclosed herein that can be expressed in the EVs disclosed herein (e.g., exosomes) is also anchored or linked to the EV via a scaffold moiety (e.g., either on the exterior surface or on the luminal surface). In some aspects, each of the additional exogenous biologically active molecules expressed in an EV (e.g., antigen, adjuvant, or immune modulator) is anchored or linked to the EV via a scaffold moiety. In certain aspects, scaffold moieties are polypeptides (“exosome proteins”). In other aspects, scaffold moieties are non-polypeptide moieties. In some aspects, exosome proteins include various membrane proteins, such as transmembrane proteins, integral proteins and peripheral proteins, enriched on the exosome membranes. They can include various CD proteins, transporters, integrins, lectins, and cadherins. In certain aspects, a scaffold moiety (e.g., exosome protein) comprises Scaffold X. In other aspects, a scaffold moiety (e.g., exosome protein) comprises Scaffold Y. In further aspects, a scaffold moiety (e.g., exosome protein) comprises both a Scaffold X and a Scaffold Y. Additional disclosure relating to the scaffold moieties that can be used with the present disclosure are provided throughout the present disclosure.

### III.A. Targeting Moieties

Certain aspects of the present disclosure are directed to a method of compartmentally administering to a target tissue in a subject a composition comprising an EV (e.g., an exosome), wherein the EV comprises a targeting moiety. In some aspects, the EV comprises a targeting moiety that specifically binds to a marker (or target molecule) expressed on a cell or a population of cells. In certain aspects, the marker is expressed on multiple cell types. In other aspects, the marker is expressed only on a specific population of cells.

In some aspects, a targeting moiety of the present disclosure specifically binds to a marker for a CNS cell. In certain aspects, the CNS cell is selected from a neuronal cell, a glial cell, and any combination thereof. In some aspects, the CNS cell is selected from an oligodendrocyte, an astrocyte, an ependymal cell, a microglia, and any combination thereof. In some aspects, the CNS cell is selected from a motor neuron, a sensory neuron, an interneuron, and any combination thereof. In some aspects, the targeting moiety specifically binds to a marker present on a neuron. In some aspects, the targeting moiety specifically binds to a marker present on a glial cell.

In some aspects, a targeting moiety of the present disclosure specifically binds to a marker for an eye cell. In certain aspects, the eye cell is selected from a rod cell, a cone cell, a retinal ganglion cell, and any combination thereof. In some aspects, the targeting moiety specifically binds to a marker present on a rod cell. In some aspects, the targeting moiety specifically binds to a marker present on a cone cell. In some aspects, the targeting moiety specifically binds to a marker present on a retinal ganglion cell.

In some aspects, a targeting moiety of the present disclosure specifically binds to a marker for a muscle cell. In certain aspects, the muscle cell is selected from a skeletal muscle cell, a smooth muscle cell, a cardiomyocyte, and any combination thereof. In some aspects, the targeting moiety specifically binds to a marker present on a skeletal muscle cell. In some aspects, the targeting moiety specifically binds to a marker present on a smooth muscle cell. In some aspects, the targeting moiety specifically binds to a marker present on a cardiomyocyte.

In some aspects, a targeting moiety of the present disclosure specifically binds to a tumor antigen. Any tumor antigen known in the art can be used in the methods disclosed herein. In some aspects, the tumor antigen is expressed on the surface of a cancer cell. In some aspects, the tumor antigen is present in the tumor microenvironment.

In some aspects, a targeting moiety of the present disclosure specifically binds to a marker for an immune cell. In some aspects, the immune cell is a T cell. In certain aspects, the T cell is a CD4+ T cell. In some aspects, the T cell is a CD8+ T cell.

In some aspects, a targeting moiety disclosed herein binds to human CD3 protein or a fragment thereof. Sequences for human CD3 protein are known in the art. In some aspects, a targeting moiety disclosed herein can bind to both human and mouse CD3, including any variants thereof. In some aspects, a targeting moiety of the present disclosure can bind to CD3 from other species, including but not limited to chimpanzee, rhesus monkey, dog, cow, horse, or rat. Sequences for such CD3 protein are also known in the art.

In some aspects, the immune cell is a B cell. In certain aspects, the targeting moiety comprises CD40L. In certain aspects, the targeting moiety comprises a fragment of CD40L, wherein the fragment is capable of binding the CD40 receptor.

In some aspects, the immune cell is a macrophage. In certain aspects, the targeting moiety specifically binds to a marker on a macrophage. In some aspects, the targeting moiety facilitates uptake of the EV by the macrophage. In some aspects, uptake of the EV activates the macrophage. In some aspects, the EV comprises a biologically active molecule that is capable of repolarizing a macrophage. In some aspects, uptake of the EV comprising the biologically active molecule repolarizes the macrophage from an M2 phenotype to an M1 phenotype. In some aspects, the biologically active molecule is an M2 polarization agent. In some aspects, the biologically active molecule is an M1 polarization agent. In some aspects, uptake of the EV comprising the biologically active molecule repolarizes the macrophage from an M1 phenotype to an M2 phenotype.

In some aspects, a targeting moiety disclosed herein can allow for greater uptake of an EV by a cell expressing a marker specific for the targeting moiety. In certain aspects, the uptake of an EV is increased by at least about 5%, at least about 10%, at least about 20%, at least about 30%, at least about 40%, at least about 50%, at least about 60%, at least about 70%, at least about 80%, at least about 90%, at least about 100% or more, compared to a reference. In some aspects, a reference comprises an EV that does not express a targeting moiety disclosed herein.

In some aspects, a targeting moiety disclosed herein allows for greater uptake of an EV by a CD4+ T cell. In certain aspects, the CD4+ T cell is a naive CD4+ T cell. In some aspects, the uptake of an EV is increased by at least about 5%, at least about 10%, at least about 20%, at least about 30%, at least about 40%, at least about 50%, at least about 60%, at least about 70%, at least about 80%, at least about 90%, at least about 100% or more, compared to a reference. In some aspects, a reference comprises an EV that does not express a targeting moiety disclosed herein.

In some aspects, the increased uptake of an EV disclosed herein can allow for greater immune response. Accordingly, in certain aspects, an EV expressing a targeting moiety disclosed herein can increase an immune response (e.g., against a tumor antigen loaded onto the exosome) by at least about 5%, at least about 10%, at least about 20%, at least about 30%, at least about 40%, at least about 50%, at least about 60%, at least about 70%, at least about 80%, at least about 90%, at least about 100% or more, compared to a reference. In some aspects, a reference comprises an EV that does not express a targeting moiety disclosed herein. In certain aspects, an immune response is mediated by T cells (e.g., CD8+ T cells or CD4+ T cells) and/or B cells.

As described supra, a targeting moiety disclosed herein can comprise a peptide, an antibody or an antigen-binding fragment thereof, a chemical compound, or any combination thereof. In some aspects, the targeting moiety is a peptide that can specifically bind to CD3. For example, in certain aspects, the peptide comprises a soluble fragment of CD3. In certain aspects, the peptide comprises a ligand (natural or synthetic) of CD3.

In some aspects, the targeting moiety is an antibody or an antigen-binding fragment thereof. In certain aspects, a targeting moiety is a single-chain Fv antibody fragment. In certain aspects, a targeting moiety is a single-chain F(ab) antibody fragment. In certain aspects, a targeting moiety is a nanobody. In certain aspects, a targeting moiety is a monobody.

In some aspects, the targeting moiety is an anti-CD3 antibody. The EVs expressing an anti-CD3 antibody are capable of targeting T cells, e.g., CD4+ T cells and/or CD8+ T cells. In certain aspects, EVs expressing an anti-CD3 antibody can specifically target naive CD4+ T cells.

In some aspects, an EV disclosed herein comprises one or more (e.g., 2, 3, 4, 5, or more) targeting moieties. In certain aspects, the one or more targeting moieties are expressed in combination with other exogenous biologically active molecules disclosed herein (e.g., therapeutic molecule, adjuvant, or immune modulator). In some aspects, the one or more targeting moieties can be expressed on the exterior surface of the EV, e.g., exosome. Accordingly, in certain aspects, the one or more targeting moieties are linked to a scaffold moiety (e.g., Scaffold X) on the exterior surface of the EV, e.g., exosome. When the one or more targeting moieties are expressed in combination with other exogenous biologically active molecules (e.g., therapeutic molecule, adjuvant, or immune modulator), the other exogenous biologically active molecules can be expressed on the surface (e.g., exterior surface or luminal surface) or in the lumen of the EV, e.g., exosome.

### III.B. Therapeutic Molecules /Biologically Active Molecules

In some aspects, an EV disclosed herein has been engineered or modified to deliver one or more (e.g., two, three, four, five or more) therapeutic molecules to a target. Any therapeutic molecule can be administered using the methods disclosed herein. In some aspects, the therapeutic molecule comprises a biologically active molecule. In some aspects, the biologically active molecule comprises a polypeptide. In some aspects, the biologically active molecule comprises a structural protein, an enzyme, a cytokine (such as an interferon and/or an interleukin) an antibiotic, a polyclonal or monoclonal antibody, or an effective part thereof, such as an Fv fragment, which antibody or part thereof can be natural, synthetic or humanized, a peptide hormone, a receptor, a signaling molecule or other protein.

In some aspects, the biologically active molecule comprises a nucleic acid. In some aspects, the biologically active molecule comprises an oligonucleotide or modified oligonucleotide, an antisense oligonucleotide or modified antisense oligonucleotide, cDNA, genomic DNA, an artificial or natural chromosome (e.g. a yeast artificial chromosome) or a part thereof, RNA, including mRNA, tRNA, rRNA or a ribozyme, or a peptide nucleic acid (PNA). In certain aspects, the biologically active molecule comprises a modified antisense oligonucleotide.

In some aspects, the biologically active molecule comprises a virus or a virus-like particle. In some aspects, the biologically active molecule comprises an AAV particle. In some aspects, the AAV particle comprises a gene of interest.

In some aspects, the biologically active molecule comprises a nucleotide, a ribonucleotide, or synthetic analogue thereof. In certain aspects, the nucleotide or the ribonucleotide is modified. In some aspects, the biologically active molecule comprises a non-peptide (e.g., steroid) hormone. In some aspects, the biologically active molecule comprises a proteoglycan. In some aspects, the biologically active molecule comprises a lipid. In some aspects, the biologically active molecule comprises or a carbohydrate. In certain aspects, the biologically active molecule comprises an antigen. In some aspects, the biologically active molecule comprises a targeting moiety (e.g., an antibody or an antigen-binding fragment thereof). In some aspects, the biologically active molecule comprises an adjuvant. In some aspects, the biologically active molecule comprises an immune modulator. In some aspects, the biologically active molecule comprises a macromolecule (e.g., a protein, an antibody, an enzyme, a peptide, DNA, RNA, or any combination thereof). In some aspects, the biologically active molecule comprises a small molecule (e.g., a miRNA, a dsDNA, a lncRNA, an antisense oligomer (ASO), a siRNA, a phosphorodiamidate morpholino oligomer (PMO), or a peptide-conjugated phosphorodiamidate morpholino oligomer (PPMO), a STING agonist, a pharmaceutical drug, or any combination thereof). In some aspects, the biologically active molecules are exogenous to the exosome, i.e., not naturally found in the exosome.

In some aspects, the therapeutic molecule comprises an agent that activates an immune response. In some aspects, the therapeutic molecule comprises a cytotoxic agent.

In certain aspects, a therapeutic molecule comprises an antigen. According, in certain aspects, an EV disclosed herein comprises a targeting moiety and an antigen.

In some aspects, an antigen that can be delivered using an EV disclosed herein comprises a tumor antigen. Non-limiting examples of tumor antigens include: alpha-fetoprotein (AFP), carcinoembryonic antigen (CEA), epithelial tumor antigen (ETA), mucin 1 (MUC1), Tn-MUC1, mucin 16 (MUC16), tyrosinase, melanoma-associated antigen (MAGE), tumor protein p53 (p53), CD4, CD8, CD45, CD80, CD86, programmed death ligand 1 (PD-L1), programmed death ligand 2 (PD-L2), NY-ESO-1, PSMA, TAG-72, HER2, GD2, cMET, EGFR, Mesothelin, VEGFR, alpha-folate receptor, CE7R, IL-3, Cancer-testis antigen (CTA), MART-1 gp100, TNF-related apoptosis-inducing ligand, Brachyury (preferentially expressed antigen in melanoma (PRAME)), or combinations thereof. In further aspects, an antigen can comprise a neoantigen. As used herein, the term “neoantigen,” refers to antigens encoded by tumor-specific mutated genes. In some aspects, the antigen is derived from a bacterium, a virus, fungus, protozoa, or any combination thereof. In some aspects, the antigen is derived from an oncogenic virus. In further aspects, the antigen is derived from a group comprising: a Human Gamma herpes virus 4 (Epstein Barr virus), influenza A virus, influenza B virus, cytomegalovirus, staphylococcus aureus, mycobacterium tuberculosis, chlamydia trachomatis, HIV-1, HIV-2, corona viruses (e.g., MERS-CoV and SARS CoV), filoviruses (e.g., Marburg and Ebola), Streptococcus pyogenes, Streptococcus pneumoniae, Plasmodia species (e.g., vivax and falciparum), Chikungunya virus, Human Papilloma virus (HPV), Hepatitis B, Hepatitis C, human herpes virus 8, herpes simplex virus 2 (HSV2), Klebsiella sp., Pseudomonas aeruginosa, Enterococcus sp., Proteus sp., Enterobacter sp., Actinobacter sp., coagulase-negative staphylococci (CoNS), Mycoplasma sp., or combinations thereof.

In some aspects, a therapeutic molecule comprises an immunosuppressive agent. Accordingly, in certain aspects, an EV disclosed herein comprises a targeting moiety and an immunosuppressive agent.

Non-limiting examples of other suitable therapeutic molecules include pharmacologically active drugs and genetically active molecules, including antineoplastic agents, anti-inflammatory agents, hormones or hormone antagonists, ion channel modifiers, and neuroactive agents. Examples of suitable payloads of therapeutic agents include those described in, “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming organs; Hormones and Hormone Antagonists; Vitamins, Dermatology; and Toxicology, all incorporated herein by reference. Suitable payloads further include toxins, and biological and chemical warfare agents, for example see Somani, S. M. (ed.), Chemical Warfare Agents, Academic Press, New York (1992)).

In certain aspects, an EV (e.g., exosomes) disclosed herein have been engineered or modified to comprise two or more therapeutic molecules (e.g., antigen or immunosuppressive agent), a first therapeutic molecule and a second therapeutic molecule (e.g., in addition to a targeting moiety disclosed herein). In some aspects, the first therapeutic molecule is linked to a first Scaffold Y on the luminal surface of the EV and the second therapeutic molecule is linked to a second Scaffold Y on the luminal surface of the EV, e.g., exosome. In some aspects, the first therapeutic molecule is linked to a Scaffold Y on the luminal surface of the EV and the second therapeutic molecule is in the lumen of the EV not linked to any scaffold moiety. In some aspects, the first therapeutic molecule is in the lumen of the EV not linked to any scaffold moiety, and the second therapeutic molecule is linked to a Scaffold Y on the luminal surface of the EV, e.g., exosome. In some aspects, the first therapeutic molecule is linked to a Scaffold Y on the luminal surface of the EV and the second therapeutic molecule is linked to a Scaffold X on the exterior surface of the EV, e.g., exosome. In some aspects, the first therapeutic molecule is in the lumen of the EV not linked to any scaffold moiety, and the second therapeutic molecule is linked to a Scaffold X on the exterior surface of the EV, e.g., exosome. In some aspects, the first therapeutic molecule is linked to a Scaffold Y on the luminal surface of the EV and the second therapeutic molecule is linked to a Scaffold X on the luminal surface of the EV, e.g., exosome. In some aspects, the first therapeutic molecule is in the lumen of the EV not linked to any scaffold moiety, and the second therapeutic molecule is linked to a Scaffold X on the luminal surface of the EV, e.g., exosome. In some aspects, the first therapeutic molecule is linked to a Scaffold X on the luminal surface of the EV and the second therapeutic molecule is linked to the Scaffold X on the exterior surface of the EV, e.g., exosome. In some aspects, the first therapeutic molecule is linked to a first Scaffold X on the exterior surface of the EV and the second therapeutic molecule is linked to a second Scaffold X on the exterior surface of the EV, e.g., exosome. In some aspects, the first therapeutic molecule is linked to a Scaffold X on the exterior surface of the EV and the second therapeutic molecule is linked to a Scaffold Y on the luminal surface of the EV, e.g., exosome. In some aspects, the first therapeutic molecule is linked to a Scaffold X on the exterior surface of the EV and the second therapeutic molecule is in the lumen of the EV not linked to any scaffold moiety. In some aspects, the first therapeutic molecule is linked to a Scaffold X on the exterior surface of the EV and the second therapeutic molecule is linked to the Scaffold X on the luminal surface of the EV, e.g., exosome. In some aspects, the first therapeutic molecule is linked to a first Scaffold X on the luminal surface of the EV and the second therapeutic molecule is linked to a second Scaffold X on the luminal surface of the EV, e.g., exosome. In some aspects, the first therapeutic molecule is linked to a Scaffold X on the luminal surface of the EV and the second therapeutic molecule is linked to a Scaffold Y on the luminal surface of the EV, e.g., exosome. In some aspects, the first therapeutic molecule is linked to a Scaffold X on the luminal surface of the EV and the second therapeutic molecule is in the lumen of the EV not linked to any scaffold moiety. In some aspects, the first therapeutic molecule is linked to a first Scaffold X on the exterior surface of the EV and the second therapeutic molecule is linked to a second Scaffold X on the luminal surface of the EV, e.g., exosome. In some aspects, the first therapeutic molecule is linked to a first Scaffold X on the luminal surface of the EV and the second therapeutic molecule is linked to a second Scaffold X on the exterior surface of the EV, e.g., exosome. In some aspects, the first therapeutic molecule is in the lumen of the EV not linked to any scaffold moiety, and the second therapeutic molecule is in the lumen of the EV not linked to any scaffold moiety.

In some aspects, a therapeutic molecule comprises a self-antigen. As used herein, the term “self-antigen” refers to an antigen that is expressed by a host cell or tissue. Under normal healthy state, such antigens are recognized by the body as self and do not elicit an immune response. However, under certain diseased conditions, a body’s own immune system can recognize self-antigens as foreign and mount an immune response against them, resulting in autoimmunity. In certain aspects, EVs of the present disclosure can comprise a self-antigen (i.e., the self (germline) protein to which T cell responses have been induced and resulted in autoimmunity). Such EVs can be used to target the autoreactive T cells and suppress their activity. Non-limiting examples of self-antigens (including the associated disease or disorder) include beta-cell proteins (type I diabetes), myelin oligodendrocyte glycoprotein (MOG, multiple sclerosis), synovial proteins (rheumatoid arthritis), or combinations thereof. In some aspects, self-antigen comprises CD47. In some aspects, the self-antigen comprises a fragment of CD47. In some aspects, self-antigen comprises CD24. In some aspects, the self-antigen comprises a fragment of CD24. In some aspects, the therapeutic molecule comprises an antibody or antigen-binding fragment thereof. In some aspects, the therapeutic molecule comprises at least 2, at least 3, at least 4, or at least 5 antibodies or antigen-binding fragments thereof. In some aspects, the antibody or antigen-binding fragment thereof comprises a scFv, scFab, scFab-Fc, nanobody, or any combination thereof. In some aspects, the antibody or antigen-binding fragment thereof comprises an agonist antibody, blocking antibody, a targeting antibody, a fragment thereof, or a combination thereof. In some aspects, the agonist antibody is a CD40L agonist. In some aspects, the blocking antibody binds a target protein selected from programmed death 1 (PD-1), programmed death ligand 1 (PD-L1), cytotoxic T-lymphocyte-associated protein 4, and any combination thereof. In some aspects, the EV comprises an anti-IL12 antibody or an antigen-binding fragment thereof and an anti-CD40L antibody or antigen-binding fragment thereof.

In some aspects, the EVs targeting CNS further comprise one or more payloads, e.g., one or more biologically active moieties. Payloads (e.g., biologically active moieties, e.g., therapeutic molecule) comprise a small molecule, a therapeutic protein, an antigen, an adjuvant, an immune modulator, or any combination thereof.

In some aspects, an EV disclosed herein is capable of delivering a payload (a biologically active molecule attached to the EV via a maleimide moiety) to a target. The payload is an agent that acts on a target (e.g., a target cell) that is contacted with the EV. Contacting can occur in vitro or in a subject. Non-limiting examples of payloads that can attached to an EV via a maleimide moiety include agents such as, nucleotides (e.g., nucleotides comprising a detectable moiety or a toxin or that disrupt transcription), nucleic acids (e.g., DNA or mRNA molecules that encode a polypeptide such as an enzyme, or RNA molecules that have regulatory function such as miRNA, dsDNA, lncRNA, or siRNA), amino acids (e.g., amino acids comprising a detectable moiety or a toxin that disrupt translation), polypeptides (e.g., enzymes), lipids, carbohydrates, and small molecules (e.g., small molecule drugs and toxins). In some aspects, a payload is in the lumen of the EV. In some aspects, an EV can comprise more than one payload, e.g., a first payload in solution the lumen of EV, and a second payload attached, e.g., to the external surface of the EV via a maleimide moiety.

In some aspects, the payload is a small molecule. In some aspects, the small molecule is a proteolysis-targeting chimera (PROTAC). In some aspects, the payload comprises a nucleotide, wherein the nucleotide is a stimulator of interferon genes protein (STING) agonist. STING is a cytosolic sensor of cyclic dinucleotides that is typically produced by bacteria. Upon activation, it leads to the production of type I interferons and initiates an immune response.

In some aspects, the EV of the present disclosure further comprises one or more STING agonists covalently linked to the EV via a maleimide moiety. In some aspects, the STING agonist comprises a cyclic nucleotide STING agonist or a non-cyclic dinucleotide STING agonist. For example, non limiting examples of cyclic nucleotides STING agonist include any CDN disclosed in WO 2016/096174A1, which is incorporated by reference in its entirety.

In some aspects, the STING agonist useful for the present disclosure comprises a compound disclosed in WO 2014/093936, WO 2014/189805, and WO 2015/077354, each content of which is incorporated herein by reference in its entirety. See also Cell reports 11, 1018-1030 (2015).

In some aspects, the STING agonist useful for the present disclosure comprises c-di-AMP, c-di-GMP, c-di-IMP, c-AMP-GMP, c-AMP-IMP, and c-GMP-IMP, described in WO 2013/185052 and Sci. Transl. Med. 283,283ra52 (2015), which are incorporated herein by reference in their entireties.

In some aspects, the STING agonist useful for the present disclosure comprises a compound disclosed in WO 2014/189806, WO 2015/185565, WO 2014/179760, WO 2014/179335, WO 2015/017652, WO 2016/096577, WO 2011/003025, WO 2016/145102, WO 2017/027646, WO 2017/075477, WO 2017/027645, WO 2018/100558, WO 2017/175147, and WO 2017/175156, each content of which is incorporated herein by reference in its entirety.

In some aspects, the EV comprises a cyclic dinucleotide STING agonist and/or a non-cyclic dinucleotide STING agonist. In some aspects, when several cyclic dinucleotide STING agonist are present on an EV disclosed herein, such STING agonists can be the same or they can be different. In some aspects, when several non-cyclic dinucleotide STING agonist are present, such STING agonists can be the same or they can be different. In some aspects, an EV composition of the present disclosure can comprise two or more populations of EVs wherein each population of EVs comprises a different STING agonist or combination thereof.

In some aspects, the payload (e.g., a biologically active molecule) is a TLR agonist. Non-limiting examples of TLR agonists can be found at WO2008115319A2, US20130202707A1, US20120219615A1, US20100029585A1, WO2009030996A1, WO2009088401A2, and WO2011044246A1, each of which are incorporated by reference in its entirety.

Non-limiting examples of TLR agonists include: TLR2 agonist (e.g., lipoteichoic acid, atypical LPS, MALP-2 and MALP-404, OspA, porin, LcrV, lipomannan, GPI anchor, lysophosphatidylserine, lipophosphoglycan (LPG), glycophosphatidylinositol (GPI), zymosan, hsp60, gH/gL glycoprotein, hemagglutinin), a TLR3 agonist (e.g., double-stranded RNA, e.g., poly(I:C)), a TLR4 agonist (e.g., lipopolysaccharides (LPS), lipoteichoic acid, β-defensin 2, fibronectin EDA, HMGB1, snapin, tenascin C), a TLR5 agonist (e.g., flagellin), a TLR6 agonist, a TLR⅞ agonist (e.g., single-stranded RNA, CpG-A, Poly G10, Poly G3, Resiquimod), a TLR9 agonist (e.g., unmethylated CpG DNA), and combinations thereof.

In some aspects, the payload is an antibody or antigen binding fragment thereof. In some aspects, the payload is an ADC. In some aspects, the payload is a small molecule comprising a synthetic antineoplastic agent (e.g., monomethyl auristatin E (MMAE) (vedotin)), a cytokine release inhibitor (e.g., MCC950), an mTOR inhibitor (e.g,. Rapamycin and its analogs (Rapalogs)), an autotaxin inhibitor (e.g., PAT409), a lysophosphatidic acid receptor antagonist (e.g.,BMS-986020), or any combination thereof.

In some aspects, the payload in an antisense oligonucleotide, a phosphorodiamidate Morpholino oligomer (PMO), or a peptide-conjugated phosphorodiamidate morpholino oligomer (PPMO). In some aspects, the payload is a fusogenic peptide.

### III.B.1. Macrophage-Targeting Biologically Active Molecules

In some aspects, the biologically active molecule targets macrophages. In other aspects, the biologically active molecule induces macrophage polarization. Macrophage polarization is a process by which macrophages adopt different functional programs in response to the signals from their microenvironment. This ability is connected to their multiple roles in the organism: they are powerful effector cells of innate immune system, but also important in removal of cellular debris, embryonic development and tissue repair.

By simplified classification, macrophage phenotype has been divided into 2 groups: M1 (classically activated macrophages) and M2 (alternatively activated macrophages). This broad classification was based on in vitro studies, in which cultured macrophages were treated with molecules that stimulated their phenotype switching to particular state. In addition to chemical stimulation, it has been shown that the stiffness of the underlying substrate a macrophage is grown on can direct polarization state, functional roles and migration mode. M1 macrophages were described as the pro-inflammatory type, important in direct host-defense against pathogens, such as phagocytosis and secretion of pro-inflammatory cytokines and microbicidal molecules. M2 macrophages were described to have quite the opposite function: regulation of the resolution phase of inflammation and the repair of damaged tissues. Later, more extensive in vitro and ex vivo studies have shown that macrophage phenotypes are much more diverse, overlapping with each other in terms of gene expression and function, revealing that these many hybrid states form a continuum of activation states which depend on the microenvironment. Moreover, in vivo, there is a high diversity in gene expression profile between different populations of tissue macrophages. Macrophage activation spectrum is thus considered to be wider, involving complex regulatory pathway to response to plethora of different signals from the environment. The diversity of macrophage phenotypes still remain to be fully characterized in vivo.

The imbalance of the macrophage types is related to a number of immunity-related diseases. For example, increased M1/M2 ratio can correlate with development of inflammatory bowel disease, as well as obesity in mice. On the other side, in vitro experiments implicated M2 macrophages as the primary mediators of tissue fibrosis. Several studies have associated the fibrotic profile of M2 macrophages with the pathogenesis of systemic sclerosis. Non-limiting examples of the macrophage targeting biologically active molecules are: P13Kγ (phosphatidylinositol-4,5-bisphosphate 3-kinase catalytic subunit gamma), RIP1 (Receptor Interacting Protein (RIP) kinase 1, RIPK1), HIF-1α (Hypoxia-inducible factor 1-alpha), AHR1 (Adhesion and hyphal regulator 1), miR146a, miR155, IRF4 (Interferon regulatory factor 4), PPARγ (Peroxisome proliferator-activated receptor gamma), IL-4RA (Interleukin-4 receptor subunit alpha), TLR8 (Toll-like receptor 8), and TGF-β1 (Transforming growth factor beta-1 proprotein).

### III.B.2. Oligonucleotides

In some aspects, the payload for the EVs is an antisense oligonucleotide, a phosphorodiamidate Morpholino oligomer (PMO), or a peptide-conjugated phosphorodiamidate morpholino oligomer (PPMO), an antisense oligonucleotide (ASO), a siRNA, a miRNA, a shRNA, a nucleic acid, or any combination thereof.

In some aspects, the ASO is targets PMP22. In humans, the PMP22 gene is located on chromosome 17p11.2 and spans approximately 40 kb. The gene contains six exons conserved in both humans and rodents, two of which are 5′ untranslated exons (1a and 1b) and result in two different RNA transcripts with identical coding sequences. The two transcripts differ in their 5′ untranslated regions and have their own promoter regulating expression. The remaining exons (2 to 5) include the coding region of the PMP22 gene, and are joined together after post-transcriptional modification (i.e. alternative splicing). The PMP22 protein is characterized by four transmembrane domains, two extracellular loops (ECL1 and ECL2), and one intracellular loop. ECL1 has been suggested to mediate a homophilic interaction between two PMP22 proteins, whereas ECL2 has been shown to mediate a heterophilic interaction between PMP22 protein and Myelin protein zero (MPZ or MP0).

Improper gene dosage of the PMP22 gene can cause aberrant protein synthesis and function of myelin sheath. Since the components of myelin are stoichiometrically set, any irregular expression of a component can cause destabilization of myelin and neuropathic disorders. Alterations of PMP22 gene expression are associated with a variety of neuropathies, such as Charcot-Marie-Tooth type 1A (CMT1A), Dejerine-Sottas disease, and Hereditary Neuropathy with Liability to Pressure Palsy (HNPP). Too much PMP22 (e.g. caused by gene duplication) results in CMT1A. Gene duplication of PMP22 is the most common genetic cause of CMT where the overproduction of PMP22 results in defects in multiple signaling pathways and dysfunction of transcriptional factors like KNOX20, SOX10 and EGR2.

The sequence for the human PMP22 gene can be found under publicly available as NCBI RefSeq Acc. No. NM_000304. Alternative RefSeq mRNA transcripts have accession numbers NM_001281455, NM-001281456, NM-153321, and NM_153322, respectively. The human PMP22 gene is found at chromosome location 17p12 at 15,229,777-15,265,326.

The sequence for the human PMP22 pre-mRNA transcript corresponds to the reverse complement of residues 15,229,777-15,265,326, of chromosome location 17p12. The sequence for human PMP22 protein can be found under publicly available Uniprot Accession Number Q01453. Potential PMP22 isoforms have Uniprot Accession Numbers A8MU75, J3KQW0, A0A2R8Y5L5, J3KT36, and J3QS08, respectively. The publicly available contents of the database entries corresponding to accession numbers disclosed herein are incorporated by reference in their entireties.

In some aspects, the ASO targets a transcript, which is a STAT6 transcript, a CEBP/β transcript, a STAT3 transcript, a KRAS transcript, a NRAS transcript, an NLPR3 transcript, or any combination thereof.

STAT6 (STAT6) is also known as signal transducer and activator of transcription 6. Synonyms of STAT6/STAT6 are known and include IL-4 STAT; STAT, Interleukin4-Induced; Transcription Factor IL-4 STAT; STAT6B; STAT6C; and D12S1644. The sequence for the human STAT6 gene can be found under publicly available GenBank Accession Number NC_000012.12:c57111413-57095404. The human STAT6 gene is found at chromosome location 12q13.3 at 57111413-57095404, complement.

CEBP/β (CEBP/β) is also known as CCAAT/enhancer-binding protein beta. Synonyms of CEBP/β/CEBP/β are known and include C/EBP beta; Liver activator protein; LAP; Liver-enriched inhibitory protein; LIP; Nuclear factor NF-IL6; transcription factor 5; TCF-5; CEBPB; CEBPb; CEBPβ; CEBP/B; and TCF5. The sequence for the human CEBP/β gene can be found under publicly available GenBank Accession Number NC_000020.11 (50190583..50192690). The human CEBP/β gene is found at chromosome location 20q13.13 at 50190583-50192690.

NRas is an oncogene encoding a membrane protein that shuttles between the Golgi apparatus and the plasma membrane. NRas-encoding genomic DNA can be found at Chromosomal position 1p13.2 (i.e., nucleotides 5001 to 17438 of GenBank Accession No. NG_007572). N-ras mutations have been described in melanoma, thyroid carcinoma, teratocarcinoma, fibrosarcoma, neuroblastoma, rhabdomyosarcoma, Burkitt lymphoma, acute promyelocytic leukemia, T cell leukemia, and chronic myelogenous leukemia. Oncogenic N-Ras can induce acute myeloid leukemia (AML)- or chronic myelomonocytic leukemia (CMML)-like disease in mice. Neuroblastoma RAS viral oncogene (NRas) is known in the art by various names. Such names include: GTPase NRas, N-ras protein part 4, neuroblastoma RAS viral (v-ras) oncogene homolog neuroblastoma RAS viral oncogene homolog, transforming protein N-Ras, and v-ras neuroblastoma RAS viral oncogene homolog.

Signal Transducer and Activator of Transcription 3 (STAT3) is a signal transducer and activator of transcription that transmits signals from cell surface receptors to the nucleus. STAT3 is frequently hyperactivated in many human cancers. STAT3-encoding genomic DNAcan be found at Chromosomal position 17q21.2 (i.e., nucleotides 5,001 to 80,171 of GenBank Accession No. NG_007370.1) NLRP3 (NLRP3) is also known as NLR family pyrin domain containing 3. Synonyms of NLRP3/NLRP3 are known and include NLRP3; Clorf7; CIASl; NALP3; PYPAFl: nucleotide-binding oligomerization domain, leucine rich repeat and pyrin domain containing 3; cold-induced autoinflammatory syndrome 1 protein; cryopyin; NACHT, LRR and PYD domains-containing protein 3; angiotensin/vasopressin receptor AII/AVP-like; caterpillar protein 1.1; CLR1.1; cold-induced autoinflammatory syndrome 1 protein; and PYRIN-containing APAF1-like protein 1. The sequence for the human NLRP3 gene can be found under publicly available GenBank Accession Number NC_000001.11:247416156-247449108. The human NLRP3 gene is found at chromosome location 1q44 at 247,416,156-247,449,108.

KRAS is known in the art by various names. Such names include: KRAS Proto-Oncogene, GTPase; V-Ki-Ras2 Kirsten Rat Sarcoma 2 Viral Oncogene Homolog; GTPase KRas; C-Ki-Ras; K-Ras 2; KRAS2; RASK2; V-Ki-Ras2 Kirsten Rat Sarcoma Viral Oncogene Homolog; Kirsten Rat Sarcoma Viral Proto-Oncogene; Cellular Transforming Proto-Oncogene; Cellular C-Ki-Ras2 Proto-Oncogene; Transforming Protein P21; PR310 C-K-Ras Oncogene; C-Kirsten-Ras Protein; K-Ras P21 Protein; and Oncogene KRAS2. The sequence for the human KRAS gene can be found at chromosomal location 12p12.1 and under publicly available GenBank Accession Number NC_000012 (25,204,789 - 25,250,936). The genomic sequence for human wild-type KRAS transcript corresponds to the reverse complement of residues 25,204,789 - 25,250,936 of NC_000012.

### III.B.3. Therapeutic Protein

In some aspects, the payload for the EVs, exosomes, administered compartmentally and/or targeting CNS comprises a therapeutic protein. In some aspects, the therapeutic protein comprises a clotting factor, an immunomodulator, a growth factor, or any combination thereof.

In some aspects, a payload that can be delivered by the EVs comprises a clotting factor. In some aspects, the therapeutic protein comprises a FIX polypeptide. In some aspects, the FIX polypeptide comprises FIX or a variant or fragment thereof, wherein the FIX or the variant or fragment thereof has a FIX activity. In some aspects, the therapeutic protein comprises a FVIII polypeptide. In some aspects, the FVIII polypeptide comprises FVIII or a variant or fragment thereof, wherein the FVIII or the variant or fragment thereof has a FVIII activity. In some aspects, the FVIII protein is a B domain deleted FVIII.

In some aspects, a payload that can be delivered by the EVs targeting CNS comprises a growth factor. The growth factor can be selected from any growth factor known in the art. In some aspects, the growth factor is a hormone. In some aspects, the growth factor is a cytokine. In some aspects, the growth factor is a chemokine.

In some aspects, the growth factor is adrenomedullin (AM). In some aspects, the growth factor is angiopoietin (Ang). In some aspects, the growth factor is autocrine motility factor. In some aspects, the growth factor is a Bone morphogenetic protein (BMP). In some aspects, the BMP is selects from BMP2, BMP4, BMP5, and BMP7. In some aspects, the growth factor is a ciliary neurotrophic factor family member. In some aspects, the ciliary neurotrophic factor family member is selected from ciliary neurotrophic factor (CNTF), leukemia inhibitory factor (LIF), interleukin-6 (IL-6). In some aspects, the growth factor is a colony-stimulating factor. In some aspects, the colony-stimulating factor is selected from macrophage colony-stimulating factor (m-CSF), granulocyte colony-stimulating factor (G-CSF), and granulocyte macrophage colony-stimulating factor (GM-CSF). In some aspects, the growth factor is an epidermal growth factor (EGF). In some aspects, the growth factor is an ephrin. In some aspects, the ephrin is selected from ephrin A1, ephrin A2, ephrin A3, ephrin A4, ephrin A5, ephrin B1, ephrin B2, and ephrin B3. In some aspects, the growth factor is erythropoietin (EPO). In some aspects, the growth factor is a fibroblast growth factor (FGF). In some aspects, the FGF is selected from FGF1, FGF2, FGF3, FGF4, FGF5, FGF6, FGF7, FGF8, FGF9, FGF10, FGF11, FGF12, FGF13, FGF14, FGF15, FGF16, FGF17, FGF18, FGF19, FGF20, FGF21, FGF22, and FGF23. In some aspects, the growth factor is foetal bovine somatotrophin (FBS). In some aspects, the growth factor is a GDNF family member. In some aspects, the GDNF family member is selected from glial cell line-derived neurotrophic factor (GDNF), neurturin, persephin, and artemin. In some aspects, the growth factor is growth differentiation factor-9 (GDF9). In some aspects, the growth factor is hepatocyte growth factor (HGF). In some aspects, the growth factor is hepatoma-derived growth factor (HDGF). In some aspects, the growth factor is insulin. In some aspects, the growth factor is an insulin-like growth factor. In some aspects, the insulin-like growth factor is insulin-like growth factor-1 (IGF-1) or IGF-2. In some aspects, the growth factor is an interleukin (IL). In some aspects, the IL is selected from IL-1, IL-2, IL-3, IL-4, IL-5, IL-6, and IL-7. In some aspects, the growth factor is keratinocyte growth factor (KGF). In some aspects, the growth factor is migration-stimulating factor (MSF). In some aspects, the growth factor is macrophage-stimulating protein (MSP or hepatocyte growth factor-like protein (HGFLP)). In some aspects, the growth factor is myostatin (GDF-8). In some aspects, the growth factor is a neuregulin. In some aspects, the neuregulin is selected from neuregulin 1 (NRG1), NRG2, NRG3, and NRG4. In some aspects, the growth factor is a neurotrophin. In some aspects, the growth factor is brain-derived neurotrophic factor (BDNF). In some aspects, the growth factor is nerve growth factor (NGF). In some aspects, the NGF is neurotrophin-3 (NT-3) or NT-4. In some aspects, the growth factor is placental growth factor (PGF). In some aspects, the growth factor is platelet-derived growth factor (PDGF). In some aspects, the growth factor is renalase (RNLS). In some aspects, the growth factor is T-cell growth factor (TCGF). In some aspects, the growth factor is thrombopoietin (TPO). In some aspects, the growth factor is a transforming growth factor. In some aspects, the transforming growth factor is transforming growth factor alpha (TGF-α) or TGF-β. In some aspects, the growth factor is tumor necrosis factor-alpha (TNF-α). In some aspects, the growth factor is another vascular endothelial growth factor (VEGF).

In some aspects, the therapeutic protein comprises a subunit of the Rab geranylgeranyltransferase (GGTase) complex. In some aspects, the therapeutic protein comprises Rab proteins GGTase component A 1 (REP1). In some aspects, the REP1 comprises an amino acid sequence at least about 70%, at least about 75%, at least about at least about 80%, at least about 85%, at least about 90%, at least about 95%, at least about 96%, at least about 97%, at least about 98%, at least about 99%, or about 100% identical to SEQ ID NO: 263. REP1 deficiency is associated with Choroideremia (CHM), a rare X-linked progressive degeneration of the choroid, retinal pigment epithelium and photoreceptors of the eye. The typical natural history in afflicted males is onset of nightblindness during teenage years, and then progressive loss of peripheral vision during the 20’s and 30’s leading to complete blindness in the 40’s. Female carriers have mild symptoms most notably nightblindness but can occasionally have a more severe phenotype.

TABLE 1

```
REP1 Amino Acid Sequence (SEQ ID NO: 263)
MADTLPSEFDVIVIGTGLPESIIAAACSRSGRRVLHVDSRSYYGGNWASFSFSGLLSWLKEYQENSDIVSDSPVWQ
      DQTLENEEAIALSRKDKTIQHVEVFCYASQDLHEDVEEAGALQKNHALVTSANSTEAADSAFLPTEDESLSTM
      SCEMLTEQTPSSDPENALEVNGAEVTGEKENHCDDKTCVPSTSAEDMSENVPIAEDTTEQPKKNRITYSQIIK
      EGRRFNIDLVSKLLYSRGLLIDLLIKSNVSRYAEFKNITRILAFREGRVEQVPCSRADVFNSKQLTMVEKRML
      MKFLTFCMEYEKYPDEYKGYEEITFYEYLKTQKLTPNLQYIVMHSIAMTSETASSTIDGLKATKNFLHCLGRY
      GNTPFLFPLYGQGELPQCFCRMCAVFGGIYCLRHSVQCLVVKESRKCKAIIDQFGQRIISEHFLVEDSYFPE
      NMCSRVQYRQISRAVLITDRSVLKTDSDQQISILTVPAEEPGTFAVRVIELCSSTMTCMKGTYLVHLTCTSSK
      TAREDLESVVQKLFVPYTEMEIENEQVEKPRILWALYFNMRDSSDISRSCYNDLPSNVYVCSGPDCGLGNDNA
      VKQAETLFQEICPNEDFCPPPPNPEDIILDGDSLQPEASESSAIPEANSETFKESTNLGNLEESSE
```

The disease is caused by mutations in the REP1 gene, (Rab escort protein 1), which is located on the X chromosome 21q region. In most cells in the body, the REP2 protein, which is 75% homologous to REP1, compensates for the REP1 deficiency. In the eye, however, for reasons that are not yet clear, REP2 is unable to compensate for the REP1 deficiency. Hence in the eye, REP polypeptide activity is insufficient to maintain normal prenylation of the target proteins (Rab GTPases) leading to cellular dysfunction and ultimate death, primarily affecting the outer retina and choroid.

In some aspects, the payload targets a tumor antigen. Non-limiting examples of tumor antigens include: alpha-fetoprotein (AFP), carcinoembryonic antigen (CEA), epithelial tumor antigen (ETA), mucin 1 (MUC1), Tn-MUC1, mucin 16 (MUC16), tyrosinase, melanoma-associated antigen (MAGE), tumor protein p53 (p53), CD4, CD8, CD45, CD80, CD86, programmed death ligand 1 (PD-L1), programmed death ligand 2 (PD-L2), NY-ESO-1, PSMA, TAG-72, HER2, GD2, cMET, EGFR, Mesothelin, VEGFR, alpha-folate receptor, CE7R, IL-3, Cancer-testis antigen (CTA), MART-1 gp100, TNF-related apoptosis-inducing ligand, or combinations thereof.

In some aspects, the payload targeting a tumor antigen comprises a peptide, an antibody or an antigen-binding fragment thereof, a chemical compound, an RNA aptamer, or any combination thereof that targets or antagonizes the tumor antigen. In some aspects, the payload targeting a tumor antigen comprises a microprotein, a designed ankyrin repeat protein (darpin), an anticalin, an adnectin, an aptamer, a peptide mimetic molecule, a natural ligand for a receptor, a camelid nanobody, or any combination thereof.

In some aspects, the payload targeting a tumor antigen comprises a full-length antibody, a single domain antibody, a heavy chain only antibody (VHH), a single chain antibody, a shark heavy chain only antibody (VNAR), an scFv, a Fv, a Fab, a Fab′, a F(ab′)2, or any combination thereof.

### III.B.4. Adjuvants

As described supra, EVs of the present disclosure can comprise one or more exogenous biologically active molecules. In some aspects, an exogenous biologically active molecule that can be expressed in an EV is an adjuvant. Accordingly, in certain aspects, an EV disclosed herein comprises a targeting moiety and an adjuvant. In some aspects, EVs (e.g., exosome) disclosed herein comprises two, three, four, five or more different adjuvants. As used herein, the term “adjuvant” refers to any substance that enhances the therapeutic effect of the payload.

In some aspects, the adjuvant increases the bioavailability of the EV, and its payload. Various polypeptides are known in the art that can extend, e.g., the half-life of an agent in vivo. For example, in some aspects, the EV comprises one or more surface antigen that inhibits uptake of the EV by a macrophage. In some aspects, the surface antigen is associated with the exterior surface of the EV. In certain aspects, the surface antigen comprises CD47, e.g., human CD47. In some aspects, the surface antigen comprises a fragment of CD47, e.g., human CD47. In certain aspects, the surface antigen comprises CD24, e.g., human CD24. In some aspects, the surface antigen comprises a fragment of CD24, e.g., human CD24.In some aspects, the surface antigen increases the bioavailability of the EV by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 450%, at least about 500%, at least about 600%, at least about 700%, at least about 800%, at least about 900%, at least about 1000%, or at least about 2000%, relative to the bioavailability of an EV lacking the surface antigen. In some aspects, the surface antigen increases the bioavailability of the EV by at least about 1.5-fold, at least about 2.0-fold, at least about 2.5-fold, at least about 3.0-fold, at least about 3.5-fold, at least about 4.0-fold, at least about 4.5-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, at least about 50-fold, at least about 60-fold, at least about 70-fold, at least about 80-fold, at least about 90-fold, or at least about 100-fold, relative to the bioavailability of an EV lacking the surface antigen.

In some aspects, the adjuvant increases an immune response to an antigen. Accordingly, EVs described herein are capable of increasing an immune response to an antigen by at least about 5%, at least about 10%, at least about 20%, at least about 30%, at least about 40%, at least about 50%, at least about 60%, at least about 70%, at least about 80%, at least about 90%, at least about 100% or more, compared to a reference (e.g., corresponding EV without the adjuvant or a non-EV delivery vehicle comprising an antigen and adjuvant). Non-limiting examples of adjuvants include Stimulator of Interferon Genes (STING) agonist, a toll-like receptor (TLR) agonist, an inflammatory mediator, and combinations thereof.

In some aspects, a targeting moiety disclosed herein can reduce the amount (i.e., dose) of adjuvant (e.g., STING agonist) required to induce an immune response to an antigen (e.g., tumor-associated antigen). In certain aspects, a targeting moiety disclosed herein reduces the amount of adjuvant required to induce a comparable immune response induced by a reference EV (i.e., comprising the same adjuvant but does not express a targeting moiety) by at least about one-fold, at least about two-fold, at least about three-fold, at least about four-fold, at least about five-fold, at least about six-fold, at least about seven-fold, at least about eight-fold, at least about nine-fold, at least about ten-fold, at least about 15-fold, at least about 20-fold, at least about 30-fold, at least about 40-fold, at least about 50-fold, at least about 60-fold, at least about 70-fold, at least about 80-fold, at least about 90-fold, or at least about 100-fold. In some aspects, a targeting moiety disclosed herein reduces the amount of adjuvant required to induce a comparable immune response induced by a reference EV (i.e., comprising the same adjuvant but does not express a targeting moiety) by about ten-fold.

In certain aspects, the present disclosure is directed to modified or engineered EVs comprising two or more exogenous biologically active molecules, wherein the two or more exogenous biologically active molecules are adjuvants, a first adjuvant and a second adjuvant (e.g., in addition to a targeting moiety disclosed herein). In some aspects, the first adjuvant is linked to a first Scaffold Y on the luminal surface of the EV and the second adjuvant is linked to a second Scaffold Y on the luminal surface of the EV, e.g., exosome. In some aspects, the first adjuvant is linked to a Scaffold Y on the luminal surface of the EV and the second adjuvant is in the lumen of the EV not linked to any scaffold moiety. In some aspects, the first adjuvant is in the lumen of the EV not linked to any scaffold moiety, and the second adjuvant is linked to a Scaffold Y on the luminal surface of the EV, e.g., exosome. In some aspects, the first adjuvant is linked to a Scaffold Y on the luminal surface of the EV and the second adjuvant is linked to a Scaffold X on the exterior surface of the EV, e.g., exosome. In some aspects, the first adjuvant is in the lumen of the EV not linked to any scaffold moiety, and the second adjuvant is linked to a Scaffold X on the exterior surface of the EV, e.g., exosome. In some aspects, the first adjuvant is linked to a Scaffold Y on the luminal surface of the EV and the second adjuvant is linked to a Scaffold X on the luminal surface of the EV, e.g., exosome. In some aspects, the first adjuvant is in the lumen of the EV not linked to any scaffold moiety, and the second adjuvant is linked to a Scaffold X on the luminal surface of the EV, e.g., exosome. In some aspects, the first adjuvant is linked to a Scaffold X on the luminal surface of the EV and the second adjuvant is linked to the Scaffold X on the exterior surface of the EV, e.g., exosome. In some aspects, the first adjuvant is linked to a first Scaffold X on the exterior surface of the EV and the second adjuvant is linked to a second Scaffold X on the exterior surface of the EV, e.g., exosome. In some aspects, the first adjuvant is linked to a Scaffold X on the exterior surface of the EV and the second adjuvant is linked to a Scaffold Y on the luminal surface of the EV, e.g., exosome. In some aspects, the first adjuvant is linked to a Scaffold X on the exterior surface of the EV and the second adjuvant is in the lumen of the EV not linked to any scaffold moiety. In some aspects, the first adjuvant is linked to a Scaffold X on the exterior surface of the EV and the second adjuvant is linked to the Scaffold X on the luminal surface of the EV, e.g., exosome. In some aspects, the first adjuvant is linked to a first Scaffold X on the luminal surface of the EV and the second adjuvant is linked to a second Scaffold X on the luminal surface of the EV, e.g., exosome. In some aspects, the first adjuvant is linked to a Scaffold X on the luminal surface of the EV and the second adjuvant is linked to a Scaffold Y on the luminal surface of the EV, e.g., exosome. In some aspects, the first adjuvant is linked to a Scaffold X on the luminal surface of the EV and the second adjuvant is in the lumen of the EV not linked to any scaffold moiety. In some aspects, the first adjuvant is linked to a first Scaffold X on the exterior surface of the EV and the second adjuvant is linked to a second Scaffold X on the luminal surface of the EV, e.g., exosome. In some aspects, the first adjuvant is linked to a first Scaffold X on the luminal surface of the EV and the second adjuvant is linked to a second Scaffold X on the exterior surface of the EV, e.g., exosome. In some aspects, the first adjuvant is in the lumen of the EV not linked to any scaffold moiety, and the second adjuvant is in the lumen of the EV not linked to any scaffold moiety.

In some aspects, an adjuvant useful for the present disclosure induces the activation of a cytosolic pattern recognition receptor. Non-limiting examples of cytosolic pattern recognition receptor includes: stimulator of interferon genes (STING), retinoic acid-inducible gene I (RIG-1), Melanoma Differentiation-Associated protein 5 (MDA5), Nucleotide-binding oligomerization domain, Leucine rich Repeat and Pyrin domain containing (NLRP), inflammasomes, or combinations thereof. In certain aspects, an adjuvant is a STING agonist. Stimulator of Interferon Genes (STING) is a cytosolic sensor of cyclic dinucleotides that is typically produced by bacteria. Upon activation, it leads to the production of type I interferons and initiates an immune response. In certain aspects, the STING agonist comprises a cyclic dinucleotide STING agonist or a non-cyclic dinucleotide STING agonist.

Cyclic purine dinucleotides such as, but not limited to, cGMP, cyclic di-GMP (c-di-GMP), cAMP, cyclic di-AMP (c-di-AMP), cyclic-GMP-AMP (cGAMP), cyclic di-IMP (c-di-IMP), cyclic AMP-IMP (cAIMP), and any analogue thereof, are known to stimulate or enhance an immune or inflammation response in a patient. The CDNs can have 2′2′, 2′3′, 2′5′, 3′3′, or 3′5′ bonds linking the cyclic dinucleotides, or any combination thereof.

Cyclic purine dinucleotides can be modified via standard organic chemistry techniques to produce analogues of purine dinucleotides. Suitable purine dinucleotides include, but are not limited to, adenine, guanine, inosine, hypoxanthine, xanthine, isoguanine, or any other appropriate purine dinucleotide known in the art. The cyclic dinucleotides can be modified analogues. Any suitable modification known in the art can be used, including, but not limited to, phosphorothioate, biphosphorothioate, fluorinate, and difluorinate modifications.

Non cyclic dinucleotide agonists can also be used, such as 5,6-Dimethylxanthenone-4-acetic acid (DMXAA), or any other non-cyclic dinucleotide agonist known in the art.

Non-limiting examples of STING agonists that can be used with the present disclosure include: DMXAA, STING agonist-1, ML RR-S2 CDA, ML RR-S2c-di-GMP, ML-RR-S2 cGAMP, 2′3′-c-di-AM(PS)2, 2′3′-cGAMP, 2′3′-cGAMPdFHS, 3′3′-cGAMP, 3′3′-cGAMPdFSH, cAIMP, cAIM(PS)2, 3′3′-cAIMP, 3′3′-cAIMPdFSH, 2′2′-cGAMP, 2′3′-cGAM(PS)2, 3′3′-cGAMP, and combinations thereof. Non-limiting examples of the STING agonists can be found at U.S. Pat. No. 9,695,212, WO 2014/189805A1, WO 2014/179335 A1, WO 2018/100558 A1, U.S. Pat. No. 10,011,630 B2, WO 2017/027646 A1, WO 2017/161349 A1, and WO 2016/096174 A1, each of which is incorporated by reference in its entirety.

In some aspects, the STING agonist useful for the present disclosure comprises a compound or a pharmaceutically acceptable salt thereof disclosed in WO 2016/096174, WO 2016/096174A1, WO 2014/093936, WO 2014/189805, WO 2015/077354, the content of which is incorporated herein by reference in its entirety. See also Cell reports 11, 1018-1030 (2015).

In some aspects, the STING agonist useful for the present disclosure comprises c-di-AMP, c-di-GMP, c-di-IMP, c-AMP-GMP, c-AMP-IMP, and c-GMP-IMP, described in WO 2013/185052 and Sci. Transl. Med. 283,283ra52 (2015), which are incorporated herein by reference in their entireties.

In some aspects, the STING agonist useful for the present disclosure comprises a compound or a pharmaceutically acceptable salt thereof disclosed in WO 2014/189806, WO 2015/185565, WO 2014/179760, WO 2014/179335, WO 2015/017652, WO 2016/096577, WO 2016/120305, WO 2016/145102, WO 2017/027646, WO 2017/075477, WO 2017/027645, WO 2018/100558, WO 2017/175147, or WO 2017/175156, each content of which is incorporated herein by reference in its entirety.

In some aspects, the STING agonist useful for the present disclosure is CL606, CL611, CL602, CL655, CL604, CL609, CL614, CL656, CL647, CL626, CL629, CL603, CL632, CL633, CL659, or a pharmaceutically acceptable salt thereof. In some aspects, the STING agonist useful for the present disclosure is CL606 or a pharmaceutically acceptable salt thereof. In some aspects, the STING agonist useful for the present disclosure is CL611 or a pharmaceutically acceptable salt thereof. In some aspects, the STING agonist useful for the present disclosure is CL602 or a pharmaceutically acceptable salt thereof. In some aspects, the STING agonist useful for the present disclosure is CL655 or a pharmaceutically acceptable salt thereof. In some aspects, the STING agonist useful for the present disclosure is CL604 or a pharmaceutically acceptable salt thereof. In some aspects, the STING agonist useful for the present disclosure is CL609 or a pharmaceutically acceptable salt thereof. In some aspects, the STING agonist useful for the present disclosure is CL614 or a pharmaceutically acceptable salt thereof. In some aspects, the STING agonist useful for the present disclosure is CL656 or a pharmaceutically acceptable salt thereof. In some aspects, the STING agonist useful for the present disclosure is CL647 or a pharmaceutically acceptable salt thereof. In some aspects, the STING agonist useful for the present disclosure is CL626 or a pharmaceutically acceptable salt thereof. In some aspects, the STING agonist useful for the present disclosure is CL629 or a pharmaceutically acceptable salt thereof. In some aspects, the STING agonist useful for the present disclosure is CL603 or a pharmaceutically acceptable salt thereof. In some aspects, the STING agonist useful for the present disclosure is CL632 or a pharmaceutically acceptable salt thereof. In some aspects, the STING agonist useful for the present disclosure is CL633 or a pharmaceutically acceptable salt thereof. In some aspects, the STING agonist useful for the present disclosure is CL659 or a pharmaceutically acceptable salt thereof.

In some aspects, the EV comprises a cyclic dinucleotide STING agonist and/or a non-cyclic dinucleotide STING agonist. In some aspects, when several cyclic dinucleotide STING agonist are present on an EV disclosed herein, such STING agonists can be the same or they can be different. In some aspects, when several non-cyclic dinucleotide STING agonist are present, such STING agonists can be the same or they can be different. In some aspects, an EV composition of the present disclosure can comprise two or more populations of EVs wherein each population of EVs comprises a different STING agonist or combination thereof.

In some aspects, one or more exogenous biologically active molecules, e.g., an adjuvant, is a TLR agonist. Non-limiting examples of TLR agonists include: TLR2 agonist (e.g., lipoteichoic acid, atypical LPS, MALP-2 and MALP-404, OspA, porin, LcrV, lipomannan, GPI anchor, lysophosphatidylserine, lipophosphoglycan (LPG), glycophosphatidylinositol (GPI), zymosan, hsp60, gH/gL glycoprotein, hemagglutinin), a TLR3 agonist (e.g., double-stranded RNA, e.g., poly(I:C)), a TLR4 agonist (e.g., lipopolysaccharides (LPS), lipoteichoic acid, β-defensin 2, fibronectin EDA, HMGB 1, snapin, tenascin C), a TLR5 agonist (e.g., flagellin), a TLR6 agonist, a TLR⅞ agonist (e.g., single-stranded RNA, CpG-A, Poly G10, Poly G3, Resiquimod), a TLR9 agonist (e.g., unmethylated CpG DNA), and combinations thereof. Non-limiting examples of TLR agonists can be found at WO2008115319A2, US20130202707A1, US20120219615A1, US20100029585A1, WO2009030996A1, WO2009088401A2, and WO2011044246A1, each of which are incorporated by reference in its entirety.

In some aspects, an EV comprising a targeting moiety (e.g., those disclosed herein) and an adjuvant can comprise additional exogenous biologically active molecules (e.g., immune modulators).

### III.B.5. Immune Modulator

In some aspects, an EV of the present disclosure have been modified or engineered to comprise one or more (e.g., two, three, four, five or more) immune modulators. In certain aspects, the one or more immune modulators are expressed in combination with other exogenous biologically active molecules disclosed herein (e.g., targeting moiety, therapeutic molecule, and/or adjuvant).

In some aspects, the present disclosure is directed to modified or engineered EVs comprising two or more exogenous biologically active molecules, wherein the two or more exogenous biologically active molecules are immune modulators, a first immune modulator and a second immune modulator (e.g., in addition to a targeting moiety disclosed herein). In some aspects, an immune modulator that can be used with the EVs described herein has antitumor activity. In other aspects, an immune modulator useful for the present disclosure has tolerogenic activity. In some aspects, an immune modulator can regulate innate immune response. In certain aspects, an immune modulator regulates innate immune response by targeting natural killer cells. In some aspects, an immune modulator can regulate adaptive immune response. In some aspects, the immune modulator regulates adaptive immune response by targeting cytotoxic T cells. In further aspects, the immune modulator regulates adaptive immune response by targeting B cells. In certain aspects, an immune modulator disclosed herein can modulate the distribution of an exosome to a cytotoxic T cell or a B cell (i.e., biodistribution modifying agent).

In some aspects, an immune modulator comprises an inhibitor for a negative checkpoint regulator or an inhibitor for a binding partner of a negative checkpoint regulator. In certain aspects, the negative checkpoint regulator comprises cytotoxic T-lymphocyte-associated protein 4 (CTLA-4), programmed cell death protein 1 (PD-1), lymphocyte-activated gene 3 (LAG-3), T-cell immunoglobulin mucin-containing protein 3 (TIM-3), B and T lymphocyte attenuator (BTLA), T cell immunoreceptor with Ig and ITIM domains (TIGIT), V-domain Ig suppressor of T cell activation (VISTA), adenosine A2a receptor (A2aR), killer cell immunoglobulin like receptor (KIR), indoleamine 2,3-dioxygenase (IDO), CD20, CD39, CD73, or any combination thereof.

In some aspects, the immune modulator is an inhibitor of cytotoxic T-lymphocyte-associate protein 4 (CTLA-4). In certain aspects, the CTLA-4 inhibitor is a monoclonal antibody of CTLA-4 (“anti-CTLA-4 antibody”). In certain aspects, the inhibitor is a fragment of a monoclonal antibody of CTLA-4. In certain aspects, the antibody fragment is a scFv, (scFv)₂, Fab, Fab′, and F(ab′)₂, F(ab1)₂, Fv, dAb, or Fd of a monoclonal antibody of CTLA-4. In certain aspects, the inhibitor is a nanobody, a bispecific antibody, or a multispecific antibody against CTLA-4. In some aspects, the anti-CTLA-4 antibody is ipilimumab. In other aspects, the anti-CTLA-4 antibody is tremelimumab.

In some aspects, the immune modulator is an inhibitor of programmed cell death protein 1 (PD-1). In some aspects, the immune modulator is an inhibitor of programmed death-ligand 1 (PD-L 1). In some aspects, the immune modulator is an inhibitor of programmed death-ligand 2 (PD-L2). In certain aspects, the inhibitor of PD-1, PD-L1, or PD-L2 is a monoclonal antibody of PD-1 (“anti-PD-1 antibody”), PD-L1 (“anti-PD-L1 antibody”), or PD-L2 (“anti-PD-L2 antibody”). In some aspects, the inhibitor is a fragment of an anti-PD-1 antibody, anti-PD-L1 antibody, or anti-PD-L2 antibody. In certain aspects, the antibody fragment is a scFv, (scFv)₂, Fab, Fab′, and F(ab′)₂, F(ab1)₂, Fv, dAb, or Fd of a monoclonal antibody of PD-1, PD-L1, or PD-L2. In certain aspects, the inhibitor is a nanobody, a bispecific antibody, or a multispecific antibody against PD-1, PD-L1, or PD-L2. In some aspects, the anti-PD-1 antibody is nivolumab. In some aspects, the anti-PD-1 antibody is pembrolizumab. In some aspects, the anti-PD-1 antibody is pidilizumab. In some aspects, the anti-PD-L1 antibody is atezolizumab. In other aspects, the anti-PD-L1 antibody is avelumab.

In some aspects, the immune modulator is an inhibitor of lymphocyte-activated gene 3 (LAG3). In certain aspects, the inhibitor of LAG3 is a monoclonal antibody of LAG3 (“anti-LAG3 antibody”). In some aspects, the inhibitor is a fragment of an anti-LAG3 antibody, e.g., scFv, (scFv)₂, Fab, Fab′, and F(ab′)₂, F(ab1)₂, Fv, dAb, or Fd. In certain aspects, the inhibitor is a nanobody, a bispecific antibody, or a multispecific antibody against LAG3.

In some aspects, the immune modulator is an inhibitor of T-cell immunoglobulin mucin-containing protein 3 (TIM-3). In some aspects, the immune modulator is an inhibitor of B and T lymphocyte attenuator (BTLA). In some aspects, the immune modulator is an inhibitor of T cell immunoreceptor with Ig and ITIM domains (TIGIT). In some aspects, the immune modulator is an inhibitor of V-domain Ig suppressor of T cell activation (VISTA). In some aspects, the immune modulator is an inhibitor of adenosine A2a receptor (A2aR). In some aspects, the immune modulator is an inhibitor of killer cell immunoglobulin like receptor (KIR). In some aspects, the immune modulator is an inhibitor of indoleamine 2,3-dioxygenase (IDO). In some aspects, the immune modulator is an inhibitor of CD20, CD39, or CD73.

In some aspects, the immune modulator comprises an activator for a positive co-stimulatory molecule or an activator for a binding partner of a positive co-stimulatory molecule. In certain aspects, the positive co-stimulatory molecule comprises a TNF receptor superfamily member (e.g., CD120a, CD120b, CD18, OX40, CD40, Fas receptor, M68, CD27, CD30, 4-1BB, TRAILR1, TRAILR2, TRAILR3, TRAILR4, RANK, OCIF, TWEAK receptor, TACI, BAFF receptor, ATAR, CD271, CD269, AITR, TROY, CD358, TRAMP, and XEDAR). In some aspects, the activator for a positive co-stimulatory molecule is a TNF superfamily member (e.g., TNFα, TNF-C, OX40L, CD40L, FasL, LIGHT, TL1A, CD27L, Siva, CD153, 4-1BB ligand, TRAIL, RANKL, TWEAK, APRIL, BAFF, CAMLG, NGF, BDNF, NT-3, NT-4, GITR ligand, and EDA-2).

In some aspects, the immune modulator is an activator of TNF Receptor Superfamily Member 4 (OX40). In certain aspects, the activator of OX40 is an agonistic anti-OX40 antibody. In further aspects, the activator of OX40 is a OX40 ligand (OX40L).

In some aspects, the immune modulator is an activator of CD27. In certain aspects, the activator of CD27 is an agonistic anti-CD27 antibody. In other aspects, the activator of CD27 is a CD27 ligand (CD27L).

In some aspects, the immune modulator is an activator of CD40. In certain aspects, the activator of CD40 is an agonistic anti-CD40 antibody. In some aspects, the activator of CD40 is a CD40 ligand (CD40L). In certain aspects, the CD40L is a monomeric CD40L. In other aspects, the CD40L is a trimeric CD40L.

In some aspects, the immune modulator is an activator of glucocorticoid-induced TNFR-related protein (GITR). In certain aspects, the activator of GITR is an agonistic anti-GITR antibody. In other aspects, the activator of GITR is a natural ligand of GITR.

In some aspects, the immune modulator is an activator of 4-1BB. In specific aspects, the activator of 4-1BB is an agonistic anti-4-1BB antibody. In certain aspects, the activator of 4-1BB is a natural ligand of 4-1BB.

In some aspects, the immune modulator is a Fas receptor (Fas). In such aspects, the Fas receptor is displayed on the surface of the EV, e.g., exosome. In some aspects, the immune modulator is Fas ligand (FasL). In certain aspects, the Fas ligand is displayed on the surface of the EV, e.g., exosome. In some aspects, the immune modulator is an anti-Fas antibody or an anti-FasL antibody.

In some aspects, the immune modulator is an activator of a CD28-superfamily co-stimulatory molecule. In certain aspects, the CD28-superfamily co-stimulatory molecule is ICOS or CD28. In certain aspects, the immune modulator is ICOSL, CD80, or CD86.

In some aspects, the immune modulator is an activator of inducible T cell co-stimulator (ICOS). In certain aspects, the activator of ICOS is an agonistic anti-ICOS antibody. In other aspects, the activator of ICOS is a ICOS ligand (ICOSL).

In some aspects, the immune modulator is an activator of CD28. In some aspects, the activator of CD28 is an agonistic anti-CD28 antibody. In other aspects, the activator of CD28 is a natural ligand of CD28. In certain aspects, the ligand of CD28 is CD80.

In some aspects, the immune modulator comprises a cytokine or a binding partner of a cytokine. In certain aspects, the cytokine comprises IL-2, IL-4, IL-7, IL-10, IL-12, IL-15, IL-21, or IFN-y. In some aspects, the immune modulator comprises FLT-3 (CD135).

In some aspects, an EVs described herein comprises a first scaffold moiety. In certain aspects, a first exogenous biologically active molecule (e.g., targeting moiety, therapeutic molecule, adjuvant, or immune modulator) is linked to the first scaffold moiety. In other aspects, a second exogenous biologically active molecule (e.g., targeting moiety, therapeutic molecule, adjuvant, or immune modulator) is linked to the first scaffold moiety. In further aspects, both the first and second exogenous biologically active molecules are linked to the first scaffold moiety. In some aspects, an EVs further comprises a second scaffold moiety. In certain aspects, the first exogenous biologically active molecule is linked to the first scaffold moiety, and the second exogenous biologically active molecule is linked to the second scaffold moiety. In some aspects, the first scaffold moiety and the second scaffold moiety are the same (e.g., both Scaffold X or both Scaffold Y). In other aspects, the first scaffold moiety and the second scaffold moiety are different (e.g., first scaffold moiety is Scaffold X and the second scaffold moiety is Scaffold Y; or first scaffold moiety is Scaffold Y and the second scaffold moiety is Scaffold X).

In some aspects, the one or more exogenous biologically active molecule disclosed herein (e.g., targeting moiety, therapeutic molecule, immune modulator, or adjuvant) can be modified to increase encapsulation in an EV, e.g., exosome. This modification can include the addition of a lipid binding tag by treating the agonist with a chemical or enzyme, or by physically or chemically altering the polarity or charge of the exogenous biologically active molecule (e.g., adjuvant and/or antigen). The exogenous biologically active molecule can be modified by a single treatment, or by a combination of treatments, e.g., adding a lipid binding tag only, or adding a lipid binding tag and altering the polarity. The previous example is meant to be a non-limiting illustrative instance. It is contemplated that any combination of modifications can be practiced. The modification can increase encapsulation of the exogenous biologically active molecule in the EV, e.g., exosome by between 2-fold and 10,000 fold, between 10-fold and 1,000 fold, or between 100-fold and 500-fold compared to encapsulation of an unmodified exogenous biologically active molecule. The modification can increase encapsulation of the exogenous biologically active molecule in the EV, e.g., exosome by at least 2-fold, 5-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, 6000-fold, 7000-fold, 8000-fold, 9000-fold, or 10,000-fold compared to encapsulation of an unmodified exogenous biologically active molecule.

Additional non-limiting examples of specific aspects include EVs comprising (i) one or more targeting moieties, (ii) one or more therapeutic molecules (e.g., tumor antigens), and (iii) one or more adjuvants (e.g., a STING agonist or a TLR agonist) and/or immune modulators, wherein:
(a) the one or more targeting moieties are linked to a first Scaffold X on the exterior surface of the EV the one or more therapeutic molecules are linked to a second Scaffold X on the exterior surface of the EV and the one or more adjuvants and/or immune modulators are (a1) in the lumen of the EV not linked to any scaffold moiety, or (a2) linked to a third scaffold moiety, e.g., a Scaffold X on the exterior surface of the exosome or on the luminal surface of the exosome or a Scaffold Y on the luminal surface of the EV, e.g., exosome;
(b) the one or more targeting moieties are linked to a Scaffold X on the exterior surface of the EV the one or more therapeutic molecules are linked to a Scaffold Y on the luminal surface of the EV and the one or more adjuvants and/or immune modulators are (b1) in the lumen of the EV not linked to any scaffold moiety, or (b2) linked to a third scaffold moiety, e.g., a Scaffold X on the exterior surface of the exosome or on the luminal surface of the exosome or a Scaffold Y on the luminal surface of the EV, e.g., exosome;
(c) the one or more targeting moieties are linked to a Scaffold X on the exterior surface of the EV the one or more therapeutic molecules are in the lumen of the EV not linked to any scaffold moiety, and the one or more adjuvants and/or immune modulators are (c1) in the lumen of the EV not linked to any scaffold moiety, or (c2) linked to a scaffold moiety, e.g., a Scaffold X on the exterior surface of the exosome or on the luminal surface of the exosome or a Scaffold Y on the luminal surface of the EV, e.g., exosome;
(d) the one or more targeting moieties are linked to a Scaffold X on the exterior surface of the EV the one or more therapeutic molecules are linked to the Scaffold X on the luminal surface of the EV and the one or more adjuvants and/or immune modulators are (d1) in the lumen of the EV not linked to any scaffold moiety, or (d2) linked to a scaffold moiety, e.g., a Scaffold X on the exterior surface of the exosome or on the luminal surface of the exosome or a Scaffold Y on the luminal surface of the EV, e.g., exosome; or
(e) the one or more targeting moieties are linked to a first Scaffold X on the exterior surface of the EV the one or more therapeutic molecules are linked to a second Scaffold X on the luminal surface of the EV and the one or more adjuvants and/or immune modulators are (e1) in the lumen of the EV not linked to any scaffold moiety, or (e2) linked to a third scaffold moiety, e.g., a Scaffold X on the surface of the exosome or in the lumen of the exosome or a Scaffold Y on the luminal surface of the EV, e.g., exosome.

In some aspects, the immune modulator comprises a protein that supports intracellular interactions required for germinal center responses. In certain aspects, such a protein comprises a signaling lymphocyte activation molecule (SLAM) family member or a SLAM-associated protein (SAP). In some aspects, a SLAM family members comprises SLAM, CD48, CD229 (Ly9), Ly108, 2B4, CD84, NTB-A, CRACC, BLAME, CD2F-10, or combinations thereof.

In some aspects, the immune modulator comprises a T-cell receptor (TCR) or a derivative thereof. In certain aspects, the immune modulator is a TCR α-chain or a derivative thereof. In other aspects, the immune modulator is a TCR β-chain or a derivative thereof. In further aspects, the immune modulator is a co-receptor of the T-cell or a derivative thereof.

In some aspects, the immune modulator comprises a chimeric antigen receptor (CAR) or a derivative thereof. In certain aspects, the CAR binds to one or more of the therapeutic molecules disclosed herein (e.g., tumor antigen, e.g., alpha-fetoprotein (AFP), carcinoembryonic antigen (CEA), epithelial tumor antigen (ETA), mucin 1 (MUC1), Tn-MUC1, mucin 16 (MUC16), tyrosinase, melanoma-associated antigen (MAGE), tumor protein p53 (p53), CD4, CD8, CD45, CD80, CD86, programmed death ligand 1 (PD-L1), programmed death ligand 2 (PD-L2), NY-ESO-1, PSMA, TAG-72, HER2, GD2, cMET, EGFR, Mesothelin, VEGFR, alpha-folate receptor, CE7R, IL-3, Cancer-testis antigen, MART-1 gp100, and TNF-related apoptosis-inducing ligand).

In certain aspects, the immune modulator is an activator of CD28. In certain aspects, the activator is a fragment of a monoclonal antibody of CD28. In certain aspects, the antibody fragment is a scFv, (scFv)₂, Fab, Fab′, and F(ab′)₂, F(ab1)₂, Fv, dAb, or Fd of a monoclonal antibody of CD28. In certain aspects, the activator is a nanobody, a bispecific antibody, or a multispecific antibody against CD28.

In some aspects, the immune modulator comprises a NF-κB inhibitor. Non-limiting examples of NF-xB inhibitors that can be used with the present disclosure includes: IKK complex inhibitors (e.g.,TPCA-1, NF-κB Activation Inhibitor VI (BOT-64), BMS 345541, Amlexanox, SC-514 (GK 01140), IMD 0354, IKK-16), IκB degradation inhibitor (e.g., BAY 11-7082, MG-115, MG-132, Lactacystin, Epoxomicin, Parthenolide, Carfilzomib, MLN-4924 (Pevonedistat)), NF-xB nuclear translocation inhibitor (e.g., JSH-23, Rolipram), p65 acetylation inhibitor (e.g., Gallic acid, Anacardic acid), NF-κB-DNA binding inhibitor (e.g., GYY 4137, p-XSC, CV 3988, Prostaglandin E2 (PGE2)), NF-κB transactivation inhibitor (e.g., LY 294002, Wortmannin, Mesalamine), or combinations thereof. See also Gupta, S.C., et al., Biochim Biophys Acta 1799:775-787 (2010), which is herein incorporated by reference in its entirety. In further aspects, an immune modulator comprises a COX-2 inhibitor, mTOR inhibitor (e.g., rapamycin and derivatives), prostaglandins, nonsteroidal anti-inflammatory agents (NSAIDS), antileukotriene, or combinations thereof.

In some aspects, the immune modulator is an agonist. In certain aspects, the agonist is an endogenous agonist, such as a hormone, or a neurotransmitter. In other aspects, the agonist is an exogenous agonist, such as a drug. In some aspects, the agonist is a physical agonist, which can create an agonist response without binding to the receptor. In some aspects, the agonist is a superagonist, which can produce a greater maximal response than the endogenous agonist. In certain aspects, the agonist is a full agonist with full efficacy at the receptor. In other aspects, the agonist is a partial agonist having only partial efficacy at the receptor relative to a full agonist. In some aspects, the agonist is an inverse agonist that can inhibit the constitutive activity of the receptor. In some aspects, the agonist is a co-agonist that works with other co-agonists to produce an effect on the receptor. In certain aspects, the agonist is an irreversible agonist that binds permanently to a receptor through formation of covalent bond. In certain aspects, the agonist is selective agonist for a specific type of receptor

In some aspects, the immune modulator is an antagonist. In specific aspects, the antagonist is a competitive antagonist, which reversibly binds to the receptor at the same binding site as the endogenous ligand or agonist without activating the receptor. Competitive antagonist can affect the amount of agonist necessary to achieve a maximal response. In other aspects, the antagonist is a non-competitive antagonist, which binds to an active site of the receptor or an allosteric site of the receptor. Non-competitive antagonist can reduce the magnitude of the maximum response that can be attained by any amount of agonist. In further aspects, the antagonist is an uncompetitive antagonist, which requires receptor activation by an agonist before its binding to a separate allosteric binding site.

In some aspects, the immune modulator comprises an antibody or an antigen-binding fragment. The immune modulator can be a full-length protein or a fragment thereof. The antibody or antigen-binding fragment can be derived from natural sources, or partly or wholly synthetically produced. In some aspects, the antibody is a monoclonal antibody. In some of these aspects, the monoclonal antibody is an IgG antibody. In certain aspects, the monoclonal antibody is an IgG1, IgG2, IgG3, or IgG4. In some other aspects, the antibody is a polyclonal antibody. In certain aspects, the antigen-binding fragment is selected from Fab, Fab′, and F(ab′)₂, F(ab1)₂, Fv, dAb, and Fd fragments. In certain aspects, the antigen-binding fragment is an scFv or (scFv)₂ fragment. In certain other aspects, the antibody or antigen-binding fragment is a NANOBODY® (single-domain antibody). In some aspects, the antibody or antigen-binding fragment is a bispecific or multispecific antibody.

In various aspects, the antibody or antigen-binding fragment is fully human. In some aspects, the antibody or antigen-binding fragment is humanized. In some aspects, the antibody or antigen-binding fragment is chimeric. In some of these aspects, the chimeric antibody has non-human V region domains and human C region domains. In some aspects, the antibody or antigen-binding fragment is non-human, such as murine or veterinary.

In certain aspects, the immune modulator is a polynucleotide. In some of these aspects, the polynucleotide includes, but is not limited to, an mRNA, a miRNA, an siRNA, an antisense RNA, an shRNA, a lncRNA, and a dsDNA. In some aspects, the polynucleotide is an RNA (e.g., an mRNA, a miRNA, an siRNA, an antisense RNA, an shRNA, or an lncRNA). In some of these aspects, when the polynucleotide is an mRNA, it can be translated into a desired polypeptide. In some aspects, the polynucleotide is a microRNA (miRNA) or pre-miRNA molecule. In some of these aspects, the miRNA is delivered to the cytoplasm of the target cell, such that the miRNA molecule can silence a native mRNA in the target cell. In some aspects, the polynucleotide is a small interfering RNA (siRNA) or a short hairpin RNA (shRNA) capable of interfering with the expression of an oncogene or other dysregulating polypeptides. In some of these aspects, the siRNA is delivered to the cytoplasm of the target cell, such that the siRNA molecule can silence a native mRNA in the target cell. In some aspects, the polynucleotide is an antisense RNA that is complementary to an mRNA. In some aspects, the polynucleotide is a long non-coding RNA (lncRNA) capable of regulating gene expression and modulating diseases. In some aspects, the polynucleotide is a DNA that can be transcribed into an RNA. In some of these aspects, the transcribed RNA can be translated into a desired polypeptide.

In some aspects, the immune modulator is a protein, a peptide, a glycolipid, or a glycoprotein.

In various aspects, the composition comprises two or more above-mentioned immune modulators, including mixtures, fusions, combinations and conjugates, of atoms, molecules, etc. In some aspects, the composition comprises one, two, three, four, five, six, seven, eight, nine, ten, eleven, or twelve different immune modulators associated with the membrane or enclosed within the enclosed volume of said extracellular vesicle. In certain aspects, the composition comprises a nucleic acid combined with a polypeptide. In certain aspects, the composition comprises two or more polypeptides conjugated to each other. In certain aspects, the composition comprises a protein conjugated to an exogenous biologically active molecule. In some of these aspects, the exogenous biologically active molecule is a prodrug.

In some aspects, an EV disclosed herein comprises a targeting moiety and a STING agonist. In some aspects, an EV disclosed herein comprises a targeting moiety and a TLR agonist (e.g., TLR3 agonist). In some aspects, an EV disclosed herein comprises a targeting moiety and IFN-α or IFN-y. In some aspects, the targeting moiety specifically binds to CD3 protein (or a variant thereof). In some aspects, the exogeneous targeting moiety is injected directly into lymph nodes. In each of these aspects, a targeting moiety can comprise an antigen, an immunosuppressive agent, or both.

### III.B.6. Anti-Phagocytic Signal

Clearance of administered EVs by the body’s immune system can reduce the efficacy of an administered EV therapy. In some aspects, the surface of the EV is modified to limit or block uptake of the EV by cells of the immune system, e.g., macrophages. In some aspects, the surface of the EV is modified to express one or more surface antigen that inhibits uptake of the EV by a macrophage, i.e., an “antiphagocytic signal.” In some aspects, the surface antigen is associated with the exterior surface of the EV, (e.g., exosome).

Surface antigens useful in the present disclosure that can function as antiphagocytic signals include, but are not limited to, antigens that label a cell as a “self” cell. In some aspects, the surface antigen (antiphagocytic signal) is selected from CD47, CD24, a fragment thereof, and any combination thereof. In certain aspects, the surface antigen comprises CD24, e.g., human CD24. In some aspects, the surface antigen comprises a fragment of CD24, e.g., human CD24. In certain aspects, the EV is modified to express CD47 or a fragment thereof on the exterior surface of the EV, e.g., exosome. As used herein, a “fragment” of a CD47 refers to a portion of a CD47 protein that retains the ability to inhibit uptake by an immune cell.

CD47, also referred to as leukocyte surface antigen CD47 and integrin associated protein (IAP), as used herein, is a transmembrane protein that is found on many cells in the body. CD47 is often referred to as the “don’t eat me” signal, as it signals to immune cells, in particular myeloid cells, that a particular cell expressing CD47 is not a foreign cell. CD47 is the receptor for SIRPA, binding to which prevents maturation of immature dendritic cells and inhibits cytokine production by mature dendritic cells. Interaction of CD47 with SIRPG mediates cell-cell adhesion, enhances superantigen-dependent T-cell-mediated proliferation and costimulates T-cell activation. CD47 is also known to have a role in both cell adhesion by acting as an adhesion receptor for THBS1 on platelets, and in the modulation of integrins. CD47 also plays an important role in memory formation and synaptic plasticity in the hippocampus (by similarity). In addition, CD47 can play a role in membrane transport and/or integrin dependent signal transduction, prevent premature elimination of red blood cells, and be involved in membrane permeability changes induced following virus infection.

In some aspects, an EV disclosed herein is modified to express a human CD47 on the surface of the EV, e.g., exosome. The canonical amino acid sequence for human CD47 and various known isoforms are shown in Table 3 (UniProtKB - Q08722; SEQ ID NOs: 348-351). In some aspects, the EV is modified to express a polypeptide comprising the amino acid sequence set forth in SEQ ID NO: 400 or a fragment thereof. In some aspects, the EV is modified to express a polypeptide comprising the amino acid sequence set forth in SEQ ID NO: 401 or a fragment thereof. In some aspects, the EV is modified to express a polypeptide comprising the amino acid sequence set forth in SEQ ID NO: 402 or a fragment thereof. In some aspects, the EV is modified to express a polypeptide comprising the amino acid sequence set forth in SEQ ID NO: 403 or a fragment thereof.

TABLE 3

Human CD47 Amino Acid Sequences

| | |
|---|---|
| Canonical CD47 | MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE (SEQ ID NO: 400) |
| CD47 HUMAN Isoform OA3-293 | MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFV (SEQ ID NO: 401) |
| CD47 HUMAN Isoform OA3-305 | MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN (SEQ ID NO: 402) |
| CD47 HUMAN Isoform OA3-312 | MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLN (SEQ ID NO: 403) |

In some aspects, the EV is modified to express full length CD47 on the surface of the EV, e.g., exosome. In some aspects, the EV is modified to express a fragment of CD47 on the surface of the EV wherein the fragment comprises the extracellular domain of CD47, e.g., human CD47. Any fragment of CD47 that retains an ability to block and/or inhibit phagocytosis by a macrophage can be used in the EVs disclosed herein. In some aspects, the fragment comprises amino acids 19 to about 141 of the canonical human CD47 sequence (e.g., amino acids 19-141 of SEQ ID NO 400). In some aspects, the fragment comprises amino acids 19 to about 135 of the canonical human CD47 sequence (e.g., amino acids 19-135 of SEQ ID NO 400). In some aspects, the fragment comprises amino acids 19 to about 130 of the canonical human CD47 sequence (e.g., amino acids 19-130 of SEQ ID NO 400). In some aspects, the fragment comprises amino acids 19 to about 125 of the canonical human CD47 sequence (e.g., amino acids 19-125 of SEQ ID NO 400).

In some aspects, the EV is modified to express a polypeptide having at least about 70%, at least about 75%, at least about 80%, at least about 85%, at least about 90%, at least about 95%, at least about 96%, at least about 97%, at least about 98%, or at least about 99% sequence identity to amino acids 19 to about 141 of the canonical human CD47 sequence (e.g., amino acids 19-141 of SEQ ID NO 400). In some aspects, the EV is modified to express a polypeptide having at least about 70%, at least about 75%, at least about 80%, at least about 85%, at least about 90%, at least about 95%, at least about 96%, at least about 97%, at least about 98%, or at least about 99% sequence identity to amino acids 19 to about 135 of the canonical human CD47 sequence (e.g., amino acids 19-135 of SEQ ID NO 400). In some aspects, the EV is modified to express a polypeptide having at least about 70%, at least about 75%, at least about 80%, at least about 85%, at least about 90%, at least about 95%, at least about 96%, at least about 97%, at least about 98%, or at least about 99% sequence identity to amino acids 19 to about 130 of the canonical human CD47 sequence (e.g., amino acids 19-130 of SEQ ID NO 400). In some aspects, the EV is modified to express a polypeptide having at least about 70%, at least about 75%, at least about 80%, at least about 85%, at least about 90%, at least about 95%, at least about 96%, at least about 97%, at least about 98%, or at least about 99% sequence identity to amino acids 19 to about 125 of the canonical human CD47 sequence (e.g., amino acids 19-125 of SEQ ID NO 400).

In some aspects, the CD47 or the fragment thereof is modified to increase the affinity of CD47 and its ligand SIRPα. In some aspects, the fragment of CD47 comprises a Velcro-CD47 (see, e.g., Ho et al., JBC 290:12650-63 (2015), which is incorporated by reference herein in its entirety). In some aspects, the Velcro-CD47 comprises a C15S substitution relative to the wild-type human CD47 sequence (SEQ ID NO: 400).

In some aspects, the EV comprises a CD47 or a fragment thereof expressed on the surface of the EV at a level that is higher than an unmodified EV, e.g., exosome. In some aspects, the CD47 or the fragment thereof is fused with a scaffold protein. Any scaffold protein disclosed herein can be used to express the CD47 or the fragment thereof on the surface of the EV, e.g., exosome. In some aspects, the EV is modified to express a fragment of CD47 fused to the N-terminus of a Scaffold X protein. In some aspects, the EV is modified to express a fragment of CD47 fused to the N-terminus of PTGFRN.

In some aspects, the EV comprises at least about 20 molecules, at least about 30 molecules, at least about 40, at least about 50, at least about 75, at least about 100, at least about 125, at least about 150, at least about 200, at least about 250, at least about 300, at least about 350, at least about 400, at least about 450, at least about 500, at least about 750, or at least about 1000 molecules of CD47 on the surface of the EV, e.g., exosome. In some aspects, the EV comprises at least about 20 molecules of CD47 on the surface of the EV, e.g., exosome. In some aspects, the EV comprises at least about 30 molecules of CD47 on the surface of the EV, e.g., exosome. In some aspects, the EV comprises at least about 40 molecules of CD47 on the surface of the EV, e.g., exosome. In some aspects, the EV comprises at least about 50 molecules of CD47 on the surface of the EV, e.g., exosome. In some aspects, the EV comprises at least about 100 molecules of CD47 on the surface of the EV, e.g., exosome. In some aspects, the EV comprises at least about 200 molecules of CD47 on the surface of the EV, e.g., exosome. In some aspects, the EV comprises at least about 300 molecules of CD47 on the surface of the EV, e.g., exosome. In some aspects, the EV comprises at least about 400 molecules of CD47 on the surface of the EV, e.g., exosome. In some aspects, the EV comprises at least about 500 molecules of CD47 on the surface of the EV, e.g., exosome. In some aspects, the EV comprises at least about 1000 molecules of CD47 on the surface of the EV, e.g., exosome.

In some aspects, expression CD47 or a fragment thereof on the surface of the EV results in decreased uptake of the EV by myeloid cells as compared to an EV not expressing CD47 or a fragment thereof. In some aspects, uptake by myeloid cells of the EV expressing CD47 or a fragment thereof is decreased by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or at least about 95%, relative to uptake by myeloid cells of EVs that do not express CD47 or a fragment thereof.

In some aspects, expression CD47 or a fragment thereof on the surface of the EV results in decreased localization of the EV to the liver, as compared to an EV not expressing CD47 or a fragment thereof. In some aspects, localization to the liver of EVs expressing CD47 or a fragment thereof is decreased by at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or at least about 95%, relative to the localization to the liver of EVs not expressing CD47 or a fragment thereof.

In some aspects, the in vivo half-life of an EVexpressing CD47 or a fragment thereof is increased relative to the in vivo half-life of an EV that does not express CD47 or a fragment thereof. In some aspects, the in vivo half-life of an EV expressing CD47 or a fragment thereof is increased by at least about 1.5-fold, at least about 2-fold, at least about 2.5-fold, at least about 3-fold, at least about 3.5-fold, at least about 4-fold, at least about 4.5-fold, at least about 5-fold, at least about 6-fold, at least about 7-fold, at least about 8-fold, at least about 9-fold, or at least about 10-fold, relative to the in vivo half-life of an EV that does not express CD47 or a fragment thereof.

In some aspects, an EV expressing CD47 or a fragment thereof has an increased retention in circulation, e.g., plasma, relative to the retention of an EV that does not express CD47 or a fragment thereof in circulation, e.g., plasma. In some aspects, retention in circulation, e.g., plasma, of an EV expressing CD47 or a fragment thereof is increased by at least about 1.5-fold, at least about 2-fold, at least about 2.5-fold, at least about 3-fold, at least about 3.5-fold, at least about 4-fold, at least about 4.5-fold, at least about 5-fold, at least about 6-fold, at least about 7-fold, at least about 8-fold, at least about 9-fold, or at least about 10-fold, relative to the retention in circulation, e.g., plasma, of an EV that does not express CD47 or a fragment thereof.

In some aspects, an EV expressing CD47 or a fragment thereof has an altered biodistribution when compared with an exosome that does not express CD47 or a fragment. In some aspects, the altered biodistribution leads to increased uptake into endothelial cells, T cells, or increased accumulation in various tissues, including, but not limited to skeletal muscle, cardiac muscle, diaphragm, kidney, bone marrow, central nervous system, lungs, cerebral spinal fluid (CSF), or any combination thereof.

### III.B.7. Transmembrane Scaffold- (Scaffold X-) Engineered EVs, e.g., Exosomes

In some aspects, EVs of the present disclosure comprise a membrane modified in its composition. For example, their membrane compositions can be modified by changing the protein, lipid, or glycan content of the membrane.

In some aspects, the surface-engineered EVs are generated by chemical and/or physical methods, such as PEG-induced fusion and/or ultrasonic fusion. In other aspects, the surface-engineered EVs are generated by genetic engineering. EVs produced from a genetically-modified producer cell or a progeny of the genetically-modified cell can contain modified membrane compositions. In some aspects, surface-engineered EVs have scaffold moiety (e.g., exosome protein, e.g., Scaffold X) at a higher or lower density (e.g., higher number) or include a variant or a fragment of the scaffold moiety. In certain aspects, surface-engineered EVs can comprise multiple (e.g., two or more) scaffold moieties on their exterior surface. In some aspects, each of the multiple scaffold moieties are the same. In other aspects, one or more of the multiple scaffold moieties are different.

For example, surface (e.g., Scaffold X)-engineered EVs, can be produced from a cell (e.g., HEK293 cells) transformed with an exogenous sequence encoding a scaffold moiety (e.g., exosome proteins, e.g., Scaffold X) or a variant or a fragment thereof. EVs including scaffold moiety expressed from the exogenous sequence can include modified membrane compositions.

Various modifications or fragments of the scaffold moiety can be used for the aspects of the present disclosure. For example, one or more scaffold moieties modified to have enhanced affinity to a binding agent can be used for generating surface-engineered EV that can be purified using the binding agent. Scaffold moieties modified to be more effectively targeted to EVs and/or membranes can be used. Scaffold moieties modified to comprise a minimal fragment required for specific and effective targeting to exosome membranes can be also used. In some aspects, scaffold moieties can be linked to the maleimide moiety as described herein. In other aspects, scaffold moieties are not linked to the maleimide moiety.

Scaffold moieties can be engineered synthetically or recombinantly, e.g., to be expressed as a fusion molecule or protein, e.g., fusion molecule/protein of Scaffold X to another moiety (e.g., one or more exogenous biologically active molecules (e.g., those disclosed herein, e.g., a therapeutic molecule (e.g., an antigen), an adjuvant, and/or an immune modulator)). For example, the fusion molecule can comprise a scaffold moiety disclosed herein (e.g., Scaffold X, e.g., PTGFRN, BSG, IGSF2, IGSF3, IGSF8, ITGB1, ITGA4, SLC3A2, ATP transporter, or a fragment or a variant thereof) linked to another moeity (e.g., a therapeutic molecule (e.g., antigen), an adjuvant, and/or an immune modulator). In case of the fusion molecule, the therapeutic molecule, adjuvant, and/or immune modulator can be a natural peptide, a recombinant peptide, a synthetic peptide, or any combination thereof. In other aspects, the scaffold moieties can be CD9, CD63, CD81, PDGFR, GPI anchor proteins, lactadherin, LAMP2, or LAMP2B, or any combination thereof. Non-limiting examples of other scaffold moieties that can be used with the present disclosure include: aminopeptidase N (CD13); Neprilysin, AKA membrane metalloendopeptidase (MME); ectonucleotide pyrophosphatase/phosphodiesterase family member 1 (ENPP1); Neuropilin-1 (NRP1); or any combination thereof.

In some aspects, the fusion molecule can comprise a scaffold protein disclosed herein (e.g., PTGFRN, BSG, IGSF2, IGSF3, IGSF8, ITGB1, ITGA4, SLC3A2, ATP transporter, or a fragment or a variant thereof) linked to biologically action molecule either directly or through an intermediate (e.g., a chemically inducible dimer, an antigen binding domain, or a receptor).

In some aspects, the surface (e.g., Scaffold X)-engineered EVs described herein demonstrate superior characteristics compared to EVs known in the art. For example, surface (e.g., Scaffold X)-engineered contain modified proteins more highly enriched on the external surface or luminal surface of the EV than naturally occurring EVs or the EVs produced using conventional exosome proteins. In some aspects, surface (e.g., Scaffold X)-engineered EVs described herein can express greater number (e.g., 2, 3, 4, 5 or more) of exogenous biologically active molecules, such that multiple EVs are not required. Moreover, the surface (e.g., Scaffold X)-engineered EVs of the present disclosure can have greater, more specific, or more controlled biological activity compared to naturally occurring EVs or the EVs produced using conventional exosome proteins.

In some aspects the scaffold moiety, e.g., Scaffold X, comprises Prostaglandin F2 receptor negative regulator (the PTGFRN polypeptide). The PTGFRN polypeptide can be also referred to as CD9 partner 1 (CD9P-1), Glu-Trp-Ile EWI motif-containing protein F (EWI-F), Prostaglandin F2-alpha receptor regulatory protein, Prostaglandin F2-alpha receptor-associated protein, or CD315. The full length amino acid sequence of the human PTGFRN polypeptide (Uniprot Accession No. Q9P2B2) is shown at Table 1 as SEQ ID NO: 1. The PTGFRN polypeptide contains a signal peptide (amino acids 1 to 25 of SEQ ID NO: 1), the extracellular domain (amino acids 26 to 832 of SEQ ID NO: 1), a transmembrane domain (amino acids 833 to 853 of SEQ ID NO: 1), and a cytoplasmic domain (amino acids 854 to 879 of SEQ ID NO: 1). The mature PTGFRN polypeptide consists of SEQ ID NO: 1 without the signal peptide, i.e., amino acids 26 to 879 of SEQ ID NO: 1. In some aspects, a PTGFRN polypeptide fragment useful for the present disclosure comprises a transmembrane domain of the PTGFRN polypeptide. In other aspects, a PTGFRN polypeptide fragment useful for the present disclosure comprises the transmembrane domain of the PTGFRN polypeptide and (i) at least about five, at least about 10, at least about 15, at least about 20, at least about 25, at least about 30, at least about 40, at least about 50, at least about 70, at least about 80, at least about 90, at least about 100, at least about 110, at least about 120, at least about 130, at least about 140, at least about 150 amino acids at the N terminus of the transmembrane domain, (ii) at least about five, at least about 10, at least about 15, at least about 20, or at least about 25 amino acids at the C terminus of the transmembrane domain, or both (i) and (ii).

In some aspects, the fragments of PTGFRN polypeptide lack one or more functional or structural domains, such as IgV.

In other aspects, the scaffold moiety, e.g., Scaffold X, comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 26 to 879 of SEQ ID NO: 1. In other aspects, the scaffold moiety, e.g., Scaffold X, comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to SEQ ID NO: 2, a fragment of the PTGFRN polypeptide (corresponding to positions 687 to 878 of SEQ ID NO: 1).

In other aspects, the scaffold moiety, e.g., Scaffold X, comprises the amino acid sequence of SEQ ID NO: 2, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, the scaffold moiety, e.g., Scaffold X, comprises the amino acid sequence of SEQ ID NO: 2 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NO: 2.

In other aspects, the scaffold moiety, e.g., Scaffold X, comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to amino acids 26 to 879 of SEQ ID NO: 1, amino acids 833 to 853 of SEQ ID NO: 1, SEQ ID NO: 2, or SEQ ID NO: 1. In other aspects, the Scaffold X comprises the amino acid sequence of amino acids 26 to 879 of SEQ ID NO: 1, amino acids 833 to 853 of SEQ ID NO: 1, SEQ ID NO: 2, or SEQ ID NO: 1, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof.

In some aspects, the scaffold moiety, e.g., Scaffold X, comprises the amino acid sequence of amino acids 26 to 879 of SEQ ID NO: 1, amino acids 833 to 853 of SEQ ID NO: 1, SEQ ID NO: 2, or SEQ ID NO: 1, and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of amino acids 26 to 879 of SEQ ID NO: 1, amino acids 833 to 853 of SEQ ID NO: 1, SEQ ID NO: 2, or SEQ ID NO: 1.

In other aspects, the scaffold moiety, e.g., Scaffold X, comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to SEQ ID NO: 33. In other aspects, the Scaffold X comprises the amino acid sequence of SEQ ID NO: 33, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, the Scaffold X comprises the amino acid sequence of SEQ ID NO: 33 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NO: 33.

In other aspects, the Scaffold X comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to SEQ ID NO: 2, 3, 4, 5, 6, or 7. In other aspects, the Scaffold X comprises the amino acid sequence of SEQ ID NO: 2, 3, 4, 5, 6, or 7, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, the Scaffold X comprises the amino acid sequence of SEQ ID NO: 2, 3, 4, 5, 6, or 7 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NO: 2, 3, 4, 5, 6, or 7.

TABLE 1

Exemplary Scaffold X Protein Sequences

| | |
|---|---|
| Protein | Sequence |
| The PTGFRN Protein (SEQ ID NO: 1) | MGRLASRPLLLALLSLALCRGRVVRVPTATLVRGTELVIPCNVSDYDGPSEQNFDWSF SSLGSSFVELASTWEVGFPAQLYQERLQRGEILLRRTANDAVELHIKNVQPSDQGHYKCS TPSTDATVQGNYEDTVQVKVLADSLHVGPSARPPPSLSLREGEPFELRCTAASASPLHTH LALLWEVHRGPARRSVLALTHEGRFHPGLGYEQRYHSGDVRLDTVGSDAYRLSVSRALSA DQGSYRCIVSEWIAEQGNWQEIQEKAVEVATVVIQPSVLRAAVPKNVSVAEGKELDLTCN ITTDRADDVRPEVTWSFSRMPDSTLPGSRVLARLDRDSLVHSSPHVALSHVDARSYHLLV RDVSKENSGYYYCHVSLWAPGHNRSWHKVAEAVSSPAGVGVTWLEPDYQVYLNASKVPGF ADDPTELACRWDTKSGEANVRFTVSWYYRMNRRSDNWTSELLAVMDGDWTLKYGERSK QRAQDGDFIFSKEHTDTFNFRIQRTTEEDRGNYYCVVSAWTKQRNNSWVKSKDVFSKPVN IFWALEDSVLVVKARQPKPFFAAGNTFEMTCKVSSKNIKSPRYSVLIMAEKPVGDLSSPN ETKYIISLDQDSWKLENWTDASRVDGWLEKVQEDEFRYRMYQTQVSDAGLYRCMVTAW SPVRGSLWREAATSLSNPIEIDFQTSGPIFNASVHSDTPSVIRGDLIKLFCIITVEGAAL DPDDMAFDVSWFAVHSFGLDKAPVLLSSLDRKGIVTTSRRDWKSDLSLERVSVLEFLLQV HGSEDQDFGNYYCSVTPWVKSPTGSWQKEAEIHSKPVFITVKMDVLNAFKYPLLIGVGLS TVIGLLSCLIGYCSSHWCCKKEVQETRRERRRLMSMEMD |
| The PTGFRN protein Fragment (SEQ ID NO: 33) | GPIFNASVHSDTPSVIRGDLIKLFCIITVEGAALDPDDMAFDVSWFAVHSFGLDKAPVLL SSLDRKGIVTTSRRDWKSDLSLERVSVLEFLLQVHGSEDQDFGNYYCSVTPWVKSPTGSW QKEAEIHSKPVFITVKMDVLNAFKYPLLIGVGLSTVIGLLSCLIGYCSSHWCCKKEVQET RRERRRLMSMEM 687-878 of SEQ ID NO: 1 |
| The BSG protein (SEQ ID NO: 9) | MAAALFVLLG FALLGTHGAS GAAGFVQAPL SQQRWVGGSV ELHCEAVGSP VPEIQWWFEG QGPNDTCSQL WDGARLDRVH IHATYHQHAA STISIDTLVE EDTGTYECRA SNDPDRNHLT RAPRVKWVRA QAVVLVLEPG TVFTTVEDLG SKILLTCSLN DSATEVTGHR WLKGGWLKE DALPGQKTEF KVDSDDQWGE YSCVFLPEPM GTANIQLHGP PRVKAVKSSE HINEGETAML VCKSESVPPV TDWAWYKITD SEDKALMNGS ESRFFVSSSQ GRSELHIENL NMEADPGQYR CNGTSSKGSD QAIITLRVRS HLAALWPFLG IVAEVLVLVT IIFIYEKRRK PEDVLDDDDA GSAPLKSSGQ HQNDKGKNVR QRNSS |
| The IGSF8 protein (SEQ ID NO: 14) | MGALRPTLLP PSLPLLLLLM LGMGCWAREV LVPEGPLYRV AGTAVSISCN VTGYEGPAQQ NFEWFLYRPE APDTALGIVS TKDTQFSYAV FKSRVVAGEV QVQRLQGDAV VLKIARLQAQ DAGIYECHTP STDTRYLGSY SGKVELRVLP DVLQVSAAPP GPRGRQAPTS PPRMTVHEGQ ELALGCLART STQKHTHLAV SFGRSVPEAP VGRSTLQEW GIRSDLAVEA GAPYAERLAA GELRLGKEGT DRYRMVVGGA QAGDAGTYHC TAAEWIQDPD GSWAQIAEKR AVLAHVDVQT LSSQLAVTVG PGERRIGPGE PLELLCNVSG ALPPAGRHAA YSVGWEMAPA GAPGPGRLVA QLDTEGVGSL GPGYEGRHIA MEKVASRTYR LRLEAARPGD AGTYRCLAKA YVRGSGTRLR EAASARSRPL PVHVREEGW LEAVAWLAGG TVYRGETASL LCNISVRGGP PGLRLAASWW VERPEDGELS SVPAQLVGGV GQDGVAELGV RPGGGPVSVE LVGPRSHRLR LHSLGPEDEG VYHCAPSAWV QHADYSWYQA GSARSGPVTV YPYMHALDTL FVPLLVGTGV ALVTGATVLG |
| | TITCCFMKRL RKR |
| The ITGB1 protein (SEQ ID NO: 21) | MNLQPIFWIG LISSVCCVFA QTDENRCLKA NAKSCGECIQ AGPNCGWCTN STFLQEGMPT SARCDDLEAL KKKGCPPDDI ENPRGSKDIK KNKNVTNRSK GTAEKLKPED ITQIQPQQLV LRLRSGEPQT FTLKFKRAED YPIDLYYLMD LSYSMKDDLE NVKSLGTDLM NEMRRITSDF RIGFGSFVEK TVMPYISTTP AKLRNPCTSE QNCTSPFSYK NVLSLTNKGE VFNELVGKQR ISGNLDSPEG GFDAIMQVAV CGSLIGWRNV TRLLVFSTDA GFHFAGDGKL GGIVLPNDGQ CHLENNMYTM SHYYDYPSIA HLVQKLSENN IQTIFAVTEE FQPVYKELKN LIPKSAVGTL SANSSNVIQL IIDAYNSLSS EVILENGKLS EGVTISYKSY CKNGVNGTGE NGRKCSNISI GDEVQFEISI TSNKCPKKDS DSFKIRPLGF TEEVEVILQY ICECECQSEG IPESPKCHEG NGTFECGACR CNEGRVGRHC ECSTDEVNSE DMDAYCRKEN SSEICSNNGE CVCGQCVCRK RDNTNEIYSG ASNGQICNGR GICECGVCKC TDPKFQGQTC EMCQTCLGVC AEHKECVQCR AFNKGEKKDT CTQECSYFNI TKVESRDKLP QPVQPDPVSH CKEKDVDDCW FYFTYSVNGN NEVMVHWEN PECPTGPDII PIVAGVVAGI VLIGLALLLI WKLLMIIHDR REFAKFEKEK MNAKWDTGEN PIYKSAVTTV VNPKYEGK |
| The ITGA4 protein (SEQ ID NO: 22) | MAWEARREPG PRRAAVRETV MLLLCLGVPT GRPYNVDTES ALLYQGPHNT LFGYSVVLHS HGANRWLLVG APTANWLANA SVINPGAIYR CRIGKNPGQT CEQLQLGSPN GEPCGKTCLE ERDNQWLGVT LSRQPGENGS IVTCGHRWKN IFYIKNENKL PTGGCYGVPP DLRTELSKRI APCYQDYVKK FGENFASCQA GISSFYTKDL IVMGAPGSSY WTGSLFVYNI TTNKYKAFLD KQNQVKFGSY LGYSVGAGHF RSQHTTEVVG GAPQHEQIGK AYIFSIDEKE LNILHEMKGK KLGSYFGASV CAVDLNADGF SDLLVGAPMQ STIREEGRVF VYINSGSGAV MNAMETNLVG SDKYAARFGE SIVNLGDIDN DGFEDVAIGA PQEDDLQGAI YIYNGRADGI SSTFSQRIEG LQISKSLSMF GQSISGQIDA DNNGYVDVAV GAFRSDSAVL LRTRPVVIVD ASLSHPESVN RTKFDCVENG WPSVCIDLTL CFSYKGKEVP GYIVLFYNMS LDVNRKAESP PRFYFSSNGT SDVITGSIQV SSREANCRTH QAFMRKDVRD ILTPIQIEAA YHLGPHVISK RSTEEFPPLQ PILQQKKEKD IMKKTINFAR FCAHENCSAD LQVSAKIGFL KPHENKTYLA VGSMKTLMLN VSLFNAGDDA YETTLHVKLP VGLYFIKILE LEEKQINCEV TDNSGVVQLD CSIGYIYVDH LSRIDISFLL DVSSLSRAEE DLSITVHATC ENEEEMDNLK HSRVTVAIPL KYEVKLTVHG FVNPTSFVYG SNDENEPETC MVEKMNLTFH VINTGNSMAP NVSVEIMVPN SFSPQTDKLF NILDVQTTTG ECHFENYQRV CALEQQKSAM QTLKGIVRFL SKTDKRLLYC IKADPHCLNF LCNFGKMESG KEASVHIQLE GRPSILEMDE TSALKFEIRA TGFPEPNPRV IELNKDENVA HVLLEGLHHQ RPKRYFTIVI ISSSLLLGLI VLLLISYVMW KAGFFKRQYK SILQEENRRD SWSYINSKSN DD |
| The SLC3A2 Protein, where the first Met is processed. (SEQ ID NO: 23) | MELQPPEASI AVVSIPRQLP GSHSEAGVQG LSAGDDSELG SHCVAQTGLE LLASGDPLPS ASQNAEMIET GSDCVTQAGL QLLASSDPPA LASKNAEVTG TMSQDTEVDM KEVELNELEP EKQPMNAASG AAMSLAGAEK NGLVKIKVAE DEAEAAAAAK FTGLSKEELL KVAGSPGWVR TRWALLLLFW LGWLGMLAGA VVIIVRAPRC RELPAQKWWH TGALYRIGDL QAFQGHGAGN LAGLKGRLDY LSSLKVKGLV LGPIHKNQKD DVAQTDLLQI DPNFGSKEDF DSLLQSAKKK SIRVILDLTP NYRGENSWFS TQVDTVATKV KDALEFWLQA GVDGFQVRDI ENLKDASSFL AEWQNITKGF SEDRLLIAGT NSSDLQQILS LLESNKDLLL TSSYLSDSGS TGEHTKSLVT QYLNATGNRW CSWSLSQARL LTSFLPAQLL RLYQLMLFTL PGTPVFSYGD EIGLDAAALP GQPMEAPVML WDESSFPDIP GAVSANMTVK GQSEDPGSLL SLFRRLSDQR SKERSLLHGD FHAFSAGPGL FSYIRHWDQN ERFLVVLNFG DVGLSAGLQA SDLPASASLP AKADLLLSTQ PGREEGSPLE LERLKLEPHE GLLLRFPYAA |

In other embodiments, the scaffold moiety, e.g., Scaffold X, comprises the BSG protein, the IGSF8 protein, the IGSF3 protein, the ITGB1 protein, the SLC3A2 protein, the ITGA4 protein, the ATP1A1 protein, the ATP1A2 protein, the ATP1A3 protein, the ATP1A4 protein, the ATP1A5 protein, the ATP2B1 protein, the ATP2B2 protein, the ATP2B3 protein, the ATP2B4 protein, or the IGSF2 protein, which comprises an amino acid sequence at least about 70%, at least about 75%, at least about 80%, at least about 85%, at least about 90%, at least about 95%, at least about 96%, at least about 97%, at least about 98%, at least about 99%, or about 100% identical to the corresponding mature BSG protein, IGSF8 protein, IGSF3 protein, ITGB1 protein, SLC3A2 protein, ITGA4 protein, ATP1A1 protein, ATP1A2 protein, ATP1A3 protein, ATP1A4 protein, ATP1A5 protein, ATP2B1 protein, ATP2B2 protein, ATP2B3 protein, ATP2B4 protein, or IGSF2 protein (without the signal peptide). In some aspects, the BSG protein, the IGSF8 protein, the IGSF3 protein, the ITGB1 protein, the SLC3A2 protein, the ITGA4 protein, the ATP1A1 protein, the ATP1A2 protein, the ATP1A3 protein, the ATP1A4 protein, the ATP1A5 protein, the ATP2B1 protein, the ATP2B2 protein, the ATP2B3 protein, the ATP2B4 protein, or the IGSF2 protein lacks one or more functional or structural domains, such as IgV.

In some aspects, a scaffold moiety, e.g., Scaffold X, comprises Basigin (the BSG protein), represented by SEQ ID NO: 9. The BSG protein is also known as 5F7, Collagenase stimulatory factor, Extracellular matrix metalloproteinase inducer (EMMPRIN), Leukocyte activation antigen M6, OK blood group antigen, Tumor cell-derived collagenase stimulatory factor (TCSF), or CD147. The Uniprot number for the human BSG protein is P35613. The signal peptide of the BSG protein is amino acid 1 to 21 of SEQ ID NO: 9. Amino acids 138-323 of SEQ ID NO: 9 is the extracellular domain, amino acids 324 to 344 is the transmembrane domain, and amino acids 345 to 385 of SEQ ID NO: 9 is the cytoplasmic domain.

In some aspects, a scaffold moiety, e.g., Scaffold X, comprises Immunoglobulin superfamily member 8 (IgSF8 or the IGSF8 protein), which is also known as CD81 partner 3, Glu-Trp-Ile EWI motif-containing protein 2 (EWI-2), Keratinocytes-associated transmembrane protein 4 (KCT-4), LIR-D1, Prostaglandin regulatory-like protein (PGRL) or CD316. The full length human IGSF8 protein is accession no. Q969P0 in Uniprot and is shown as SEQ ID NO: 14 herein. The human IGSF8 protein has a signal peptide (amino acids 1 to 27 of SEQ ID NO: 14), an extracellular domain (amino acids 28 to 579 of SEQ ID NO: 14), a transmembrane domain (amino acids 580 to 600 of SEQ ID NO: 14), and a cytoplasmic domain (amino acids 601 to 613 of SEQ ID NO: 14).

Non-limiting examples of other Scaffold X proteins can be found at U.S. Pat. No. US10195290B1, issued Feb. 5, 2019, which is incorporated by reference in its entireties.

In some aspects, the sequence encodes a fragment of the scaffold moiety lacking at least about 5, at least about 10, at least about 50, at least about 100, at least about 200, at least about 300, at least about 400, at least about 500, at least about 600, at least about 700, or at least about 800 amino acids from the N-terminus of the native protein. In some aspects, the sequence encodes a fragment of the scaffold moiety lacking at least about 5, at least about 10, at least about 50, at least about 100, at least about 200, at least about 300, at least about 400, at least about 500, at least about 600, at least about 700, or at least about 800 amino acids from the C-terminus of the native protein. In some aspects, the sequence encodes a fragment of the scaffold moiety lacking at least about 5, at least about 10, at least about 50, at least about 100, at least about 200, at least about 300, at least about 400, at least about 500, at least about 600, at least about 700, or at least about 800 amino acids from both the N-terminus and C-terminus of the native protein. In some aspects, the sequence encodes a fragment of the scaffold moiety lacking one or more functional or structural domains of the native protein.

In some aspects, the scaffold moiety, e.g., Scaffold X, e.g., a PTGFRN protein, is linked to one or more heterologous proteins. The one or more heterologous proteins can be linked to the N-terminus of the scaffold moiety. The one or more heterologous proteins can be linked to the C-terminus of the scaffold moiety. In some aspects, the one or more heterologous proteins are linked to both the N-terminus and the C-terminus of the scaffold moiety. In some aspects, the heterologous protein is a mammalian protein. In some aspects, the heterologous protein is a human protein.

In some aspects, the scaffold moiety, e.g., Scaffold X, can be used to link any moiety to the luminal surface and the external surface of the EV (e.g., exosome) at the same time. For example, the PTGFRN polypeptide can be used to link one or more biologically active molecules indirectly through a maleimide moiety or directly to a maleimide moiety or a linker to the luminal surface in addition to the external surface of the EV (e.g., exosome). Therefore, in certain aspects, Scaffold X can be used for dual purposes.

In other aspects, the EVs useful to practice the methods for delivery to the CNS disclosed herein comprise a higher number of Scaffold X proteins compared to the naturally-occurring EVs, e.g., exosomes. In some aspects, the EVs of the disclosure comprise at least about 5 fold, at least about 10 fold, at least about 20 fold, at least about 30 fold, at least about 40 fold, at least about 50 fold, at least about 60 fold, at least about 70 fold, at least about 80 fold, at least about 90 fold, at least about 100 fold, at least about 110 fold, at least about 120 fold, at least about 130 fold, at least about 140 fold, at least about 150 fold, at least about 160 fold, at least about 170 fold, at least about 180 fold, at least about 190 fold, at least about 200 fold, at least about 210 fold, at least about 220 fold, at least about 230 fold, at least about 240 fold, at least about 250 fold, at least about 260 fold, at least about 270 fold higher number of Scaffold X (e.g., a PTGFRN polypeptide) compared to the naturally-occurring EV.

The number of scaffold moieties, e.g., Scaffold X, such as, a PTGFRN polypeptide, on the EV of the present disclosure is at least about 100, at least about 200, at least about 300, at least about 400, at least about 500, at least about 600, at least about 700, at least about 800, at least about 900, at least about 1000, at least about 1100, at least about 1200, at least about 1300, at least about 1400, at least about 1500, at least about 1600, at least about 1700, at least about 1800, at least about 1900, at least about 2000, at least about 2100, at least about 2200, at least about 2300, at least about 2400, at least about 2500, at least about 2600, at least about 2700, at least about 2800, at least about 2900, at least about 3000, at least about 4000, at least about 5000, at least about 6000, at least about 7000, at least about 8000, at least about 9000, or at least about 10000.

In some aspects, the number of scaffold moieties, e.g., Scaffold X, such as, a PTGFRN polypeptide, on the EV of the present disclosure is from about 100 to about 100,000, from about 200 to about 9000, from about 300 to about 9000, from about 400 to about 9000, from about 500 to about 9000, from about 600 to about 8000, from about 800 to about 8000, from about 900 to about 8000, from about 1000 to about 8000, from about 1100 to about 8000, from about 1200 to about 8000, from about 1300 to about 8000, from about 1400 to about 8000, from about 1500 to about 8000, from about 1600 to about 8000, from about 1700 to about 8000, from about 1800 to about 8000, from about 1900 to about 8000, from about 2000 to about 8000, from about 2100 to about 8000, from about 2200 to about 8000, from about 2300 to about 8000, from about 2400 to about 8000, from about 2500 to about 8000, from about 2600, from about 2700 to about 8000, from about 2800 to about 8000, from about 2900 to about 8000, from about 3000 to about 8000, from about 4000 to about 8000, from about 5000 to about 8000, from about 6000 to about 8000, from about 7000 to about 8000, from about 8000, from 7000 to about 9000, or from about 6000 to about 10000.

In some aspects, the number of scaffold moieties, e.g., Scaffold X, such as, a PTGFRN polypeptide, on the EV of the present disclosure is from about 5000 to about 8000, e.g., about 5000, about 6000, about 7000, or about 8000. In some aspects, the number of scaffold moieties, e.g., Scaffold X, such as, a PTGFRN polypeptide, on the EV of the present disclosure is from about 6000 to about 8000, e.g., about 6000, about 7000, or about 8000. In some aspects, the number scaffold moieties, e.g., Scaffold X, such as, a PTGFRN polypeptide, on the EV of the present disclosure is from about 4000 to about 9000, e.g., about 4000, about 5000, about 6000, about 7000, about 8000, about 9000.

In some aspects, Scaffold X can be used to link any moiety to the luminal surface and on the exterior surface of the EV at the same time. For example, the PTGFRN polypeptide can be used to link a therapeutic molecule (e.g., an antigen), an adjuvant, and/or an immune modulator inside the lumen (e.g., on the luminal surface) in addition to the exterior surface of the EV, e.g., exosome. Therefore, in certain aspects, Scaffold X can be used for dual purposes, e.g., a therapeutic molecule (e.g., an antigen) on the luminal surface and an adjuvant or immune modulator on the exterior surface of the EV a therapeutic molecule (e.g., an antigen) on the exterior surface of the EV and the adjuvant or immune modulator on the luminal surface, an adjuvant on the luminal surface and an immune modulator on the exterior surface of the EV or an immune modulator on the luminal surface and an adjuvant on the exterior surface of the EV, e.g., exosome.

### III.B.8. Luminal Scaffold- (e.g., Scaffold Y-) Engineered EVs, E.g., Exosomes

In some aspects, EVs of the present disclosure comprise an internal space (i.e., lumen) that is different from that of the naturally occurring EVs. For example, the EV can be changed such that the composition in the luminal surface of the EV has the protein, lipid, or glycan content different from that of the naturally-occurring exosomes (e.g., comprises multiple exogenous biologically active molecules disclosed herein).

In some aspects, engineered EVs can be produced from a cell transformed with an exogenous sequence encoding a scaffold moiety (e.g., exosome proteins, e.g., Scaffold Y) or a modification or a fragment of the scaffold moiety that changes the composition or content of the luminal surface of the EV, e.g., exosome. Various modifications or fragments of the exosome protein that can be expressed on the luminal surface of the EV can be used for the aspects of the present disclosure.

In some aspects, the exosome proteins that can change the luminal surface of the EVs include, but are not limited to, the myristoylated alanine rich Protein Kinase C substrate (MARCKS) protein, the myristoylated alanine rich Protein Kinase C substrate like 1 (MARCKSL1) protein, the brain acid soluble protein 1 (BASP1) protein, or any combination thereof. In certain aspects, EVs of the present disclosure comprise two or more (e.g., 2, 3, 4, 5 or more) of such exosome proteins.

In some aspects, Scaffold Y comprises the MARCKS protein (Uniprot accession no. P29966). The MARCKS protein is also known as protein kinase C substrate, 80 kDa protein, light chain. The full-length human MARCKS protein is 332 amino acids in length and comprises a calmodulin-binding domain at amino acid residues 152-176. In some aspects, Scaffold Y comprises the MARCKSL1 protein (Uniprot accession no. P49006). The MARCKSL1 protein is also known as MARCKS-like protein 1, and macrophage myristoylated alanine-rich C kinase substrate. The full-length human MARCKSL1 protein is 195 amino acids in length. The MARCKSL1 protein has an effector domain involved in lipid-binding and calmodulin-binding at amino acid residues 87-110. In some aspects, the Scaffold Y comprises the BASP1 protein (Uniprot accession number P80723). The BASP1 protein is also known as 22 kDa neuronal tissue-enriched acidic protein or neuronal axonal membrane protein NAP-22. The full-length human BASP1 protein sequence (isomer 1) is 227 amino acids in length. An isomer produced by an alternative splicing is missing amino acids 88 to 141 from SEQ ID NO: 49 (isomer 1). Table 2 provides the full-length sequences for the exemplary Scaffold Y disclosed herein (i.e., the MARCKS, MARCKSL1, and BASP1 proteins).

TABLE 2

Exemplary Scaffold Y Protein Sequences

| | |
|---|---|
| Protein | Sequence |
| The MARCKS protein (SEQ ID NO: 47) | MGAQFSKTAA KGEAAAERPG EAAVASSPSK ANGQENGHVK VNGDASPAAA ESGAKEELQA NGSAPAADKE EPAAAGSGAA SPSAAEKGEP AAAAAPEAGA SPVEKEAPAE GEAAEPGSPT AAEGEAASAA SSTSSPKAED GATPSPSNET PKKKKKRFSF KKSFKLSGFS FKKNKKEAGE GGEAEAPAAE GGKDEAAGGA AAAAAEAGAA SGEQAAAPGE EAAAGEEGAA GGDPQEAKPQ EAAVAPEKPP ASDETKAAEE PSKVEEKKAE EAGASAAACE APSAAGPGAP PEQEAAPAEE PAAAAASSAC AAPSQEAQPE CSPEAPPAEA AE |
| The MARCKSL1 protein (SEQ ID NO: 48) | MGSQSSKAPR GDVTAEEAAG ASPAKANGQE NGHVKSNGDL SPKGEGESPP VNGTDEAAGA TGDAIEPAPP SQGAEAKGEV PPKETPKKKK KFSFKKPFKL SGLSFKRNRK EGGGDSSASS PTEEEQEQGE IGACSDEGTA QEGKAAATPE SQEPQAKGAE ASAASEEEAG PQATEPSTPS GPESGPTPAS AEQNE |
| The BASP1 protein (SEQ ID NO: 49) | MGGKLSKKKK GYNVNDEKAK EKDKKAEGAA TEEEGTPKES EPQAAAEPAE AKEGKEKPDQ DAEGKAEEKE GEKDAAAAKE EAPKAEPEKT EGAAEAKAEP PKAPEQEQAA PGPAAGGEAP KAAEAAAAPA ESAAPAAGEE PSKEEGEPKK TEAPAAPAAQ ETKSDGAPAS DSKPGSSEAA PSSKETPAAT EAPSSTPKAQ GPAASAEEPK PVEAPAANSD QTVTVKE |

The mature BASP1 protein sequence is missing the first Met from SEQ ID NO: 49 and thus contains amino acids 2 to 227 of SEQ ID NO: 49. Similarly, the mature MARCKS and MARCKSL1 proteins also lack the first Met from SEQ ID NOs: 47 and 48, respectively. Accordingly, the mature MARCKS protein contains amino acids 2 to 332 of SEQ ID NO: 47. The mature MARCKSL1 protein contains amino acids 2 to 227 of SEQ ID NO: 48.

In other aspects, Scaffold Y useful for the present disclosure comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 2 to 227 of SEQ ID NO: 49. In other aspects, the Scaffold Y comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to any one of SEQ ID NOs: 50-155. In other aspects, a Scaffold Y useful for the present disclosure comprises the amino acid sequence of SEQ ID NO: 49, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, a Scaffold Y useful for the present disclosure comprises the amino acid sequence of any one of SEQ ID NOs: 50-155 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NOs: 50-155.

In some aspects, the protein sequence of any of SEQ ID NOs: 47-155 is sufficient to be a Scaffold Y for the present disclosure (e.g., scaffold moiety linked to a targeting moiety and/or a therapeutic molecule and/or an adjuvant and/or an immune modulator).

In other aspects, the Scaffold Y comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to any one of SEQ ID NO: 47-155.

Scaffold Y-engineered EVs, e.g., exosomes described herein can be produced from a cell transformed with a sequence set forth in SEQ ID NOs: 47-155.

In some aspects, the Scaffold Y protein comprises an amino acid sequence selected from the group consisting of (i) GGKLSKK (SEQ ID NO: 211), (ii) GAKLSKK (SEQ ID NO: 212), (iii) GGKQSKK (SEQ ID NO: 213), (iv) GGKLAKK (SEQ ID NO: 214), or (v) any combination thereof.

In some aspects, the Scaffold Y protein useful for the present disclosure does not contain an N-terminal Met. In some aspects, the Scaffold Y protein comprises a lipidated amino acid, e.g., a myristoylated amino acid, at the N-terminus of the scaffold protein, which functions as a lipid anchor. In some aspects, the amino acid residue at the N-terminus of the scaffold protein is Gly. The presence of an N-terminal Gly is an absolute requirement for N-myristoylation. In some aspects, the amino acid residue at the N-terminus of the scaffold protein is synthetic. In some aspects, the amino acid residue at the N-terminus of the scaffold protein is a glycine analog, e.g., allylglycine, butylglycine, or propargylglycine.

In some aspects, the scaffold moiety, e.g., Scaffold Y, comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to any one of the sequences disclosed in US 10,195,290B1, issued Feb. 5, 2019.

Scaffold Y-engineered exosomes described herein can be produced from a cell transformed with any sequence set forth in PCT/US2018/061679 (SEQ ID NO: 4-109 from PCT/US2018/061679).

In other aspects, the lipid anchor can be any lipid anchor known in the art, e.g., palmitic acid or glycosylphosphatidylinositols. Under unusual circumstances, e.g., by using a culture medium where myristic acid is limiting, some other fatty acids including shorter-chain and unsaturated, can be attached to the N-terminal glycine. For example, in BK channels, myristate has been reported to be attached posttranslationally to internal serine/threonine or tyrosine residues via a hydroxyester linkage. Membrane anchors known in the art are presented in the following table:

| | |
|---|---|
| Modification | ModifyingGroup |
| S-Palmitoylation | 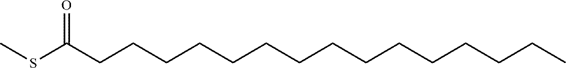 [SMILES: CCCCCCCCCCCCCCCC(=O)SC] |
| N-Palmitoylation | 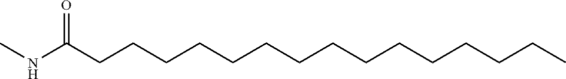 [SMILES: CCCCCCCCCCCCCCCC(=O)NC] |
| N-Myristoylation | 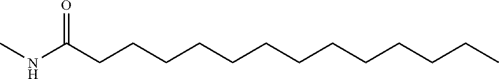 [SMILES: CCCCCCCCCCCCCC(=O)NC] |
| O-Acylation | 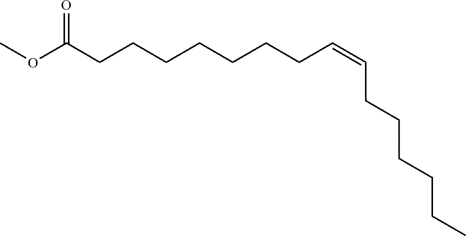 [SMILES: CCCCCC/C=C\CCCCCCCC(=O)OC] |
| Farnesylation | 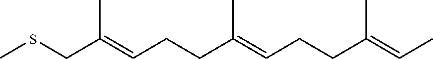 [SMILES: C/C=C(\C)CC/C=C(\C)CC/C=C(\C)CSC] |
| Geranylgeranylation | 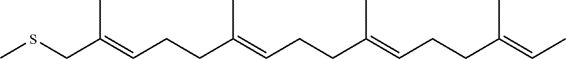 [SMILES: C/C=C(\C)CC/C=C(\C)CC/C=C(\C)CC/C=C(\C)CSC] |
| Cholesterol | 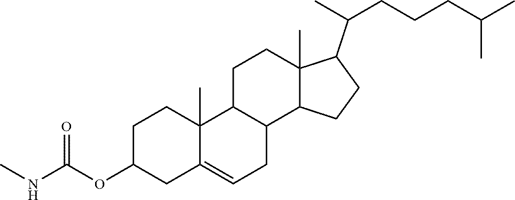 [SMILES: CNC(=O)OC1CCC2(C)C(=CCC3C2CCC2(C)C(C(C)CCCC(C)C)CCC32)C1] |

### III.B.9. Lipid Anchoring Moieties

Suitable anchoring moieties capable of anchoring a biologically active molecule to the surface of an EV, e.g., an exosome, via a maleimide moiety comprise for example sterols (e.g., cholesterol), phospholipid, lysophospholipids, fatty acids, or fat-soluble vitamins, as described in detail below.

In some aspects, the anchoring moiety can be a lipid. A lipid anchoring moiety can be any lipid known in the art, e.g., palmitic acid or glycosylphosphatidylinositols. In some aspects, the lipid, is a fatty acid, phosphatide, phospholipid (e.g., phosphatidyl choline, phosphatidyl serine, or phosphatidyl ethanolamine), or analogue thereof (e.g. phosphatidylcholine, lecithin, phosphatidylethanolamine, cephalin, or phosphatidylserine or analogue or portion thereof, such as a partially hydrolyzed portion thereof).

The anchoring moiety can be conjugated using a maleimide moiety to a biologically active molecule directly or indirectly via a linker combination, at any chemically feasible location, e.g., at the 5′ and/or 3′ end of a nucleotide sequence, e.g., of a biologically active molecule (e.g, an ASO). In one aspect, the anchoring moiety is conjugated only to the 3′ end of the biologically active molecule. In one aspect, the anchoring moiety is conjugated only to the 5′ end of a nucleotide sequence, e.g., of a biologically active molecule (e.g, an ASO). In one aspect, the anchoring moiety is conjugated at a location which is not the 3′ end or 5′ end of a nucleotide sequence, e.g., of a biologically active molecule (e.g, an ASO).

In some aspects, a biologically active molecule can be conjugated directly or indirectly via a maleimide group to, e.g., any of the lipid anchors disclosed above (for example, palmitic acid, myristic acid, fatty acid, farnesyl, geranyl-geranyl, or cholesterol). In some aspects, an anchoring moiety can comprise two or more types of anchoring moieties disclosed herein. For example, in some aspects, an anchoring moiety can comprise two lipids, e.g., a phospholipids and a fatty acid, or two phospholipids, or two fatty acids, or a lipid and a vitamin, or cholesterol and a vitamin, etc. which taken together have 6-80 carbon atoms (i.e., an equivalent carbon number (ECN) of about 6 to about 80).

### III.B.10. Scaffold Protein Fusion Constructs

In some aspects, the scaffold moiety is linked to one or more heterologous proteins. The one or more heterologous proteins can be linked to the N-terminus of the scaffold moieties. The one or more heterologous proteins can be linked to the C-terminus of the scaffold moieties. In some aspects, the one or more heterologous proteins are linked to both the N-terminus and the C-terminus of the scaffold moieties. In some aspects, the heterologous protein is a mammalian protein. In some aspects, the heterologous protein is a human protein.

In some aspects, the scaffold moiety can be used to link any moiety to the luminal surface and/or the external surface of the EV, e.g., exosome. For example, the PTGFRN polypeptide can be used to link a biologically active molecule inside the lumen (e.g., on the luminal surface) in addition to the external surface of the EV, e.g., exosome. Therefore, in certain aspects, the scaffold moiety can be used for dual purposes, e.g., a biologically active molecule on the luminal surface and a second biologically active molecule or other payload on the external surface of the EV or a biologically active molecule on the external surface of the exosome and a second biologically active molecule or other payload on the luminal surface of the EV, e.g., exosome.

### III.B.11. Linkers

As described supra, extracellular vesicles (EVs) of the present disclosure (e.g., exosomes and nanovesicles) can comprises one or more linkers that link one or more exogenous biologically active molecules disclosed herein (e.g., targeting moiety, therapeutic molecule (e.g., antigen), adjuvant, or immune modulator) to the EVs (e.g., to the exterior surface or on the luminal surface). In some aspects, the one or more exogenous biologically active molecules (e.g., targeting moiety, therapeutic molecule, adjuvant, or immune modulator) are linked to the EVs directly or via one or more scaffold moieties (e.g., Scaffold X or Scaffold Y). For example, in certain aspects, one or more exogenous biologically active molecules are linked to the exterior surface of an exosome via Scaffold X. In further aspects, one or more exogenous biologically active molecules are linked to the luminal surface of an exosome via Scaffold X or Scaffold Y. The linker can be any chemical moiety known in the art.

As used herein, the term “linker” refers to a peptide or polypeptide sequence (e.g., a synthetic peptide or polypeptide sequence) or to a non-polypeptide, e.g., an alkyl chain. In some aspects, two or more linkers can be linked in tandem. When multiple linkers are present, each of the linkers can be the same or different. Generally, linkers provide flexibility or prevent/ameliorate steric hindrances. Linkers are not typically cleaved; however in certain aspects, such cleavage can be desirable. Accordingly, in some aspects, a linker can comprise one or more protease-cleavable sites, which can be located within the sequence of the linker or flanking the linker at either end of the linker sequence.

In some aspects, the linker is a peptide linker. In some aspects, the peptide linker can comprise at least about two, at least about three, at least about four, at least about five, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 amino acids.

In some aspects, the peptide linker is synthetic, i.e., non-naturally occurring. In one aspect, a peptide linker includes peptides (or polypeptides) (e.g., natural or non-naturally occurring peptides) which comprise an amino acid sequence that links or genetically fuses a first linear sequence of amino acids to a second linear sequence of amino acids to which it is not naturally linked or genetically fused in nature. For example, in one aspect the peptide linker can comprise non-naturally occurring polypeptides which are modified forms of naturally occurring polypeptides (e.g., comprising a mutation such as an addition, substitution or deletion).

Linkers can be susceptible to cleavage (“cleavable linker”) thereby facilitating release of the exogenous biologically active molecule (e.g., targeting moiety, therapeutic molecule, adjuvant, or immune modulator).

In some aspects, the linker is a “reduction-sensitive linker.” In some aspects, the reduction-sensitive linker contains a disulfide bond. In some aspects, the linker is an “acid labile linker.” In some aspects, the acid labile linker contains hydrazone. Suitable acid labile linkers also include, for example, a cis-aconitic linker, a hydrazide linker, a thiocarbamoyl linker, or any combination thereof.

In some aspects, the linker comprises a non-cleavable linker.

### III.B.12. Tropism

In some aspects, an EV disclosed herein can be surface engineered to adjust its properties, e.g., biodistribution, e.g., via incorporation of immuno-affinity ligands or cognate receptor ligands. For example, EV, e.g., exosomes, disclosed herein can be surface engineered to direct them to a specific cellular type, e.g., Schwann cells, sensory neurons, motor neurons, or meningeal macrophages, or can be surface engineered to enhance their migration to a specific compartment, e.g., to the CNS in order to improve intrathecal compartment retention.

In some aspects, an EV for delivery, e.g., compartmental delivery, to the CNS disclosed herein comprises a bio-distribution modifying agent or targeting moiety. As used here, the terms “bio-distribution modifying agent” and “targeting moiety” are used interchangeably and refer to an agent that can modify the distribution of extracellular vesicles (e.g., exosomes, nanovesicles) in vivo or in vitro (e.g., in a mixed culture of cells of different varieties). In some aspects, the targeting moiety alters the tropism of the EV, i.e., the target moiety is a “tropism moiety”. As used herein, the term “tropism moiety” refers to a targeting moiety that when expressed on an EV alters and/or enhances the natural movement of the EV. For example, in some aspects, a tropism moiety can promote the EV to be taken up by a particular cell, tissue, or organ.

EVs exhibit preferential uptake in discrete cell types and tissues, and their tropism can be directed by adding proteins to their surface that interact with receptors on the surface of target cells. The tropism moiety can comprise a biological molecule, such as a protein, a peptide, a lipid, or a carbohydrate, or a synthetic molecule. For example, in some aspects the tropism moiety can comprise an affinity ligand, e.g., an antibody (such as an anti-CD19 nanobody, an anti-CD22 nanobody, an anti-CLEC9A nanobody, or an anti-CD3 nanobody), a VHH domain, a phage display peptide, a fibronectin domain, a camelid nanobody, and/or a vNAR. In some aspects, the tropism moiety can comprise, e.g., a synthetic polymer (e.g., PEG), a natural ligand/molecule (e.g., CD40L, albumin, CD47, CD24, CD55, CD59), and/or a recombinant protein (e.g., XTEN).

In some aspects, a tropism moiety can increase uptake of the EV, e.g., an exosome, by a cell. In some aspects, the tropism moiety that can increase uptake of the EV, e.g., an exosome, by a cell comprises a lymphocyte antigen 75 (also known as DEC205 or CD205), C-type lectin domain family 9 member A (CLEC9A), C-type lectin domain family 6 (CLEC6), C-type lectin domain family 4 member A (also known as DCIR or CLEC4A), Dendritic Cell-Specific Intercellular adhesion molecule-3-Grabbing Non-integrin (also known as DC-SIGN or CD209), lectin-type oxidized LDL receptor 1(LOX-1), macrophage receptor with collagenous structure (MARCO), C-type lectin domain family 12 member A (CLEC12A), C-type lectin domain family 10 member A (CLEC10A), DC-asialoglycoprotein receptor (DC-ASGPR), DC immunoreceptor 2 (DCIR2), Dectin-1, macrophage mannose receptor (MMR), BDCA-2 (CD303, CLEC4C), Dectin-2, BST-2 (CD317), Langerin, CD206, CD11b, CD11c, CD123, CD304, XCR1, AXL, SIGLEC 6, CD209, SIRPA, CX3CR1, GPR182, CD14, CD16, CD32, CD34, CD38, CD10, anti-CD3 antibody, or any combination thereof.

In some aspects, when tropism to the central nervous system is desired, an EV of the present disclosure can comprise a tissue or cell-specific target ligand, which increases EV tropism to a specific central nervous system tissue or cell. In some aspects, the cell is a glial cell. In some aspects, the glial cell is an oligodendrocyte, an astrocyte, an ependymal cell, a microglia cell, a Schwann cell, a satellite glial cell, an olfactory ensheathing cell, or a combination thereof. In some aspects, the cell is a neural stem cell. In some aspects, the cell-specific target ligand, which increases EV tropism to a Schwann cells binds to a Schwann cell surface marker such as Myelin Basic Protein (MBP), Myelin Protein Zero (P0), P75NTR, NCAM, PMP22, or any combination thereof. In some aspects, the cell-specific tropism moiety comprises an antibody or an antigen-binding portion thereof, an aptamer, or an agonist or antagonist of a receptor expressed on the surface of the Schwann cell.

In principle, the EVs, e.g., exosomes of the present disclosure comprising at least one tropism moiety that can direct the EV to a specific target cell or tissue (e.g., a cell in the CNS or a Schwann cell in peripheral nerves) can be administered using any suitable administration method known in the art (e.g., intravenous injection or infusion) since the presence of the tropism moiety (alone or in combination with the presence of an antiphagocytic signal such as CD47 and the use of a specific administration route) will induce a tropism of the EVs towards the desired target cell or tissue. In some aspects, the tropism of the EV, e.g., exosome is enhanced by compartmental administration (e.g., intrathecal administration or intraocular administration to improve tropism to the central nervous system) of the EV as disclosed herein.

In certain aspects, the tropism moiety is linked, e.g., chemically linked via a maleimide moiety, to a scaffold moiety, e.g., a Scaffold X protein or a fragment thereof, on the exterior surface of the EV, e.g., exosome. Tropism can be further improved by the attachment of an anti-phagocytic signal (e.g., CD47 and/or CD24), a half-life extension moiety (e.g., albumin or PEG), or any combination thereof to the external surface of an EV, e.g., exosome of the present disclosure. In certain aspects, the anti-phagocytic signal is linked, e.g., chemically linked via a maleimide moiety, to a scaffold moiety, e.g., a Scaffold X protein or a fragment thereof, on the exterior surface of the EV, e.g., exosome.

Pharmacokinetics, biodistribution, and in particular tropism and retention in the desired tissue or anatomical location can also be accomplish by selecting the appropriate administration route (e.g., intrathecal administration or intraocular administration to improve tropism to the central nervous system).

In some aspects, the EV comprises at least two different tropism moieties. In some aspects, the EV comprises three different tropism moieties. In some aspects, the EV comprises four different tropism moieties. In some aspects, the EV comprises five or more different tropism moieties. In some aspects, one or more of the tropism moieties increases uptake of the EV by a cell. In some aspects, each tropism moiety is attached to a scaffold moiety, e.g., a Scaffold X protein or a fragment thereof. In some aspects, multiple tropism moieties can be attached to the same scaffold moiety, e.g., a Scaffold X protein or a fragment thereof. In some aspects, several tropism moieties can be attached in tandem to a scaffold moiety, e.g., a Scaffold X protein or a fragment thereof. In some aspects, a tropism moiety disclosed herein or a combination thereof is attached to a scaffold moiety, e.g., a Scaffold X protein or a fragment thereof, via a linker or spacer. In some aspects, a linker or spacer or a combination thereof is interposed between two tropism moieties disclosed herein.

Non-limiting examples of tropism moieties capable of directing EVs of the present disclosure to different nervous system cell types are disclosed below.

Tropism moieties targeting Schwann cells: In some aspects, a tropism moiety can target a Schwann cell. In some aspects, the tropism moiety that directs an EV disclosed herein to a Schwann cell targets, e.g., a transferrin receptor (TfR), apolipoprotein D (ApoD), Galectin 1 (LGALS1), Myelin proteolipid protein (PLP), Glypican 1, or Syndecan 3. In some aspects, the tropism moiety directing an EV of the present disclosure to a Schwann cell is a transferrin, or a fragment, variant or derivative thereof. In some aspects, the tropism moiety that directs an EV disclosed herein to a Schwann cell comprises basigin-1. In some aspects, the tropism moiety that directs an EV disclosed herein to a Schwann cell comprises m. Leperae peptide.

In some aspects, a tropism moiety of the present disclosure targets a transferrin receptor (TfR). Transferrin receptors, e.g., TfR1 or TfR2, are carrier proteins for transferrin. Transferrin receptors import iron by internalizing the transferrin-ion complex through receptor-mediated endocytosis.

TfR1 (see, e.g., UniProt P02786 TFR1_Human) or transferrin receptor 1 (also known as cluster of differentiation 71 or CD71) is expressed on the endothelial cells of the blood-brain barrier (BBB). TfR1 is known to be expressed in a variety of cells such as red blood cells, monocytes, hepatocytes, intestinal cells, and erythroid cells, and is upregulated in rapidly dividing cells such as tumor cells (non small cell lung cancer, colon cancer, and leukemia) as well as in tissue affected by disorders such as acute respiratory distress syndrome (ARDS). TfR2 is primarily expressed in liver and erythroid cells, is found to a lesser extent in lung, spleen and muscle, and has a 45% identity and 66% similarity with TfR1. TfR1 is a transmembrane receptor that forms a homodimer of 760 residues with disulfide bonds and a molecular weight of 90 kDa. Affinity for transferrin varies between the two receptor types, with the affinity for TfR1 being at least 25-30 fold higher than that of TfR2.

Binding to TfR1 allows the transit of large molecules, e.g., antibodies, into the brain. Some TfR1-targeting antibodies have been shown to cross the blood-brain barrier, without interfering with the uptake of iron. Amongst those are the mouse anti-rat-TfR antibody OX26 and the rat anti-mouse-TfR antibody 8D3. The affinity of the antibody-TfR interaction is important to determine the success of transcytotic transport over endothelial cells of the BBB. Monovalent TfR interaction favors BBB transport due to altered intracellular sorting pathways. Avidity effects of bivalent interactions redirecting transport to the lysosome. Also, reducing TfR binding affinity directly promote dissociation from the TfR which increase brain parenchymal exposure of the TfR binding antibody. See, e.g., U.S. Pat. No. 8,821,943, which is herein incorporated by reference in its entirety. Accordingly, in some aspects, a tropism moiety of the present disclosure can comprise a ligand that can target TfR, e.g., target TfR1, such as transferrin, or an antibody or other binding molecule capable of specifically binding to TfR. In some aspects, the antibody targeting a transferrin receptor is a low affinity anti-transferring receptor antibody (see, e.g., US20190202936A1, which is herein incorporated by reference in its entirety).

In some aspects, the tropism moiety comprises all or a portion (e.g., a binding portion) of a ligand for a transferrin receptor, for example a human transferrin available in GenBank as Accession numbers NM001063, XM002793, XM039847, NM002343 or NM013900, among others, or a variant, fragment, or derivative thereof.

In some aspects, the tropism moiety comprises a transferrin-receptor-targeting moiety, i.e., a targeting moiety directed to a transferrin receptor. Suitable transferrin-receptor-targeting moieties include a transferrin or transferrin variant, such as, but not limited to, a serum transferrin, lacto transferrin (lactoferrin) ovotransferrin, or melanotransferrin. Transferrins are a family of nonheme iron-binding proteins found in vertebrates, including serum transferrins, lacto transferrins (lactoferrins), ovotransferrins, and melanotransferrins. Serum transferrin is a glycoprotein with a molecular weight of about 80 kDa, comprising a single polypeptide chain with two N-linked polysaccharide chains that are branched and terminate in multiple antennae, each with terminal sialic acid residues. There are two main domains, the N domain of about 330 amino acids, and the C domain of about 340 amino acids, each of which is divided into two subdomains, N1 and N2, and C1 and C2. Receptor binding of transferrin occurs through the C domain, regardless of glycosylation.

In some aspects, the tropism moiety is a serum transferrin or transferrin variant such as, but not limited to a hexasialo transferrin, a pentasialo transferrin, a tetrasialo transferrin, a trisialo transferrin, a disialo transferrin, a monosialo transferrin, or an asialo transferrin, or a carbohydrate-deficient transferrin (CDT) such as an asialo, monosialo or disialo transferrin, or a carbohydrate-free transferrin (CFT) such as an asialo transferrin. In some aspects, the tropism moiety is a transferrin variant having the N-terminal domain of transferrin, the C-terminal domain of transferrin, the glycosylation of native transferrin, reduced glycosylation as compared to native (wild-type) transferrin, no glycosylation, at least two N terminal lobes of transferrin, at least two C terminal lobes of transferrin, at least one mutation in the N domain, at least one mutation in the C domain, a mutation wherein the mutant has a weaker binding avidity for transferrin receptor than native transferrin, and/or a mutation wherein the mutant has a stronger binding avidity for transferrin receptor than native transferrin, or any combination of the foregoing.

In some aspects, the tropism moiety targeting a transferrin receptor comprises an anti-trasferrin receptor variable new antigen receptor (vNAR), e.g., a binding domain with a general motif structure (FW1-CDR1-FW2-3-CDR3-FW4). See, e.g., U.S. 2017-0348416, which is herein incorporated by reference in its entirety. vNARs are key component of the adaptive immune system of sharks. At only 11 kDa, these single-domain structures are the smallest IgG-like proteins in the animal kingdom and provide an excellent platform for molecular engineering and biologics drug discovery. vNAR attributes include high affinity for target, ease of expression, stability, solubility, multi-specificity, and increased potential for solid tissue penetration. See Ubah et al. Biochem. Soc. Trans. (2018) 46(6): 1559-1565.

In some aspects, the tropism moiety comprises a vNAR domain capable of specifically binding to TfR1, wherein the vNAR domain comprises or consists essentially of a vNAR scaffold with any one CDR1 peptide in Table 1 of U.S. 2017-0348416 in combination with any one CDR3 peptide in Table 1 of U.S. 2017-0348416.

In some aspects, a tropism moiety of the present disclosure targets ApoD. Unlike other lipoproteins, which are mainly produced in the liver, apolipoprotein D is mainly produced in the brain, cerebellum, and peripheral nerves. ApoD is 169 amino acids long, including a secretion peptide signal of 20 amino acids. It contains two glycosylation sites (aspargines 45 and 78) and the molecular weight of the mature protein varies from 20 to 32 kDa. ApoD binds steroid hormones such as progesterone and pregnenolone with a relatively strong affinity, and to estrogen with a weaker affinity. Arachidonic acid (AA) is an ApoD ligand with a much better affinity than that of progesterone or pregnenolone. Other ApoD ligands include E-3-methyl-2-hexenoic acid, retinoic acid, sphingomyelin and sphingolipids. Accordingly, in some aspects, a tropism moiety of the present disclosure comprises a ligand that can target ApoD, e.g., an antibody or other binding molecule capable of specifically binding to ApoD.

In some aspects, a tropism moiety of the present disclosure targets Galectin 1. The galectin-1 protein is 135 amino acids in length. Accordingly, in some aspects, a tropism moiety of the present disclosure comprises a ligand that can target Galectin 1, e.g., an antibody or other binding molecule capable of specifically binding to Galectin 1.

In some aspects, a tropism moiety of the present disclosure targets PLP. PLP is the major myelin protein from the CNS. It plays an important role in the formation or maintenance of the multilamellar structure of myelin. The myelin sheath is a multi-layered membrane, unique to the nervous system that functions as an insulator to greatly increase the efficiency of axonal impulse conduction. PLP is a highly conserved hydrophobic protein of 276 to 280 amino acids which contains four transmembrane segments, two disulfide bonds and which covalently binds lipids (at least six palmitate groups in mammals). Accordingly, in some aspects, a tropism moiety of the present disclosure comprises a ligand that can target PLP, e.g., an antibody or other binding molecule capable of specifically binding to PLP.

In some aspects, a tropism moiety of the present disclosure targets Glypican 1. Accordingly, in some aspects, a tropism moiety of the present disclosure comprises a ligand that can target Glypican 1, e.g, an antibody or other binding molecule capable of specifically binding to Glypican 1. In some aspects, a tropism moiety of the present disclosure targets Syndecan 3. Accordingly, in some aspects, a tropism moiety of the present disclosure comprises a ligand that can target Syndecan 3, e.g., an antibody or other binding molecule capable of specifically binding to Syndecan 3.

Tropism moieties targeting sensory neurons: In some aspects, a tropism moiety disclosed herein can direct an EV, e.g, exosome, disclosed herein to a sensory neuron. In some aspects, the tropism moiety that directs an EV, e.g, exosome, disclosed herein to a sensory neuron targets a Trk receptor, e.g., TrkA, TrkB, TrkC, or a combination thereof.

Trk (tropomyosin receptor kinase) receptors are a family of tyrosine kinases that regulates synaptic strength and plasticity in the mammalian nervous system. The common ligands of Trk receptors are neurotrophins, a family of growth factors critical to the functioning of the nervous system. The binding of these molecules is highly specific. Each type of neurotrophin has different binding affinity toward its corresponding Trk receptor. Accordingly, in some aspects, the tropism moiety directing an EV, e.g, exosome, disclosed herein to a sensory neuron, comprises a neurotrophin.

Neurotrophins bind to Trk receptors as homodimers. Accordingly, in some aspects, the tropism moiety comprises at least two neurotrophins disclosed herein, e.g., in tandem. In some aspects, the tropism moiety comprises at least two neurotrophins disclosed herein, e.g., in tandem, that are attached to a scaffold protein, for example, Protein X, via a linker. In some aspects, the linker connecting the scaffold protein, e.g., Protein X, to the neurotrophin (e.g., a neurotrophin homodimer) has a length of at least 10 amino acids. In some aspects, the linker connecting the scaffold protein, e.g., Protein X, to the neurotrophin (e.g., a neurotrophin homodimer) has a length of at least about 25 amino acids, about 30 amino acids, about 35 amino acids, about 40 amino acids, about 45 amino acids, or about 50 amino acids.

In some aspects, the neurotrophin is a neurotrophin precursor, i.e., a proneurotrophin, which is later cleaved to produce a mature protein.

Nerve growth factor (NGF) is the first identified and probably the best characterized member of the neurotrophin family. It has prominent effects on developing sensory and sympathetic neurons of the peripheral nervous system. Brain-derived neurotrophic factor (BDNF) has neurotrophic activities similar to NGF, and is expressed mainly in the CNS and has been detected in the heart, lung, skeletal muscle and sciatic nerve in the periphery (Leibrock, J. et al., Nature, 341:149-152 (1989)). Neurotrophin-3 (NT-3) is the third member of the NGF family and is expressed predominantly in a subset of pyramidal and granular neurons of the hippocampus, and has been detected in the cerebellum, cerebral cortex and peripheral tissues such as liver and skeletal muscles (Emfors, P. et al., Neuron 1: 983-996 (1990)). Neurotrophin-4 (also called NT-415) is the most variable member of the neurotrophin family. Neurotrophin-6 (NT-5) was found in teleost fish and binds to p75 receptor.

In some aspects, the neurotrophin targeting TrkB comprises, e.g., NT-4 or BDNF, or a fragment, variant, or derivative thereof. In some aspects, the neurotrophin targeting TrkA comprises, e.g., NGF or a fragment, variant, or derivative thereof. In some aspects, the neurotrophin targeting TrkC comprises, e.g., NT-3 or a fragment, variant, or derivative thereof.

In some aspects, the tropism moiety comprises brain derived neurotrophic factor (BDNF). In some aspects, the BDNF is a variant of native BDNF, such as a two amino acid carboxyl-truncated variant. In some aspects, the tropism moiety comprises the full length 119 amino acid sequence of BDNF (HSDPARRGELSVCTlSISEWVTAADKKTAVDMSGGTVTVLEKVPVSKGQLKQYFYETK CNPMGYTKEGCRGIDKRHWNSQCRTTQSYVRALTMDSKKRIGWRFIRIDTSCVCTLTIK RGR; SEQ ID NO: 404). In some aspects, a one amino-acid carboxy-truncated variant of BDNF is utilized (amino acids 1-118 of SEQ ID NO: 404).

In some aspects, the tropism moiety comprises a carboxy-truncated variant of the native BDNF, e.g., a variant in which 1, 2, 3, 4, 5, 6, 7, 8, 9, 10, or more than 10 amino acids are absent from the carboxy-terminus of the BDNF. BDNF variants include the complete 119 amino acid BDNF, the 117 or 118 amino acid variant with a truncated carboxyl terminus, variants with a truncated amino terminus, or variants with up to about 20%, about 30, or about 40% change in amino acid composition, as long as the protein variant still binds to the TrkB receptor with high affinity.

In some aspects, the tropism moiety comprises a two amino-acid carboxy-truncated variant of BDNF (amino acids 1-117 of SEQ ID NO: 404). In some aspects, the tropism moiety comprises a three amino-acid carboxy-truncated variant of BDNF (amino acids 1-116 of SEQ ID NO: 404). In some aspects, the tropism moiety comprises a four amino-acid carboxy-truncated variant of BDNF (amino acids 1-115 of SEQ ID NO: 404). In some aspects, the tropism moiety comprises a five amino-acid carboxy-truncated variant of BDNF (amino acids 1-114 of SEQ ID NO: 404). In some aspects, the tropism moiety comprises a BDNF that is at least about 60%, at least about 65%, at least about 70%, at least about 75%, at least about 80%, at least about 85%, at least about 90%, at least about 95%, at least about 99%, or about 100% identical with the sequence of SEQ ID NO: 404, or a truncated version thereof, e.g., the 117 or 118 amino acid variant with a one- or two-amino acid truncated carboxyl terminus, or variants with a truncated amino terminus. See, e.g., U.S. Pat. No. 8,053,569B2, which is herein incorporated by reference in its entirety.

In some aspects, the tropism moiety comprises nerve growth factor (NGF). In some aspects, the NGF is a variant of native NGF, such as a truncated variant. In some aspects, the tropism moiety comprises the 26-kDa beta subunit of protein, the only component of the 7S NGF complex that is biologically active. In some aspects, the tropism moiety comprises the full-length 120 amino acid sequence of beta NGF (SSSHPIFHRGEFSVCDSVSVWVGDKTTATDIKGKEVMVLGEVNINNSVFKQYFFETKCR DPNPVDSGCRGIDSKHWNSYCTTTHTFVKALTMDGKQAAWRFIRIDTACVCVLSRKAV RRA; SEQ ID NO: 405). In some aspects, the tropism moiety comprises a carboxy-truncated variant of the native NGF, e.g., a variant in which 1, 2, 3, 4, 5, 6, 7, 8, 9, 10, or more than 10 amino acids are absent from the carboxy-terminus of NGF. NGF variants include the complete 120 amino acid NGF, the shorter amino acid variants with a truncated carboxyl terminus, variants with a truncated amino terminus, or variants with up to about 20%, about 30%, or about 40% change in amino acid composition, as long as the tropism moiety still binds to the TrkB receptor with high affinity. In some aspects, the tropism moiety comprises an NGF that is at least about 60%, at least about 65%, at least about 70%, at least about 75%, at least about 80%, at least about 85%, at least about 90%, at least about 95%, at least about 99%, or about 100% identical with the sequence of SEQ ID NO: 405, or a truncated version thereof.

In some aspects, the tropism moiety comprises neurotrophin-3 (NT-3). In some aspects, the NT-3 is a variant of native NT-3, such as a truncated variant. In some aspects, the tropism moiety comprises the full-length 119 amino acid sequence of NT-3 (YAEHKSHRGEYSVCDSESLWVTDKSSAIDIRGHQVTVLGEIKTGNSPVKQYFYETRCKE ARPVKNGCRGIDDKHWNSQCKTSQTYVRALTSENNKLVGWRWIRIDTSCVCALSRKIG RT; SEQ ID NO: 406). In some aspects, the tropism moiety comprises a carboxy-truncated variant of the native NT-3, e.g., a variant in which 1, 2, 3, 4, 5, 6, 7, 8, 9, 10, or more than 10 amino acids are absent from the carboxy-terminus of NT-3. NT-3 variants include the complete 119 amino acid NT-3, the shorter amino acid variants with a truncated carboxyl terminus, variants with a truncated amino terminus, or variants with up to about 20%, about 30%, or about 40% change in amino acid composition, as long as the tropism moiety still binds to the TrkC receptor with high affinity. In some aspects, the tropism moiety comprises an NT-3 that is at least about 60%, at least about 65%, at least about 70%, at least about 75%, at least about 80%, at least about 85%, at least about 90%, at least about 95%, at least about 99%, or about 100% identical with the sequence of SEQ ID NO: 406, or a truncated version thereof.

In some aspects, the tropism moiety comprises neurotrophin-4 (NT-4). In some aspects, the NT-4 is a variant of native NT-4, such as a truncated variant. In some aspects, the tropism moiety comprises the full-length 130 amino acid sequence of NT-4 (GVSETAPASRRGELAVCDAVSGWVTDRRTAVDLRGREVEVLGEVPAAGGSPLRQYFFE TRCKADNAEEGGPGAGGGGCRGVDRRHWVSECKAKQSYVRALTADAQGRVGWRWIR IDTACVCTLLSRTGRA; SEQ ID NO: 407). In some aspects, the tropism moiety comprises a carboxy-truncated variant of the native NT-4, e.g., a variant in which 1, 2, 3, 4, 5, 6, 7, 8, 9, 10, or more than 10 amino acids are absent from the carboxy-terminus of NT-4. NT-4 variants include the complete 130 amino acid NT-4, the shorter amino acid variants with a truncated carboxyl terminus, variants with a truncated amino terminus, or variants with up to about 20%, about 30%, or about 40% change in amino acid composition, as long as the tropism moiety still binds to the TrkB receptor with high affinity. In some aspects, the tropism moiety comprises an NT-4 that is at least about 60%, at least about 65%, at least about 70%, at least about 75%, at least about 80%, at least about 85%, at least about 90%, at least about 95%, at least about 99%, or about 100% identical with the sequence of SEQ ID NO: 407, or a truncated version thereof.

Structure/function relationship studies of NGF and NGF-related recombinant molecules demonstrated that mutations in NGF region 25-36, along with other β-hairpin loop and non-loop regions, significantly influenced NGF/NGF-receptor interactions (Ibanez et al., EMBO J., 10, 2105-2110, (1991)). Small peptides derived from this region have been demonstrated to mimic NGF in binding to Mock receptor and affecting biological responses (LeSauteur et al. J. Biol. Chem. 270, 6564-6569, 1995). Dimers of cyclized peptides corresponding to β-loop regions of NGF were found to act as partial NGF agonists in that they had both survival-promoting and NGF-inhibiting activity while monomer and linear peptides were inactive (Longo et al., J. Neurosci. Res., 48, 1-17, 1997). Accordingly, in some aspects, a tropism moiety of the present disclosure comprises such peptides.

Cyclic peptides have also been designed and synthesized to mimic the β-loop regions of NGF, BDNF, NT3 and NT-⅘. Certain monomers, dimers or polymers of these cyclic peptides can have a three-dimensional structure, which binds to neurotrophin receptors under physiological conditions. All of these structural analogs of neurotrophins that bind to nerve cell surface receptors and are internalized can serve as the binding agent B of the compound according to the present disclosure to deliver the conjugated therapeutic moiety TM to the nervous system. Accordingly, in some aspects, a tropism moiety of the present disclosure comprises such cyclic peptides or combinations thereof.

In some aspects, antibodies against nerve cell surface receptors that are capable of binding to the receptors and being internalized can also serve as tropism moieties binding to a Trk receptor. For example, monoclonal antibody (MAb) 5C3 is specific for the NGF docking site of the human p140 TrkA receptor, with no cross-reactivity with human TrkB receptor. MAb 5C3 and its Fab mimic the effects of NGF in vitro, and image human Trk-A positive tumors in vivo (Kramer et al., Eur. J. Cancer, 33, 2090-2091, (1997)). Molecular cloning, recombination, mutagenesis and modeling studies of Mab 5C3 variable region indicated that three or less of its complementarity determining regions (CDRs) are relevant for binding to TrkA. Assays with recombinant CDRs and CDR-like synthetic polypeptides demonstrated that they had agonistic bioactivities similar to intact Mab 5C3. Monoclonal antibody MC192 against p75 receptor has also been demonstrated to have neurotrophic effects. Therefore, these antibodies and their functionally equivalent fragments can also serve as tropism moieties of the present disclosure.

In some aspects, peptidomimetics that are synthesized by incorporating unnatural amino acids or other organic molecules can also serve tropism moieties of the present disclosure.

Other neurotrophins are known in the art. Accordingly, in some aspects, the target moiety comprises a neurotrophin selected from the group consisting of fibroblast growth factor (FGF)-2 and other FGFs, erythropoietin (EPO), hepatocyte growth factor (HGF), epidermal growth factor (EGF), transforming growth factor (TGF)-a, TGF-(3, vascular endothelial growth factor (VEGF), interleukin-1 receptor antagonist (IL- lra), ciliary neurotrophic factor (CNTF), glial-derived neurotrophic factor (GDNF), neurturin, platelet-derived growth factor (PDGF), heregulin, neuregulin, artemin, persephin, interleukins, granulocyte-colony stimulating factor (CSF), granulocyte-macrophage-CSF, netrins, cardiotrophin-1, hedgehogs, leukemia inhibitory factor (LIF), midlcine, pleiotrophin, bone morphogenetic proteins (BMPs), netrins, saposins, semaphorins, and stem cell factor (SCF).

In some aspects, the tropism moiety directing an EV, e.g, exosome, disclosed herein to a sensory neuron, comprises a varicella zoster virus (VZV) peptide.

Tropism moieties targeting motor neurons: In some aspects, a tropism moiety disclosed herein can direct an EV, e.g, exosome, disclosed herein to a motor neuron. In some aspects, the tropism moiety that directs an EV, e.g, exosome, disclosed herein to a motor comprises a Rabies Virus Glycoprotein (RVG) peptide, a Targeted Axonal Import (TAxI) peptide, a P75R peptide, or a Tet-C peptide.

In some aspects, the tropism moiety comprises a Rabies Virus Glycoprotein (RVG) peptide. See, e.g., U.S. Pat. App. Publ. 2014-00294727, which is herein incorporated by reference in its entirety. In some aspects, the RVG peptide comprises amino acid residues 173-202 of the RVG (YTIWMPENPRPGTPCDIFTNSRGKRASNG; SEQ ID NO: 408) or a variant, fragment, or derivative thereof. In some aspects, the tropism moiety is a fragment of SEQ ID NO: 408. Such a fragment of SEQ ID NO: 408 can have, for example, 1, 2, 3, 4, 5, 6, 7, 8, 9, or 10 amino acids deleted from the N-terminal and/or the C-terminal of SEQ ID NO: 408. A functional fragmentderived from SEQ ID NO: 408 can be identified by sequentially deleting N- and/or C-terminal amino acids from SEQ ID NO: 408 and assessing the function of the resulting peptide fragment, such as function of the peptide fragment to bind acetylcholine receptor and/or ability to transmit through the blood brain barrier. In some aspects, the tropism moiety comprises a fragment of SEQ ID NO: 408 28, 27, 26, 25, 24, 23, 22, 21, 20, 19, 18, 17, 16 or 15 amino acids in length. In some aspects, the tropism moiety comprises a fragment of SEQ ID NO: 408 less than 15 peptides in length.

A “variant” of a RGV peptide, for example SEQ ID NO:408, is meant to refer to a molecule substantially similar in structure and function, i.e., where the function is the ability to pass or transit through the BBB, to either the entire molecule, or to a fragment thereof. A variant of an RVG peptide can contain a mutation or modification that differs from a reference amino acid in SEQ ID NO:408. In some aspects, a variant of SEQ ID NO:408 is a fragment of SEQ ID NO:408 as disclosed herein. In some aspects, an RVG variant can be a different isoform of SEQ ID NO:408 or can comprise different isomer amino acids. Variants can be naturally-occurring, synthetic, recombinant, or chemically modified polynucleotides or polypeptides isolated or generated using methods well known in the art. RVG variants can include conservative or non-conservative amino acid changes. See, e.g., U.S. Pat. No. 9,757,470, which is herein incorporated by reference in its entirety.

In some aspects, the tropism moiety comprises a Targeted Axonal Import (TAxI) peptide. In some aspects, the TAxI peptide is cyclized TAxI peptide of sequence SACQSQSQMRCGGG (SEQ ID NO:409). See, e.g., Sellers et al. (2016) Proc. Natl. Acad. Sci. USA 113:2514-2519, and U.S. Pat. No. 9,056,892, which are herein incorporated by reference in their entireties. TAxI transport peptides as described herein may be of any length. Typically, the transport peptide will be between 6 and 50 amino acids in length, more typically between 10 and 20 amino acids in length. In some aspects, the TAxI transport peptide comprises the amino acid sequence QSQSQMR (SEQ ID NO: 410), ASGAQAR (SEQ ID NO: 411), PF, or TSTAPHLRLRLTSR (SEQ ID NO: 412). Optionally, the TAxI transport peptide further includes a flanking sequence to facilitate incorporation into a delivery construct or carrier, e.g., a linker. In one aspect, the peptide is flanked with cysteines. In some aspects, the TAxI transport peptide further comprises additional sequence selected to facilitate delivery into nuclei. For example, a peptide that facilitates nuclear delivery is a nuclear localizing signal (NLS). Typically, this signal consists of a few short sequences of positively charged lysines or arginines, such as PPKKRKV (SEQ ID NO: 413). In one aspect, the NLS has the amino acid sequence PKKRKV (SEQ ID NO: 414).

In some aspects, a tropism moiety of the present disclosure comprises a peptide BBB shuttle disclosed in the table below. See, e.g., Oller-Salvia et al. (2016) Chem. Soc. Rev. 45, 4690-4707, and Jafari et al. (2019) Expert Opinion on Drug Delivery 16:583-605 which are herein incorporated by reference in their entireties.

TABLE 5

| SEQ ID NO | Peptide | Sequence |
|---|---|---|
| 415 | Angiopep-2 | TFFYGGSRGKRNNFKTEEY-OH |
| 416 | ApoB (3371-3409) | SSVIDALQYKLEGTTRLTRK-RGLKLATALSLSNKFVEGS |
| 417 | ApoE (159-167)₂ | (LRKLRKRLL)₂ |
| 418 | Peptide-22 | Ac-C(&)MPRLRGC(&)-NH₂ |
| 419 | THR | THRPPMWSPVWP-NH₂ |
| 420 | THR retro-enantio | pwvpswmpprht-NH₂ |
| 421 | CRT | C(&)RTIGPSVC(&) |
| 422 | Leptin30 | YQQILTSMPSRNVIQISND-LENLRDLLHVL |
| 423 | RVG29 | YTIWMPENPRPGTPCDIFT-NSRGKRASNG-OH |
| 424 | ^{D}CDX | GreirtGraerwsekf-OH |
| 425 | Apamin | C(&₁)NC(&₂)KAPETALC(&₁)-AR-RC(&₂)QQH-NH₂ |
| 426 | MiniAp-4 | [Dap](&)KAPETALD(&) |
| 427 | GSH | γ-L-gluta myI-CG-OH |
| 428 | G23 | HLNILSTLWKYRC |
| 429 | g7 | GFtGFLS(O-β-Glc)-NH₂ |
| 430 | TGN | TGNYKALHPHNG |
| 431 | TAT (47-57) | YGRKKRRQRRR-NH₂ |
| 432 | SynB1 | RGGRLSYSRRRFSTSTGR |
| 433 | Diketopiperazines | &(N-MePhe)-(N-MePhe)Diketo-piperazines |
| 434 | PhPro | (Phenylproline)₄-NH₂ |

Nomenclature for cyclic peptides (&) is adapted to the 3-letter amino acid code from the one described by Spengler et al-. Pept. Res., 2005, 65, 550-555 [Dap] stands for diaminopropionic acid.

### IV. Pharmaceutical Compositions

Certain aspects of the present disclosure are directed to methods of compartmentally administering a composition comprising an EV. In some aspects, the composition comprising the EV further comprises a pharmaceutically acceptable carrier or excipient.

The present disclosure also provides pharmaceutical compositions comprising EVs described herein that are suitable for administration to a subject according to the methods of administration targeting the CNS disclosed herein. The pharmaceutical compositions generally comprise a plurality of EVs comprising a biologically active molecule covalently linked to the plurality of EVs via a maleimide moiety and a pharmaceutically-acceptable excipient or carrier in a form suitable for administration to a subject. Pharmaceutically acceptable excipients or carriers are determined in part by the particular composition being administered, as well as by the particular method used to administer the composition. Accordingly, there is a wide variety of suitable formulations of pharmaceutical compositions comprising a plurality of EVs, e.g., exosomes. See, e.g., Remington’s Pharmaceutical Sciences, Mack Publishing Co., Easton, Pa. 18th ed. (1990).

The pharmaceutical compositions are generally formulated sterile and in full compliance with all Good Manufacturing Practice (GMP) regulations of the U.S. Food and Drug Administration. In some aspects, the pharmaceutical composition comprises one or more chemical compounds, such as for example, small molecules covalently linked to an EV described herein.

In some aspects, a pharmaceutical composition comprises one or more therapeutic agents and an EV described herein. In certain aspects, the EVs are co-administered with of one or more additional therapeutic agents, in a pharmaceutically acceptable carrier. In some aspects, the pharmaceutical composition comprising the EV is administered prior to administration of the additional therapeutic agents. In other aspects, the pharmaceutical composition comprising the EV is administered after the administration of the additional therapeutic agents. In further aspects, the pharmaceutical composition comprising the EV is administered concurrently with the additional therapeutic agents.

Provided herein are pharmaceutical compositions comprising an EV of the present disclosure having the desired degree of purity, and a pharmaceutically acceptable carrier or excipient, in a form suitable for administration to a subject.

Pharmaceutically acceptable excipients or carriers can be determined in part by the particular composition being administered, as well as by the particular method used to administer the composition. Accordingly, there is a wide variety of suitable formulations of compositions (e.g., pharmaceutical compositions) comprising a plurality of extracellular vesicles. (See, e.g., Remington’s Pharmaceutical Sciences, Mack Publishing Co., Easton, Pa. 21st ed. (2005)). The pharmaceutical compositions are generally formulated sterile and in full compliance with all Good Manufacturing Practice (GMP) regulations of the U.S. Food and Drug Administration.

In some aspects, a pharmaceutical composition comprises one or more therapeutic agents and an exosome described herein. In certain aspects, the EVs are co-administered with of one or more additional therapeutic agents, in a pharmaceutically acceptable carrier. In some aspects, the pharmaceutical composition comprising the EV, e.g., exosome is administered prior to administration of the additional therapeutic agents. In other aspects, the pharmaceutical composition comprising the EV, e.g., exosome is administered after the administration of the additional therapeutic agents. In further aspects, the pharmaceutical composition comprising the EV, e.g., exosome is administered concurrently with the additional therapeutic agents.

Acceptable carriers, excipients, or stabilizers are nontoxic to recipients (e.g., animals or humans) at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Examples of carriers or diluents include, but are not limited to, water, saline, Ringer’s solutions, dextrose solution, and 5% human serum albumin. The use of such media and compounds for pharmaceutically active substances is well known in the art. Except insofar as any conventional media or compound is incompatible with the extracellular vesicles described herein, use thereof in the compositions is contemplated. Supplementary therapeutic agents can also be incorporated into the compositions. Typically, a pharmaceutical composition is formulated to be compatible with its intended route of administration. The EVs can be administered by parenteral, topical, intravenous, oral, subcutaneous, intra-arterial, intradermal, transdermal, rectal, intracranial, intraperitoneal, intranasal, intratumoral, intramuscular route or as inhalants. In certain aspects, the pharmaceutical composition comprising exosomes is administered intravenously, e.g. by injection. The EVs can optionally be administered in combination with other therapeutic agents that are at least partly effective in treating the disease, disorder or condition for which the EVs are intended.

Solutions or suspensions can include the following components: a sterile diluent such as water, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The preparation can be enclosed in ampoules, disposable syringes or multiple dose vials made of glass or plastic.

Pharmaceutical compositions suitable for injectable use include sterile aqueous solutions (if water soluble) or dispersions and sterile powders. For intravenous administration, suitable carriers include physiological saline, bacteriostatic water, Cremophor EL™ (BASF, Parsippany, N.J.) or phosphate buffered saline (PBS). The composition is generally sterile and fluid to the extent that easy syringeability exists. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. If desired, isotonic compounds, e.g., sugars, polyalcohols such as mannitol, sorbitol, and sodium chloride can be added to the composition. Prolonged absorption of the injectable compositions can be brought about by including in the composition a compound which delays absorption, e.g., aluminum monostearate and gelatin.

Sterile injectable solutions can be prepared by incorporating the EVs in an effective amount and in an appropriate solvent with one or a combination of ingredients enumerated herein, as desired. Generally, dispersions are prepared by incorporating the EVs into a sterile vehicle that contains a basic dispersion medium and any desired other ingredients. In the case of sterile powders for the preparation of sterile injectable solutions, methods of preparation are vacuum drying and freeze-drying that yields a powder of the active ingredient plus any additional desired ingredient from a previously sterile-filtered solution thereof. The EVs can be administered in the form of a depot injection or implant preparation which can be formulated in such a manner to permit a sustained or pulsatile release of the EV, e.g., exosomes.

Systemic administration of compositions comprising EVs of the present disclosure can also be by transmucosal means. For transmucosal administration, penetrants appropriate to the barrier to be permeated are used in the formulation. Such penetrants are generally known in the art, and include, e.g., for transmucosal administration, detergents, bile salts, and fusidic acid derivatives. Transmucosal administration can be accomplished through the use of, e.g., nasal sprays.

In certain aspects the pharmaceutical composition comprising EVs of the present disclosure is administered intravenously into a subject that would benefit from the pharmaceutical composition. In certain other aspects, the composition is administered to the lymphatic system, e.g., by intralymphatic injection or by intranodal injection (see e.g., Senti et al., PNAS 105(46): 17908 (2008)), or by intramuscular injection, by subcutaneous administration, by intratumoral injection, by direct injection into the thymus, or into the liver.

In certain aspects, the pharmaceutical composition comprising exosomes is administered as a liquid suspension. In certain aspects, the pharmaceutical composition is administered as a formulation that is capable of forming a depot following administration. In certain preferred aspects, the depot slowly releases the EVs into circulation, or remains in depot form.

Typically, pharmaceutically-acceptable compositions are highly purified to be free of contaminants, are biocompatible and not toxic, and are suited to administration to a subject. If water is a constituent of the carrier, the water is highly purified and processed to be free of contaminants, e.g., endotoxins.

The pharmaceutically-acceptable carrier can be lactose, dextrose, sucrose, sorbitol, mannitol, starch, gum acacia, calcium phosphate, alginates, gelatin, calcium silicate, microcrystalline cellulose, polyvinylpyrrolidone, cellulose, water, syrup, methyl cellulose, methylhydroxy benzoate, propylhydroxy benzoate, talc, magnesium stearate, and/or mineral oil, but is not limited thereto. The pharmaceutical composition can further include a lubricant, a wetting agent, a sweetener, a flavor enhancer, an emulsifying agent, a suspension agent, and/or a preservative.

The pharmaceutical compositions described herein comprise the EVs described herein and optionally a pharmaceutically active or therapeutic agent. The therapeutic agent can be a biological agent, a small molecule agent, or a nucleic acid agent.

Dosage forms are provided that comprise a pharmaceutical composition comprising the EVs described herein. In some aspects, the dosage form is formulated as a liquid suspension for intravenous injection. In some aspects, the dosage form is formulated as a liquid suspension for intratumoral injection.

In certain aspects, the preparation of exosomes is subjected to radiation, e.g., X rays, gamma rays, beta particles, alpha particles, neutrons, protons, elemental nuclei, UV rays in order to damage residual replication-competent nucleic acids.

In certain aspects, the preparation of EVs of the present disclosure is subjected to gamma irradiation using an irradiation dose of more than about 1, about 5, about 10, about 15, about 20, about 25, about 30, about 35, about 40, about 50, about 60, about 70, about 80, about 90, about 100, or more than 100 kGy.

In certain aspects, the preparation of EVs of the present disclosure is subjected to X-ray irradiation using an irradiation dose of more than about 0.1, about 0.5, about 1, about 5, about 10, about 15, about 20, about 25, about 30, about 35, about 40, about 50, about 60, about 70, about 80, about 90, about 100, about 200, about 300, about 400, about 500, about 600, about 700, about 800, about 900, about 1000, about 2000, about 3000, about 4000, about 5000, about 6000, about 7000, about 8000, about 9000, or about 10000.

The EVs of the present disclosure can be used concurrently with other drugs. To be specific, the EVs of the present disclosure can be used together with medicaments such as hormonal therapeutic agents, chemotherapeutic agents, immunotherapeutic agents, medicaments inhibiting the action of cell growth factors or cell growth factor receptors and the like.

### VII. Kits

The present disclosure also provides kits, or products of manufacture comprising one or more EVs of the present disclosure and optionally instructions for use according to the methods of administration targeted to the CNS disclosed herein.

In some aspects, the kit, or product of manufacture contains a pharmaceutical composition described herein which comprises at least one EV of the present disclosure, and instructions for use according to the methods of administration targeted to the CNS disclosed herein.. In some aspects, the kit, or product of manufacture comprises at least one EV of the present disclosure or a pharmaceutical composition comprising the EVs in one or more containers. One skilled in the art will readily recognize that the EVs of the present disclosure, pharmaceutical composition comprising the EVs of the present disclosure, or combinations thereof can be readily incorporated into one of the established kit formats which are well known in the art.

In some aspects, the kit comprises reagents to conjugate a biologically active molecule to an EV via a maleimide moiety, and instructions to conduct the conjugation.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986); ); Crooke, Antisense drug Technology: Principles, Strategies and Applications, 2^{nd} Ed. CRC Press (2007) and in Ausubel et al. (1989) Current Protocols in Molecular Biology (John Wiley and Sons, Baltimore, Md.).

All of the references cited above, as well as all references cited herein, are incorporated herein by reference in their entireties.

The following examples are offered by way of illustration and not by way of limitation.

### EXAMPLES

### Example 1: Exosome Isolation and Loading

Exosome isolation: Exosomes were collected from the supernatant of high density suspension cultures of HEK293 SF cells after 7-9 days. Cell culture medium was serially centrifuged, with the supernatant of the previous spin serving as the input for the subsequent spin: cell culture medium was centrifuged at 5,000 x g for 30 minutes, the supernatant collected and the pellet discarded; the supernatant was then centrifuged at 16,000 x g for 30 minutes and the supernatant collected and the pellet discarded; the supernatant was then centrifuged at 133,900 x g for 3 hours, and the resulting supernatant discarded and the pellet collected and resuspended in 1 mL of PBS. The resuspended 133,900 x g pellet was further purified by running in an OPTIPREP™ Iodixanol gradient: a 4-tier sterile gradient was prepared by mixing 3 mL of OPTIPREP™ (60% Iodixanol) with 1 mL of resuspended pellet to generate 4 mL of 45% Iodixanol, then overlaid serially with 3 mL 30% Iodixanol, 2 mL 22.5% Iodixanol, 2 mL 17.5% Iodixanol, and 1 mL PBS in a 12 mL Ultra-Clear (344059) tube for a SW 41 Ti rotor. The gradient was ultracentrifuged at 150,000 x g for 16 hours at 4° C. Ultracentrifugation resulted in a Top Fraction known to contain exosomes, a Middle Fraction containing cell debris of moderate density, and a Bottom Fraction containing high density aggregates and cellular debris. The exosome layer was then gently collected from the top ~2 mL of the tube.

The exosome fraction was diluted in ~32 mL PBS in a 38.5 mL Ultra-Clear (344058) tube and centrifuged at 10,000 x g for 30 minutes, the supernatant collected and ultracentrifuged at 133,900 x g for 3 hours at 4° C. to pellet the purified exosomes. The pelleted exosomes were then resuspended in a minimal volume of PBS (~200 µL) and stored at 4° C. Final purified concentration of exosomes was determined using nanoparticle tracking analysis (NTA).

Exosome Loading: To load exosomes with maleimide conjugates, exosomes were chemically reduced using TCEP (Tris(2-carboxyethyl)phosphine hydrochloride) at concentrations from 1 to 50 mM; in some cases, the reduction step includes, or is preceded by treatment with, 1-2 M Guanidine hydrochloride for one hour at room temperature. Exosomes were exchanged into PBS by diluting to 1 mL in PBS, centrifuging at 100,000 x g for 20 minutes (TLA 120.2 rotor, Beckman) to pellet exosomes, the supernatant was removed and discarded, and the pellet resuspended in 1 mL PBS; this was repeated once to ensure complete buffer exchange. The final exosome pellet was resuspended in 0.1 mL PBS, to which the compound to be loaded was added to a final concentration of up to 300 µM. Exosomes were incubated overnight at 4° C., followed by washing with PBS to remove compound not conjugated to exosomes (diluting to 1 mL in PBS, centrifuging at 100,000 x g for 20 minutes (TLA 120.2 rotor, Beckman) to pellet exosomes, the supernatant was removed and discarded, and the pellet resuspended in 1 mL PBS; this was repeated once to ensure complete buffer exchange).

### Example 2: Cell Tropism

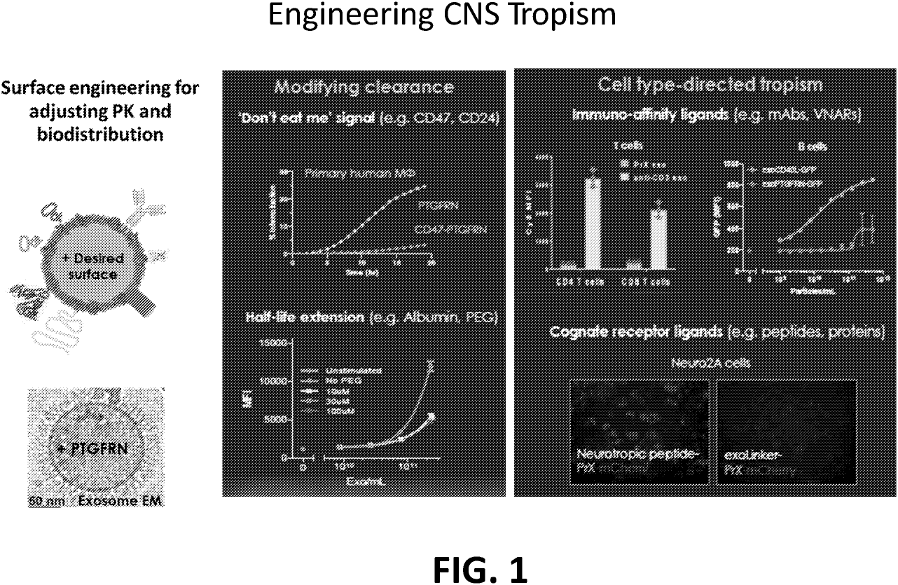

Cell tropism (FIG. 1) is selectively directed by the expression of PTGFRN to target and CD47 to avoid macrophages respectively. Expression of anti-CD3 antibody (targeting T-cells), CD40 ligand (targeting B-cells) or rabies viral glycoprotein (RVG; neuronal cells) are other ways of driving cell-specific exosome tropism.

### Example 3: Neuronal Exosome Tropism

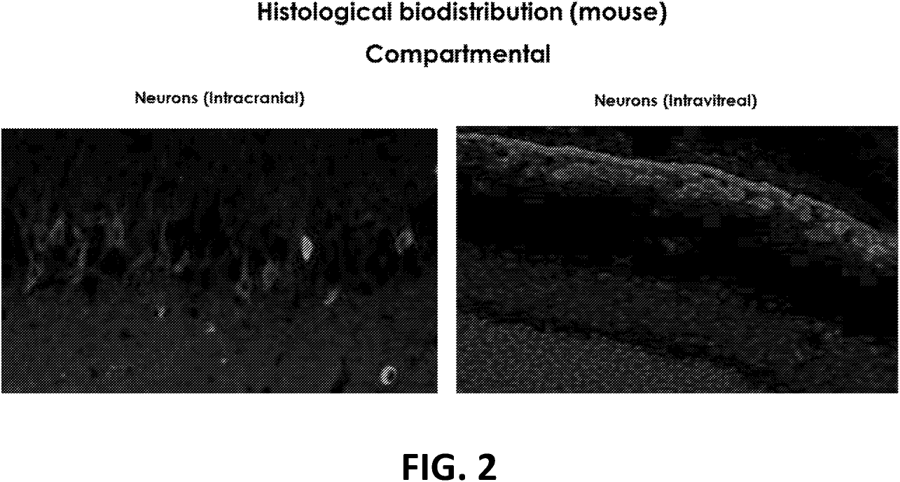

Compartmental dosing of PTGFRN-over-expressing exosomes was used to show colocalization of exosomes with neuronal cells in formalin-fixed paraffin-embedded (PPFE) 10 µm tissue sections (FIG. 2). The Left hand panel shows costaining of a subset of neurons in the mouse hippocampus 2-4 hours following intrahippocampal administration of approximately 100 ng PTGFRN-exosomes and as indicated by overlapping signals from multiplex immunofluorescence staining of neuronal cells with 4′,6-diamidino-2-phenylindole (DAPI) and 1G11 to detect exosomes. Similarly, the right-hand panel shows localization of exosomes, using 1G11 staining in the retinal ganglion layer following intravitreal administration of approximately 50-100 ng PTGFRN-exosomes in rats.

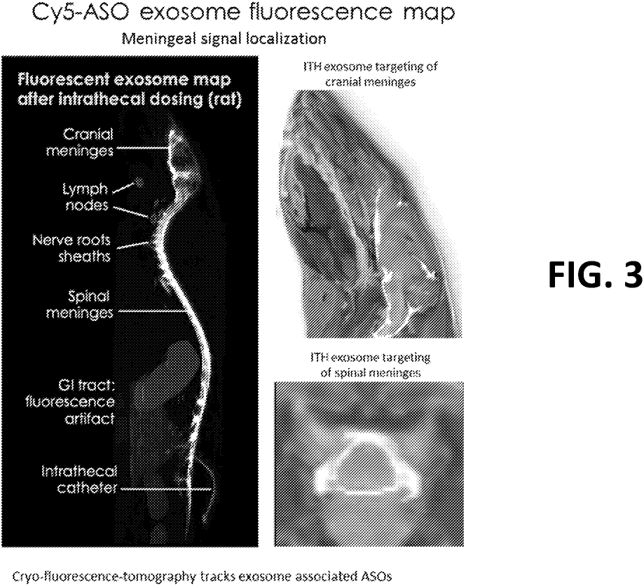

Cryofluorescence Tomography (CFT) was used to track antisense oligonucleotide (ASO)-associated exosomes following intrathecal dosing (FIG. 3). Here exosomes (50-100 ng) were administered via an intrathecal catheter into rats and 4 hrs later the animals were processed for CFT. In short, rats were euthanized and frozen in a dry ice/hexane bath prior to executing the CFT process which combines white light and fluorescence imaging in a slice-by-slice method with subsequent 3D reconstruction of post-processed images. The left-hand panel essentially reveals CFT imaging of the mapping of the biodistribution of CY5-ASO-labeled exosomes at the cellular level, and revealing primarily meningeal localization of intrathecally-administered exosomes, but also to include nerve roots and lymph nodes. The lower signal observed in the GI tract is a known fluorescence artifact. The top and bottom images of the right-hand panel provide confirmation of the targeting of cranial and spinal meninges following intrathecal administration.

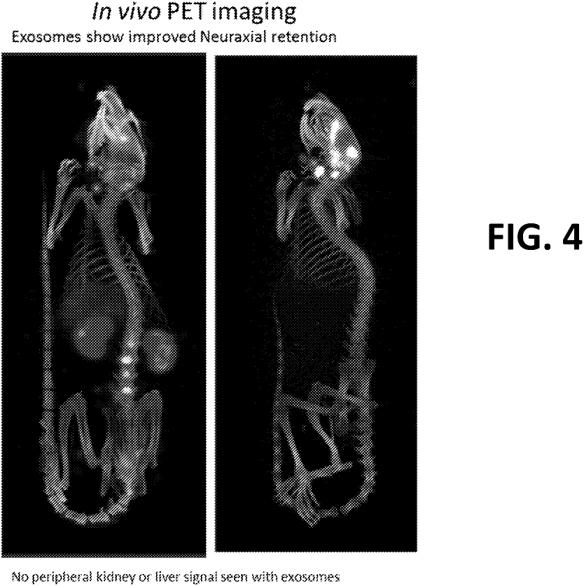

Intrathecal dosing [⁸⁹Zr]-DFO labeled exosomes provided a means to non-invasively track labeled exosome biodistribution with high sensitivity using small animal Positron Emission Tomography (mPET). Here the labeled exosomes showed improved neuraxial retention over time and importantly the data shows a lack of signal in peripheral organs such as kidney or liver suggesting that the labeled exosomes remain in the administered compartment over time (FIG. 4).

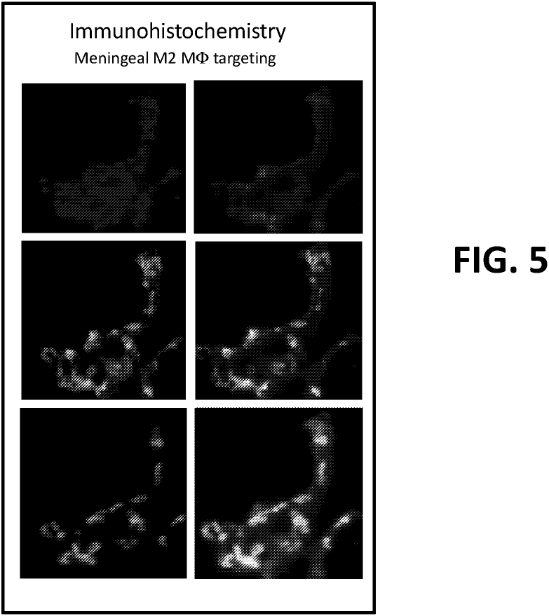

Multiplex immunofluorescence staining revealed meningeal M2 macrophage (MF) targeting following compartmental dosing into the cerebrospinal fluid (CSF). The panels show colocalization of the M2 MF marker CD206 with exosomes immunostained using 1G11 (FIG. 5).

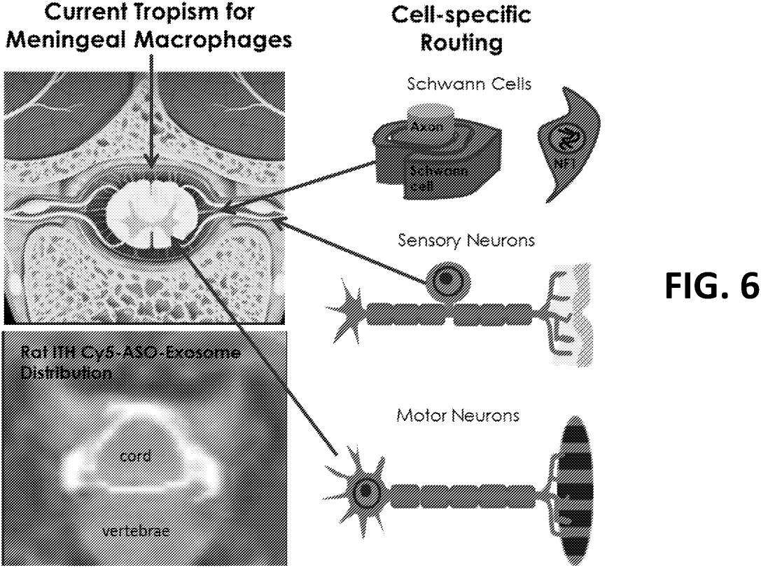

Neuronal exosome tropism is selectively directed by the expression of surface ligands or antibodies targeting sensory neuron targets such as the receptors for neurotrophic factors (NGF, BDNF, NT3), or motor neuron targets such as acetylcholine receptor subtypes (FIG. 6). Schwann cells can be specifically targeted by exosome surface engineering of ligands or antibodies targeting the transferrin receptor or other targets highly expressed on these cells.

### Example 4: Construction and Characterization of Exosomes Expressing CD47

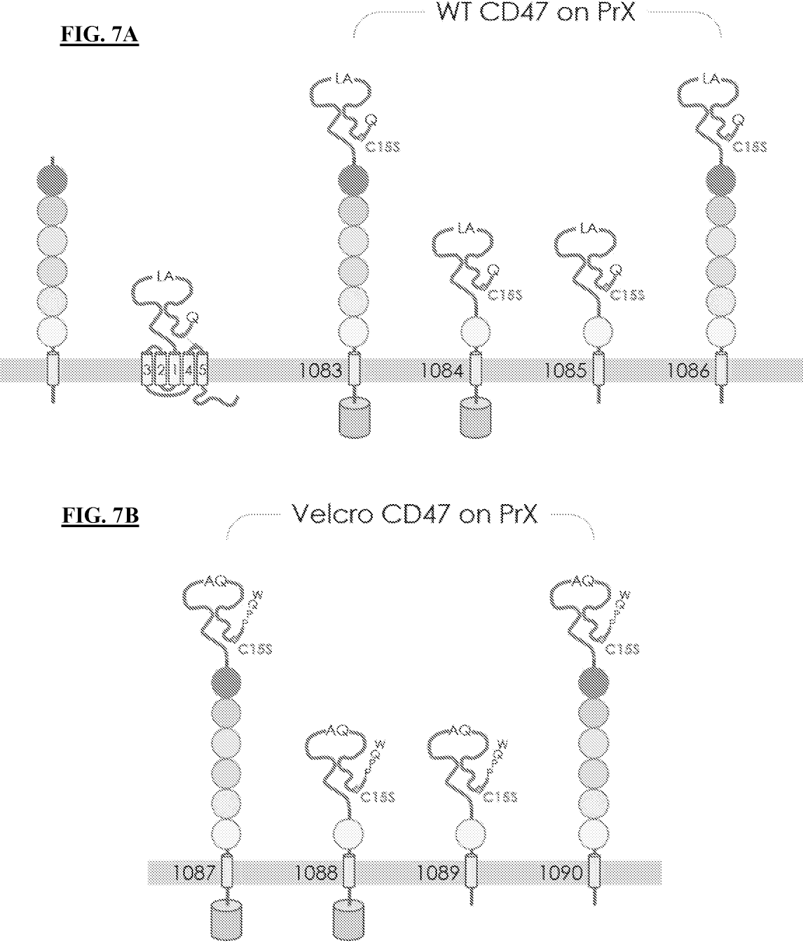

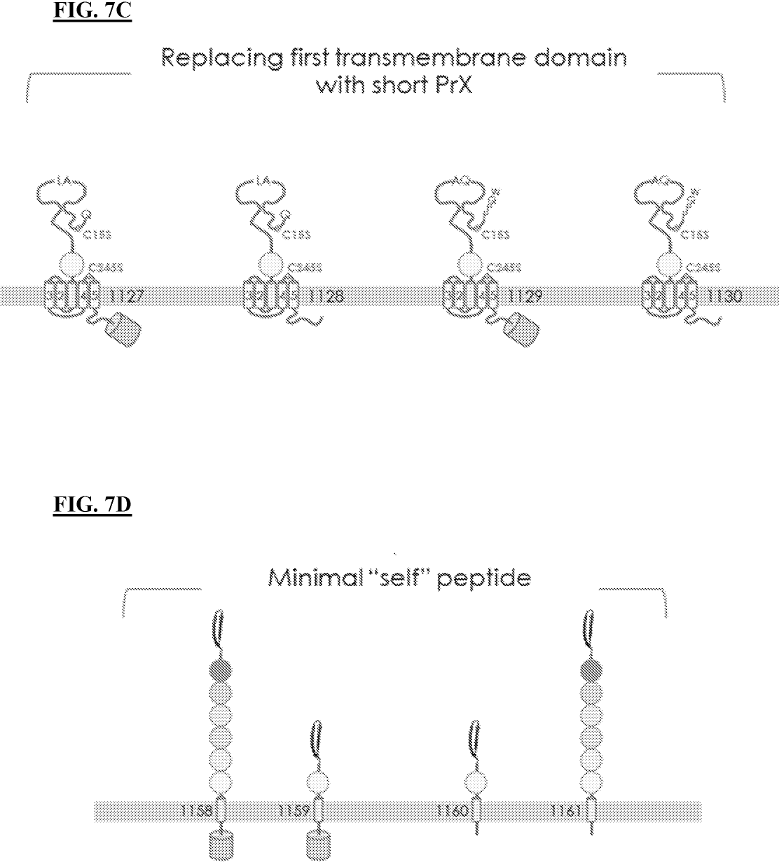

In order to minimize the uptake of administered exosomes by native myeloid cells, various constructs were created to express human CD47 or a fragment thereof on the surface of exosomes. The extracellular domain of human wild type CD47, having a C15S substitution, or Velcro-CD47 was fused to Scaffold X or a fragment thereof and expressed in exosome-producing cells (FIGS. 7A-7B). In addition, exosomes were produced expressing a modified CD47 having a truncated Scaffold X protein inserted in the first domain of of human wild type CD47, having a C15S substitution, or Velcro-CD47 (FIG. 7C). Further exosomes were generated expressing a minimal “self” peptide (GNYTCEVTELTREGETIIELK; SEQ ID NO: 382) fused to Scaffold X or a fragment thereof (FIG. 7D; see, e.g., Rodriguez et al., Science 339:971-75 (February 2013)).

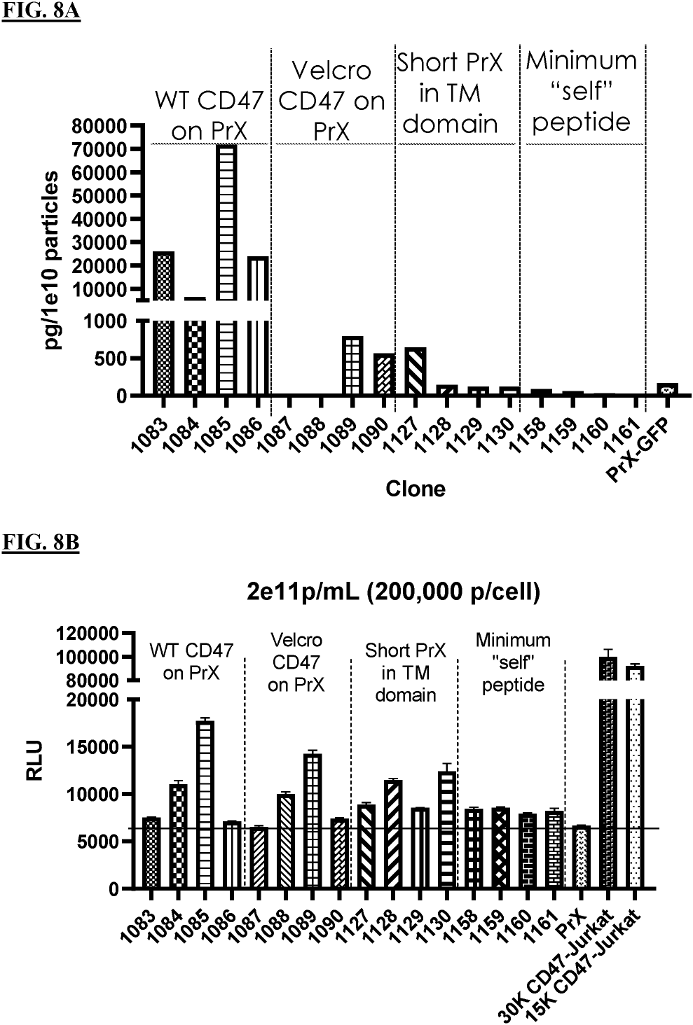

Exosomes expressing each construct were assayed for CD47 expression by ELISA using an anti-CD47 antibody targeted to a specific epitope of CD47 (FIG. 8A) or by binding to SIRPα using a SIRPα (human) signaling reporter cell bioassay (DiscoverX) (FIG. 8B) or using Octet analysis (FIGS. 9A-9C). Because the ELISA antibody recognized a specific epitope of CD47, some constructs were not recognized in the ELISA experiments. The results of each method of assaying CD47 expression are summarized in Table 6.

TABLE 6

Summary of CD47 Exosome Expression Assays

| | | | |
|---|---|---|---|
| Construct | ELISA | Bioassay | Octet |
| 1083 | Y | | Y |
| 1084 | Y | Y | Y |
| 1085 | Y | Y | Y |
| 1086 | Y | | Y |
| 1087 | | | Y |
| 1088 | | Y | Y |
| 1089 | Y | Y | Y |
| 1090 | Y | | Y |
| 1127 | Y | Low | Y |
| 1128 | | Y | Y |
| 1129 | | Low | Y |
| 1130 | | Y | Y |
| 1158 | | Low | Y |
| 1159 | | Low | Y |
| 1160 | | Low | Y |
| 1161 | | Low | Y |
| PrX | | | |

### Example 5: In Vitro Analysis of Uptake of Exosomes Expressing CD47

Monocytes were isolated from blood and differentiated to macrophages by culturing for 7-8 days in M-CSF. For each CD47 construct, exosomes were labeled with pHrodo-Red with NHS chemistry (3 rounds of 30 minutes). Exosomes were passed through a 70 µm qEV column and subjected through ultracentrifugation to further clean and concentrate the exosomes. Particle concentration was determined by NTA. Macrophages were re-plated, and exosomes were added to the macrophage culture at varying concentrations. Cells were imaged using IncuCyte, with 3 fields of view per well.

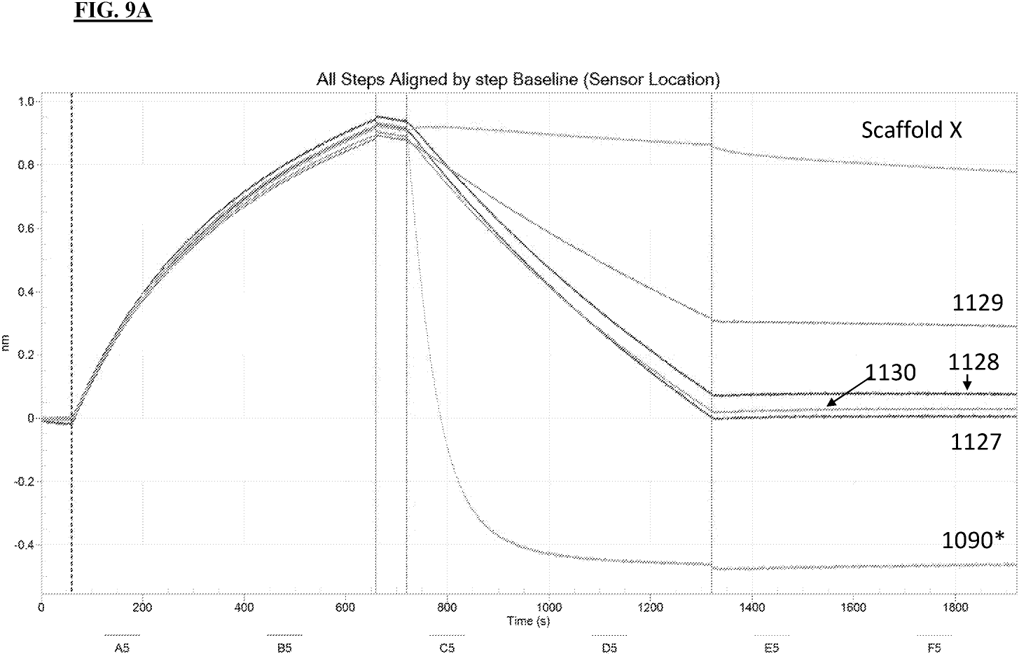

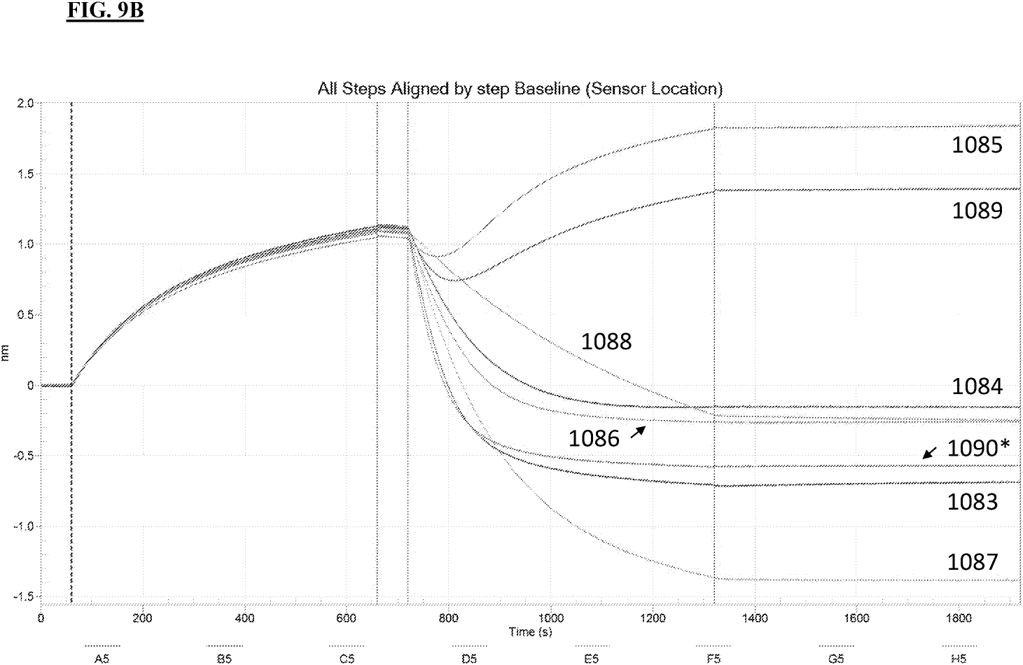

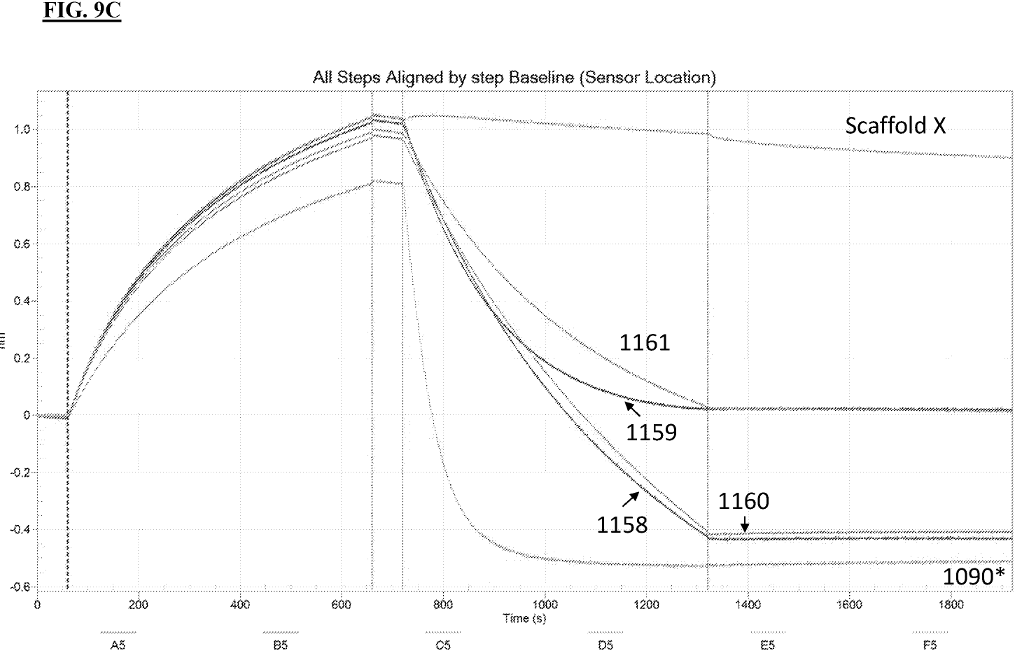

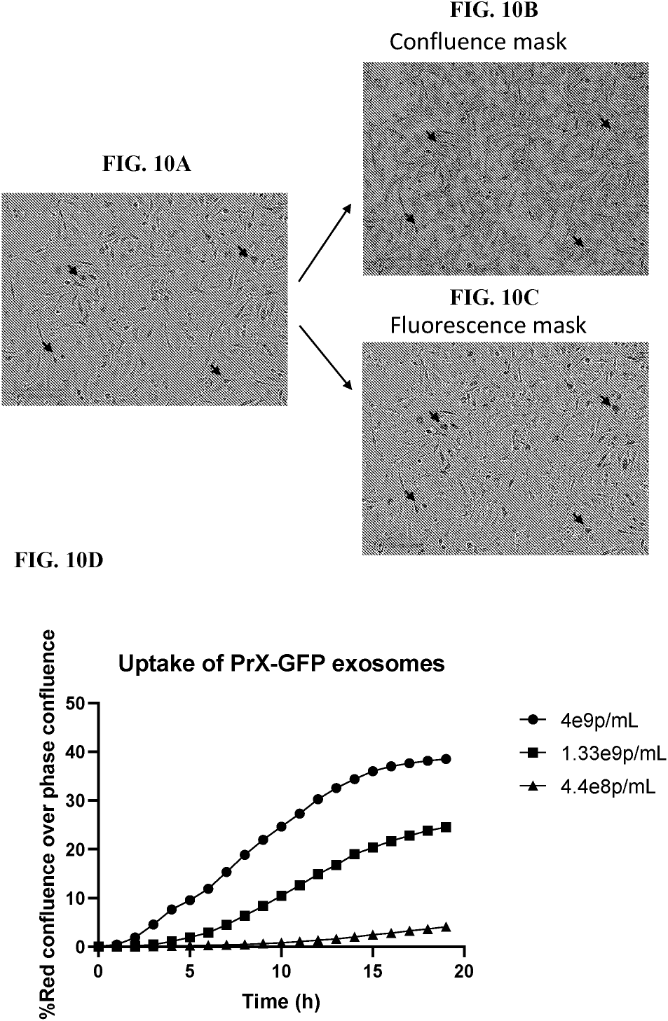

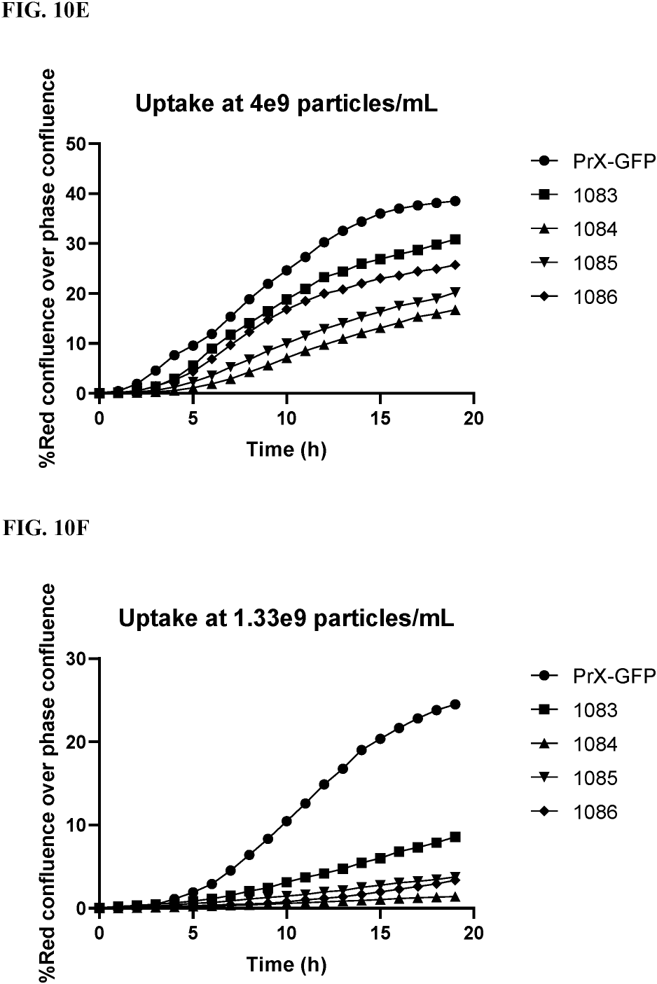

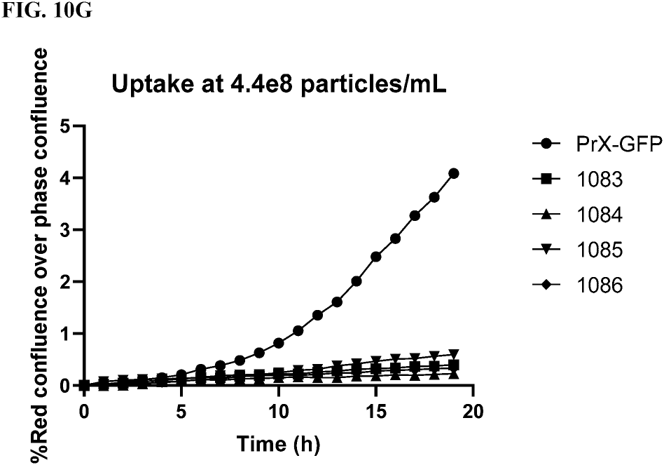

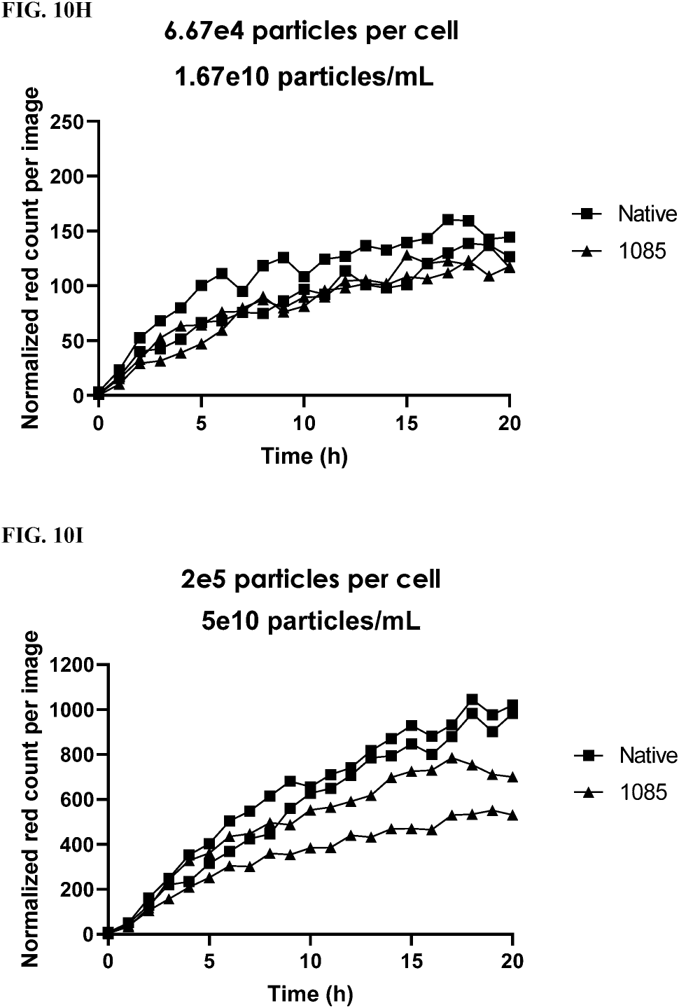

Dose-dependent uptake of control exosomes expressing GFP fused to Scaffold X by primary human monocyte-derived M0 macrophages was observed (FIGS. 10A-10D). However, expression of CD47 on the exosome surface resulted in decreased uptake of the exosomes by primary human monocyte-derived M0 macrophages, relative to the Scaffold X-GFP controls (FIGS. 10E-10H), with little to no effect on the uptake of the exosomes by adherent HEK cells, which do not express SIRPα (FIGS. 10I-10J).

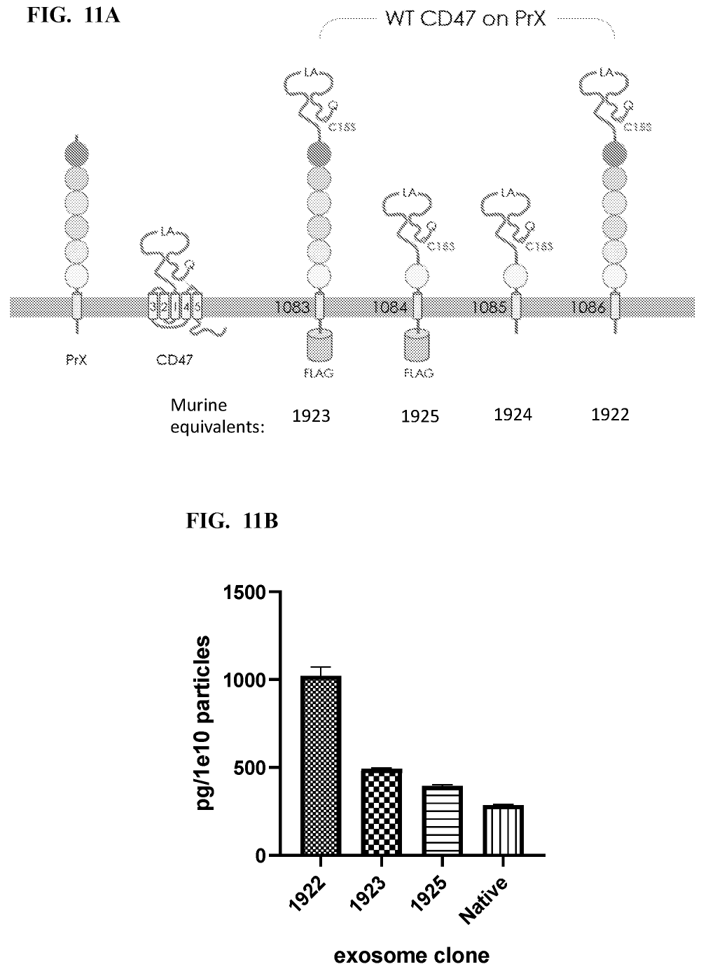

To test the effect of surface-expressed CD47 in mouse models, flag-tagged (1923 and 1925) and non-flag-tagged (1922 and 1924) constructs were created by fusing the extracellular domain of murine CD47^{C15S} to a full length scaffold X protein (1922 and 1923) or a truncated scaffold X protein (1924 and 1925) (FIG. 11A). The constructs were expressed in exosome producing cells, and exosomes were isolated and screened for CD47 protein expression and activity. Surface-expression of CD47 was observed for each of the constructs (FIGS. 11B-11C). Octet assay showed binding of both human and mouse CD47 exosomes to mouse SIRPα (FIG. 12). With the exception of hCD47 construct pCB-1085, the mouse and other human CD47 exosomes showed binding to mouse SIRPα.

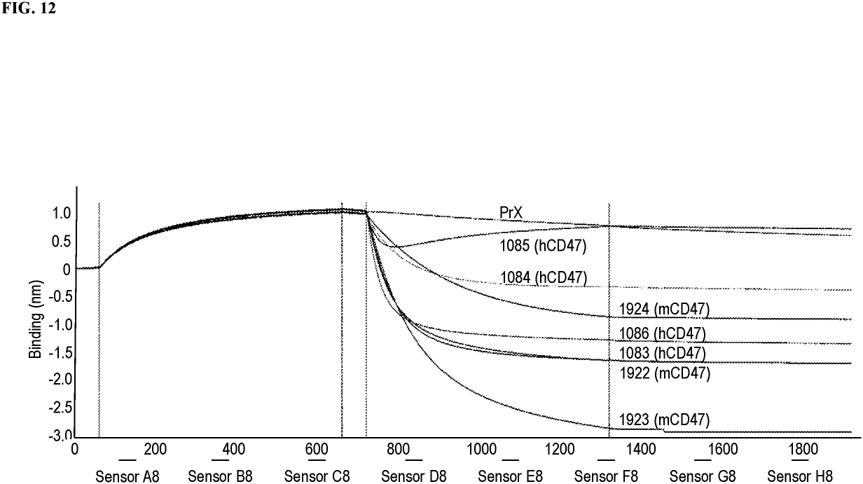

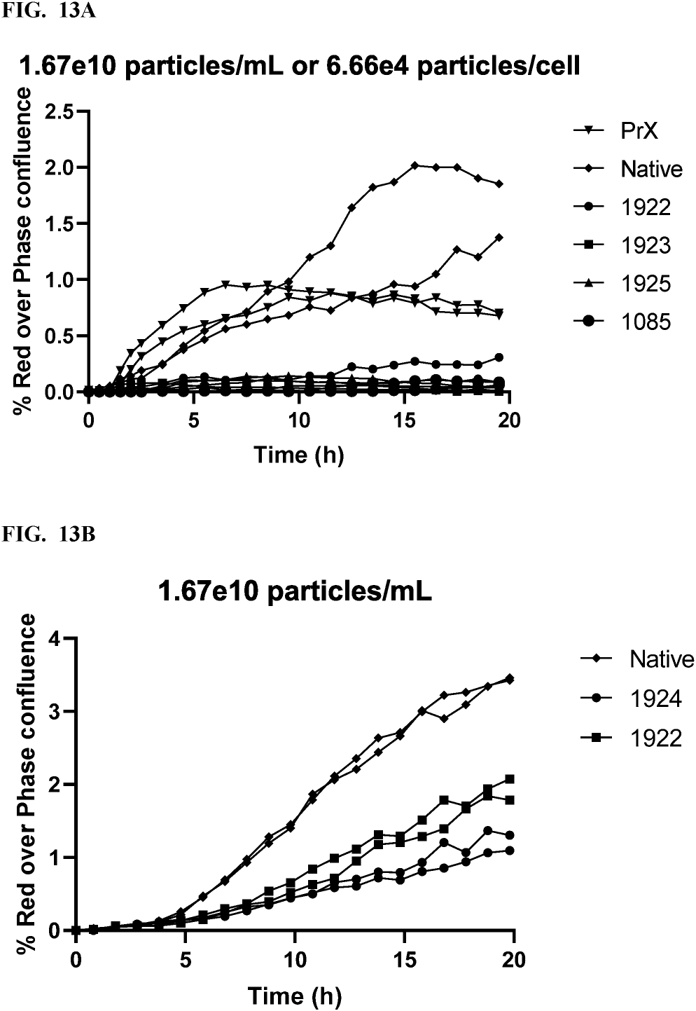

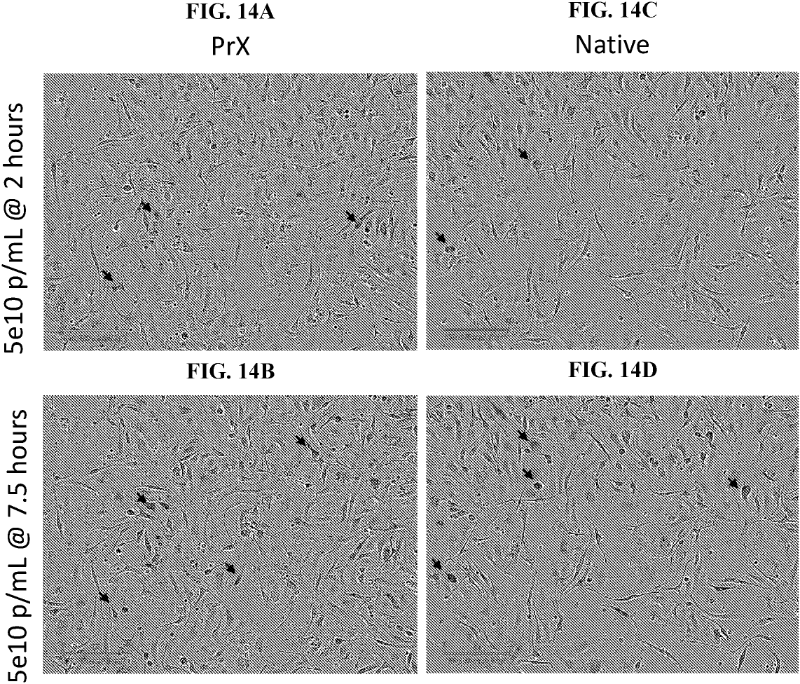

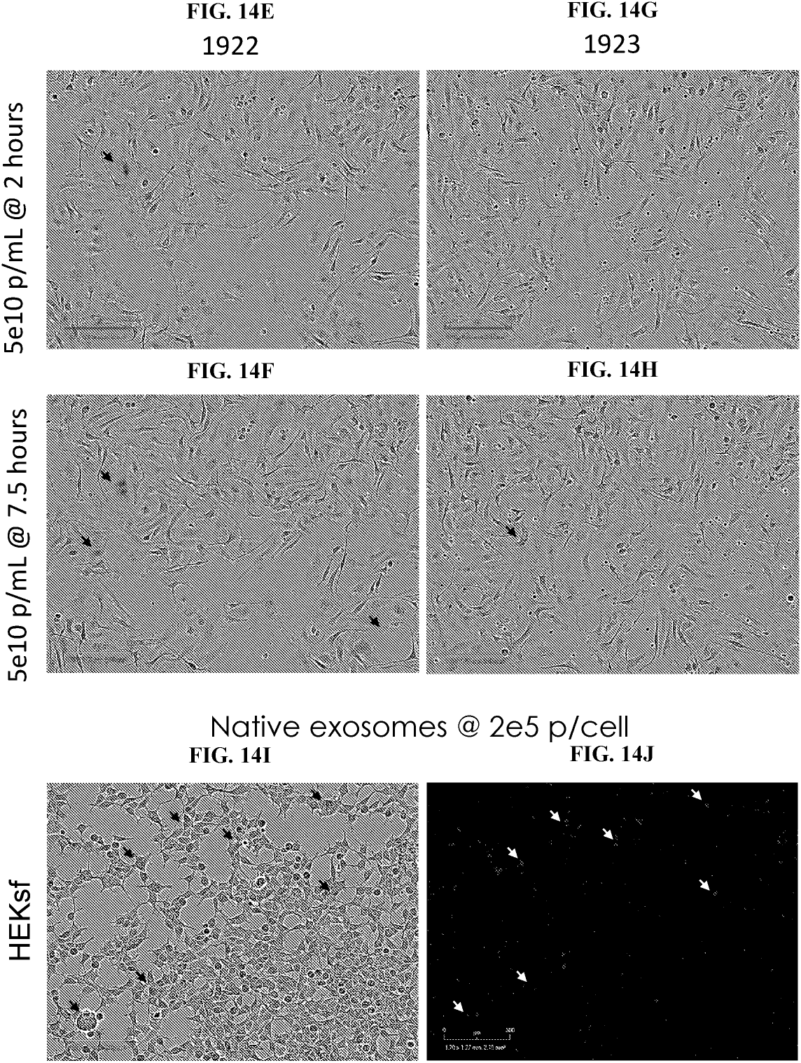

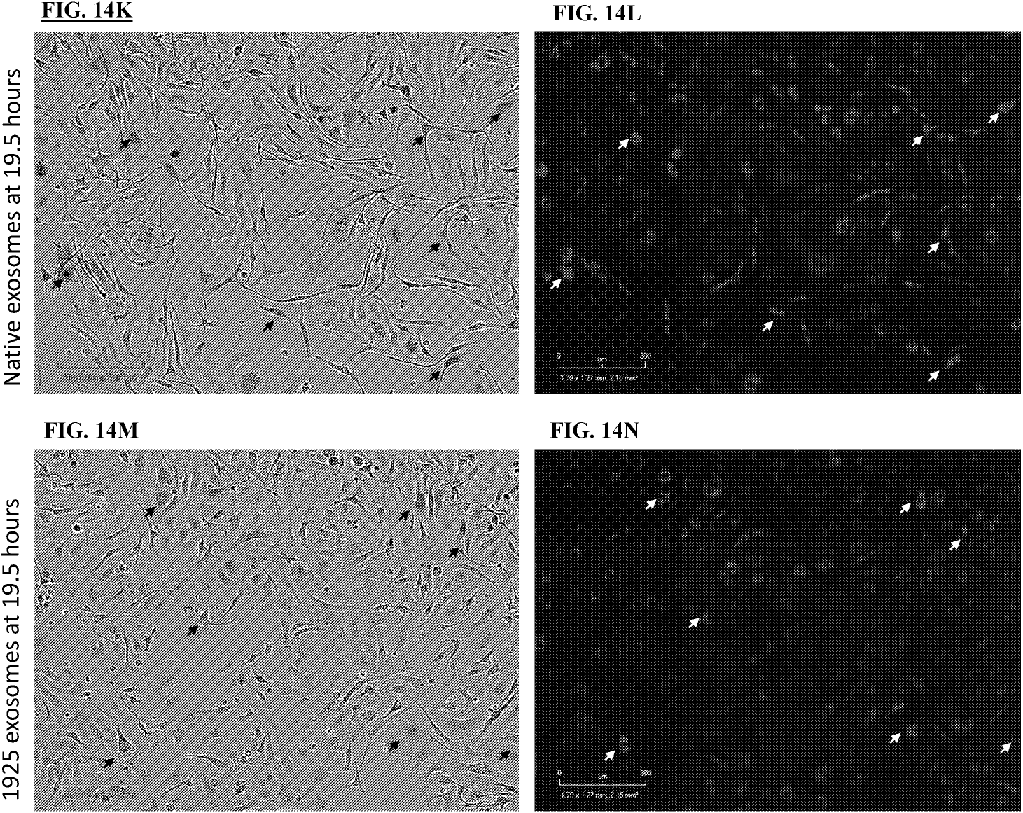

In vitro, mCD47-expressing exosomes displayed decreased uptake by SIRPα⁺ mouse bone marrow-derived macrophages (BMDM; FIGS. 13A-13B; 14A-14N). In addition, the human construct 1085 also reduce uptake of exosomes in the mouse bone marrow-derived macrophages.

### Example 6: Macrophage Evasion of Exosomes

Macrophage clearance of exosomes can limit the bioavailability of exosomes and their cargo in vivo. In order to limit macrophage clearance, exosomes were modified to express CD47 fused to Scaffold X (PTGFRN) and their uptake by macrophages in cell culture was assayed. In vitro, primary human macrophages readily internalized exosomes expressing control PTGFRN, reaching approximately 25% internalization by about 20 hours (FIG. 15A; circles). However, internalization of exosomes expressing CD47 fused to PTGFRN was greatly reduced for the duration of the culture, reaching a maximum of less than 5% at about 20 hours (FIG. 15A; diamonds). These data suggest that expression of CD47 on the surface of exosomes inhibits uptake by macrophages, potentially reducing macrophage clearance in vivo.

To further extend the bioavailability, exosomes were linked to increasing concentrations of polyethylene glycol (PEG). Increased half-life was observed, relative to exosomes lacking any PEG, for each of the concentrations tested (10 µM, 30 µM, and 100 µM; FIG. 15B).

In another in vitro analysis of uptake of exosomes, monocytes were isolated from blood and differentiated to macrophages by culturing for 7-8 days in M-CSF. For each CD47 construct, exosomes were labeled with pHrodo-Red with NHS chemistry (3 rounds of 30 minutes). Exosomes were passed through a 70 µm qEV column and subjected through ultracentrifucation to further clean and concentrate the exosomes. Particle concentration was determined by NTA. Macrophages were re-plated, and exosomes were added to the macrophage culture at varying concentrations. Cells were imaged using IncuCyte, with 3 fields of view per well.

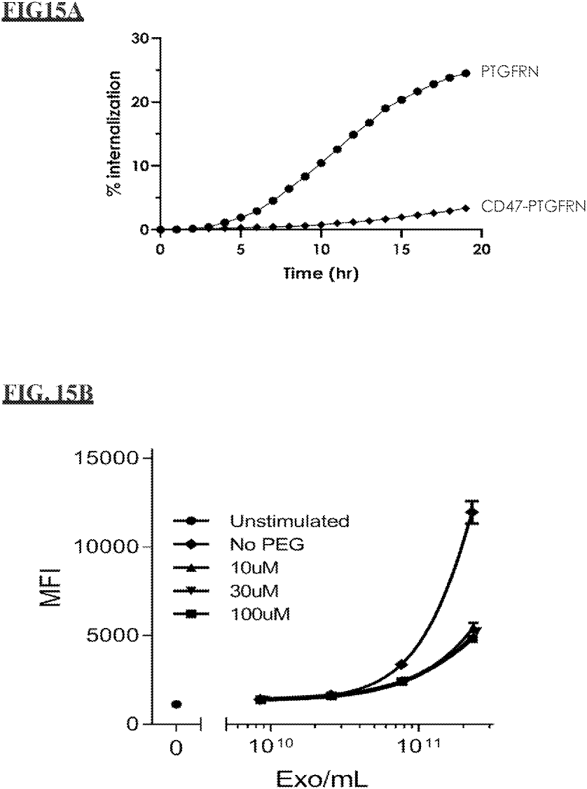

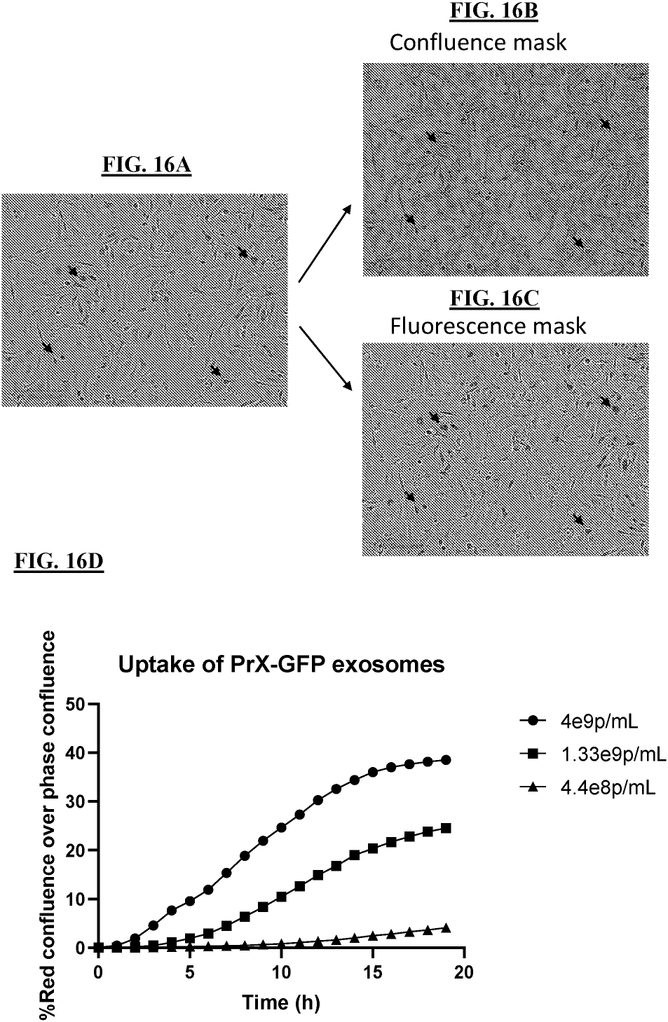

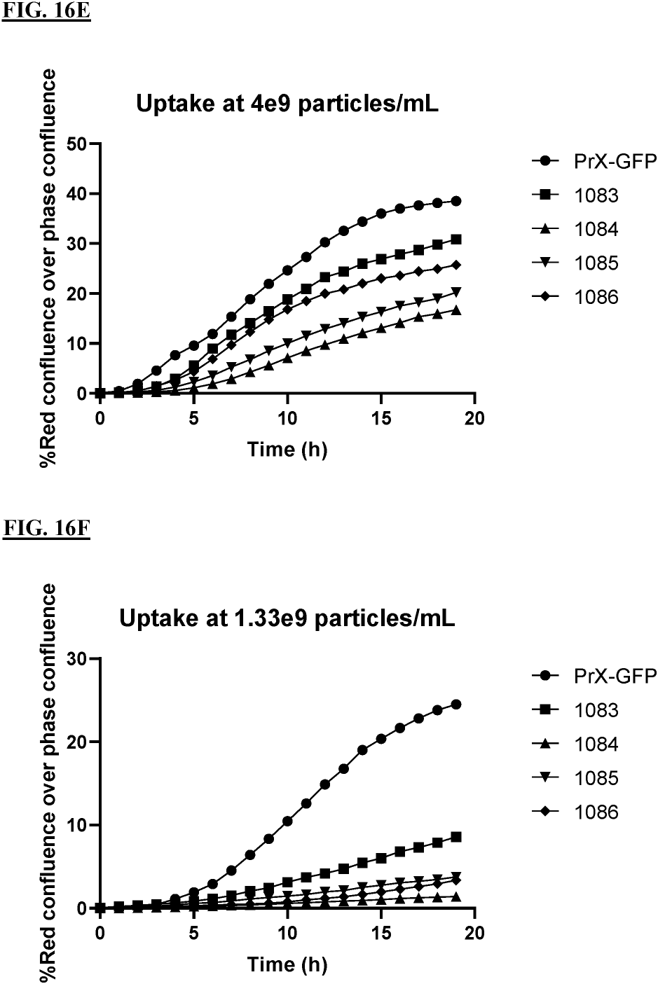

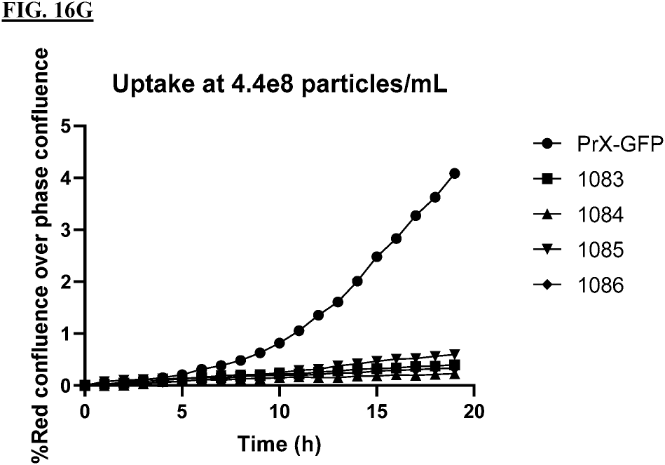

Dose-dependent uptake of control exosomes expressing GFP fused to Scaffold X by primary human monocyte-derived M0 macrophages was observed (FIGS. 16A-16D). However, expression of CD47 on the exosome surface resulted in decreased uptake of the exosomes by primary human monocyte-derived M0 macrophages, relative to the Scaffold X-GFP controls (FIGS. 16E-16H), with little to no effect on the uptake of the exosomes by adherent HEK cells, which do not express SIRPα (FIGS. 16I-16J).

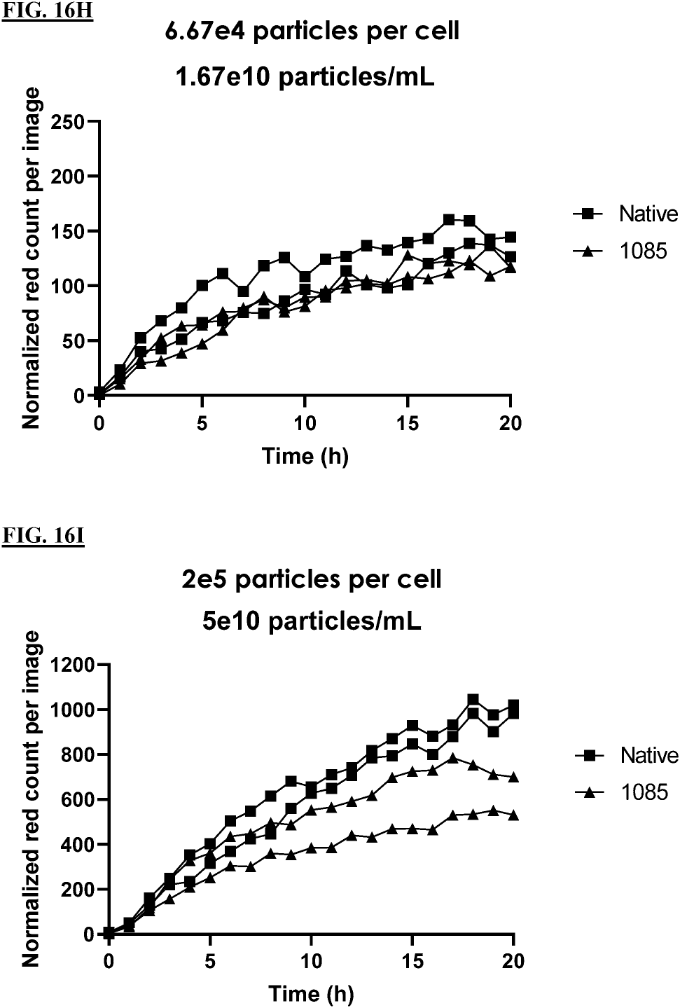

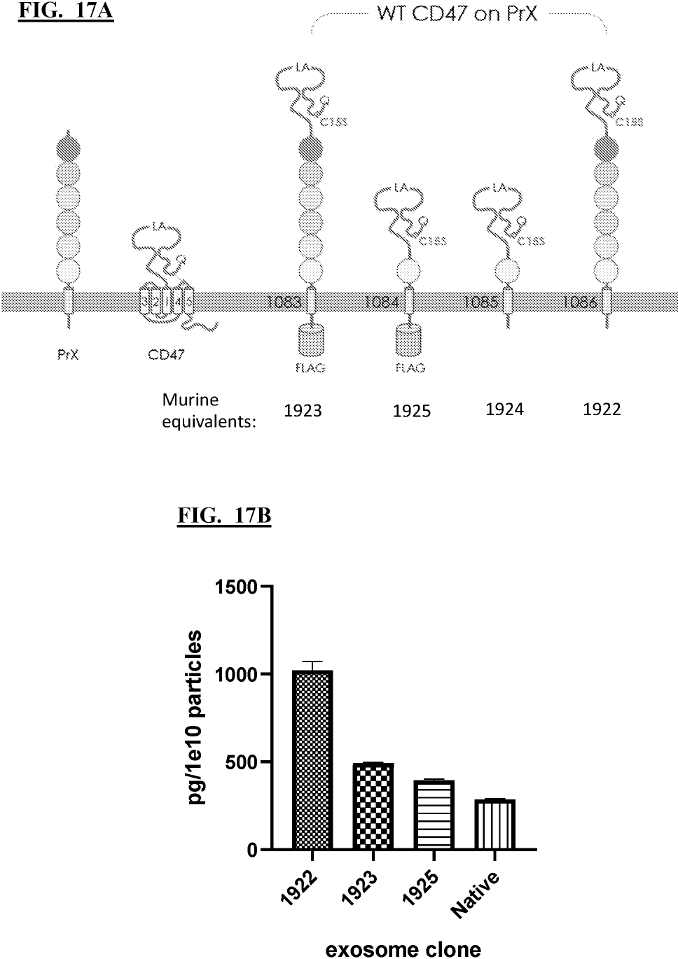

To test the effect of surface-expressed CD47 in mouse models, flag-tagged (1923 and 1925) and non-flag-tagged (1922 and 1924) constructs were created by fusing the extracellular domain of murine CD47^{C15S} to a full length scaffold X protein (1922 and 1923) or a truncated scaffold X protein (1924 and 1925) (FIG. 17A). The constructs were expressed in exosome producing cells, and exosomes were isolated and screened for CD47 protein expression and activity. Surface-expression of CD47 was observed for each of the constructs (FIGS. 17B-17C). Octet assay showed binding of both human and mouse CD47 exosomes to mouse SIRPα (FIG. 18). With the exception of hCD47 construct pCB-1085, the mouse and other human CD47 exosomes showed binding to mouse SIRPα.

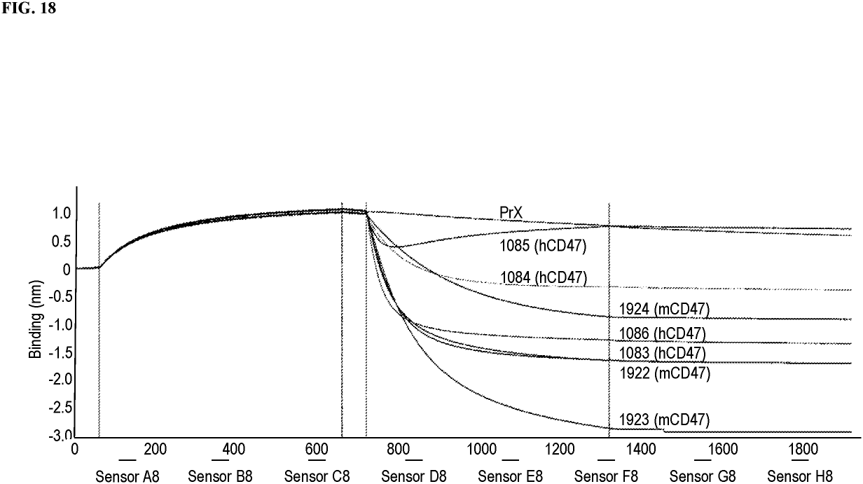

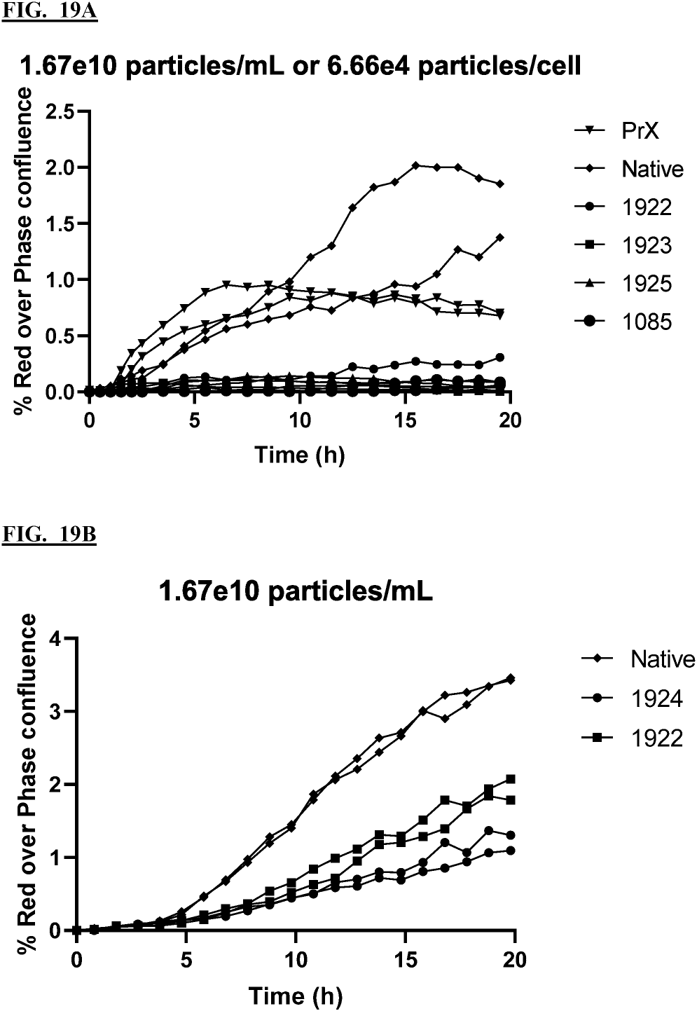

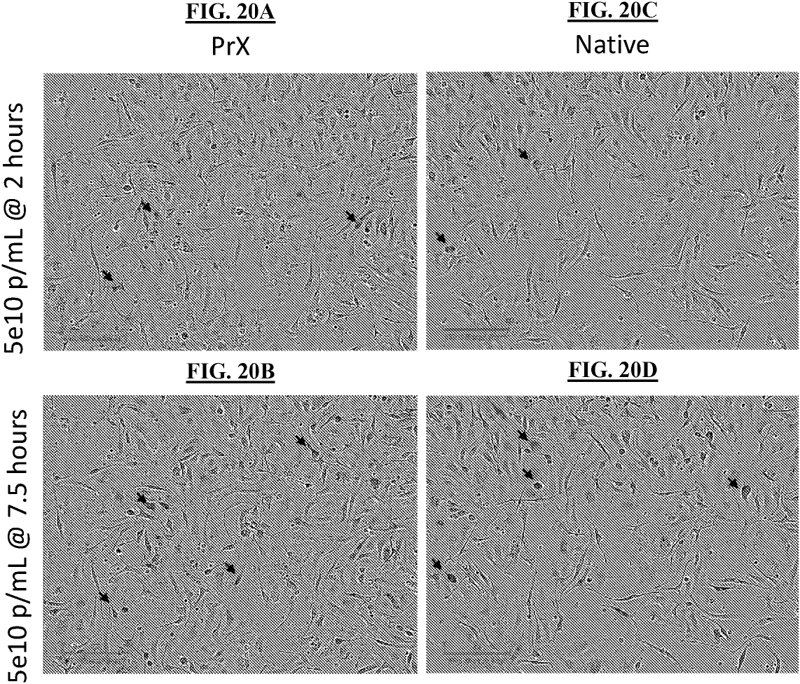

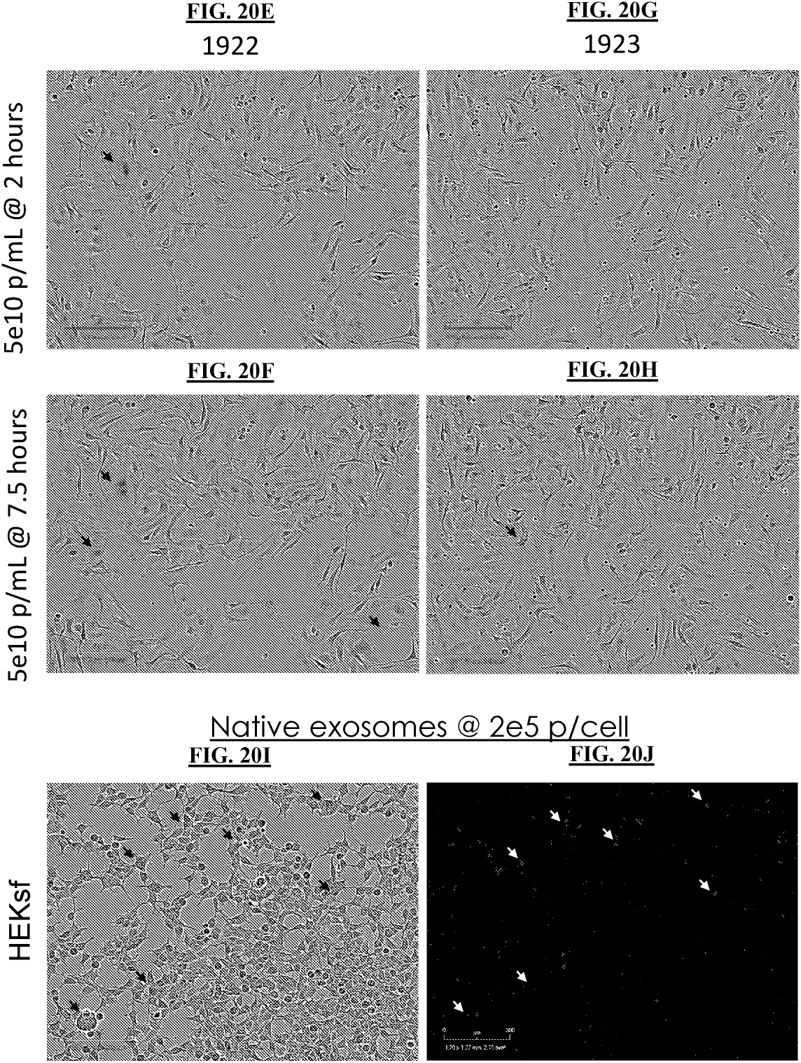

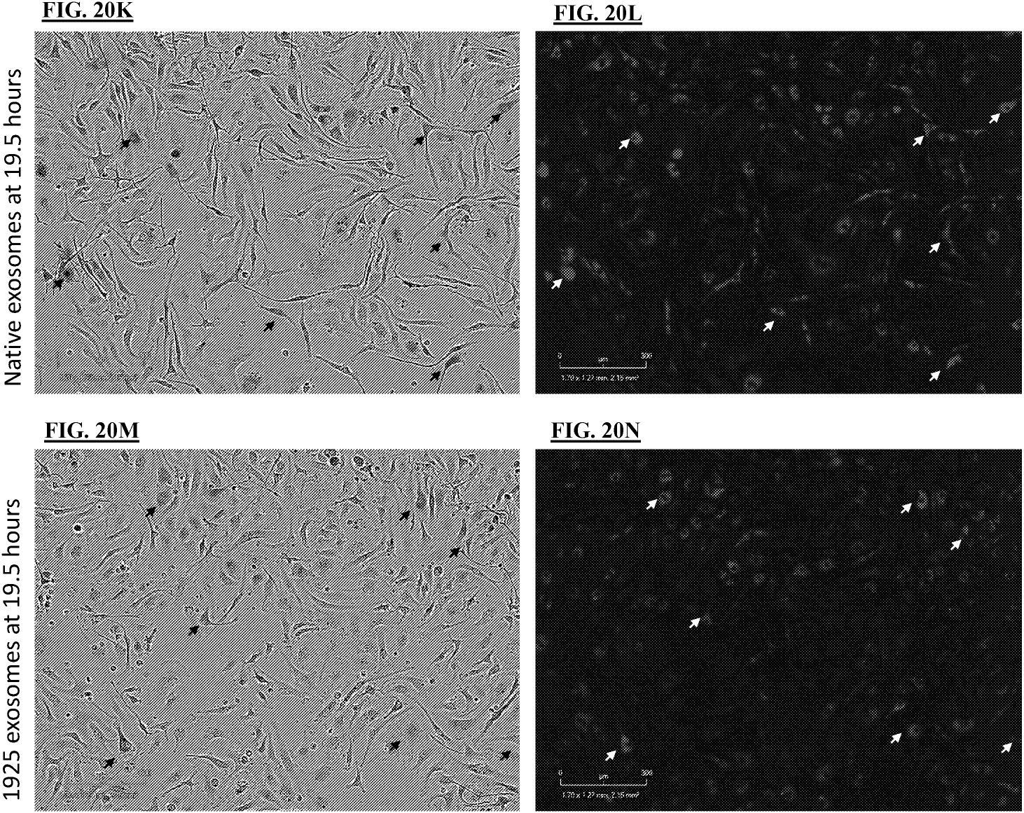

In vitro, mCD47-expressing exosomes displayed decreased uptake by SIRPα⁺ mouse bone marrow-derived macrophages (BMDM; FIGS. 19A-19B; 20A-20N). In addition, the human construct 1085 also reduce uptake of exosomes in the mouse bone marrow-derived macrophages.

### Example 7: Exosome Biodistribution by Compartmental Administration

To determine the effect of dosing route on in vivo biodistribution, non-human primates (FIGS. 21A and 21D), rats (FIG. 21B), and mice (FIGS. 21C and 21E) were administered ⁸⁹Zr-labelled exosomes intravenously (FIGS. 21A-21C) or intraperitoneally (FIGS. 21D-21E), and live, non-invasive PET imaging was conducted 2 hours after dosing. Intravenous delivery shows primary localization to the liver and spleen (FIGS. 21A-21C and 11A-11B), and intraperitoneally delivery shows somewhat differential localization, including to the lymph nodes (FIGS. 21D-21E and 22C).

Targeted, compartmental administration of exosomes yielded increased localization to target tissue. Exosomes delivered to mice intracranially (FIG. 22D) or intravitreally (FIG. 22E) resulted in localization of the exosomes to the neurons; exosomes delivered to mice by inhalation resulted in localization of the exosomes to the lungs (FIG. 22F); exosomes delivered to mice intramuscularly resulted in localization of the exosomes to muscle cells (FIG. 22G); and exosomes delivered to mice orally resulted in localization of the exosomes to at least the colon (FIG. 22H). In addition, intra-tumor delivery into a live tumor resulted in localization of the exosomes to the tumor tissue (FIG. 22I). Taken together, these results illustrate that compartmental delivery of exosomes allows for localization of delivered exosomes to a target tissue to a greater extent than intravenous or intraperitoneal delivery.

### Example 8: Intrathecal Administration of Exosomes

To target the central nervous system (CNS), radiolabeled exosomes were administered intrathecally to mice. Intrathecal administration of ⁸⁹Zr-labelled exosomes to mice resulted in localization of the exosomes to the retuculoendothelial system (FIG. 23B), with strong signal observed in the cranial meninges, lymph nodes, nerve root sheaths, and spinal meninges (FIGS. 23C-23E). Unlike a radio-labelled antisense oligonucleotide control (¹²⁵I-ASO; FIG. 23A), which was not packaged in an exosome, exosomes localization was not observed in the peripheral kidney or liver, indicating strong restriction to the neural axis.

Immunohistochemistry of meningeal M2 macrophages shows costaining of CD206 (an antibody that binds a marker of M2 macrophages) and 1G11 (a monoclonal antibody that binds exosomes), indicating that intrathecal delivery of exosomes can be used to target meningeal M2 macrophages (FIGS. 24A-24F).

### Example 9: Exosome Biodistribution by Targeting Moiety

To further direct exosomes to a target tissue, exosomes were engineered to express a targeting peptides on the surface of the exosome. Fusion of an anti-CD3 antibody to Scaffold X resulted in increased localization of the targeted exosomes to CD4 and CD8 T cells (FIG. 25A); expression of a GFP-tagged CD40L similarly resulted in increased localization of exosomes to B cells (FIG. 25B); and expression of a neurotropic peptide fused to Scaffold X resulted in increased uptake by neuro2A cells (FIG. 25C) relative to a negative control (FIG. 25D).

### Example 10: Construction and Characterization of Exosomes With Tropism Moieties

In order to direct EVs to specific cellular types, various constructs were created to express different tropism moieties. To determine whether the RVG peptide could be used to direct EVs to neurons, several constructs were tested. The constructs tested were: RVG-PrX-mCherry-FLAG-HiBiT (construct 2021), linker-PrX-mCherry-FLAG-HiBiT (construct 2022), RVG-LAMP2B-mCherry-FLAG-HiBiT (construct 2023), and linker-LAMP2B-mCherry-FLAG-HiBiT (construct 2024).

“RVG” refers to a tropism moiety of having the amino acid sequence YTIWMPENPRPGTPCDIFTNSRGKRASNG (SEQ ID NO: 408). “Linker” refers to a linker having the amino acid sequence GGSSGSGSGSGGGGSGGGGTGTSSSGTGT (SEQ ID NO: 435). “FLAG” refers to a FLAG® epitope tag. “HiBiT” refers to a nano luciferase peptide. “mCherry” is a red fluorescent protein. “LAMP2B” and “PrX” are protein scaffolds, e.g., as described above. “ExoRVG” EVs are exosomes comprising an RVG tropism moiety.

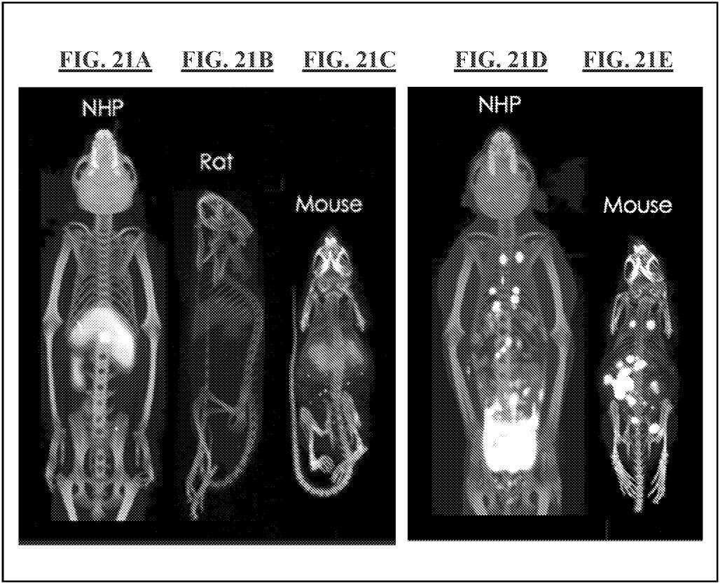

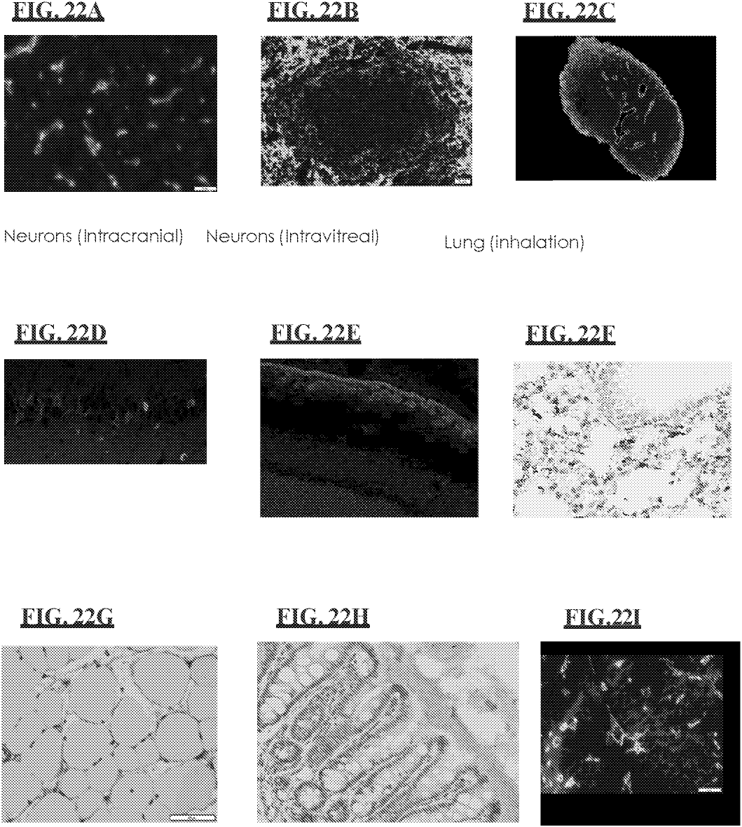

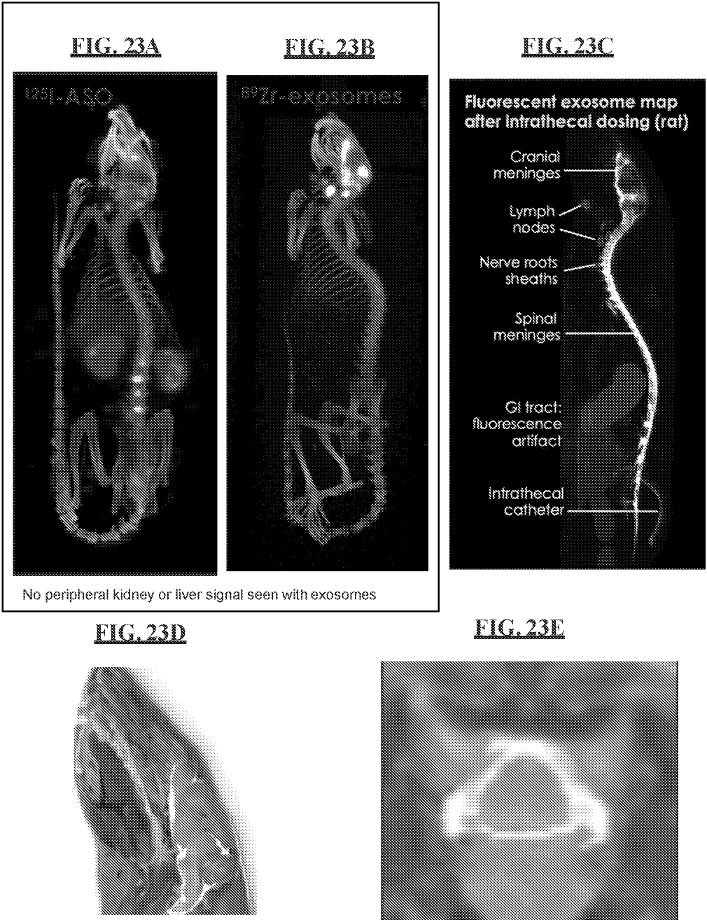

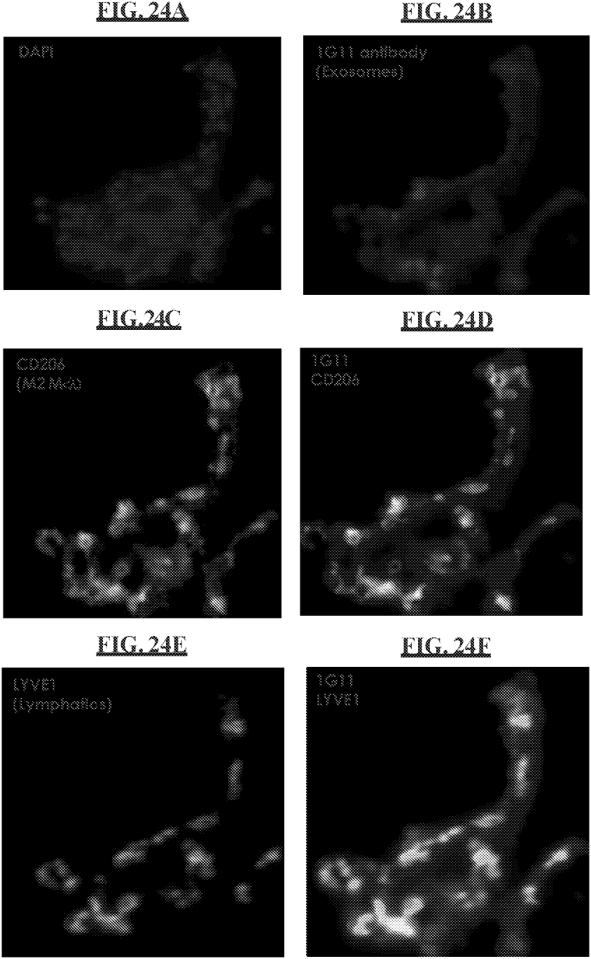

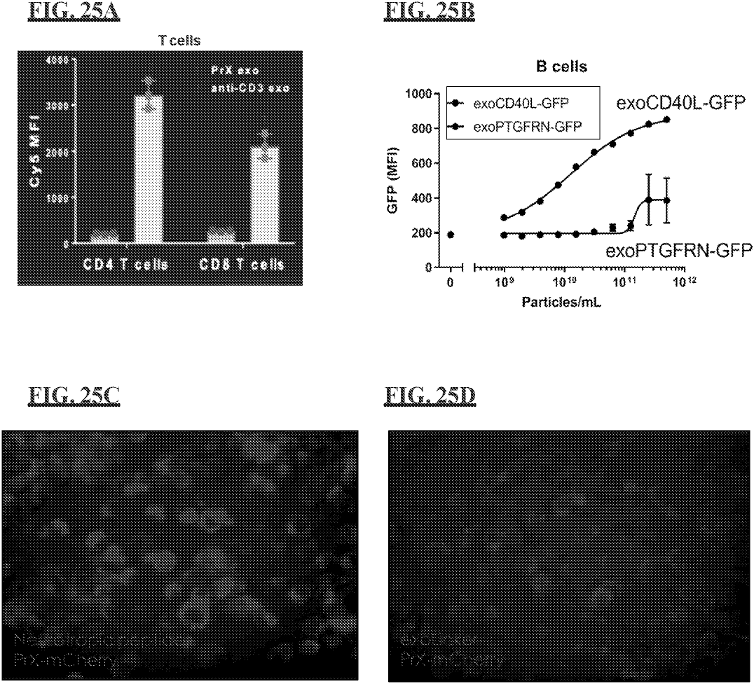

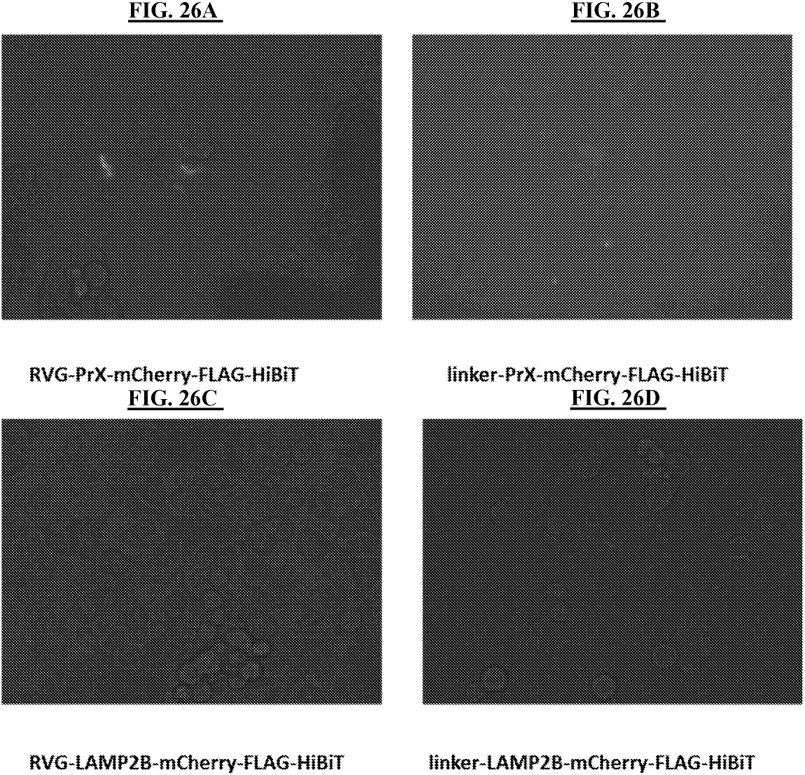

Neuro2A cells were incubated with 10⁵, 5x10⁴, 10⁴, 5x10³, or 10³ EV particles comprising the constructs disclosed above per neuron2A cell, and mCherry fluorescence was measured using microscopy. No obvious signal was observed at 1 hour or 2 hours after adding the EVs. However, EV uptake was observed at 5 hours with 10⁵ EV particles/neuro2A cell (FIGS. 26A-26D). Only the constructs comprising RVG showed uptake by the neuro2A cells. Increased uptake was observed after 18 hours (FIGS. 27A-27B). Flow cytometry showed significant uptake of UVs comprising RVG after 24 hours, both at 10⁵ EV particles/neuro2A cell and at 5x10⁴ EV particles/neuro2A cell (FIGS. 28A-28X and 29). These results indicated that attaching an RVG peptide to the external surface of an EV, e.g., an exosome, can target the EVs to neurons.

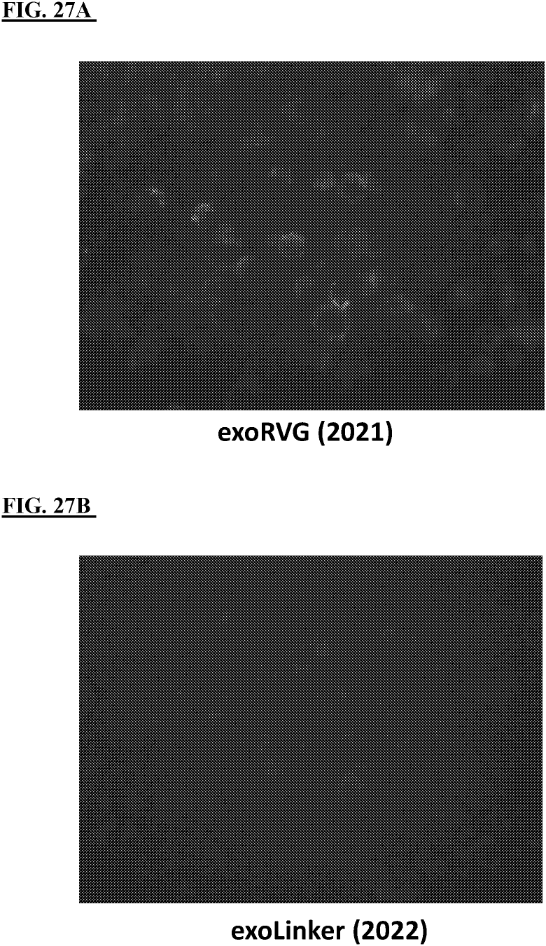

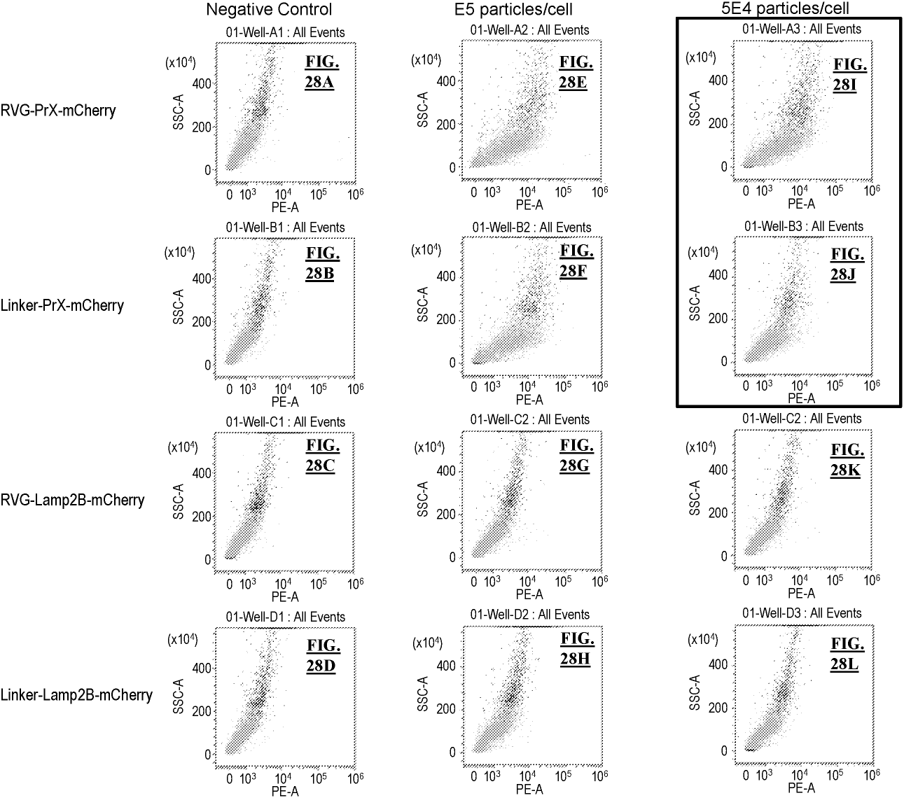

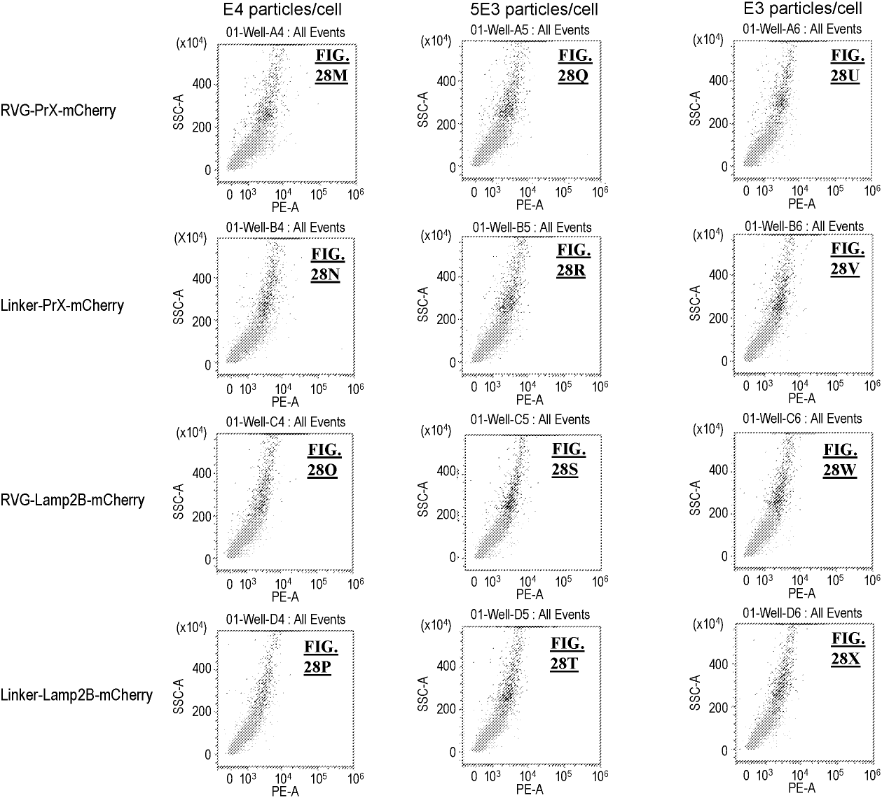

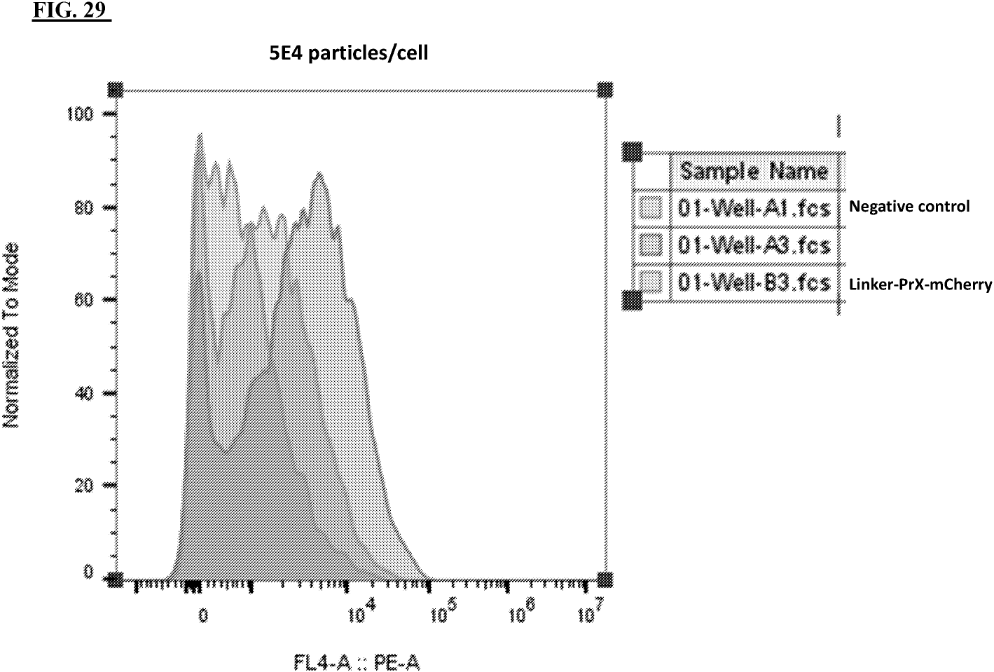

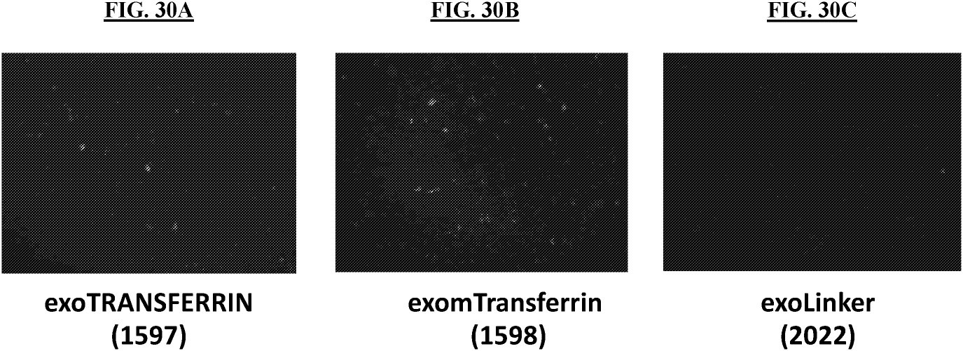

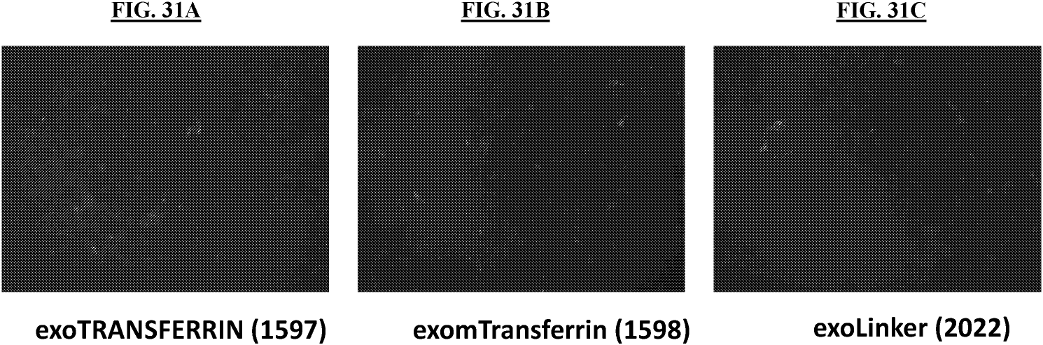

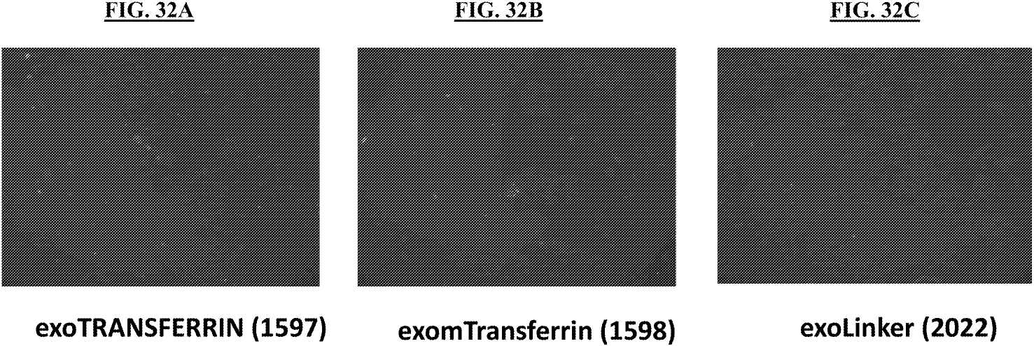

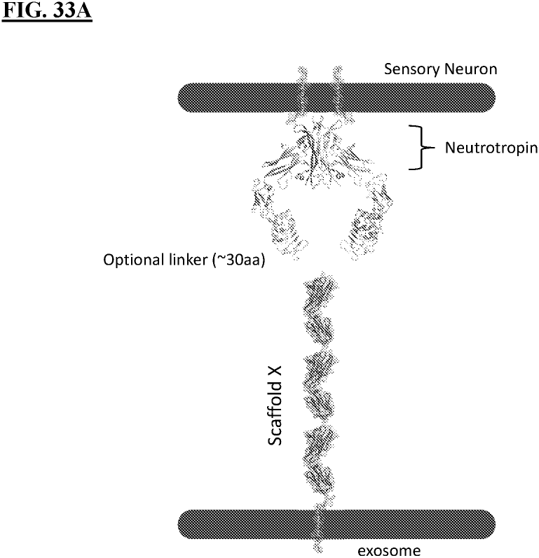

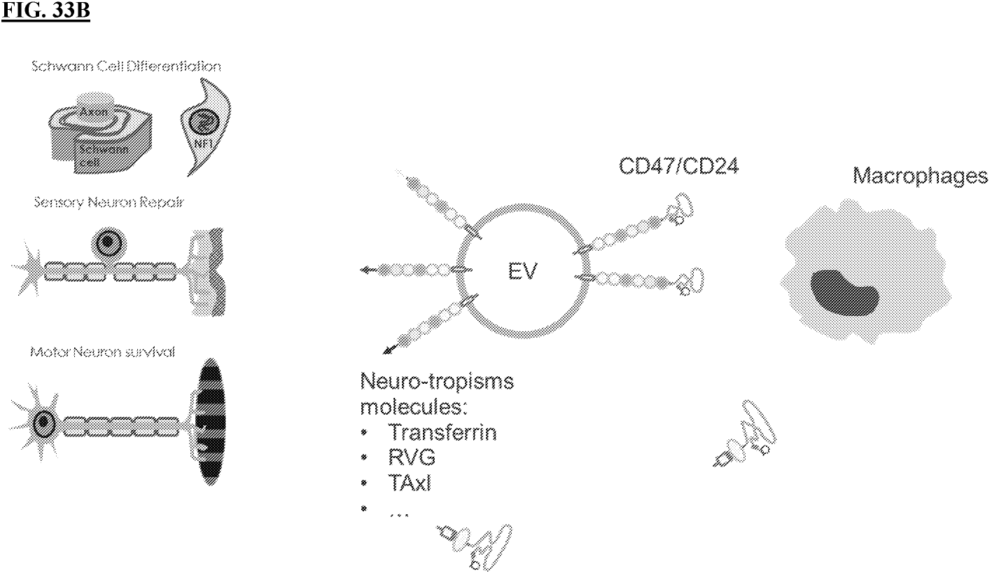

A second tropism moiety, transferrin, was also evaluated. Several constructs were tested: Transferrin-PrX-mCherry-FLAG (comprising human transferrin) (construct 1597), mTransferrin-PrX-mCherry-FLAG (comprising mouse transferrin) (construct 1598); and linker-PrX-mCherry-FLAG-HiBiT (construct 2022). 5x10⁵ EV particles per cell were used. Uptake was measured 3 hours after EV particle incubation started. Uptake was measured using microscopy. EV uptake by HeLa cells (FIGS. 30A-30C), Hep3B cells (FIGS. 31A-31C) and Hep3G2 cells (FIGS. 32A-32C) was observed for both human and mouse transferrin-containing EVs, indicating that transferrin can be use to target EVs to these three cell types.

### Example 11: Intrathecal (ITH) and Intra-Cisterna Magna (ICM) Delivery of Radiolabeled and Fluorescence-Tagged PrX Exosomes in Non-Human Primates

### In-Life Preparation

Subjects were placed in lateral recumbency and the area over the lumbar region was clipped and prepared with chlorhexidine scrub and solution, then maintained under anesthesia throughout the course of dose administration whilst providing appropriate prophylactic analgesics and antibiotics per Study Specific Procedure.

Group 1 (PET) following ITh Administration: Using a coaxial needle technique, a Gertie Marx spinal needle (20 g outer, 22 g inner needle) was introduced into the L3/L4 intrathecal space using aseptic technique verified by the presence of CSF pre- and post-injection. Needle placement was verified using fluoroscopy to confirm placement of needle in the intrathecal space. Fluoroscopic cine images were acquired at various intervals throughout dose administration to confirm needle placement was maintained in the intrathecal space. Subsequently 1x10⁸ [⁸⁹Zr]PrX exosomes formulated in 2.4 mL aCSF, were slowly injected over a period of approximately 3 min with a target dose rate of approximately 1.0 mL/min via hand-administration, and a total injection volume of 2.4 mL. After completion of administration, the syringe and needle were removed, and the animal transported to the imaging suite for initiation of PET image acquisition involving a 0.5-1.5 h dynamic scan followed by static scans at 6 h and 24 h.

Group 2 (CFT) following ITh Administration: The method described above was used to administer 4x10¹² Cy7-ExoASOscramble- PrX exosomes formulated in 2.4 mL aCSF into the lumbar intrathecal space. After completion of administration, the syringe and needle were removed, and the animal euthanized with spinal column and whole head or processed for subsequent CFT imaging involving freezing in chilled hexane ready to then be imaged using a CGT protocol.

Group 3 (IHC) following ITh Administration: The method described above was used to administer 4x10¹² Cy7-ExoASO_{scramble}- PrX exosomes formulated in 2.4 mL aCSF into the lumbar intrathecal space. After completion of administration, the syringe and needle were removed, and the animal euthanized with spinal cord and brain regions dissected and fixed for FFPE processing and subsequent IHC staining.

Group 4 (PET/CFT) following ICM Administration: Animal 1 was re-used at a later date using a combination of the steps above for ultimate PET and then CFT imaging. Briefly, the animal was positioned in lateral recumbency, chin to chest, and the area over the cisterna magna clipped and prepared with chlorhexidine scrub and solution. A 22G Quinke needle was then advanced percutaneously into the cisterna magna. Once CSF flow was confirmed, a mixture of [⁸⁹Zr]PrX and Cy7-ExoASO_{scramble}- PrX exosomes was infused by hand as a slow bolus at a rate of 0.7 - 1.0 mL/min; total injection volume of 2.4 mL. The animal was then scanned for PET imaging as described above followed by euthanasia and prepping for CFT also as described above

The biodistribution of [⁸⁹Zr]DFO-PrX exosomes was evaluated via PET/CT imaging following intrathecal administration to monkeys (N=5; n=3 male, n=2 female; cynomolgus macaques, 2-5 years, 2-5 kg), as described above. A Cy7-ASOscramble version of the exosome (Cy7ASOscr PrX exosomes) was also administered IT for assessment of biodistribution via cryofluorescent tomography (CFT) and immunohistochemistry (IHC). Study design summary is shown in Table 4.

The biodistribution of [⁸⁹Zr]DFO-PrX exosomes was evaluated via PET/CT imaging following intrathecal administration to monkeys (N=5; n=3 male, n=2 female; cynomolgus macaques, 2-5 years, 2-5 kg). A Cy7-ASOscramble version of the exosome (Cy7ASOscr PrX exosomes) was also administered IT for assessment of biodistribution via cryofluorescent tomography (CFT) and immunohistochemistry (IHC). Study design summary is shown in Table 4.

TABLE 4

Study Design

| Group | Number of Animals, Sex | Animal ID | Test Article & Dose Route | Dose Level (Particles/Subject) | Dose Radioactivity (mCi/Animal) | Dose Volume (mL) | Endpoint/Samples Collected |
|---|---|---|---|---|---|---|---|
| 1 | 1 M | A9201 (PET/CT only) | ⁸⁹Zr-DFO PrX exosome + PrX Exosome, Ith | 1 x 10⁸ | 1-2 | 2.4 | PET/CT Image Data (0.5-1.5 h dynamic, 1 h static at 6 and 24 h) Blood Samples, CSF Samples |
| 2 | 1 F | A9204 (CFT only) | Cy7 ASO Scramble PrX Exosome, Ith | 4x 10¹² | N/A | 2.4 | Tissues (CFT) |
| 4 | 1 M | A9201 (PET/CT and CFT; repeat animal) | ⁸⁹Zr-DFO PrX exosome + Cy7 ASO Scramble PrX Exosome, ICM | 1 x 10⁸+ 1 x 10¹² | 1-2 | 2.4 | PET/CT Image Data (0.5-1.5 h dynamic, 1 h static at 6 and 24 h) Blood Samples, CSF Samples, Tissues (CFT; G4 only) |
| 3 | 1 F | A9205 (IHC only) | Cy7 ASO Scramble PrX Exosome, ITh | 4 x 10¹² | N/A | 2.4 | Tissues (IHC) |
| 5 | 1 M | TBD | TBD | TBD | TBD | TBD | TBD |

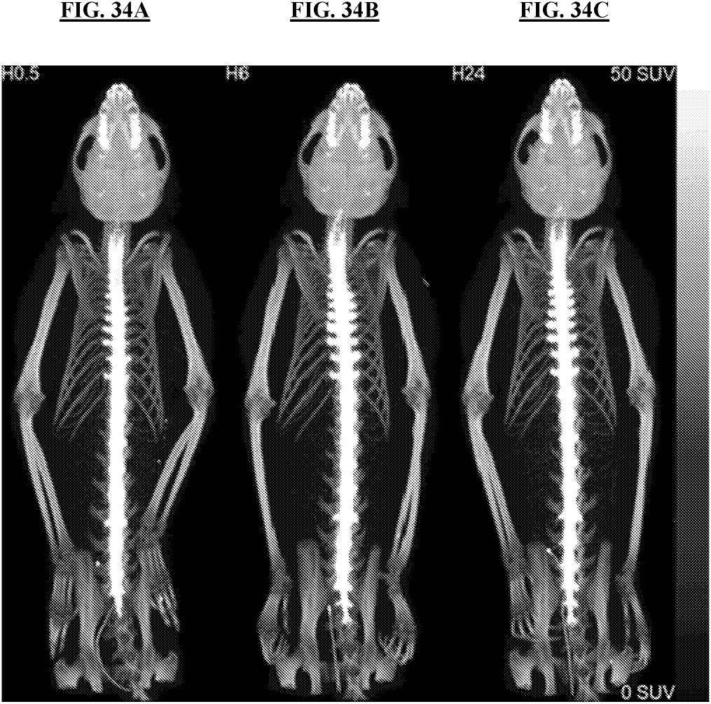

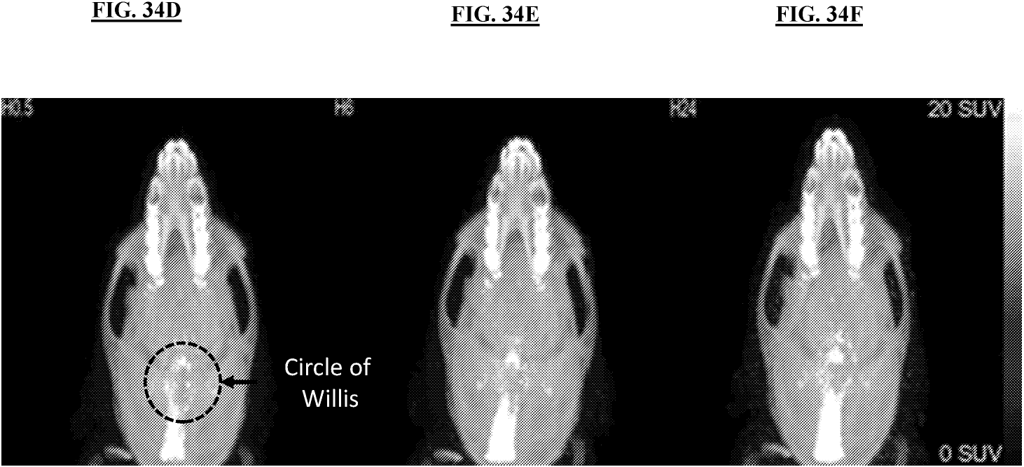

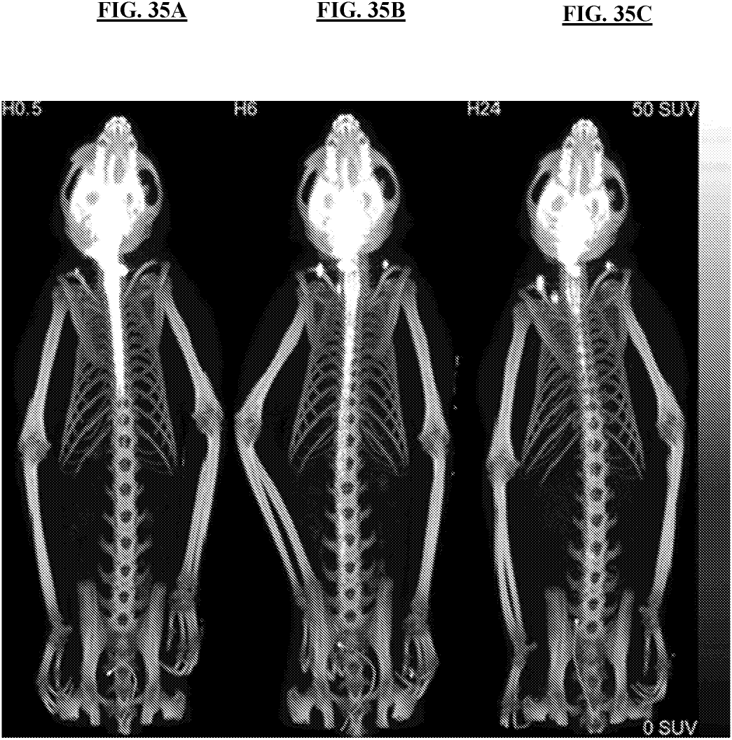

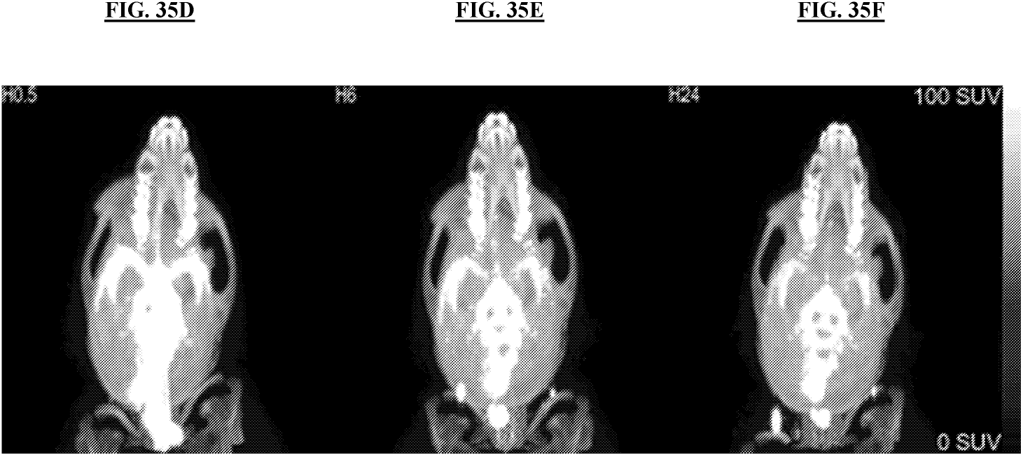

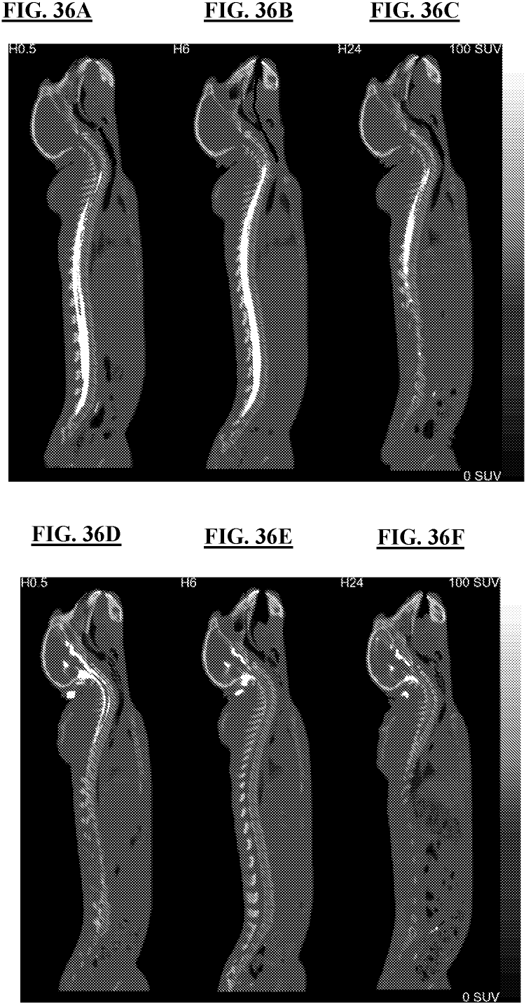

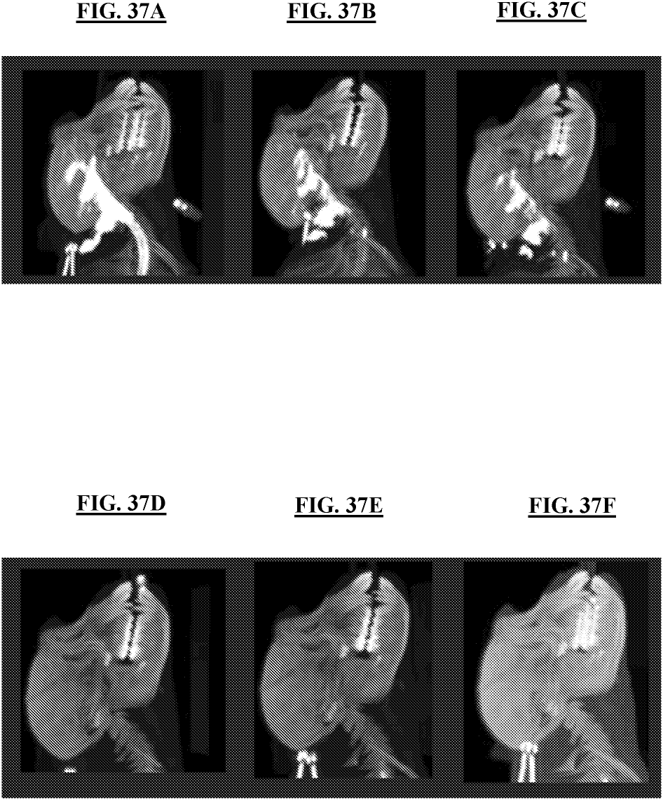

Maximum intensity projections (MIPs) (FIGS. 34A-34C) and cropped head MIPs (FIGS. 34D-34F) for [⁸⁹Zr]DFO-PrX exosome biodistribution in subject A9201 over 24 hour post-ITH administration were visualized. Maximum intensity projections (MIPs) (FIGS. 35A-35C) and cropped head MIPs (FIGS. 35D-35F) for [⁸⁹Zr]DFO-PrX exosome biodistribution in subject A9201 over 24 hour post-ICM administration were visualized. Cropped head slices for ICM (FIGS. 37A-37C) and ITH (FIGS. 37D-37F) for [⁸⁹Zr]DFO-PrX exosome biodistribution in subject A9201 over 24 hour post-ICM administration were visualized.

### Example 11: Characterization of Exosomes With Tropism Moieties

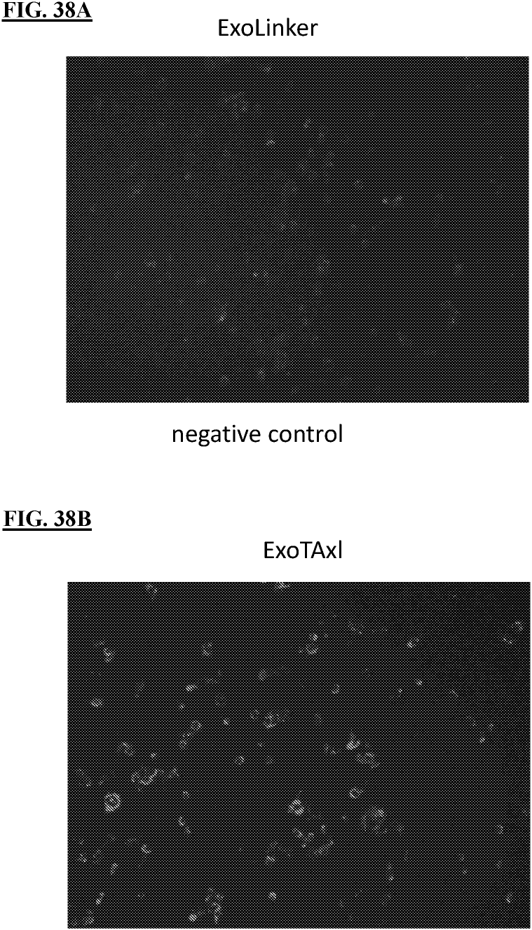

In order to direct EVs to specific cellular types, various constructs were created to express different tropism moieties. To determine whether the exoTAxl peptide could be used to direct EVs to neurons, Neuro2A cells were incubated with 5x10⁵ EV particles/cell of EVs comprising exoTAxl peptide. EVs were engineered with mCherry tag, and the mCherry fluorescence intensity was examined using fluorescent microscopy (FIGS. 38A-38B). exoTAxl showed significantly more uptake by the neuro2A cells than the exoLinker negative control. These results indicated that attaching an TAxl peptide to the external surface of an EV can enhance the EVs uptake in neuronal cells.

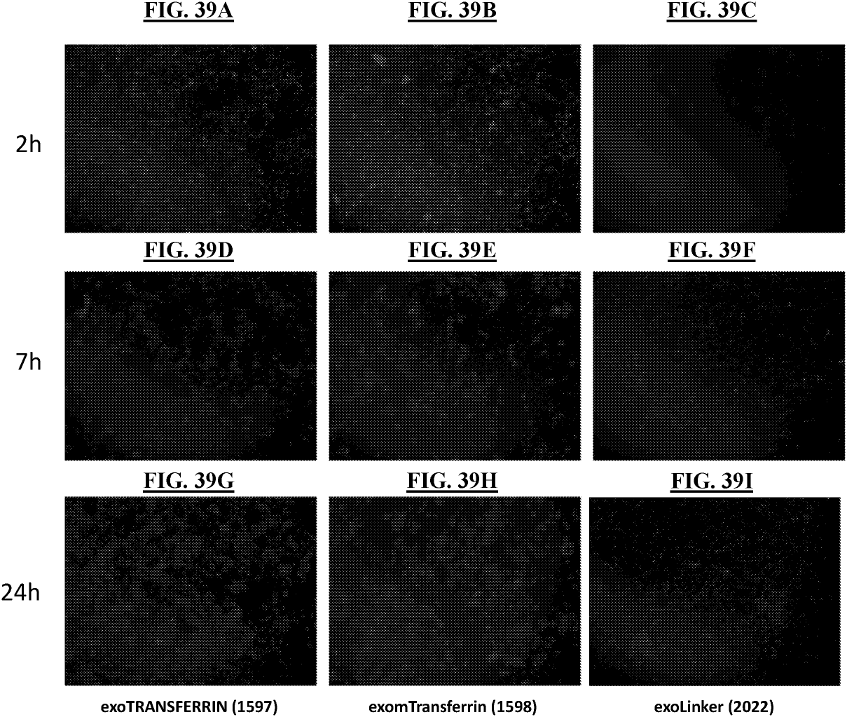

Neuro2A cells were seeded at E5 cells/well in 24-well plate, incubated EVs expressing exoTransferrin at 5x10⁵ EV particles/cell. EVs were engineered with mCherry tag, and mCherry fluorescence was examined by fluorescent microscopy (FIGS. 39A-39I). Exo-mTransferrin and exoTransferrin showed faster (starting after 2 h, increased at 7 h and even high at 24 h)) and greater uptake by the neuro2A cells than the exoLinker negative control. These results indicated that attaching Transferrin to the external surface of an EV, can enhance the EVs uptake in neuronal cells.

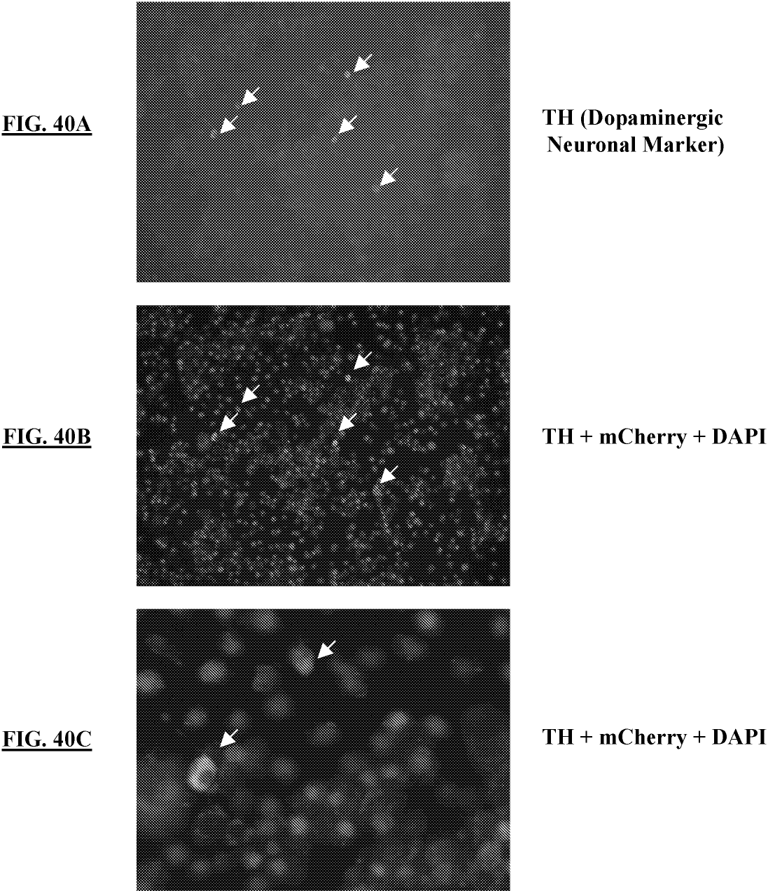

Further, exo-Transferrin showed uptake by the differentiated neuro2A cells overnight (FIGS. 40A-40C). These results indicated that attaching transferrin to the external surface of an EV, can enhance the EVs uptake in neuronal cells.

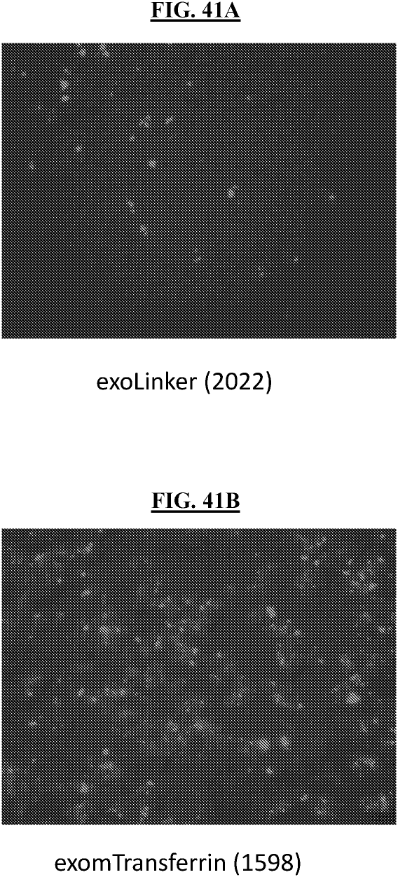

Human neuroblastoma cells, SH-SY-5Y, were seeded at E5 cells per well in 24-well plate and incubated with EV samples for 24 h. Exo-mTransferrin showed greater uptake by the SH-SY-5Y cells than the exoLinker negative control (FIGS. 41A-41B). These results indicate that attaching Transferrin to the external surface of an EV, can enhance the EVs uptake in human neuronal cells.

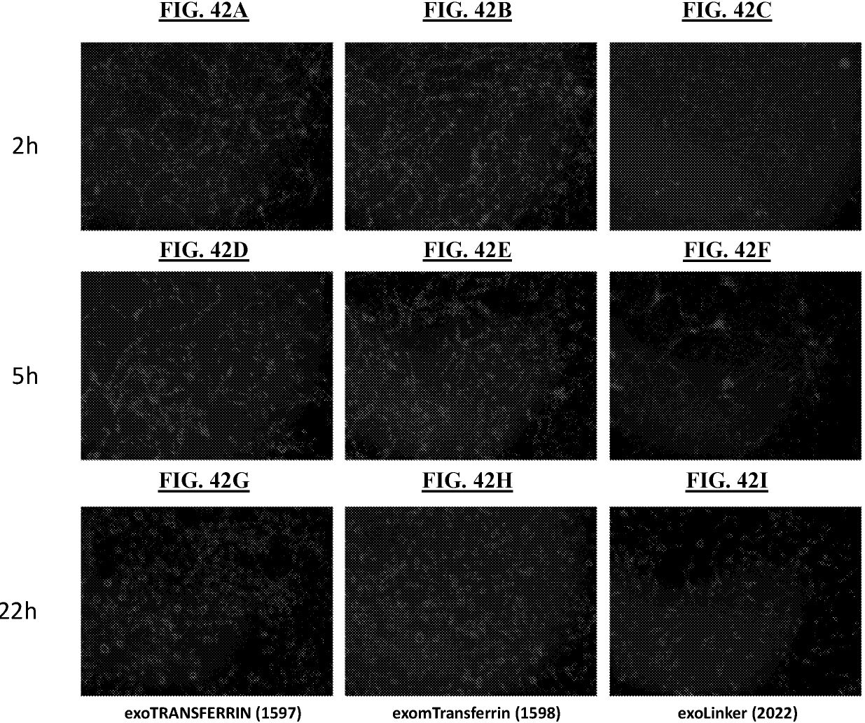

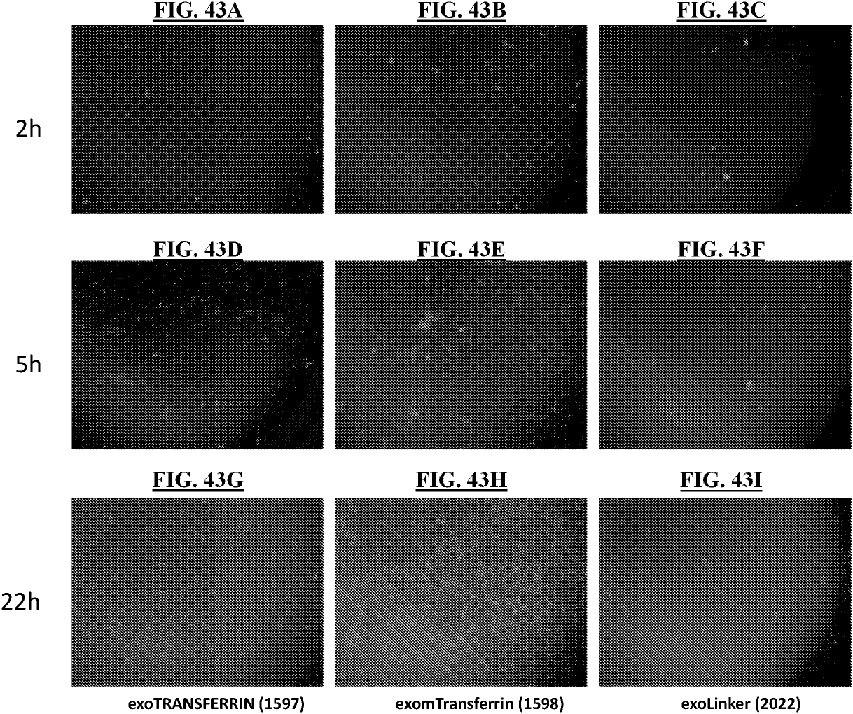

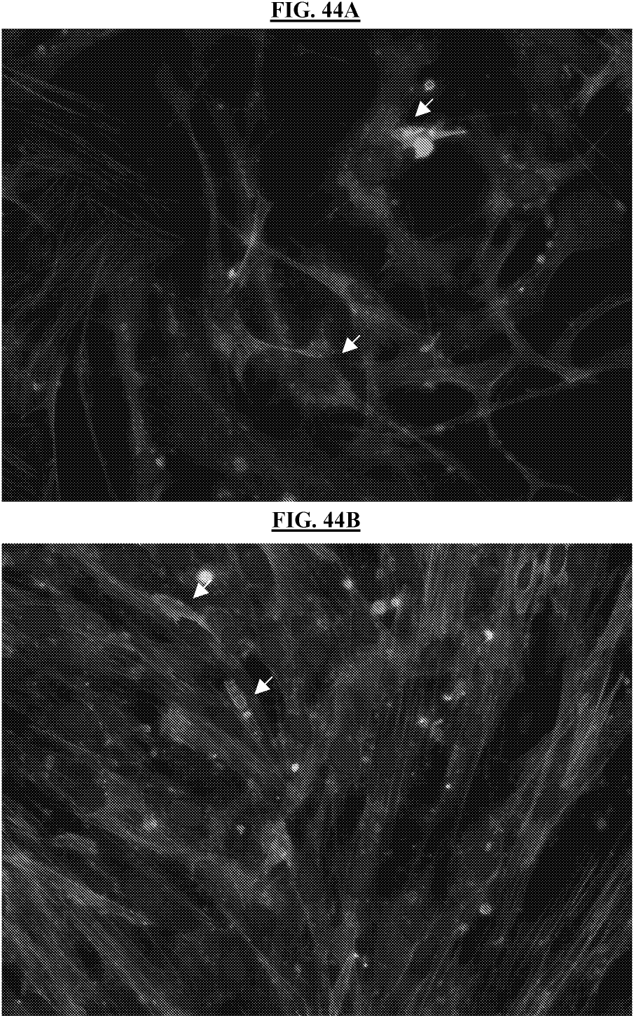

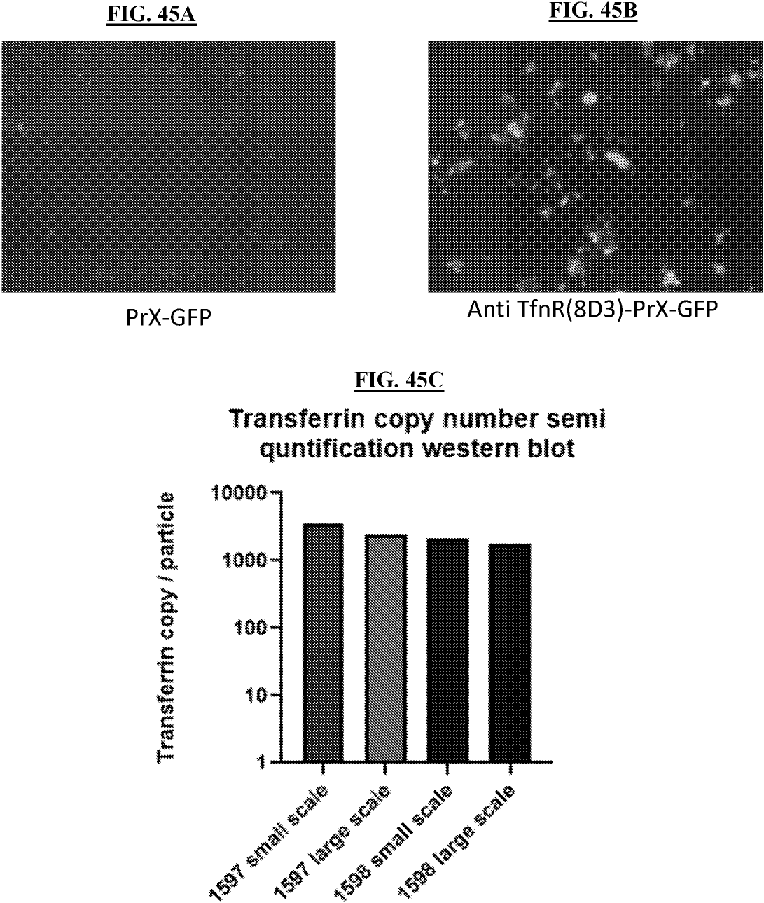

Primary mouse Schwann cells (ScienCell) were seeded at E5 cells/well into 24 well plate, incubated with 5x10⁵ EV particles/cell. EVs were engineered with mCherry tag and mCherry fluorescence were examined by fluorescent microscopy. Exo-mTransferrin and exoTransferrin showed faster (starting after 2 h, increased at 7 h and 22 h)) and greater uptake by the mouse Schwann cells than the exoLinker negative control (FIGS. 42A-42I). These results indicated that attaching Transferrin to the external surface of an EV, can enhance the EVs uptake in Schwann cells. Primary human Schwann cells (ScienCell) were then seeded at E5 cells/well into 24-well plate, incubated with 5x10⁵ EV particles/cell. Evs were engineered with mCherry tag and mCherry fluorescence was examined by fluorescent microscopy. Exo-mTransferrin and exoTransferrin showed greater uptake by the human Schwann cells compared to the exoLinker negative control at 5 h and 22 h time points (FIGS. 43A-43I). These results indicated that attaching Transferrin to the external surface of an EV, can enhance the Evs uptake in human Schwann cells. ExoTransferrin showed uptake in both mouse and human Schwann cells. Fixed cells were stained with anti-cytoskeleton-marker antbody and DAPI, and imaging reveals that the exoTransferrin EVs were taken up more by mouse Schwann cells (FIG. 44A) than human Schwann cells (FIG. 44B).

The anti-Transferrin receptor antibody (8D3) was tested as a means of targeting EVs to human neuroblast cells. SH-SY-5Y cells were cultured a E5 cells/well in the presense of EVs expressing PrX-GFP (negative control) or anti-TfnR(8D3)-PrX-GFP (FIG. 45C) overnight. anti-TfnR(8D3)-PrX-GFP EVs were readily taken up by the neuroblast cells (FIG. 45B).

### INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### EQUIVALENTS

While various specific aspects have been illustrated and described, the above specification is not restrictive. It will be appreciated that various changes can be made without departing from the spirit and scope of the disclosure(s). Many variations will become apparent to those skilled in the art upon review of this specification.

What is claimed is:

1. A method of treating a disease or disorder in a subject in need thereof, comprising compartmentally administering to the subject an effective amount of a composition comprising an extracellular vesicle (EV) which comprises a biologically active molecule.

2. The method of claim 1, wherein the compartmental administration localizes the EV to a target tissue.

3. A method of directing an extracellular vesicle (EV) which comprises a biologically active molecule to a target tissue in a subject in need thereof, comprising compartmentally administering an effective amount of a composition comprising the EV to the subject.

4. The method of any one of claims 1 to 3, wherein the compartmental administration comprises administering the composition by a route selected from intraperitoneal, inhalation, oral, intramuscular, intrathecal, intracranial, intraocular, intradermal, sub-cutaneous, and any combination thereof.

5. The method of any one of claims 2 to 4, wherein the target tissue comprises the central nervous system (CNS).

6. The method claim 5, wherein the EV is administered intrathecally.

7. The method of claim 5 or 6, wherein the EV is administered intra-cranially.

8. The method of any one of claims 2 to 4, wherein the target tissue comprises the eye.

9. The method of claim 8, wherein the EV is administered intraocularly.

10. The method of claim 9, wherein the intraocular administration is selected from the group consisting of intravitreal, intracameral, subconjunctival, subretinal, subscleral, intrachoroidal, suprachoroidal, and any combination thereof.

11. The method of any one of claims 2 to 4, wherein the target tissue comprises a muscle.

12. The method of claim 11, wherein the EV is administered intramuscularly.

13. The method of any one of claims 2 to 4, wherein the target tissue comprises the lungs.

14. The method of claim 13, wherein the EV is administered by inhalation.

15. The method of any one of claims 2 to 4, wherein the target tissue comprises a lymph node.

16. The method of claim 15, wherein the EV is administered intraperitoneally.

17. The method of any one of claims 2 to 4, wherein the target tissue comprises the colon.

18. The method of claim 17, wherein the EV is administered orally.

19. The method of any one of claims 1 to 18, wherein the compartmental administration comprises the injection of the composition.

20. The method of any one of claims 1 to 19, wherein the compartmental administration comprises the implantation of a delivery device comprising the composition.

21. The method of claim 20, wherein the delivery device comprises an implanted pump or a sustained delivery device.

22. The method of any one of claims 1 to 21, wherein the EV comprises an exogenous targeting moiety that specifically binds to a marker present on a cell in the target tissue.

23. The method of claim 22, wherein the exogenous targeting moiety comprises a peptide, an antibody or an antigen-binding fragment thereof, a chemical compound, or any combination thereof.

24. The method of claim 22 or 23, wherein the exogenous targeting moiety comprises an antibody or antigen-binding fragment thereof.

25. The method of claim 23 or 24, wherein the antibody or antigen-binding fragment thereof comprises a full-length antibody, a single domain antibody, a heavy chain only antibody (VHH), a single chain antibody, a shark heavy chain only antibody (VNAR), an scFv, a Fv, a Fab, a Fab′, a F(ab′)2, or any combination thereof.

26. The method of any one of claims 23 to 25, wherein the antibody is a single chain antibody.

27. The method of any one of claims 22 to 26, wherein the exogenous targeting moiety comprises a microprotein, a designed ankyrin repeat protein (darpin), an anticalin, an adnectin, an aptamer, a peptide mimetic molecule, a natural ligand for a receptor, a camelid nanobody, or any combination thereof.

28. The method of any one of claims 22 to 27, wherein the exogenous targeting moiety specifically binds to a marker on a CNS cell.

29. The method of claim 28, wherein the CNS cell is a selected from a neuronal cell, a glial cell, and any combination thereof.

30. The method of any one of claims 22 to 29, wherein the CNS cell is a selected from an oligodendrocyte, an astrocyte, an ependymal cell, a microglia, and any combination thereof.

31. The method of any one of claims 22 to 29, wherein the CNS cell is a selected from a motor neuron, a sensory neuron, an interneuron, and any combination thereof.

32. The method of any one of claims 22 to 31, wherein the exogenous targeting moiety specifically binds to a marker on an eye cell.

33. The method of claim 32, wherein the eye cell is selected from a rod cell, a cone cell, a retinal ganglion cell, and any combination thereof.

34. The method of any one of claims 22 to 33, wherein the exogenous targeting moiety specifically binds to a marker on a muscle cell.

35. The method of claim 34, wherein the muscle cell is selected from a skeletal muscle cell, a smooth muscle cell, a cardiomyocyte, and any combination thereof.

36. The method of any one of claims 22 to 35, wherein the exogenous targeting moiety specifically binds to a marker on an immune cell.

37. The method of claim 36, wherein the immune cell is selected from the group consisting of a CD4 T cell, a CD8 T cell, a B cell, and any combination thereof.

38. The method of claim 36 or 37, wherein the exogenous targeting moiety binds CD3.

39. The method of claim 36 or 37, wherein the exogenous targeting moiety comprises CD40L.

40. The method of any one of claims 22 to 39, wherein the exogenous targeting moiety specifically binds to a marker on a macrophage.

41. The method of claim 40, wherein the exogenous targeting moiety increases uptake of the EV by a macrophage.

42. The method of claim 41, wherein uptake of the EV by the macrophage activates the macrophage.

43. The method of any one of claims 1 to 42, wherein the biologically active molecule is capable of repolarizing a macrophage.

44. The method of claim 43, wherein the macrophage is repolarized from an M2 to an M1 phenotype.

45. The method of any one of claims 1 to 44, wherein the EV comprises a surface antigen that inhibits uptake of the EV by a macrophage.

46. The method of claim 45, wherein the surface antigen is selected from CD47, CD24, a fragment thereof, and any combination thereof.

47. The method of claim 45 or 46, wherein the surface antigen is associated with the exterior surface of the EV.

48. The method of any one of claims 1 to 47, wherein the biologically active molecule, the exogenous targeting moiety, or both are linked to the EV by a scaffold protein.

49. The method of claim 48, wherein the scaffold protein is a Scaffold X protein.

50. The method of claim 49, wherein the Scaffold X protein comprises prostaglandin F2 receptor negative regulator (the PTGFRN protein); basigin (the BSG protein); immunoglobulin superfamily member 2 (the IGSF2 protein); immunoglobulin superfamily member 3 (the IGSF3 protein); immunoglobulin superfamily member 8 (the IGSF8 protein); integrin beta-1 (the ITGB1 protein); integrin alpha-4 (the ITGA4 protein); 4F2 cell-surface antigen heavy chain (the SLC3A2 protein); a class of ATP transporter proteins (the ATP1A1, ATP1A2, ATP1A3, ATP1A4, ATP1B3, ATP2B1, ATP2B2, ATP2B3, ATP2B4 proteins), CD13, aminopeptidase N (ANPEP), neprilysin (membrane metalloendopeptidase; MME), ectonucleotide pyrophosphatase/phosphodiesterase family member 1 (ENPP1), neuropilin-1 (NRP1), CD9, CD63, CD81, PDGFR, GPI anchor proteins, lactadherin, LAMP2, LAMP2B, a fragment thereof, or any combination thereof.

51. The method of claim 49 or 50, wherein the Scaffold X protein comprises the amino acid sequence set forth as SEQ ID NO: 33.

52. The method of claim 49 or 50, wherein the Scaffold X protein comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 1.

53. The method of claim 48, wherein the scaffold protein is a Scaffold Y protein.

54. The method of claim 53, wherein the Scaffold Y protein comprises myristoylated alanine rich Protein Kinase C substrate (the MARCKS protein), myristoylated alanine rich Protein Kinase C substrate like 1 (the MARCKSL1 protein), brain acid soluble protein 1 (the BASP1 protein), a fragment thereof, and or any combination thereof.

55. The method of claim 53 or 54, wherein the Scaffold Y protein is BASP1 protein or a fragment thereof.

56. The method of any one of claims 53 to 55, wherein the Scaffold Y protein comprises an N-terminus domain (ND) and an effector domain (ED), wherein the ND and/or the ED are associated with the luminal surface of the EV.

57. The method of claim 56, wherein the ND is associated with the luminal surface of the exosome via myristoylation.

58. The method of claim 56 or 57, wherein the ED is associated with the luminal surface of the exosome by an ionic interaction.

59. The method of any one of claims 56 to 58, wherein the ED comprises (i) a basic amino acid or (ii) two or more basic amino acids in sequence, wherein the basic amino acid is selected from the group consisting of Lys, Arg, His, and any combination thereof.

60. The method of claim 59, wherein the basic amino acid is (Lys)n, wherein n is an integer between 1 and 10.

61. The method of any one of claims 56 to 60, wherein the ED comprises Lys (K), KK, KKK, KKKK (SEQ ID NO: 205), KKKKK (SEQ ID NO: 206), Arg (R), RR, RRR, RRRR (SEQ ID NO: 207); RRRRR (SEQ ID NO: 208), KR, RK, KKR, KRK, RKK, KRR, RRK, (K/R)(K/R)(K/R)(K/R) (SEQ ID NO: 209), (K/R)(K/R)(K/R)(K/R)(K/R) (SEQ ID NO: 210), or any combination thereof.

62. The method of any one of claims 56 to 61, wherein the ND comprises an amino acid sequence selected from the group consisting of (i) GGKLSKK (SEQ ID NO: 211), (ii) GAKLSKK (SEQ ID NO: 212), (iii) GGKQSKK (SEQ ID NO: 213), (iv) GGKLAKK (SEQ ID NO: 214), and (vi) any combination thereof.

63. The method of any one of claims 48 to 62, wherein the scaffold protein comprises (i) GGKLSKKKKGYNVN (SEQ ID NO: 246), (ii) GAKLSKKKKGYNVN (SEQ ID NO: 247), (iii) GGKQSKKKKGYNVN (SEQ ID NO: 248), (iv) GGKLAKKKKGYNVN (SEQ ID NO: 249), (v) GGKLSKKKKGYSGG (SEQ ID NO: 250), (vi) GGKLSKKKKGSGGS (SEQ ID NO: 251), (vii) GGKLSKKKKSGGSG (SEQ ID NO: 252), (viii) GGKLSKKKSGGSGG (SEQ ID NO: 253), (ix) GGKLSKKSGGSGGS (SEQ ID NO: 254), (x) GGKLSKSGGSGGSV (SEQ ID NO: 255), or (xi) GAKKSKKRFSFKKS (SEQ ID NO: 256).

64. The method of any one of claims 1 to 63, wherein the composition comprising the EV further comprises a therapeutic molecule, an immune modulator, an adjuvant, or any combination thereof.

65. The method of claim 64, wherein the therapeutic molecule comprises an antigen.

66. The method of claim 64, wherein the adjuvant comprises a Stimulator of Interferon Genes (STING) agonist, a toll-like receptor (TLR) agonist, an inflammatory mediator, or any combination thereof.

67. The method of claim 64 or 66, wherein the adjuvant comprises a STING agonist.

68. The method of claim 67, wherein the STING agonist comprises a cyclic dinucleotide STING agonist or a non-cyclic dinucleotide STING agonist.

69. The method of claim 64, wherein the adjuvant is a TLR agonist.

70. The method of claim 64 or 69, wherein the TLR agonist comprises a TLR2 agonist (e.g., lipoteichoic acid, atypical LPS, MALP-2 and MALP-404, OspA, porin, LcrV, lipomannan, GPI anchor, lysophosphatidylserine, lipophosphoglycan (LPG), glycophosphatidylinositol (GPI), zymosan, hsp60, gH/gL glycoprotein, hemagglutinin), a TLR3 agonist (e.g., double-stranded RNA, e.g., poly(I:C)), a TLR4 agonist (e.g., lipopolysaccharides (LPS), lipoteichoic acid, β-defensin 2, fibronectin EDA, HMGB1, snapin, tenascin C), a TLR5 agonist (e.g., flagellin), a TLR6 agonist, a TLR⅞ agonist (e.g., single-stranded RNA, CpG-A, Poly G10, Poly G3, Resiquimod), a TLR9 agonist (e.g., unmethylated CpG DNA), or any combination thereof.

71. The method of any one of claims 64 to 70, wherein the therapeutic molecule, the immune modulator, the adjuvant, or any combination thereof, is associated with Scaffold X, Scaffold Y, or a combination thereof.

72. The method of any one of claims 64 to 71, wherein the immune modulator comprises a cytokine.

73. The method of claim 72, wherein the cytokine comprises an interferon.

74. The method of any one of claims 1 to 73, wherein the EV is an exosome.

75. An EV comprising a surface antigen fused to a scaffold protein, wherein the surface antigen comprises an anti-phagocytic signal .

76. The EV of claim 75, wherein the antigen comprises human CD47, human CD24, or a functional fragment thereof.

77. The EV of claim 75 or 76, wherein the antigen comprises a minimal “self” peptide having the amino acid sequence set forth in SEQ ID NO: 382.

78. The EV of any one of claims 75 to 77, wherein the antigen comprises the extracellular domain of human CD47.

79. The EV of any one of claims 75 to 78, wherein the antigen comprises amino acids 19 to about 141 of SEQ ID NO 400, amino acids 19-135 of SEQ ID NO 400, amino acids 19-130 of SEQ ID NO 400, or amino acids 19-125 of SEQ ID NO 400.

80. The EV of any one of claims 75 to 79, wherein the scaffold protein comprises a Scaffold X protein.

81. The EV of any one of claims 75 to 80, further comprising an exogenous targeting moeity that specifically binds to a marker present on a cell in the target tissue.

82. The method of claim 81, wherein the exogenous targeting moiety comprises a peptide, an antibody or an antigen-binding fragment thereof, a chemical compound, or any combination thereof.

83. The method of claim 82, wherein the antibody or antigen-binding fragment thereof comprises a full-length antibody, a single domain antibody, a heavy chain only antibody (VHH), a single chain antibody, a shark heavy chain only antibody (VNAR), an scFv, a Fv, a Fab, a Fab′, a F(ab′)2, or any combination thereof.

84. The method of any one of claims 81 to 83, wherein the exogenous targeting moiety comprises a microprotein, a designed ankyrin repeat protein (darpin), an anticalin, an adnectin, an aptamer, a peptide mimetic molecule, a natural ligand for a receptor, a camelid nanobody, or any combination thereof.

85. The method of any one of claims 81 to 84, wherein the exogenous targeting moiety specifically binds to a marker on a cell selected from the group consisting of a CNS cell, an eye cell, a muscle cell, an immune cell, and any combination thereof.

86. The method of claim 85, wherein the CNS cell is a selected from a neuronal cell, a glial cell, an oligodendrocyte, an astrocyte, an ependymal cell, a microglia, a motor neuron, a sensory neuron, an interneuron, and any combination thereof.

87. The method of claim 85, wherein the eye cell is selected from a rod cell, a cone cell, a retinal ganglion cell, and any combination thereof.

88. The method of claim 85, wherein the muscle cell is selected from a skeletal muscle cell, a smooth muscle cell, a cardiomyocyte, and any combination thereof.

89. The method of claim 85, wherein the immune cell is selected from the group consisting of a CD4 T cell, a CD8 T cell, a B cell, and any combination thereof.

90. The method of claim 89, wherein the exogenous targeting moiety binds CD3.

91. The method of claim 89, wherein the exogenous targeting moiety comprises CD40L.

92. The method of any one of claims 81 to 91, wherein the exogenous targeting moiety specifically binds to a marker on a macrophage.

93. The method of claim 92, wherein the exogenous targeting moiety increases uptake of the EV by a macrophage.

94. The method of claim 93, wherein uptake of the EV by the macrophage activates the macrophage.

95. A method of increasing retension of an EV in circulation, comprising expressing human CD47, human CD24, or a functional fragment thereof on the exterior surface of the EV.

96. A method of altering biodistribution of an EV in circulation, comprising expressing human CD47, human CD24, or a functional fragment thereof on the exterior surface of the EV.

97. The method of claim 95 or 96, wherein the fragment of human CD47 comprises the amino acid sequence set forth in SEQ ID NO: 382.

98. The method of any one of claims 95 to 97, wherein the fragment of human CD47 comprises the extracellular domain of human CD47.

99. The method of any one of claims 95 to 98, wherein the fragment of human CD47 comprises amino acids 19 to about 141 of SEQ ID NO 400, amino acids 19-135 of SEQ ID NO 400, amino acids 19-130 of SEQ ID NO 400, or amino acids 19-125 of SEQ ID NO 400.

100. The method of any one of claims 95 to 99, wherein the fragment of human CD47 is fused to a scaffold protein.

101. The method of claim 100, wherein the scaffold protein is a Scaffold X protein.

102. A method of targeting an extracellular vesicle to central nervous system in a subject in need thereof comprising administering a composition comprising an extracellular vesicle (EV) which comprises a biologically active molecule to the subject, wherein the administration of the composition is intrathecal, intraocular, intracranial, intranasal, or perineural.

103. A method of treating a central nervous system disease in a subject in need thereof comprising administering a composition comprising an extracellular vesicle (EV) which comprises a biologically active molecule to the subject, wherein the administration of the composition is intrathecal, intraocular, intracranial, intranasal, or perineural.

104. The method of claim 102 or 103, wherein the intrathecal administration is in the spinal canal and/or the subarachnoid space.

105. The method of claim 102 to 104, wherein the intrathecal administration is by injection.

106. The method of claims 102 to 105, wherein the intrathecal administration comprises the implantation of a delivery device comprising the composition.

107. The method of claim 106, wherein the delivery device is an intrathecal pump.

108. The method of claim 102, wherein the intraocular administration is selected from the group consisting of intravitreal, intracameral, subconjunctival, subretinal, subscleral, intrachoroidal, and any combination thereof.

109. The method of claim 108, wherein the intraocular administration comprises the injection of the composition.

110. The method of any one of claim 108, wherein the intraocular administration is intravitreal injection.

111. The method of claim 108, wherein the intraocular administration comprises the implantation of a delivery device comprising the composition.

112. The method of claim 111, wherein the delivery device is an intraocular delivery device.

113. The method of claim 112, wherein the intraocular delivery device is an intravitreal implant or a scleral plug.

114. The method of any one of claims 111 to 113, wherein the delivery device is a sustained release delivery device.

115. The method of claim 102, wherein the intracranial administration is intracisternal, subarachnoidal, intrahippocampal, intracerebroventricular, intraparenchymal, or a combination thereof.

116. The method of claim 115, wherein the intracranial administration is by injection.

117. The method of claim 115, wherein the intracranial administration is via a catheter or port.

118. The method of claim 117, wherein the catheter or port is implanted.

119. The method of claim 117 or 118, wherein a pump is connected to the catheter or port.

120. The method of claim 115, wherein the in intraparenchymal administration is Convection-Enhanced Intraparenchymal administration.

121. The method of claim 102, wherein the intranasal administration is by instillation or injection.

122. The method of claim 102, where in the perineural administration is by facial intradermal injection.

123. The method of claim 122, wherein the facial intradermal injection targets the trigeminal substructures.

124. The method of claim 123, wherein the trigeminal substructures are selected from the group consisting of trigeminal perineurium, epineurium, perivascular spaces, neurons and Schwann cells, and combinations thereof.

125. The method of any one of claims 102 to 124, wherein the EV comprises a surface anchored anti-phagocytic signal.

126. The method of claim 125, wherein the anti-phagocytic signal is CD47, CD24, a fragment or variant thereof, or a combination thereof.

127. The method of any one of claims 102 to 126, wherein the EV comprises a tissue or cell-specific target ligand which increases EV tropism to a specific CNS tissue or cell.

128. The method claim 127, wherein the cell is a glial cell.

129. The method of claim 128, wherein the glial cell is an oligodendrocyte, an astrocyte, an ependymal cell, a microglia cell, a Schwann cell, a satellite glial cell, an olfactory ensheathing cell, or a combination thereof.

130. The method of claim 127, wherein the cell is a neural stem cell.

131. The method of claim 127, wherein the cell is a neuron.

132. The method of claim 131, wherein the neuron is a motor neuron, a sensory neuron, or an interneuron.

133. The method of claim 132, wherein the tissue or cell-specific target ligand is a cell marker (e.g., a protein or receptor) present of the surface of a neuron.

134. The method of claim 127, wherein the tissue is selected from the group consisting of brain tissue, spinal cord tissue, retina, optic nerve (cranial nerve II), olfactory nerves (cranial nerve I), olfactory epithelium, meningeal tissue, or any combination thereof.

135. The method of claim 127, wherein the tissue is from a CNS area selected from the group consisting of cerebrum, cerebral cortex, basal ganglia, amygdala, hippocampus, thalamus, hypothalamus, cerebellum, brainstem, medulla, pons, midbrain, and reticular formation.

136. The method of any one of claim 102 to 135, wherein the extracellular vesicle (EV) comprises a surface anchored anti-phagocytic signal and a tissue or cell-specific target ligand which increases EV tropism to cells in the CNS.

137. The method of claim 136, wherein the cells are Schwann cells or oligodendrocytes.

138. The method of claim 136, wherein the anti-phagocytic signal is CD47, CD24, a fragment or variant thereof, or a combination thereof.

139. The method claim 127, wherein the tissue or cell-specific target ligand targets a CNS specific peripheral nerve.

140. The method of claim 139, wherein the tissue or cell-specific target ligand comprises a ligand that binds to a transferrin receptor (TfR), apolipoprotein D (ApoD), Galectin 1 (LGALS1), Myelin proteolipid protein (PLP), Glypican 1, or Syndecan 3.

141. The method of claim 140, wherein the TfR is TfR1.

142. The method of claim 140, wherein the ligand that binds to TfR1 is an antibody against TfR1 or transferrin.

143. The method of claim 142, wherein the transferrin is a serum transferrin, lacto transferrin (lactoferrin) ovotransferrin, or melanotransferrin.

144. The method of claim 141, wherein the transferrin is an asialo transferrin, a monosialo transferrin, a disialo transferrin, a trisialo transferrin, a tetrasialo transferrin, a pentasialo transferrin, an hexasialo transferrin, or a combination thereof.

145. The method of claim 139, wherein the tissue or cell-specific target ligand binds to a Schwann cell surface marker.

146. The method of claim 145, wherein the Schwann cell surface marker is selected from Myelin Basic Protein (MBP) and isoforms thereof, Myelin Protein Zero (P0), P75NTR, NCAM, PMP22, and combinations thereof.

147. The method of claim 139, wherein the tissue or cell-specific target ligand comprises an antibody or an antigen-binding portion thereof, a vNAR, an aptamer, or an agonist or antagonist of a receptor expressed on the surface of the Schwann cell.

148. The method of claims 139, wherein the tissue or cell-specific target ligand targets a sensory neuron.

149. The method of claim 148, wherein the tissue or cell-specific target ligand comprises a neurotrophin that binds to a tropomyosin receptor kinase (Trk) receptor.

150. The method of claim 149, wherein the Trk receptor is TrkA, TrB, TrkC, or a combination thereof.

151. The method of claim 150, wherein the neurotrophin is Nerve growth factor (NGF), Brain-derived neurotrophic factor (BDNF), Neurotrophin-3 (NT-3), Neurotrophin-4 (NT-4), or a combination thereof.

152. The method of claim 139, wherein the tissue or cell-specific target ligand targets a motor neuron.

153. The method of claim 152, wherein the tissue or cell-specific target ligand comprises a Rabies Virus Glycoprotein (RVG) peptide, a Targeted Axonal Import (TAxI) peptide, a P75R peptide, or a Tet-C peptide.

154. The method of any one of claims 102 to 153, wherein the biologically active molecule, the anti-phagocytic signal, the tissue or cell-specific target ligand, or any combination thereof are linked to the EV by a scaffold protein.

155. The method of claim 154, wherein the scaffold protein is a Scaffold X protein.

156. The method of claim 155, wherein the Scaffold X protein comprises prostaglandin F2 receptor negative regulator (the PTGFRN protein); basigin (the BSG protein); immunoglobulin superfamily member 2 (the IGSF2 protein); immunoglobulin superfamily member 3 (the IGSF3 protein); immunoglobulin superfamily member 8 (the IGSF8 protein); integrin beta-1 (the ITGB1 protein); integrin alpha-4 (the ITGA4 protein); 4F2 cell-surface antigen heavy chain (the SLC3A2 protein); a class of ATP transporter proteins (the ATP1A1, ATP1A2, ATP1A3, ATP1A4, ATP1B3, ATP2B1, ATP2B2, ATP2B3, ATP2B4 proteins), CD13, aminopeptidase N (ANPEP), neprilysin (membrane metalloendopeptidase; MME), ectonucleotide pyrophosphatase/phosphodiesterase family member 1 (ENPP1), neuropilin-1 (NRP1), CD9, CD63, CD81, PDGFR, GPI anchor proteins, lactadherin, LAMP2, LAMP2B, a fragment thereof, or any combination thereof.

157. The method of claim 155 or 156, wherein the Scaffold X protein comprises the amino acid sequence set forth as SEQ ID NO: 33.

158. The method of claim 155 or 156, wherein the Scaffold X protein comprises an amino acid sequence having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 1.

159. The method of claim 154, wherein the scaffold protein is a Scaffold Y protein.

160. The method of claim 159, wherein the Scaffold Y protein comprises myristoylated alanine rich Protein Kinase C substrate (the MARCKS protein), myristoylated alanine rich Protein Kinase C substrate like 1 (the MARCKSL1 protein), brain acid soluble protein 1 (the BASP1 protein), a fragment thereof, and or any combination thereof.

161. The method of claim 159 or 160, wherein the Scaffold Y protein is BASP1 protein or a fragment thereof.

162. The method of any one of claims 159 to 161, wherein the Scaffold Y protein comprises an N-terminus domain (ND) and an effector domain (ED), wherein the ND and/or the ED are associated with the luminal surface of the EV.

163. The method of claim 56, wherein the ND is associated with the luminal surface of the exosome via myristoylation.

164. The method of claim 162 or 163, wherein the ED is associated with the luminal surface of the exosome by an ionic interaction.

165. The method of any one of claims 162 to 164, wherein the ED comprises (i) a basic amino acid or (ii) two or more basic amino acids in sequence, wherein the basic amino acid is selected from the group consisting of Lys, Arg, His, and any combination thereof.

166. The method of claim 165, wherein the basic amino acid is (Lys)n, wherein n is an integer between 1 and 10.

167. The method of any one of claims 162 to 166, wherein the ED comprises Lys (K), KK, KKK, KKKK (SEQ ID NO: 205), KKKKK (SEQ ID NO: 206), Arg (R), RR, RRR, RRRR (SEQ ID NO: 207); RRRRR (SEQ ID NO: 208), KR, RK, KKR, KRK, RKK, KRR, RRK, (K/R)(K/R)(K/R)(K/R) (SEQ ID NO: 209), (K/R)(K/R)(K/R)(K/R)(K/R) (SEQ ID NO: 210), or any combination thereof.

168. The method of any one of claims 162 to 167, wherein the ND comprises an amino acid sequence selected from the group consisting of (i) GGKLSKK (SEQ ID NO: 211), (ii) GAKLSKK (SEQ ID NO: 212), (iii) GGKQSKK (SEQ ID NO: 213), (iv) GGKLAKK (SEQ ID NO: 214), and (vi) any combination thereof.

169. The method of any one of claims 154 to 168, wherein the scaffold protein comprises (i) GGKLSKKKKGYNVN (SEQ ID NO: 246), (ii) GAKLSKKKKGYNVN (SEQ ID NO: 247), (iii) GGKQSKKKKGYNVN (SEQ ID NO: 248), (iv) GGKLAKKKKGYNVN (SEQ ID NO: 249), (v) GGKLSKKKKGYSGG (SEQ ID NO: 250), (vi) GGKLSKKKKGSGGS (SEQ ID NO: 251), (vii) GGKLSKKKKSGGSG (SEQ ID NO: 252), (viii) GGKLSKKKSGGSGG (SEQ ID NO: 253), (ix) GGKLSKKSGGSGGS (SEQ ID NO: 254), (x) GGKLSKSGGSGGSV (SEQ ID NO: 255), or (xi) GAKKSKKRFSFKKS (SEQ ID NO: 256).
